(19) United States
(12) Patent Application Publication
(10) Pub. No.: US 2024/0346804 A1
(43) Pub. Date: Oct. 17, 2024

(54) PIPELINES FOR TUMOR IMMUNOPHENOTYPING

(71) Applicant: Genentech, Inc., South San Francisco, CA (US)

(72) Inventors: Jeffrey Ryan EASTHAM, Pacifica, CA (US); Hartmut KOEPPEN, San Mateo, CA (US); Xiao LI, Foster City, CA (US); Darya Yuryevna ORLOVA, Los Altos, CA (US)

(21) Appl. No.: 18/612,987

(22) Filed: Mar. 21, 2024

Related U.S. Application Data
(60) Provisional application No. 63/492,072, filed on Mar. 24, 2023.

(51) Int. Cl. G06V 10/764; G06T 7/00; G06V 10/25

(52) U.S. Cl. CPC G06V 10/764; G06T 7/0012; G06V 10/25; G06T 2207/20081; G06T 2207/30096

(57) ABSTRACT

Described herein are systems, methods, and programming describing various pipelines for determining an immunophenotype of a tumor depicted by a digital pathology image based on immune cell density in the tumor epithelium and/or the tumor stroma and/or spatial information across all or part of the image. One or more machine learning models may be implemented by some or all of the pipelines. The pipelines may include a first pipeline using density thresholds for immunophenotyping, a second pipeline using immune cell density in tumor epithelium and tumor stroma for immunophenotyping, a third pipeline using spatial information of the digital pathology image for immunophenotyping, and a fourth pipeline that combines aspects of the second and third pipelines for immunophenotyping.

### CROSS-REFERENCE TO RELATED APPLICATIONS

This application claims priority to U.S. Provisional Patent Application No. 63/492,072, entitled “Pipelines for Tumor Immunophenotyping,” filed Mar. 24, 2023, the disclosure to which is incorporated herein by reference in its entirety.

### TECHNICAL FIELD

This application relates generally to processing digital pathology images to determine tumor immunophenotypes. In particular, this application includes four pipelines of varying granularity that can be used to determine a tumor immunophenotype based on a digital pathology image.

### BACKGROUND

The groundbreaking field of immuno-oncology (IO) has revolutionized the treatment of patients having a variety of illnesses. This has included observations of long-lasting responses to patients treated with therapeutics targeting checkpoint inhibitors (CIs), such as anti-PD-1/PD-L1 antibodies (e.g., atezolizumab, nivolumab, or docetaxel). Therapeutics targeting CI molecules and/or antibodies with immunomodulatory properties, such as bi-specific antibodies, have shown signs of success in the early stages of certain clinical evaluations. However, these early signs of success have been observed in only a small fraction of patients. This has fueled clinical research into identifying predictive biomarkers for IO therapies that maximize efficacy while also identifying patients unlikely to respond, but who would still be exposed to potentially life-threating side effects.

Evaluating the density, spatial distribution, cellular composition of immune cell infiltrates, and the like, of the tumor microenvironment (TME) has been shown to provide prognostic or predictive information. While manually categorizing tumors based on the spatial distribution of immune effector cells is predictive for CI targeted therapies in certain patient cohorts, these processes are laborious and subjective. Thus, what is needed is methodologies that can standardize the assessment of the density and pattern of immune effector cells to determine tumor immunophenotype.

### SUMMARY

Described herein are techniques for determining a tumor immunophenotype based on a digital pathology image. Immunophenotyping, which is the process of determining an immunophenotype of a biological sample (e.g., a tumor), can be predictive of the possible benefits provided by CI therapy. Traditionally, trained pathologists analyze digital pathology images of biological samples to determine the tumor immunophenotype. Depending on the tumor immunophenotype, different immunotherapies or no immunotherapies may be provided to the patient. Therefore, accurately determining the tumor immunophenotype can have a direct impact on patient survival.

Determining a tumor immunophenotype manually can be labor intensive and subjective, which can lead to mis-classifying patients and/or not identifying patients who could benefit from a particular immunotherapy. Described herein are pipelines that can standardize the tumor immunophenotyping process, which can improve tumor immunophenotype classification accuracy and thereby increasing the number of patients who may receive beneficial immunotherapies. The pipelines operate at varying levels of granularity, but each may be configured to determine a tumor immunophenotype from a digital pathology image (e.g., a whole slide image (WSI) of a biological sample). The various pipelines include a first pipeline based on immune cell density thresholds, a second pipeline based on an immune cell distribution within tumor epithelium and tumor stroma; a third pipeline based on immune cell density and spatial distribution information; and a fourth pipeline that concatenates aspects of the second and third pipeline. The pipelines have varying degrees of accuracy with the first pipeline being the least accurate and the fourth pipeline being the most accurate. As a tradeoff with the increase in accuracy, the fourth pipeline is the most more computationally intensive whereas the first pipeline is the least complex to implement. In some embodiments, one or more of the pipelines may implement a machine learning model to perform certain steps, such as, for example, identifying biological objects and/or performing classifications.

In some embodiments, a first pipeline may be configured to determine a tumor immunophenotype. An image depicting a tumor may be received. The image may be a digital pathology image of a biological sample stained with one or more stains (e.g., a panCK-CD8 dual stain) highlighting different biological objects. One or more regions of the image depicting tumor epithelium may be identified. For example, certain stains (e.g., panCK) may highlight tumor epithelium. An epithelium-immune cell density for the image may be calculated based on a number of immune cells detected within the regions depicting tumor epithelium. For example, certain stains (e.g., CD8) may highlight immune cells. A tumor immunophenotype of the image may be determined based on the epithelium-immune cell density and at least a first density threshold. The first density threshold may be used to classify images into one of a set of tumor immunophenotypes, for example, desert, excluded, and inflamed.

In some embodiments, a second pipeline may be configured to determine a tumor immunophenotype. An image depicting a tumor may be received. The image may be a digital pathology image of a biological sample stained with one or more stains (e.g., a panCK-CD8 dual stain) highlighting different biological objects. The image may be divided into a plurality of tiles. The tiles may be overlapping or may be non-overlapping. Epithelium tiles and stroma tiles may be identified from the plurality of tiles. For example, a tile may be classified as an epithelium tile if at least a threshold area of the tile includes epithelium cells, and a tile may be classified as a stroma tile if at least a threshold area of the tile includes stroma cells. Therefore, some embodiments include one or more tiles that are classified as being both epithelium tiles and stroma tiles. An epithelium-immune cell density may be calculated for each of the epithelium tiles based on a number of immune cells detected within each of the epithelium tiles and a stroma-immune cell density may be calculated for each of the stroma tiles based on a number of immune cells detected within each of the stroma tiles. The epithelium tiles may be binned into an epithelium set of bins based on the epithelium-immune cell density of each epithelium tile and the stroma tiles may be binned into a stroma set of bins based on the stroma-immune cell density of each stroma tile. A density-bin representation of the epithelium set of bins and the stroma set of bins may be generated where the density-bin representation includes a plurality of elements corresponding to each bin of the epithelium set of bins and each bin of the stroma set of bins. A tumor immunophenotype of the image may be determined based on the density-bin representation. For example, a classifier may be used to predict a tumor immunophenotype based on the density-bin representation.

In some embodiments, a third pipeline may be configured to determine a tumor immunophenotype. An image depicting a tumor may be received. The image may be a digital pathology image of a biological sample stained with one or more stains (e.g., a panCK-CD8 dual stain) highlighting different biological objects. The image may be divided into a plurality of tiles. The tiles may be overlapping or may be non-overlapping. One or more tumor-associated regions of the image may be identified. For example, epithelium tiles may be identified and stroma tiles may be identified. A local density measurement may be calculated for one or more biological object types for each of the epithelium tiles and each of the stroma tiles. The local density measurement may indicate a density of epithelial cells in the epithelium tiles, a density of stroma cells in the stroma tiles, an epithelium-immune cell density based on a number of immune cells within each of the epithelium tiles, and a stroma-immune cell density based on a number of immune cells within each of the stroma tiles. One or more spatial distribution metrics may be generated based on the local density measurement calculated for each of the tiles. A spatial distribution representation may be generated based on the spatial distribution metrics. A tumor immunophenotype of the image may be determined based on the spatial distribution representation. For example, a classifier may be used to predict a tumor immunophenotype based on the spatial distribution representation.

In some embodiments, a fourth pipeline may be configured to determine a tumor immunophenotype. An image depicting a tumor may be received. The image may be a digital pathology image of a biological sample stained with one or more stains (e.g., a panCK-CD8 dual stain) highlighting different biological objects. The image may be divided into a plurality of tiles. The tiles may be overlapping or may be non-overlapping. An epithelium-immune cell density may be calculated for each of the epithelium tiles based on a number of immune cells detected within each of the epithelium tiles and a stroma-immune cell density may be calculated based on a number of immune cells detected within each of the stroma tiles. A density-bin representation may be generated based on the epithelium-immune cell density of one or more epithelium tiles and the stroma-immune cell density of one or more stroma tiles. A local density measurement may be calculated for one or more biological object types for each of the epithelium tiles and each of the stroma tiles. The local density measurement may indicate a density of epithelial cells in the epithelium tiles, a density of stroma cells in the stroma tiles, an epithelium-immune cell density based on a number of immune cells within each of the epithelium tiles, and a stroma-immune cell density based on a number of immune cells within each of the stroma tiles. One or more spatial distribution metrics may be generated based on the local density measurement calculated for each of the tiles. A spatial distribution representation may be generated based on the spatial distribution metrics. The density-bin representation and the spatial distribution representation may be concatenated to obtain a concatenated representation and a tumor immunophenotype of the image may be determined based on the concatenated representation.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed can be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example system for generating and processing digital pathology images to determine tumor immunophenotypes, in accordance with various embodiments.

FIG. 2 illustrates an example digital pathology image generation subsystem, in accordance with various embodiments.

FIG. 3 illustrates an example first pipeline subsystem for implementing a first pipeline to determine a tumor immunophenotype, in accordance with various embodiments.

FIG. 4 illustrates an example of a ranking of images based on epithelium-immune cell density to determine density thresholds for immunophenotyping, in accordance with various embodiments.

FIG. 5 illustrates an example second pipeline subsystem for implementing a second pipeline to determine a tumor immunophenotype, in accordance with various embodiments.

FIG. 6 illustrates an example process for dividing an image of a biological sample into tiles, in accordance with various embodiments.

FIG. 7 illustrates an example of an immune cell density calculation, in accordance with various embodiments.

FIG. 8 illustrates an example density-bin representing of an immune cell distribution including an epithelium set of bins and a stroma set of bins, in accordance with various embodiments.

FIG. 9 illustrates an example process for determining a tumor immunophenotype based on a density-bin representation, in accordance with various embodiments.

FIG. 10 illustrates an example third pipeline subsystem implementing a third pipeline to determine a tumor immunophenotype, in accordance with various embodiments.

FIG. 11 illustrates an example process for determining a tumor immunophenotype based on a spatial distribution representation, in accordance with various embodiments.

FIG. 12 illustrates an example fourth pipeline subsystem implementing a fourth pipeline to determine a tumor immunophenotype, in accordance with various embodiments.

FIG. 13 illustrates an example of a concatenated representation generated based on a density-bin representation and a spatial distribution representation, in accordance with various embodiments.

FIG. 14 illustrates an example process for determining a tumor immunophenotype based on a concatenated representation, in accordance with various embodiments.

FIG. 15 illustrates an example process for training a machine learning model, in accordance with various embodiments.

FIG. 16 illustrates a flowchart of an example process describing an overall workflow for predictive analysis, in accordance with various embodiments.

FIG. 17 is an illustrative flowchart of an exemplary process for determining a tumor immunophenotype using a first pipeline, in accordance with various embodiments.

FIG. 18 is an illustrative flowchart of an exemplary process for determining density thresholds used for determining a tumor immunophenotype, in accordance with various embodiments.

FIG. 19 is an illustrative flowchart of an exemplary process for determining a tumor immunophenotype using a second pipeline, in accordance with various embodiments.

FIG. 20 is an illustrative flowchart of an exemplary process for training a classifier to determine a tumor immunophenotype, in accordance with various embodiments.

FIG. 21 is an illustrative flowchart of an exemplary process for determining a tumor immunophenotype using a third pipeline, in accordance with various embodiments.

FIG. 22 is an illustrative flowchart of an exemplary process for determining a tumor immunophenotype using a fourth pipeline, in accordance with various embodiments.

FIG. 23 is an illustrative flowchart of an exemplary process for training a classifier to determine a tumor immunophenotype, in accordance with various embodiments.

FIGS. 24A-24F illustrate example whole slide images or portions thereof depicting different tumor immunophenotypes, in accordance with various embodiments.

FIG. 25 illustrates an example digital pathology image including annotations indicating tumor lesions, in accordance with various embodiments.

FIGS. 26A-26B illustrate an example of epithelium tiles and stroma tiles determined from the tiles of the digital pathology image of FIG. 25, in accordance with various embodiments.

FIG. 27 illustrates an example plot of biological object density bins by tumor immunophenotype, in accordance with various embodiments.

FIG. 28A depicts an application of an areal analysis framework implemented by an example spatial distribution metric module, in accordance with various embodiments.

FIG. 28B illustrates example hotspot data indicating regions determined to be hotspots for detected depictions of biological objects, in accordance with various embodiments.

FIG. 29 illustrates a visualization of training and use of machine-learned models implemented by an example classification module, in accordance with various embodiments.

FIG. 30 illustrates example immune cell density criteria in regions of tumor epithelium and tumor stroma for tumor immunophenotypes of desert, excluded, and inflamed, in accordance with various embodiments.

FIGS. 31A-31B illustrate plots indicating a proportion of tumor area, in accordance with various embodiments.

FIGS. 32A-32B illustrate example a tile-based analysis of immune cell density within regions of tumor epithelium and tumor stroma, as well as a tumor immunophenotype output by a classifier based on the tile-based analysis, in accordance with various embodiments.

FIG. 33 illustrate an example workflow for a third pipeline and a fourth pipeline, in accordance with various embodiments.

FIGS. 34A-34D illustrate plots representing tumor immunophenotyping using an unsupervised approach, in accordance with various embodiments.

FIGS. 35A-35C illustrate plots representing a comparison of manually assigned tumor immunophenotypes with algorithmically determined tumor immunophenotypes, in accordance with various embodiments.

FIGS. 36A-36D illustrate example plots describing the association of tumor immunophenotype classification with immunotherapy outcome, in accordance with various embodiments.

FIGS. 37A-37B illustrate plots of the Bhattacharyya coefficient for tumor immunophenotype classes for the example second and third clinical trials calculated using the fourth pipeline, and the top features identified by the fourth pipeline, in accordance with various embodiments.

FIGS. 38A-38D illustrate plots of overall survival (OS) log-rank tests for tumor immunophenotypes classified manually and using a fourth pipeline and progression-free survival (PFS) log-rank tests for tumor immunophenotypes classified manually and using a fourth pipeline, respectively, in accordance with various embodiments.

FIGS. 39A-39D illustrate plots representing a Kaplan-Meier analysis of OS for two treatment arms of a non-small cell lung cancer (NSCLC) clinical trial and plots representing a Kaplan-Meier analysis of PFS for two treatment arms of a NSCLC clinical trial, respectively, in accordance with various embodiments.

FIGS. 40A-40B illustrate plots representing a Kaplan-Meier analysis of OS for two treatment arms of a triple negative breast cancer (TNBC) clinical trial, in accordance with various embodiments.

FIGS. 41A-41B illustrate plots representing a Kaplan-Meier analysis of PFS for two treatment arms of a TNBC clinical trial, in accordance with various embodiments.

FIG. 42 illustrates an example computer system used to implement some or all of the techniques described herein.

### DETAILED DESCRIPTION

Described herein are systems, methods, and programming describing various pipelines for determining an immunophenotype of a tumor depicted by a digital pathology image based on immune cell density in the tumor epithelium and/or the tumor stroma and/or spatial information across all or part of the image. One or more machine learning models may be implemented by some or all of the pipelines.

First, an overview of the system architecture associated with pipelines for determining a tumor immunophenotype is provided. Details related to the pipelines, each having different degrees of granularity, follow, including: a first pipeline using density thresholds for immunophenotyping, a second pipeline using immune cell density in tumor epithelium and tumor stroma for immunophenotyping, a third pipeline using spatial information of the digital pathology image for immunophenotyping, and a fourth pipeline that combines aspects of the second and third pipelines for immunophenotyping. Following the details relating to each of the pipelines are flowcharts describing each pipeline's operations and training as well as example results calculated using the pipelines.

Cancer immunology has revolutionized many different cancer treatments, including non-small cell lung cancer (NSCLC). Unfortunately, most of these lung cancer patients do not respond to immunotherapy. As a result, lung cancer remains the leading cancer killer in the United States, causing over 130,000 deaths each year.

Cancer immunotherapy refers to a technique that harnesses a patient's own immune system to eliminate and/or prevent further recurrences of tumors. Immunotherapies generally include stimulating/boosting the body's natural defenses to work harder and smart to attack cancer cells or therapeutics used to restore/improve the body's immune system. An example of the latter includes checkpoint inhibitors (CIs). CIs refer to drugs designed to restore the immune system's ability to recognize and attack cancer cells. The immune system is designed to differentiate between normal cells and abnormal cells, such as germs, bacteria, and/or cancer cells. This differentiation allows the immune system to effectively attack abnormal cells. This differentiation also prevents the immune system from attacking normal cells.

The immune system performs the aforementioned cell differentiation using a variety of techniques, one of them being “checkpoint” proteins. These checkpoint proteins can switch on/off the immune system's response. Unfortunately, cancer cells can also figure out how to use these checkpoints to avoid the immune system's attacks.

Medicines exist that can block checkpoint proteins made by some types of immune system cells, such as T cells and/or some cancer cells. These checkpoint proteins can assist in keeping immune responses from being too strong. At times, these checkpoint proteins can prevent T cells from killing cancer cells. Blocking checkpoint proteins can allow T cells to better kill cancer cells. Some example checkpoint proteins include PD-1/PD-L1 and CTLA-4/B7-1/B7-2.

Accordingly, some cancer therapies (e.g., monoclonal antibodies) use immune checkpoint inhibitors. These checkpoint inhibitors do not directly kill cancer cells. Instead, checkpoint inhibitors can enable the immune system to identify cancer cells and mount an attack against the cancer cells more accurately. Therefore, it is an important aspect of cancer research to develop inexpensive, simple, and reproducible biomarkers to identify patients that are most likely to respond to checkpoint inhibition, as well as those patients that may require additional treatments. For example, some patients may require additional therapies to prepare the immune system to attack and kill cancer cells.

One checkpoint inhibition pathway is the PD1-PD-L1 interaction, which prevents cytotoxic T cells from killing tumor cells. This pathway can be a primary cause of a patient's tumor growth. To identify whether the PD1-PD-L1 interaction is the main driver, an immunohistochemistry (IHC) assay may be performed. The IHC assay may stain for the PD-L1 or PD1 molecule. A positive result may indicate that the primary driver of tumor growth is the PD1-PD-L1 interaction. Patients whose IHC assay yields a positive result are expected to respond to therapies that disrupt the PD1-PD-L1 interaction, such as atezolizumab or nivolumab. Indeed, PD-L1 IHCs was found to be predictive for checkpoint inhibition therapy in many cancers, such as NSCLC.

Some patients, however, do not respond to therapies that disrupt the PD1-PD-L1 interaction even though those patients' IHC assays are PD-L1 positive. Further complicating matters is that some patients who respond to therapies that disrupt the PD1-PD-L1 interaction have IHC assays that are PD-L1 negative. As an example, a clinical trial where patients were treated with either atezolizumab or docetaxel found that PD-L1 was not predictive for an atezolizumab response at a statistically significant level.

There are numerous theories as to why some patients do not respond to checkpoint inhibitors. One example relates to the cancer cells themselves, which can have low immunogenicity (e.g., the ability to produce an immune response to a pathogen). Cancer cells can also increase regulatory T cell (Tregs) production, which function to suppress immune response. Cancer cells can also rely on inhibitory signals other than PD1-PD-L1.

Another explanation as to why some patients do not respond to checkpoint inhibition therapy may be that there are too few immune cells, which actively kill tumor cells (e.g., CD8 cytotoxic T cells) near the tumor. Immune cells may also be unable to penetrate the tumor epithelium, instead remaining in the tumor stroma.

These immune cells can positively impact mortality rates for most cancers, such as NSCLC. A patient that does not respond to immunotherapy may not have enough CD8+ T cells in the vicinity of a tumor lesion. Alternatively, or additionally, unknown impediments can prevent T cells from reaching target tumor cells in a stromal component of a tumor lesion

Therefore, accurately predicting/identifying whether a patient will respond to one or more therapies can improve that patient's outcome. Being able to predict patient response can also help avoid overtreatment and endangering a patient's health with a therapy that may not be effective for them. Described herein are technical solutions to the aforementioned technical problems, including developing pipelines configured to determine a tumor immunophenotype based on a digital pathology image analysis.

Assays that have received regulatory approval for identifying patients most likely to respond to CIs include immunohistochemistry (IHC) for programmed death-ligand 1 (PD-L1), tumor mutational burden (TMB), and microsatellite instability (MSI). Of these, the PD-L1 IHC is the most commonly used for predicting/identifying patients who will respond to CI therapies. The other assays, TMB and MSI, have only limited diagnostic use as they apply only to a small patient population. Immunohistochemistry (IHC) assays for the PD-1/PD-L1 molecule have achieved companion diagnostic status and are commonly used to identify patients likely to respond to CI therapeutics targeting the PD-1/PD-L1 axis, including CI therapeutics targeting non-small cell lung cancer.

The success of IO therapeutics relies on generating/facilitating anti-tumor immunity in the tumor microenvironment (TME). The TME may represent the spatial structure of tissue components and their microenvironment interactions. There are numerous known interactions between immune cells and an established tumor that, in addition to the complexity and plasticity of the TME, pose a challenge to identify a single parameter with sufficient predictive power.

Density and phenotype of immune cells in the TME have been used to identify patients likely to have a better clinical outcome and/or response to a particular immunotherapy. Some studies have developed an “immunoscore” to identify patients likely to have improved immunotherapy response independent of prognostic factors (e.g., age, sex, tumor and lymph node status). The immunoscore may be computed based on the spatial distribution and density of Cluster of Differentiation 3-positive (CD3+) and CD8+ T lymphocytes. The immunoscore was developed for primary colorectal cancer.

More recent studies have shown concordance of this approach when applied to large patient cohorts across multiple clinical trials and combined with image analysis tools. A digital pathology-based machine learning model has performed superior to pathologist-based manual inspection in a small dataset. A scoring system for tumor-infiltrating lymphocytes (TILs) has been developed for ductal breast cancer and other carcinomas; using hematoxylin-eosin (H&E) stained sections to estimate a stromal density of all mononuclear cells, including plasma cells but excludes granulocytes as well as intra-epithelial immune cells. High density of stromal TILs tends to correlate with better clinical outcome but is also associated with inter-observer variability.

More recently, different approaches to assess TILs in solid tumors using digital quantification analyzed. Heterogeneity of these approaches with respect to tumor indication, methodology to identify TILs, and readouts, makes it challenging to compare results as well as emphasize standardization and validation in large and well-annotated clinical cohorts.

Recent studies have described approaches using image analysis with or without machine-learning to delineate the spatial distribution of immune effector cells—stromal vs intra-epithelial—correlating the pattern with gene expression signatures and clinical outcome for CI-treated patients.

Described herein are techniques harnessing image analysis techniques, particularly digital pathology-related image analysis, to categorize tumors depicted within digital pathology images into one of a set of tumor immunophenotypes. The set of tumor immunophenotypes, for example, includes the tumor immunophenotypes: “desert,” “excluded,” and “inflamed.” The techniques described herein may determine the tumor immunophenotype based on an immune cell density and/or spatial distribution of immune cells analyzed using digital pathology image analysis pipelines. The categorization of tumors, using digital pathology image analysis pipelines for predicting tumor immunophenotypes, as described below, provides prognostic and/or predictive information for treating patients with particular immunotherapies (e.g., CIs).

Digital pathology image analysis includes processing individual images to generate image-level results. For example, a result may be a binary result corresponding to an assessment as to whether the image includes a particular type of object or a categorization of the image as including one or more of a set of types of objects. As another example, a result may include an image-level count of a number of objects of a particular type detected within an image or a density of the distribution of the objects of the particular type. In the context of digital pathology images, a result can include a count of cells of a particular type or a display a particular indication detected within an image of a sample, a ratio of a count of one type of cell relative to a count of another type of cell across the entire image, and/or a density of a particular type of cell in particular regions of the image. This image-level approach can be convenient, as it can facilitate metadata storage and can be easily understood in terms of how the result was generated.

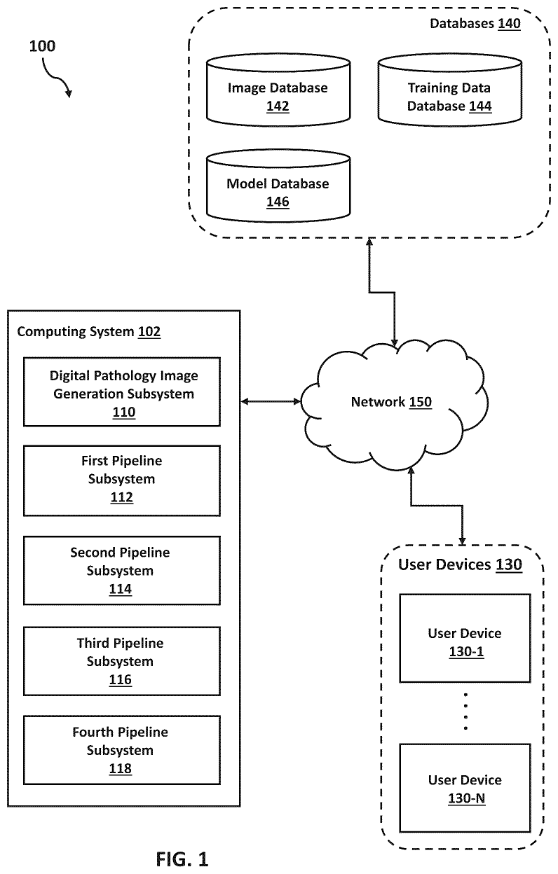

FIG. 1 illustrates an example system 100 for determining a tumor immunophenotype, in accordance with various embodiments. System 100 may include a computing system 102, user devices 130-1 to 130-N (also referred to collectively as “user devices 130” and individually as “user device 130”), databases 140 (e.g., image database 142, training data database 144, model database 146), or other components. In some embodiments, components of system 100 may communicate with one another using network 150, such as the Internet.

User devices 130 may communicate with one or more components of system 100 via network 150 and/or via a direct connection. User devices 130 may be a computing device configured to interface with various components of system 100 to control one or more tasks, cause one or more actions to be performed, or effectuate other operations. For example, user device 130 may be configured to receive and display an image of a scanned biological sample. Example computing devices that user devices 130 may correspond to include, but are not limited to, which is not to imply that other listings are limiting, desktop computers, servers, mobile computers, smart devices, wearable devices, cloud computing platforms, or other client devices. In some embodiments, each user device 130 may include one or more processors, memory, communications components, display components, audio capture/output devices, image capture components, or other components, or combinations thereof. Each user device 130 may include any type of wearable device, mobile terminal, fixed terminal, or other device.

It should be noted that while one or more operations are described herein as being performed by particular components of computing system 102, those operations may, in some embodiments, be performed by other components of computing system 102 or other components of system 100. As an example, while one or more operations are described herein as being performed by components of computing system 102, those operations may, in some embodiments, be performed by aspects of user devices 130. It should also be noted that, although some embodiments are described herein with respect to machine learning models, other prediction models (e.g., statistical models or other analytics models) may be used in lieu of or in addition to machine learning models (e.g., a statistical model replacing a machine-learning model and a non-statistical model replacing a non-machine-learning model in one or more embodiments). Still further, although a single instance of computing system 102 is depicted within system 100, additional instances of computing system 102 may be included (e.g., computing system 102 may comprise a distributed computing system).

Computing system 102 may include a digital pathology image generation subsystem 110, a first pipeline subsystem 112, a second pipeline subsystem 114, a third pipeline subsystem 116, a fourth pipeline subsystem 118, or other components. Each of digital pathology image generation subsystem 110, first pipeline subsystem 112, second pipeline subsystem 114, third pipeline subsystem 116, and fourth pipeline subsystem 118 may be configured to communicate with one another, one or more other devices, systems, and/or servers, using network 150 (e.g., the Internet, an Intranet). System 100 may also include one or more databases 140 (e.g., image database 142, training data database 144, model database 146) used to store data for training machine learning models, storing machine learning models, or storing other data used by one or more components of system 100. This disclosure anticipates the use of one or more of each type of system and component thereof without necessarily deviating from the teachings of this disclosure.

Although not illustrated, other intermediary devices (e.g., data stores of a server connected to computing system 102) can also be used. The components of system 100 of FIG. 1 can be used in a variety of contexts where scanning and evaluating digital pathology images, such as whole slide images, are essential components of the work. As an example, system 100 can be associated with a clinical environment where a user is evaluating the sample for possible diagnostic purposes. The user can review the image using user device 130 prior to providing the image to computing system 102. The user can provide additional information to computing system 102 that can be used to guide or direct the analysis of the image. For example, the user can provide a prospective diagnosis or preliminary assessment of features within the scan. The user can also provide additional context, such as the type of tissue being reviewed. As another example, system 100 can be associated with a laboratory environment where tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to computing system 102.

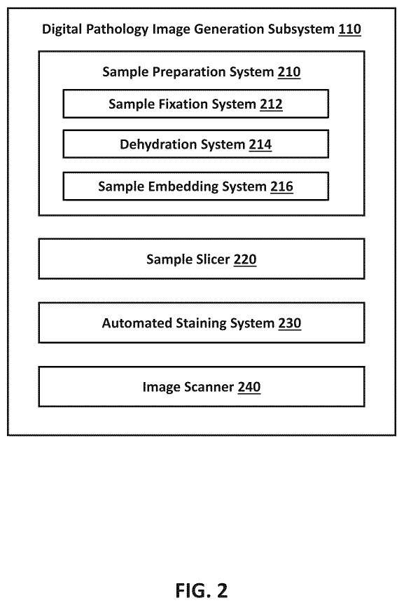

In some embodiments, digital pathology image generation subsystem 110 may be configured to generate one or more whole slide images or other related digital pathology images, corresponding to a particular sample. For example, an image generated by digital pathology image generation subsystem 110 may include a stained section of a biopsy sample. As another example, an image generated by digital pathology image generation subsystem 110 may include a slide image (e.g., a blood film) of a liquid sample. As yet another example, an image generated by digital pathology image generation subsystem 110 can include fluorescence microscopy such as a slide image depicting fluorescence in situ hybridization (FISH) after a fluorescent probe has been bound to a target DNA or RNA sequence. Additional details of digital pathology image generation subsystem 110 are described below with respect to FIG. 2.

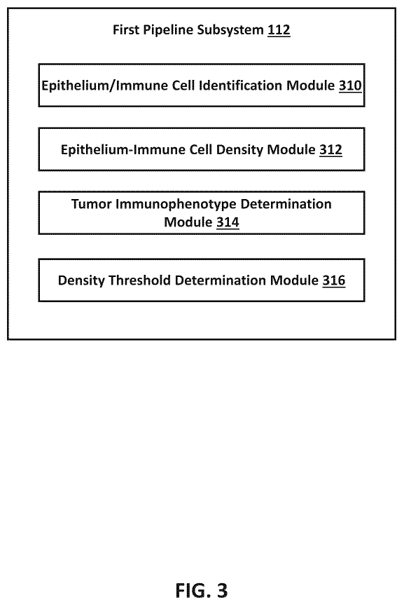

First pipeline subsystem 112 may be configured to implement a first pipeline for determining a tumor immunophenotype of a tumor depicted by a digital pathology image. In some embodiments, first pipeline subsystem 112 may identify regions of the digital pathology image depicting tumor epithelium. For each of these regions, first pipeline subsystem 112 may calculate an epithelium-immune cell density based on a number of immune cells detected therein. First pipeline subsystem 112 may be configured to determine a tumor immunophenotype of the image based on the epithelium-immune cell density and one or more density thresholds. The density thresholds may include at least a first density threshold. For example, an epithelium-immune cell density greater than or equal to the first density threshold may indicate that the digital pathology image represents a tumor of a first tumor immunophenotype (e.g., inflamed), while an epithelium-immune cell density less than the first density threshold may indicate that the digital pathology image represents a tumor of a second tumor immunophenotype (e.g., non-inflamed). In some embodiments, the density thresholds may be determined based on a ranking of epithelium-immune cell densities calculated for digital pathology images of patients participating in a clinical trial. Additional details regarding first pipeline subsystem 112 are described below with respect to FIG. 3.

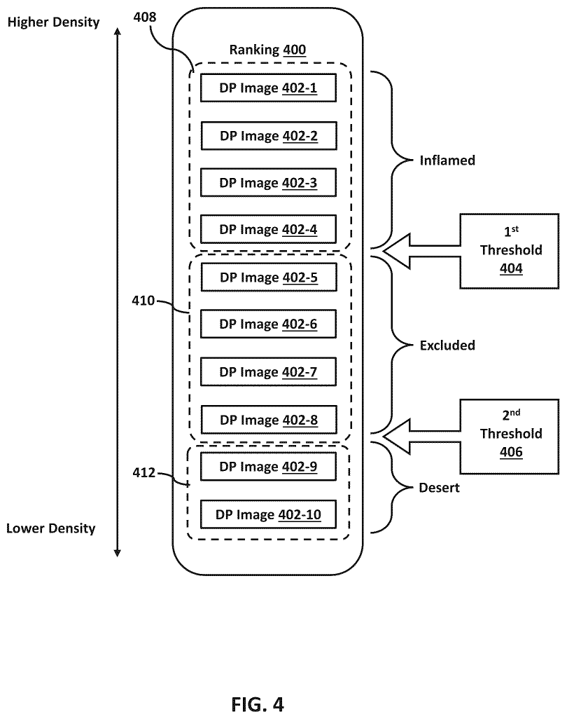

Second pipeline subsystem 114 may be configured to implement a second pipeline for determining a tumor immunophenotype of a tumor depicted by a digital pathology image. In some embodiments, second pipeline subsystem 114 may be configured to receive a digital pathology image and divide the digital pathology image into image tiles. For each of the tiles, second pipeline subsystem 114 may determine whether the tile shows epithelium, stroma, or both an epithelium and stroma. For each determination, second pipeline subsystem 114 applies a respective label to each tile, so that each tile is labeled or categorized as an epithelium tile, a stroma tile, or both an epithelium tile and a stroma tile. Second pipeline subsystem 114 may calculate an epithelium-immune cell density for each of the epithelium tiles based on a number of immune cells detected within each epithelium tile and may also calculate a stroma-immune cell density for each of the stroma tiles based on a number of immune cells detected within each stroma tile. In some embodiments, second pipeline subsystem 114 may be configured to bin the epithelium tiles into an epithelium set of bins based on the epithelium-immune cell density of each epithelium tile and bin the stroma tiles into a stroma set of bins based on the stroma-immune cell density of each stroma tile. Second pipeline subsystem 114 may be configured to generate a density-bin representation of the epithelium set of bins and the stroma set of bins. The density-bin representation may include elements corresponding to each bin of the epithelium set of bins and each bin of the stroma set of bins. In some embodiments, second pipeline subsystem 114 may be configured to determine a tumor immunophenotype of the digital pathology image based on the density-bin representation. For example, a classifier trained to predict tumor immunophenotype may be used to classify the digital pathology image into one of a set of tumor immunophenotypes based on the density-bin representation. Additional details related to second pipeline subsystem 114 are described below with respect to FIG. 4.

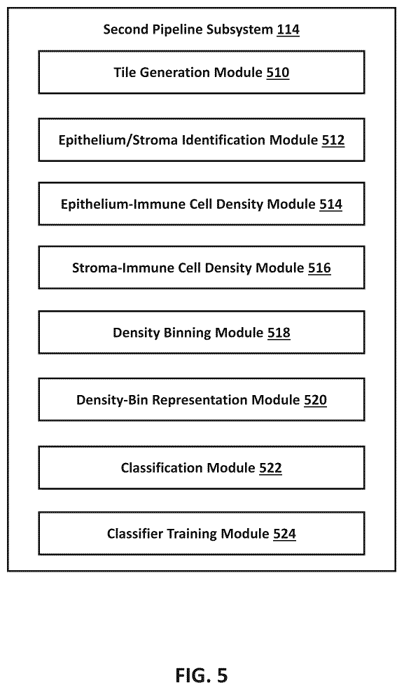

Third pipeline subsystem 116 may be configured to implement a third pipeline for determining a tumor immunophenotype of tumor depicted by a digital pathology image. In some embodiments, third pipeline subsystem 116 may be configured to receive a digital pathology image and divide the digital pathology image into tiles. For each of the tiles, third pipeline subsystem 116 may calculate local density measurements for different biological objects that may be depicted by each tile. For example, third pipeline subsystem 116 may calculate an epithelial cell density of a tile, a stromal cell density of a tile, an immune cell density within tumor epithelium (e.g., an epithelium-immune cell density), and/or an immune cell density within tumor stroma (e.g., a stroma-immune cell density). Third pipeline subsystem 116 may be configured to generate one or more spatial-distribution metrics describing the digital pathology image based on the local density measurements. Some example spatial-distribution metrics include a Jaccard index, a Sorensen index, a Bhattacharyya coefficient, a Moran's index, a Geary's contiguity ratio, a Morisita-Horn index, or a metric defined based on a hotspot/cold spot analysis. In some embodiments, third pipeline subsystem 116 may determine a tumor immunophenotype based on the spatial distribution metrics. For example, a spatial density representation (e.g., a feature vector) may be projected into a multidimensional feature space and a tumor immunophenotype may be assigned to the digital pathology image based on a distance in the multidimensional feature space between the projected spatial density representation and a cluster of representations associated with the tumor immunophenotype being less than a threshold distance. In some embodiments, a classifier may be trained to predict the tumor immunophenotype based on the spatial density representation. Additional details relating to third pipeline subsystem 116 are described with respect to FIG. 5.

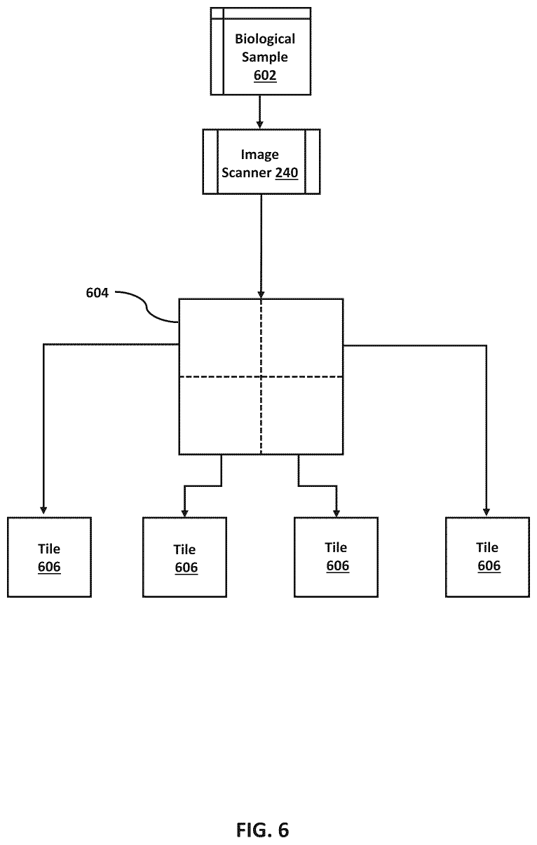

Fourth pipeline subsystem 118 may be configured to implement a fourth pipeline for determining a tumor immunophenotype of a digital pathology image. In some embodiments, fourth pipeline subsystem 118 may be configured to receive a digital pathology image and divide the digital pathology image into image tiles. Each of the tiles may depict an epithelium, stroma, or both an epithelium and stroma. For each of the tiles, fourth pipeline subsystem 118 may thus determine whether the tile is an epithelium tile, a stroma tile, or both an epithelium tile and a stroma tile. The fourth pipeline implemented by fourth pipeline subsystem 118 may include aspects from the second pipeline and the third pipeline. For example, fourth pipeline subsystem 118 may be configured to generate a density-bin representation for an image using the techniques of the second pipeline, generate a spatial distribution representation for the image using the techniques of the third pipeline, and concatenate the density-bin representation and the spatial distribution representation to obtain a concatenated representation of the image. In some embodiments, fourth pipeline subsystem 118 may be configured to determine a tumor immunophenotype of the digital pathology image based on the concatenated representation. For example, the concatenated representation may be input to a classifier trained to predict a tumor immunophenotype. Additional details relating to fourth pipeline subsystem 118 are described with respect to FIG. 6.

It should be noted that the digital pathology image generation subsystem 110 is not limited to pathology images and can be generally applied to any form of histology images. Further, it should be noted that the image tiles of the second pipeline subsystem 114, the third pipeline subsystem 116, and/or the fourth pipeline subsystem 118 can be extracted from histology images and used as inputs and/or training data for a multiple instance learning model. Multiple instance learning is a form of weakly supervised learning that leverages weakly or ambiguously annotated data to classify inputs. Multiple instance learning can involve the grouping of “instances” (e.g., inputs) into “bags” comprising multiple instances, as well as the machine-learning-driven identification of labels corresponding to the “bags.” A bag may be labeled as “positive” if at least one instance in the bag is positive, and it may be labeled as “negative” if all instances in the bag are negative. For example, in the context of classifying which tumor immunophenotype is associated with a histology image, the histology image (e.g., bag) may be divided into image tiles (e.g., instances) that are analyzed for the presence of certain histological features. If one image tile (e.g., instance) is identified as containing, for example, a breast cancer feature, the entire histology image (e.g., bag) may be labeled as “positive” for being associated with breast cancer. If no image tiles are identified as containing a breast cancer feature, the entire histology image may be labeled as “negative” for being associated with breast cancer. The goal of multiple instance learning is to predict the labels associated with each bag based on their contents (e.g., the instances). In some embodiments, one or more of the second pipeline subsystem 114, the third pipeline subsystem 116, and/or the fourth pipeline subsystem 118 can be configured to receive a histology image and divide the histology image into image tiles. Each image tile can depict a distinct structure within the imaged tissue and can serve as an “instance.” The histology image, treated as a collective entity, can serve as the “bag” encompassing multiple instances, and can be labeled as “positive” or “negative” for association with a tumor immunophenotype. In some embodiments, the tumor immunophenotype of the histology image can be automatically determined using a machine learning model trained via multiple instance learning.

In some embodiments, an attention score mechanism can be used to identify/emphasize which instances within a bag contribute significantly to making a positive prediction and/or label. This attention-score-based process can aid in emphasizing the most relevant and discriminative regions within the histology image, contributing to the model and/or algorithm's ability to discern intricate patterns associated with the tumor immunophenotype. For example, attention scores can be derived from the features of each instance (e.g., by assessing their similarity in relation to features of other instances within the same bag). Instances that are more similar to other instances may be less strongly associated with a positive label, thereby having a lower attention score. For example, in a histology image containing only healthy cells, the healthy cells may all resemble one another, and there may be no reason to pay greater attention to one image tile over another. Conversely, instances that are more distinct may be more strongly associated with a positive label, thereby having a higher attention score. For example, in a histology image containing both healthy cells and tumor cells, the tumor cells may stand out from the healthy cells, and the multiple instance machine-learning model may pay greater attention to the image tiles containing the standout tumor cells when labeling the bag as “positive.” An instance-based model and/or instance-based algorithm can be trained to predict the tumor immunophenotype based on the attention scores. For example, a histology image containing both healthy cells and tumor cells may be labeled as “positive” based on the high attention scores associated with image tiles depicting tumor cells within the histology image, and the overall histology image can be labeled as containing a specific tumor type as a result.

FIG. 2 illustrates an example digital pathology image generation subsystem 110, in accordance with various embodiments. Digital pathology image generation subsystem 110 may include one or more systems, modules, devices, or other components. As an example, with reference to FIG. 2, digital pathology image generation subsystem 110 may include a sample preparation system 210, a sample slicer 220, an automated staining system 230, an image scanner 240, or other components.

Sample preparation system 210 may be configured to prepare a biological sample for digital pathology analyses. Some example types of samples include biopsies, solid samples, samples including tissue, or other biological samples. Biological samples may be obtained for patients participating in various clinical trials. For example, biological samples may be obtained for participants of a clinical trial including patients with advanced NSCLC who had progressed on platinum-based chemotherapy and who were 1:1 randomized to receive either a first immunotherapy (e.g., atezolizumab) or a second immunotherapy (e.g., docetaxel). Biological samples may also be obtained for participants of a randomized phase 3 clinical trial including patients with advanced NSCLC receiving the same or similar immunotherapies described above. Biological samples may further be obtained for participants of a phase 3 clinical trial of treatment-naive patients with metastatic TNBC who were randomized to receive a first immunotherapy (e.g., atezolizumab plus nab-paclitaxel) or a second immunotherapy (e.g., placebo plus nab-paclitaxel). In some embodiments, the biological samples obtained included tissue sections including at least 100 viable invasive tumor cells with associated stroma for immunophenotype evaluation. In the rare case where more than one biological sample was obtained for a patient, the sample with the largest tumor area was selected for evaluation. The study was conducted according to the Declaration of Helsinki and all patients had provided written consent.

Sample preparation system 210 may be configured to fix and/or embed a sample. In some embodiments, sample preparation system 210 may facilitate infiltrating a sample with a fixating agent (e.g., liquid fixing agent, such as a formaldehyde solution) and/or embedding substance (e.g., a histological wax). Sample preparation system 210 may include one or more systems, subsystems, modules, or other components, such as a sample fixation system 212, a dehydration system 214, a sample embedding system 216, or other subsystems. Sample fixation system 212 may be configured to fix a biological sample. For example, sample fixation system 212 may expose a sample to a fixating agent for at least a threshold amount of time (e.g., at least 3 hours, at least 6 hours, at least 13 hours, etc.). Dehydration system 214 may be configured to dehydrate the biological sample. For example, dehydration system 214 may expose the fixed sample and/or a portion of the fixed sample to one or more ethanol solutions. In some embodiments, dehydration system 214 may also be configured to clear the dehydrated sample using a clearing intermediate agent. An example clearing intermediate agent may include ethanol and a histological wax. Sample embedding system 216 may be configured to infiltrate the biological sample. In some embodiments, sample embedding system 216 may infiltrate the biological sample using a heated histological wave (e.g., liquid). In some embodiments, sample embedding system 216 may perform the infiltration process one or more times for corresponding predefined time periods. The histological wax can include a paraffin wax and potentially one or more resins (e.g., styrene or polyethylene). Sample preparation system 210 may further be configured to cool the biological sample and wax or otherwise allow the biological sample and wax to be cooled. After cooling, Sample preparation system 210 may block out the wax-infiltrated biological sample.

Sample slicer 220 may be configured to receive the fixed and embedded sample and produce a set of sections. Sample slicer 220 can expose the fixed and embedded sample to cool or cold temperatures. Sample slicer 220 can then cut the chilled sample (or a trimmed version thereof) to produce a set of sections. For example, each section may have a thickness that is less than 100 μm, less than 50 μm, less than 10 μm, less than 5 μm, or other dimensions. As another example, each section may have a thickness that is greater than 0.1 μm, greater than 1 μm, greater than 2 μm, greater than 4 μm, or other dimensions. The sections may have the same or similar thickness as the other sections. For example, a thickness of each section may be within a threshold tolerance (e.g., less than 1 μm, less than 0.1 μm, less than 0.01 μm, or other values). The cutting of the chilled sample can be performed in a warm water bath (e.g., at a temperature of at least 30° C., at least 35° C., at least 40° C., or other temperatures).

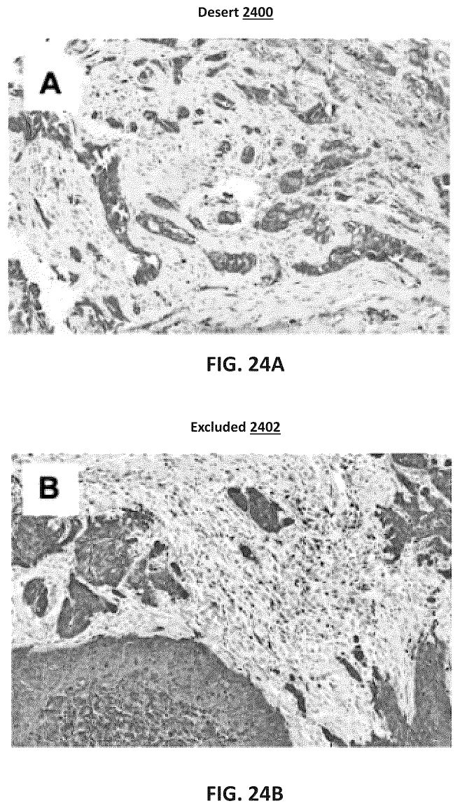

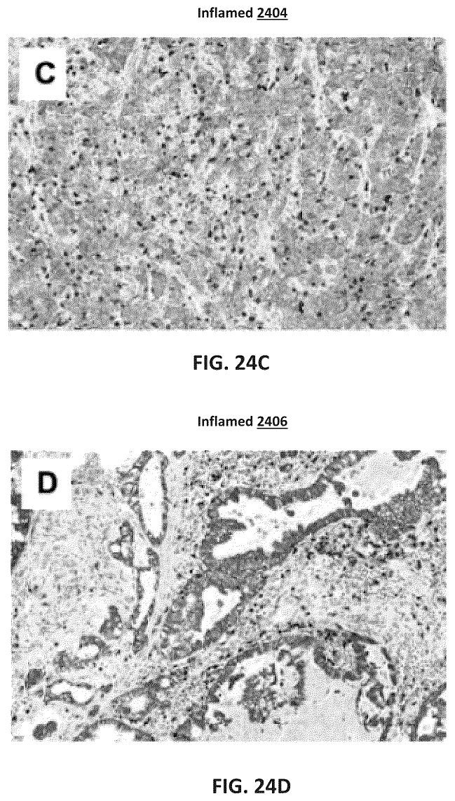

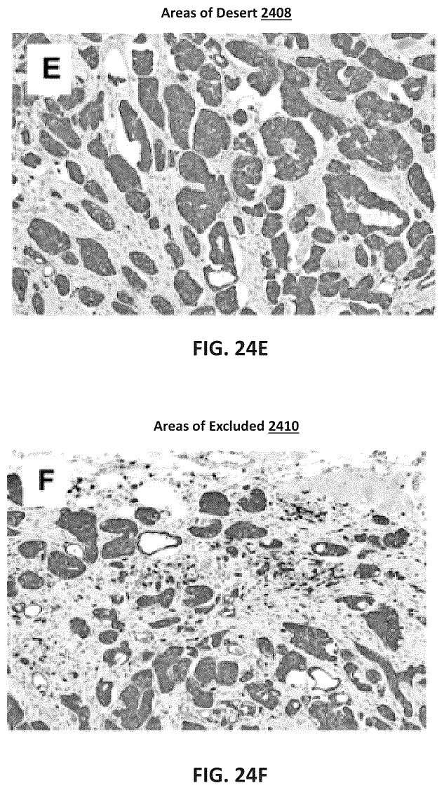

Automated staining system 230 may be configured to stain one or more of the sample sections. Automated staining system 230 may expose each section to one or more staining agents. Example staining agents may include Hematoxylin, Eosin, pan-cytokeratin (panCK), and a cluster of differentiation 8 (CD8) stain. In one example, a panCK-CD8 dual-stain may be used as the staining agent. As an example, with reference to FIGS. 24A-24E, digital pathology images 2400-2410 represent whole slide images, or a portion of whole slide images, depicting biological samples that have been stained using a panCK-CD8 dual stain. In digital pathology images 2400-2410, certain biological structures (e.g., immune cells) may be represented in a first color, e.g., by the “brown” spots highlighted from the CD8 staining agent, while other biological structures (e.g., tumor epithelium) may be represented in a second color, e.g., by the “purple” spots highlighted from the panCK staining agent. Each section can be exposed to a predefined volume of a staining agent for a predefined period of time. In some embodiments, automated staining system 230 may be configured to expose a single section concurrently or sequentially to multiple staining agents.

Each of one or more stained sections can be presented to image scanner 240, which can capture a digital image of that section. Image scanner 240 can include a microscope camera. Image scanner 240 may be configured to capture a digital image at one or more levels of magnification (e.g., using a 10× objective, a 20× objective, a 40× objective, or other magnification levels). Manipulation of the image can be used to capture a selected portion of the sample at the desired range of magnifications. In some embodiments, annotations to exclude areas of assay, scanning artifact, and/or large areas of necrosis may be performed (manually and/or with the assistance of machine learning models). Image scanner 240 can further capture annotations and/or morphometrics identified by a human operator. In some embodiments, a section may be returned to automated staining system 230 after one or more images are captured, such that the section can be washed, exposed to one or more other stains, and imaged again. In some embodiments, when multiple stains are used, these stains can be selected to have different color profiles. For example, a first region of an image corresponding to a first section that absorbed a large amount of a first staining agent can be distinguished from a second region of the image (or a different image) corresponding to a second section that absorbed a large amount of a second staining agent.

It will be appreciated that one or more components of digital pathology image generation subsystem 110 can, in some instances, operate in connection with human operators. For example, human operators can move the sample across various components of digital pathology image generation subsystem 110 and/or initiate or terminate operations of one or more subsystems, systems, or components of digital pathology image generation subsystem 110. As another example, part or all of one or more components of the digital pathology image generation system (e.g., one or more subsystems of sample preparation system 210) can be partly or entirely replaced with actions of a human operator.

Further, it will be appreciated that, while various described and depicted functions and components of digital pathology image generation subsystem 110 pertain to processing of a solid and/or biopsy sample, other embodiments can relate to a liquid sample (e.g., a blood sample). For example, digital pathology image generation subsystem 110 can receive a liquid-sample (e.g., blood or urine) slide that includes a base slide, smeared liquid sample, and a cover. In some embodiments, image scanner 240 may capture an image of the sample slide. Furthermore, some embodiments of digital pathology image generation subsystem 110 include capturing images of samples using advancing imaging techniques. For example, after a fluorescent probe has been introduced to a sample and allowed to bind to a target sequence, appropriate imaging techniques can be used to capture images of the sample for further analysis.

A given sample can be associated with one or more users (e.g., one or more physicians, laboratory technicians and/or medical providers) during processing and imaging. An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or more user devices 130 to submit one or more requests (e.g., that identify a subject) that a sample be processed by digital pathology image generation subsystem 110 and that a resulting image be processed by first pipeline subsystem 112, second pipeline subsystem 114, third pipeline subsystem 116, fourth pipeline subsystem 118, or other components of system 100, or combinations thereof.

In some embodiments, the biological samples that will be prepared for imaging by image scanner 240 may include images collected from one or more clinical trials. In one example, the clinical trials may include an NSCLC clinical trial, which may include biological samples of adenocarcinomas and squamous cell carcinoma. In some embodiments, the NSCLC clinical trials may include patients (e.g., 100 or more patients, 200 or more patients, 300 or more patients, and the like) randomized to receive either a first immunotherapy (e.g., atezolizumab) or a second immunotherapy (e.g., docetaxel). In another example, the clinical trials may include a TNBC clinical trial. In some embodiments, the TNBC clinical trials may include patients (e.g., 500 or more patients, 1,000 or more patients, 2,000 or more patients, and the like) randomized to receive either a first immunotherapy (e.g., atezolizumab) or a second immunotherapy (e.g., placebo plus nab-paclitaxel).

In some embodiments, constraints may be applied to ensure that the analyzed biological samples are statistically representative. For example, tissue sections may be used if those tissue sections depict at least a threshold number of viable invasive tumor cells with associated stroma. The threshold number of viable invasive tumor cells may be configurable. For example, the threshold number of viable invasive tumor cells may be 50 or more tumor cells, 100 or more tumor cells, 200 or more tumor cells, and the like). In some embodiments, a single tissue sample may be obtained for each patient of the clinical trial (or a subset of the patients from the clinical trial).

Digital pathology image generation subsystem 110 may be configured to transmit an image produced by image scanner 240 to user device 130. User device 130 may communicate with first pipeline subsystem 112, second pipeline subsystem 114, third pipeline subsystem 116, fourth pipeline subsystem 118, or other components of computing system 102 to initiate automated processing and analysis of the digital pathology image. In some embodiments, digital pathology image generation subsystem 110 may be configured to provide a digital pathology image produced by image scanner 240 to first pipeline subsystem 112, second pipeline subsystem 114, third pipeline subsystem 116, and/or fourth pipeline subsystem 118. For example, an image may be directed from image scanner 240 to first pipeline subsystem 112, second pipeline subsystem 114, third pipeline subsystem 116, and/or fourth pipeline subsystem 118 by a user of user device 130.

In some embodiments, a trained pathologist may determine a tumor immunophenotype of a tumor depicted by a digital pathology image. In this approach, a tumor area may be defined within the digital pathology image as regions including viable tumor cells (e.g., formed from epithelial cells) with associated tumor stroma (e.g., formed from stroma cells). Areas of necrosis or intraluminal aggregates of immune cells (e.g., CD8+ T cells) were excluded. While immune cells were detected in most, if not all, cases, the level of immune cells detected and the degree with which the immune cells infiltrate tumor stroma and tumor epithelium may be used for immunophenotype classification.

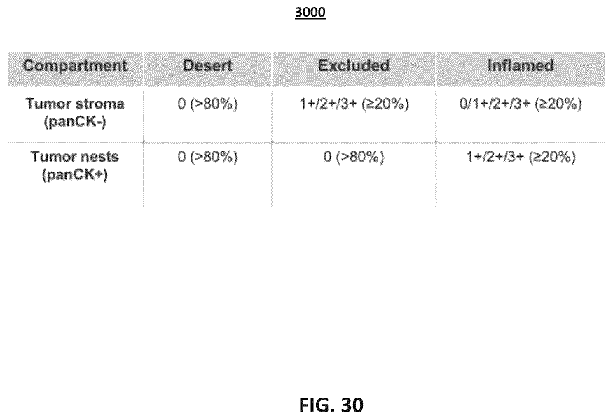

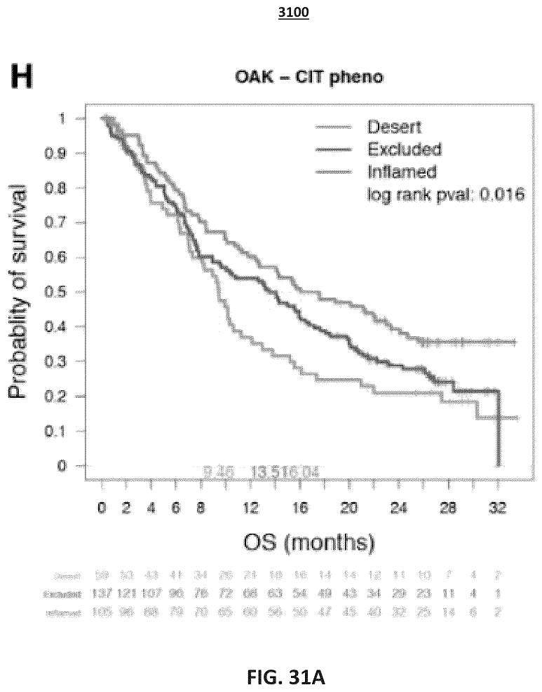

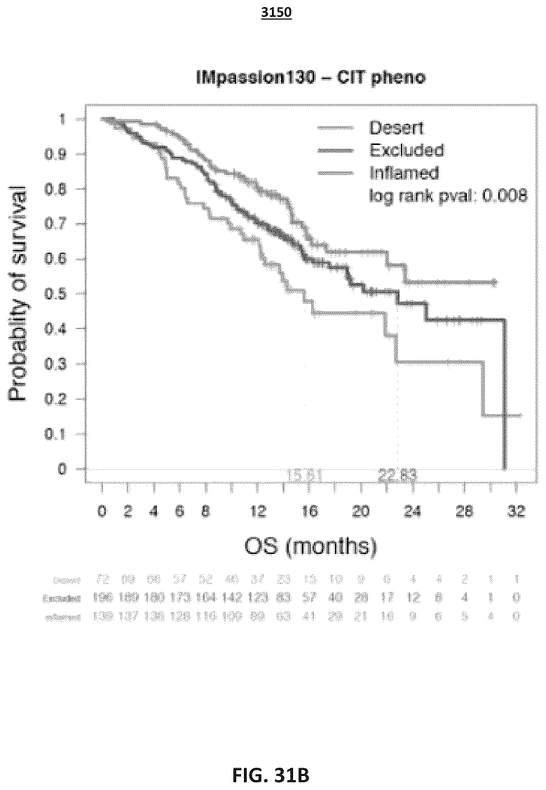

Tumors with a sparse immune cell infiltration independent of spatial distribution were classified as the tumor immunophenotype “desert,” as seen image 2400 of FIG. 24A. In these examples, the density of immune cells was too sparse to enable identification of any pattern. Tumors with a distribution of immune cells limited to regions of tumor stroma (e.g., CK− regions) were classified as the tumor immunophenotype “excluded,” as seen in image 2402 of FIG. 24B. Tumors showing co-localization of immune cells and tumor epithelium (e.g., CD8+ T cells with CK+ tumor cells) were classified as the tumor immunophenotype “inflamed,” as seen in image 2404 of FIG. 24C and image 2406 of FIG. 24D. In FIG. 24C and FIG. 24D, immune cells infiltrated regions of tumor epithelium were either seen as a diffuse infiltrate with or without involvement of stromal cells or with a predominantly stromal distribution of immune cells with “spill-over” into regions of tumor epithelium. These two patterns were not distinguished separately and both were included as instances of the tumor immunophenotype inflamed. Intra-tumoral heterogeneity of density and the pattern of the infiltration are shown by image 2408 of FIG. 24E and image 2410 of FIG. 24F. In these examples, a 20% cut-off was applied. Patterns occupying less than 20% of the tumor area were not considered for categorization. Cases showing an inflamed phenotype in >20% of the tumor area were labeled “inflamed”, independent of the pattern(s) observed in the remaining areas. Implementation of the cutoff was based on a subjective estimate by the inspecting pathologist, as seen Table 3000 of FIG. 30. Furthermore, as seen in FIGS. 31A-31B, OS plots 3100 and 3150 respectively describe a likelihood of survival based on immune cell density (e.g., 0, 1+, 2+, and 3+ indicate observed densities of CD8+ T cells).

### First Pipeline

FIG. 3 illustrates an example first pipeline subsystem 112, in accordance with various embodiments. First pipeline subsystem 112 may implement a first pipeline for determining a tumor immunophenotype of tumor depicted by a digital pathology image. First pipeline subsystem 112 may include an epithelium/immune cell identification module 310, an epithelium-immune cell density module 312, a tumor immunophenotype determination module 314, a density threshold determination module 316, or other components.

In some embodiments, epithelium/immune cell identification module 310 may be configured to receive an image of a tumor and identify one or more regions of the image depicting tumor epithelium. In some embodiments, epithelium/immune cell identification module 310 may identify the one or more regions by scanning the image using a sliding window. A portion of the image included within the sliding window may be analyzed to determine whether that portion of the image included within the sliding window depicts tumor epithelium, tumor stroma, or tumor epithelium and tumor stroma. In some embodiments, the sliding window may have a size of 280×280 pixels, and a stride of 200 pixels, however other values may be used. In some embodiments, epithelium/immune cell identification module 310 may classify each portion as a region depicting tumor epithelium based on the portion satisfying a tumor epithelium criterion and/or a region depicting tumor stroma based on the portion satisfying a tumor stroma criterion. The tumor epithelium criterion may be satisfied if at least a threshold amount of the portion of the image depicts tumor epithelium. Similarly, the stroma criterion may be satisfied if at least a threshold amount of the portion of the image depicts tumor stroma. As an example, the threshold amount may be 25% of the portion of the image. Accordingly, the portion of the image may be classified as depicting tumor epithelium, tumor stroma, or both tumor epithelium and tumor stroma.

In some embodiments, a biological sample of the tumor may be stained using one or more staining agents. The stain may be applied to a sample of the tumor prior to the image being captured. In some embodiments, the stain may include a first stain and a second stain where the first stain highlights and distinguishes epithelial cells forming tumor epithelium from stroma cells forming tumor stroma, and the second stain may highlight immune cells. An example, the first stain may be a pan-cytokeratin (panCK) stain used for highlighting epithelial cells (e.g., CK+ regions) and stroma cells (e.g., CK− regions), and the second stain may be a cluster of differentiation 8 (CD8) stain used for highlighting immune cells (e.g., CD8+ regions). Epithelium/immune cell identification module 310 may be configured to classify each portion of the image (e.g., the portion included in the sliding window) as being a region depicting tumor epithelium and/or a region depicting tumor stroma.

In some embodiments, epithelium/immune cell identification module 310 may perform a color deconvolution to the image to obtain a plurality of color channel images. The color deconvolution performed may include application of a hue-saturation-value (HSV) thresholding model, which may be used to isolate the color channel images. Each color channel image may highlight different types of biological objects. For example, a first color channel image may highlight and distinguish epithelial cells forming regions of tumor epithelium and stromal cells highlighting regions of tumor stroma and a second color channel image may highlight immune cells. Referring again to FIGS. 24A-24E, tumor epithelium and tumor stroma may be represented by the regions of purple, while immune cells may be represented by brown spots. Brown spots located within regions of tumor epithelium may correspond to immune cells that have enter the tumor epithelium, whereas brown spots within regions of tumor stroma may correspond to immune cells that have not entered the tumor epithelium. Persons of ordinary skill in the art will recognize that the color or colors of the spots representing immune cells may vary and/or be tuned to show certain features and/or show some features over other features. Further, the spots representing immune cells may present within a range of colors.

In some embodiments, epithelium/immune cell identification module 310 may be configured to determine the number of immune cells within each of the regions of the image using one or more machine learning models. The machine learning models may include a computer vision model trained to recognize epithelial cells, stromal cells, and immune cells. For example, the computer vision model may be a convolutional neural network (CNN), a U-Net, a Mask R-CNN, or other models. As an example, HoVer-Net is a model that can be used for cell segmentation and classification. The machine learning models may be trained to recognize biological objects, such as immune cells, within an image. In some embodiments, the machine learning models may be trained using training data including training images labeled to indicate whether the image includes a depiction of one or more immune cells, a location (e.g., in pixel space) of those immune cells, or other information.

Epithelium-immune cell density module 312 may be configured to calculate an epithelium-immune cell density for each region identified as depicting tumor epithelium. In some embodiments, epithelium-immune cell density module 312 may calculate the epithelium-immune cell density based on a number of immune cells within each region depicting tumor epithelium. For example, using the color channel images obtained from the color deconvolution, the regions of tumor epithelium may be identified and within each of these regions, epithelium-immune cell density module 312 may determine whether any immune cells are present and quantify those immune cells (if any). Regions of tumor epithelium having greater quantities of immune cells may have greater epithelium-immune cell densities than regions of tumor epithelium having lesser quantities of immune cells. In some embodiments, epithelium-immune cell density module 312 may be configured to compute an average epithelium-immune cell density of the digital pathology image. The average epithelium-immune cell density may be computed by determining an epithelium-immune cell density for each region depicting tumor epithelium and averaging these epithelium-immune cell densities.

In some embodiments, tumor immunophenotype determination module 314 may be configured to determine a tumor immunophenotype for the digital pathology image based on the epithelium-immune cell density and one or more density thresholds. For example, the density thresholds may include a first density threshold. Epithelium-immune cell densities that are greater than or equal to the first density threshold may be classified as being a first tumor immunophenotype (e.g., inflamed) whereas epithelium-immune cell densities that are less than the first density threshold may be classified as being a second tumor immunophenotype (e.g., non-inflamed). As another example, the density thresholds may include a first density threshold and a second density threshold. Epithelium-immune cell densities that are greater than or equal to the first density threshold may be classified as being a first tumor immunophenotype (e.g., inflamed). Epithelium-immune cell densities that are less than the first density threshold and greater than or equal to the second density threshold may be classified as being a second tumor immunophenotype (e.g., excluded). Epithelium-immune cell densities that are less than the first density threshold and the second density threshold may be classified as being a third tumor immunophenotype (e.g., desert). Examples of the tumor immunophenotypes of desert, excluded, and inflamed are illustrated in images 2400-2406 of FIGS. 24A-24D.

Density threshold determination module 316 may be configured to determine the density thresholds for tumor immunophenotyping. In some embodiments, a plurality of images of tumors may be accessed. For example, density threshold determination module 316 may access the images stored in image database 142. Each image may depict a tumor, or a portion of a tumor, obtained from a patient participating in a clinical trial. For example, the clinical trial may include patients with advanced NSCLC, where the patients are randomly selected to receive one of two (or more) immunotherapies (e.g., atezolizumab, docetaxel). In some embodiments, for some or all of the patients in the clinical trial, a digital pathology image of a biological sample (e.g., a tumor lesion) may be captured. For each digital pathology image, one or more regions of the image depicting tumor epithelium may be identified. For example, epithelium/immune cell identification module 310 may be used to determine the regions depicting tumor epithelium. For each region, an epithelium-immune cell density may be calculated. For example, epithelium-immune cell density module 312 may be used to calculate the epithelium-immune cell density of each region based on the number of immune cells detected within the regions of tumor epithelium. In some embodiments, the average epithelium-immune cell density of the image may be calculated.

Density threshold determination module 316 may be configured to generate a ranking of the plurality of images based on each image's epithelium-immune cell density. As an example, with reference to FIG. 4, ranking 400 may include images 402-1 to 402-10 (collectively “images 402”). Each of images 402 may depict a tumor from a patient participating in the clinical trial. Images 402 may be ranked based on each image's epithelium-immune cell density. In the illustrated example, image 402-1 may have a largest epithelium-immune cell density, while image 402-10 may have a smallest epithelium-immune cell density. In some embodiments, the epithelium-immune cell density of each of images 402 may be an average epithelium-immune cell density. For example, for each of images 402, the number of immune cells within each tumor epithelium region may be determined, a local epithelium-immune cell density for each region may be calculated based on the number of immune cells detected within the region, and an average epithelium-immune cell density may be calculated based on the local epithelium-immune cell densities. Therefore, ranking 400 may be generated based on the average epithelium-immune cell density of each of images 402.

In some embodiments, density threshold determination module 316 may be configured to determine a first set of images, a second set of images, and a third set of images from the plurality of images based on the ranking. Each set of images may be associated with a tumor immunophenotype. For example, the first set of images may be associated with a first tumor immunophenotype, the second set of images may be associated with a second tumor immunophenotype, and the third set of images may be associated with a third tumor immunophenotype. In some embodiments, density threshold determination module 316 may select a first percentage of images 402 to be included in the first set of images, a second percentage of images 402 to be included in the second set of images, and a third percentage of images 402 to be included in the third set of images. For example, the first percentage may refer to an upper 40% of images 402, the second percentage may refer to a next 40% of images 402, and the third percentage may refer to a remaining 20% of images 402. In the example of FIG. 4, first set of images 408 may include images 402-1 to 402-4, second set of images 410 may include images 402-5 to 402-8, and third set of images 412 may include images 402-9 to 402-10. In this example, images 402-1 to 402-4 may have epithelium-immune cell densities comprising a top 40% of the epithelium-immune cell densities of images 402 from ranking 400, images 402-5 to 402-8 may comprise a next 40% of the epithelium-immune cell densities of images 402 from ranking 400, and images 402-9 to 402-10 may a bottom 20% of the epithelium-immune cell densities of images 402 from ranking 400. Persons of ordinary skill in the art will recognize that a number of tumor immunophenotypes may differ, and the use of three tumor immunophenotypes is exemplary.

In some embodiments, density threshold determination module 316 may determine the density thresholds for immunophenotyping based on the epithelium-immune cell densities of the images included within sets of images 408, 410, 412. For example, density threshold determination module 316 may determine a first density threshold 404 based on the epithelium-immune cell densities of first set of images 408 and second set of images 410 and may also determine a second density threshold 406 based on second set of images 410 and third set of images 412. In some embodiments, density threshold determination module 316 may determine first density threshold 404 based on an epithelium-immune cell density of images 402-4 and 402-5 and may determine second density threshold 406 based on an epithelium-immune cell density of images 402-8 and 402-9.

First density threshold 404 may be used to differentiate between a first tumor immunophenotype and a second tumor immunophenotype. For example, images with epithelium-immune cell densities greater than or equal to first density threshold 404 may be determined to have the tumor immunophenotype of inflamed. Images with epithelium-immune cell densities less than first density threshold 404 may be determined to have the tumor immunophenotype of non-inflamed.

In some embodiments, first density threshold 404 and second density threshold 406 may be used to differentiate between a first, second, or third tumor immunophenotype. For example, images with epithelium-immune cell densities less than second density threshold 406 may be determined to have the tumor immunophenotype of desert. Images within epithelium-immune cell densities greater than or equal to second density threshold 406 and also less than first density threshold 404 may be determined to have the tumor immunophenotype of excluded. Images with epithelium-immune cell densities greater than or equal to first density threshold 404 may be determined to have the tumor immunophenotype of inflamed.

In some embodiments, a density ratio cutoff of the epithelium-immune cell density between images included in first set of images 408 and images included in second set of images 410 may be between 0.049 and 0.69. For example, the 20^{th} percentile density ratio cutoff of epithelium-immune cell densities may be 0.00059 (e.g., 0.00059 CD8+ area/CK+ area) between the tumor immunophenotypes of desert and excluded. In some embodiments, a density ratio cutoff of the epithelium-immune cell density between images included in second set of images 410 and images included in third set of images 412 may be between 0.4 and 0.6. For example, the 60^{th} percentile density ratio cutoff of epithelium-immune cell densities may be 0.005 (e.g., 0.005 CD8+ area/CK+ area) between the tumor immunophenotypes of excludes and inflamed.

In some embodiments, the accuracy of first density threshold 404 and second density threshold 406 may be evaluated using images depicting tumors from patients participating in a different clinical trial. For example, these images may include labels indicating a pre-determined tumor immunophenotype assigned by a trained pathologist. For each of these images, an epithelium-immune cell density may be calculated and compared to first density threshold 404 and second density threshold 406 to determine a tumor immunophenotype for that image's depicted tumor. The determined tumor immunophenotype may be compared to the pre-determined tumor immunophenotype indicated by that image's label. If first density threshold 404 and second density threshold 406 predict the tumor immunophenotype of the images with at least a threshold accuracy (e.g., 80% or greater accuracy, 90% or greater accuracy, 95% or greater accuracy, etc.), first pipeline subsystem 112 may use first density threshold 404 and second density threshold 406 for deployed instances of the first model. However, if the accuracy of first density threshold 404 and second density threshold 406 to predict the tumor immunophenotype is less than the threshold accuracy, then first density threshold 404 and second density threshold 406 may be recalculated using additional digital pathology images.

Returning to FIG. 3, tumor immunophenotype determination module 314 may use a classifier to determine the tumor immunophenotype. For example, a classifier may be trained to predict a tumor immunophenotype based on the epithelium-immune cell density of an image depicting a tumor and first density threshold 404 and/or second density threshold 406. The classifier may be trained using a plurality of epithelium-immune cell densities and labels indicating a corresponding tumor immunophenotype.

In some embodiments, first pipeline subsystem 112 may implement a first pipeline including one or more machine learning models. For example, one or more computer vision models may be trained to receive an image, identify regions of the image depicting tumor epithelium, and calculate an epithelium-immune cell density of the image based on a number of immune cells detected within the regions. The computer vision models may output the epithelium-immune cell density to a classifier, which may output a tumor immunophenotype for the image.

The machine learning techniques that can be used in the systems/subsystems/modules described herein may include, but are not limited to (which is not to suggest that any other list is limiting), any of the following: Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, or separable convolutions (e.g., depth-separable convolutions, spatial separable convolutions).

First pipeline subsystem 112 may implement a first pipeline for determining a tumor immunophenotype of a tumor depicted by a digital pathology image. The first pipeline may calculate an epithelium-immune cell density at the whole sample level. In comparison to the second pipeline implemented by second pipeline subsystem 114, the third pipeline implemented by third pipeline subsystem 116, and the fourth pipeline implemented by fourth pipeline subsystem 118, the first pipeline implemented by first pipeline subsystem 112 may have a simplest representation tumor immunophenotype and may be the easiest to implement (based on simplicity of design and computational needs). On the other hand, the results of the first pipeline may be slightly less accurate than those of the second pipeline implemented by second pipeline subsystem 114, the third pipeline implemented by third pipeline subsystem 116, and the fourth pipeline implemented by fourth pipeline subsystem 118.

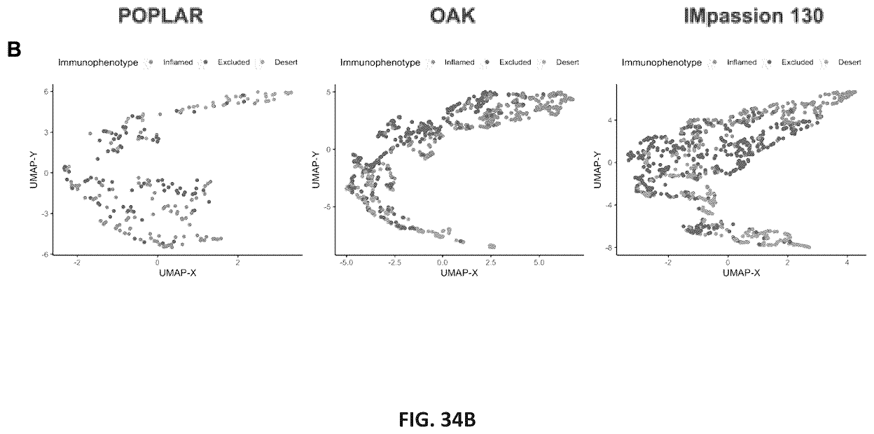

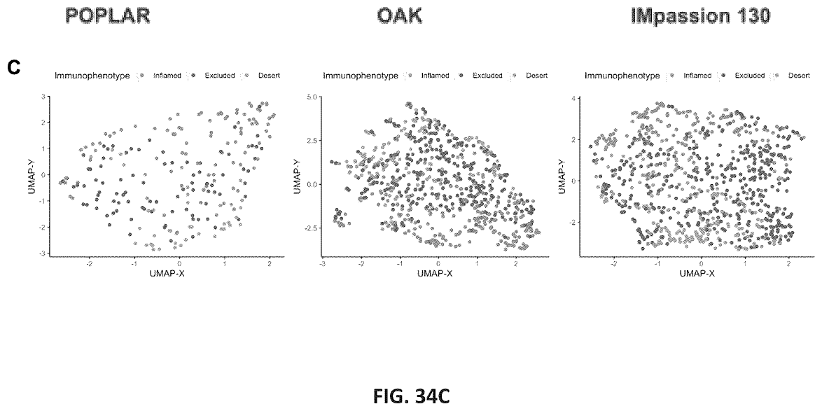

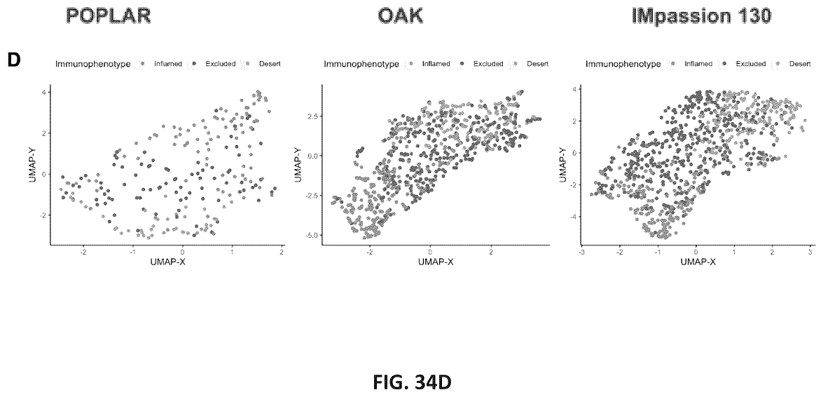

In some embodiments, the first pipeline may determine regions of tumor epithelium using machine learning models and/or via a trained pathologist. The regions may be identified by determining that a region of the image include CK+ cells and CD8+ T cells. For each of these regions, the average density of the CD8+ T cells may be determined. A unimodal distribution of epithelium-immune cell density was observed across all samples, with an enrichment of tumor immunophenotypes manually classified including desert samples at the low end, excluded in the middle, and inflamed at the high end. For example, as seen with reference to FIGS. 34A-34D, a row A of plots (as shown in FIG. 34A) illustrate distributions of biological samples with manually assigned tumor immunophenotypes determined using the first pipeline over various clinical trials. In these plots of FIGS. 34A-34D, the distribution is computed at a slide-level based on an average epithelium-immune cell density. The unimodality of these distributions suggests that a fully unsupervised classification of samples into three groups could lead to a high misclassification error.

As such, classification of samples into one of the set of tumor phenotypes (e.g., desert, excluded, inflamed) was developed in the first pipeline using a supervised fashion. In particular, density thresholds, also referred to as density cutoffs, may be applied. The density thresholds may be determined, as described above, based on manual tumor immunophenotyping images of tumors from patients participating in a first clinical trial. For example, as seen with respect to FIG. 4, set 412 may include images whose calculated epithelium-immune cell density is in the 20^{th} percentile or lower of epithelium-immune cell densities of images 402 from ranking 400. In an example clinical trial, this may lead to a density threshold for second density threshold 406 of 0.00059% for desert samples. Set 410 may include images whose calculated epithelium-immune cell density is less than the 60th percentile and greater than or equal to the 20^{th} percentile of epithelium-immune cell densities of images 402 from ranking 400. In the example clinical trial, this may lead to a density threshold for first density threshold 404 of 0.005%, where excluded samples have an epithelium-immune cell density less than first density threshold 404 and greater than or equal to second density threshold 406. Set 408 may include images whose calculated epithelium-immune cell density is in the 60th percentile or greater of epithelium-immune cell densities of images 402 from ranking 400. Inflamed samples may have an epithelium-immune cell density that is greater than or equal to first threshold 404.

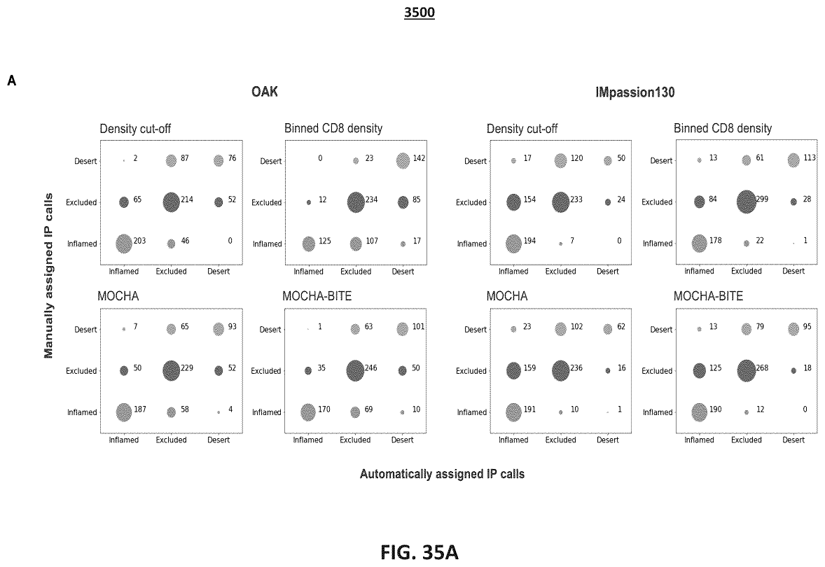

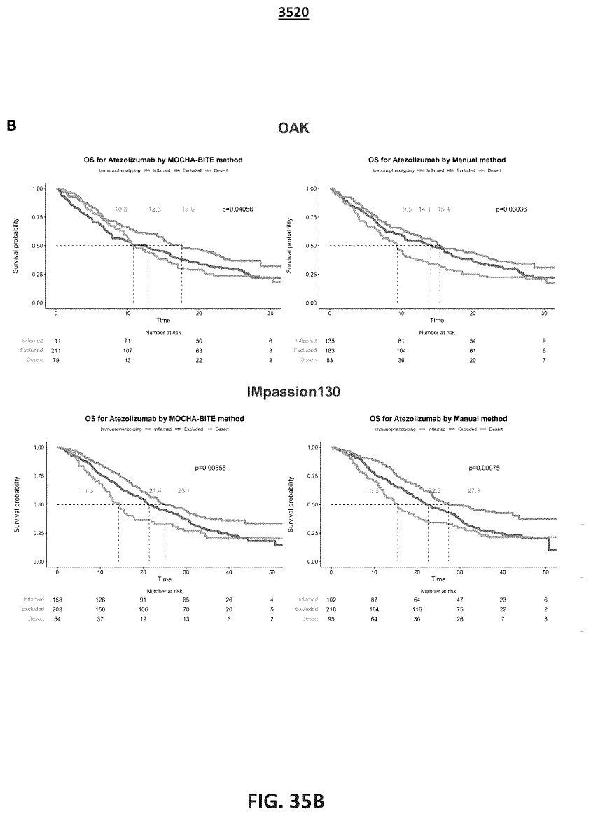

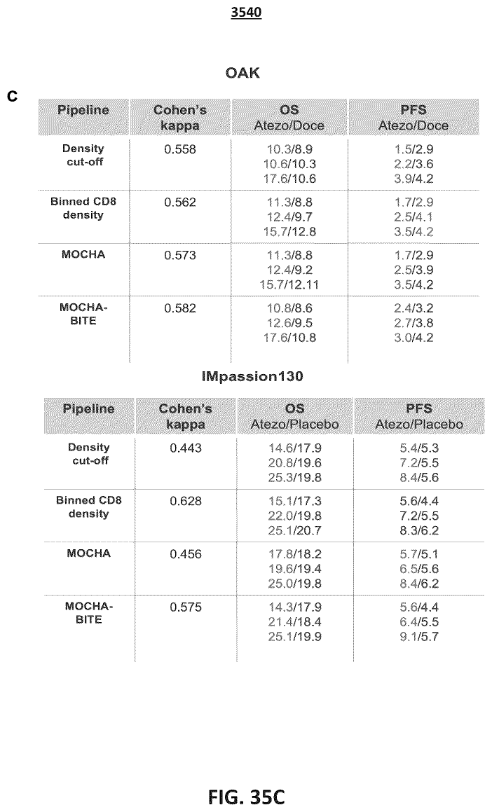

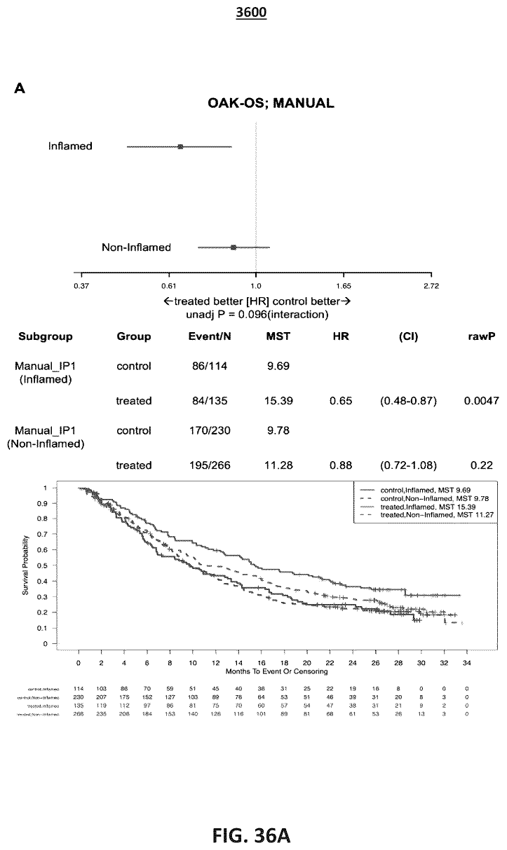

The density cutoffs specified by first density threshold 404 and second density threshold 406 (e.g., 0.005 and 0.00059, respectively) may be applied to one or more other clinical trials to validate the calculated density thresholds. For example, when formed on a first clinical trial looking at patients participating in a NSCLC trial, density thresholds produce highly accurate results when compared against a second clinical trial corresponding to a phase 3 NSCLC trial and a third clinical trial corresponding to a TNBC trial (e.g., 0.558 in the example second clinical trial, 0.443 in the example third clinical trial). Observation of such results is depicted by density plots 3500 of FIG. 35A. Plots 3500 depict confusion matrices showing the distribution of manually assigned tumor immunophenotype calls (rows) against the automated tumor immunophenotype calls (columns) for the example second clinical trial and the example third clinical trial. For example, among the example second clinical trial, 203 of the manually assigned Inflamed tumors were classified as Inflamed by the first pipeline, while 46 were classified as excluded, and 0 were classified as Desert. Graphs 3520 of FIG. 35B depict examples of the Kaplan-Meier curves for OS for atezolizumab-containing treatment arms for the example second clinical trial and the example third clinical trial according to the tumor immunophenotype classes (e.g., desert, excluded, inflamed) as determined by the fourth pipeline. Tables 3540 of FIG. 35C illustrate a performance of the first, second, third, and fourth pipelines as compared to manual tumor immunophenotype classification (serving as a ground truth). For each pipeline, the Cohen's kappa coefficient is indicated as well as the median OS and PFS times for each tumor immunophenotype of one treatment arm (e.g., atezolizumab) and the control arm of the example second and third clinical trials.

This same cutoff methodology was applied, using images depicting tumors of patients from the example first clinical trial to an immune cell density calculated across the entire tumor area without regard to regions of tumor epithelium (i.e., including regions of tumor epithelium and regions of tumor stroma). This resulted in second density threshold 406 of 0.003 for the samples representing the tumor immunophenotype desert, and a first density threshold 404 of 0.0014 for the samples representing the tumor immunophenotype excluded. While inclusion of tumor stroma decreases the overall accuracy and Cohen's kappa of the first pipeline's ability to classify tumor immunophenotype in images of tumors from the example second clinical trial, the difference is ˜4% for each measure. Excluding stromal cells, and thus stroma-immune cell densities, from the first pipeline's implementation simplifies the staining and analysis protocol but at a cost to accuracy (KM curves and other data not shown).

### Second Pipeline

FIG. 5 illustrates an example second pipeline subsystem 114, in accordance with various embodiments. Second pipeline subsystem 114 may implement a second pipeline for determining a tumor immunophenotype of a tumor depicted by a digital pathology image. Second pipeline subsystem 114 may include a tile generation module 510, an epithelium/stroma identification module 512, an epithelium-immune cell density module 514, a stroma-immune cell density module 516, a density binning module 518, a density-bin representation module 520, a classification module 522, a classifier training module 524, or other components.

In some embodiments, tile generation module 510 may be configured to receive an image depicting a tumor and divide the image into a plurality of tiles. The image may be a digital pathology image captured using a digital pathology imaging system, such as scanner 240. As an example, with reference to FIG. 6, tile generation module 510 may receive a digital pathology image 604 of a biological sample 602 that has been imaged by image scanner 240 (previously described with reference to FIG. 2). Tile generation module 510 may segment digital pathology image 604 into tiles 606. In some embodiments, tiles 606 can be non-overlapping (e.g., each tile includes pixels of digital pathology image 604 not included in any other tile) or overlapping (e.g., each tile includes some portion of pixels of digital pathology image 604 that are included in at least one other tile). Features such as whether tiles 606 overlap, in addition to a size of each tile and the stride window (e.g., the image distance or number of pixels between a tile and a subsequent tile) can increase or decrease the data set for analysis, with more tiles (e.g., through overlapping or smaller tiles) increasing the potential resolution of eventual outputs and visualizations. In some embodiments, tile generation module 510 may define a set of tiles for an image where each tile is of a predefined size and/or an offset between tiles is predefined. Furthermore, tile generation module 510 may generate multiple sets of tiles of varying size, overlap, step size, etc., for each digital pathology image. In some embodiments, digital pathology image 604 itself can contain tile overlap, which may result from the imaging technique. In some embodiments, tile segmentation without overlapping tiles can balance tile processing requirements and can avoid influencing embedding generation and/or weighting value generation. A tile size or tile offset can be determined, for example, by calculating one or more performance metrics (e.g., precision, recall, accuracy, and/or error) for each size/offset and by selecting a tile size and/or offset associated with one or more performance metrics above a predetermined threshold and/or associated with one or more optimal (e.g., high precision, highest recall, highest accuracy, and/or lowest error) performance metric(s).

Tile generation module 510 may further be configured to define a tile size. The tile size may be determined based on a type of abnormality being detected. For example, tile generation module 510 may be configured to set the tile size for segmentation of digital pathology image 604 based on the types of tissue abnormalities present in biological sample 602. Tile generation module 510 may also customize the tile size based on the tissue abnormalities to be detected/searched for to optimize detection. In some embodiments, tile generation module 510 may determine that, when the tissue abnormalities include inflammation or necrosis in lung tissue, the tile size should be reduced to increase the scanning rate. In some embodiments, tile generation module 510 may determine that, when the tissue abnormalities include abnormalities with Kupffer cells in liver tissues, the tile size should be increased to increase the opportunities for second pipeline subsystem 114 to analyze the Kupffer cells holistically. In some embodiments, tile generation module 510 may define a set of tiles where a number of tiles in the set, a size of the tiles of the set, a resolution of the tiles for the set, or other related properties, for each image may be defined and held constant for each of one or more images. As an example, each of tiles 606 may have a size of approximately 16,000 μm².

In some embodiments, tile generation module 510 may be configured to receive digital pathology image 604 of biological sample 602 (e.g., a tumor). Digital pathology image 604 of biological sample 602 may include (at least a portion of) a whole slide image (WSI). For example, as mentioned above, digital pathology image generation subsystem 110 may be configured to produce a WSI of a biological sample (e.g., a tumor). In some embodiments, digital pathology image generation subsystem 110 may generate multiple digital pathology images of biological sample 602 at different settings. For example, image scanner 240 may capture images of biological sample 602 at multiple magnification levels (e.g., 5×, 10×, 20×, 40×, etc.). These images may be provided to second pipeline subsystem 114 as a stack, and an operator may determine which image or images from the stack to be used for the subsequent analysis. The biological sample may be prepared, sliced, stained, and subsequently imaged to produce the WSI. The biological sample may include a biopsy of a tumor. For example, the biological sample may include tumors from NSCLC clinical trials and/or TNBC clinical trials.

In some embodiments, a region of interest of digital pathology image 604 may be identified prior to tiling. For example, a pathologist may manually define the region of interest (ROI) in the tumor. The ROI may be defined using a digital pathology image viewing system at a particular magnification (e.g., 4×). As another example, a machine learning model may be used to define the ROI in the tumor lesion. In this example, a human (e.g., pathologist) may be able to review the machine-defined ROI (e.g., to confirm that the defined ROI is accurate). The defined ROI may exclude areas of necrosis. This is important because some staining agents can label normal epithelial cells, not just tumor epithelium.

In some embodiments, one or more stains may be applied to biological sample 602 prior to digital pathology image 604 being captured by image scanner 240. The stains cause different objects of biological sample 602 to turn different colors. For example, one stain (e.g., CD8) may cause immune cells to turn one color, while another stain (e.g., panCK) may cause tumor epithelium and tumor stroma to turn another color. Therefore, the first stain may highlight immune cells, whereas the second stain may highlight, as well as distinguish, tumor epithelium and tumor stroma.

In some embodiments, a color deconvolution may be performed to digital pathology image 604. The color deconvolution may separate out each color channel images from digital pathology image 604, obtaining a plurality of color channel images. In this example, tiles 606 can be produced for each color channel image. In some embodiments, a hue-saturation-value (HSV) thresholding model may be used to isolate the color channel images. Each color channel image may highlight different biological objects. For example, a first color channel image may highlight and distinguish epithelial cells forming tumor epithelium and stromal cells forming tumor stroma and a second color channel image may highlight immune cells. Referring again to FIGS. 24A-24E, tumor epithelium and tumor stroma may be represented by the regions of purple, while immune cells may be represented by brown spots. Brown spots located within regions of tumor epithelium may correspond to immune cells that have enter the tumor epithelium, whereas brown spots within regions of tumor stroma may correspond to immune cells that have not entered the tumor epithelium.

Returning to FIG. 5, epithelium/stroma identification module 512 may be configured to identify epithelium tiles and stroma tiles from tiles 606. In some embodiments, epithelium/stroma identification module 512 may scan the digital pathology image using a sliding window to determine whether a portion of the digital pathology image included within the sliding window depicts tumor epithelium, tumor stroma, or both tumor epithelium and tumor stroma. In some embodiments, epithelium/stroma identification module 512 may analyze each tile 606 to determine whether that tile includes depictions of epithelial cells (e.g., CK+ cells), stroma cells (e.g., CK− cells), immune cells (e.g., CD8+ T cells), or other cells, or combinations thereof.

Epithelium/stroma identification module 512 may be configured to identify one or more of tiles 606 as being an epithelium tile based on a portion of that tile satisfying an epithelium-tile criterion. The epithelium-tile criterion may be satisfied if the portion of the tile depicting tumor epithelium (e.g., an area of the tile encompassing pixels highlighting epithelial cells) is greater than or equal to a first threshold area (e.g., 10% or more of a tile's area, 25% or more of a tile's area, 40% or more of a tile's area, 50% or more of a tile's area, etc.). For example, if 25% or more of a tile's area is determined to include depictions of epithelial cells, that tile may be classified as an epithelium tile. Each of tiles 606 determined to be an epithelium tile may be tagged with an epithelium tile label (e.g., metadata indicating that the corresponding tile is an epithelium tile). The metadata may also indicate spatial information about the epithelium tile, such as the tile's position relative to digital pathology image 604 and the other tiles 606 and/or a location of depictions of particular types of biological objects, such as epithelial cells (e.g., pixel coordinates of a pixel having a pixel hue, saturation, and/or value associated with that of an epithelial cell).

Epithelium/stroma identification module 512 may be configured to identify one or more of tiles 606 as being a stroma tile based on a portion of that tile satisfying a stroma-tile criterion. The stroma-tile criterion may be satisfied if the portion of the tile depicting tumor stroma (e.g., an area of the tile encompassing pixels highlighting stromal cells) is greater than or equal to a first threshold area (e.g., 10% or more of a tile's area, 25% or more of a tile's area, 40% or more of a tile's area, 50% or more of a tile's area, etc.). For example, if 25% or more of a tile's area is determined to include depictions of stromal cells, that tile may be classified as a stroma tile. Each of tiles 606 determined to be a stroma tile may be tagged with a stroma tile label (e.g., metadata indicating that the corresponding tile is a stroma tile). The metadata may also indicate spatial information about the stroma tile, such as the tile's position relative to digital pathology image 604 and the other tiles 606 and/or a location of depictions of particular types of biological objects, such as stromal cells (e.g., pixel coordinates of a pixel having a pixel hue, saturation, and/or value associated with that of a stromal cell).

In some embodiments, a tile may be classified as being both an epithelium tile and a stroma tile. A tile may satisfy both the epithelium-tile criterion and the stroma-tile criterion. For example, a portion of a tile depicting tumor epithelium (e.g., an area of the tile encompassing pixels highlighting epithelial cells) being greater than or equal to a first threshold area (e.g., 10% or more of a tile's area, 25% or more of a tile's area, 40% or more of a tile's area, 50% or more of a tile's area, etc.) and the same or different portion of the tile depicting tumor stroma (e.g., an area of the tile encompassing pixels highlighting stromal cells) being greater than or equal to a second threshold area (e.g., 10% or more of a tile's area, 25% or more of a tile's area, 40% or more of a tile's area, 50% or more of a tile's area, etc.) may indicate that this tile should be classified as being an epithelium tile and a stroma tile. As an example, a tile that is determined to have an area that is 50% CK+ may be classified as both an epithelium tile and a stroma tile. In some embodiments, the first threshold area and the second threshold area may be the same or they may differ. Metadata may be stored in association with a tile. The metadata may indicate that the tile has been classified as being both an epithelium tile and a stroma tile. Furthermore, some embodiments may include at least some of the epithelium tiles depicting regions of tumor stroma and at least some of the stroma tiles depicting regions of tumor epithelium.

Returning to FIG. 5, epithelium-immune cell density module 514 may be configured calculate an epithelium-immune cell density for each of the epithelium tiles. In some embodiments, the epithelium-immune cell density for an epithelium tile may be calculated based on a number of immune cells detected within the epithelium tiles. Epithelium tiles having greater quantities of immune cells within tumor epithelium may have a greater epithelium-immune cell density than epithelium tiles having fewer immune cells. In some embodiments, epithelium-immune cell density module 514 may be implement one or more machine learning models to determine the number of immune cells within each epithelium tile. In some embodiments, the machine learning models may include a computer vision model trained to recognize biological objects, such as immune cells, within an image tile. For example, the machine learning model may be a CNN trained to detect immune cells within an image. The machine learning models may be stored in model database 146. In some embodiments, epithelium-immune cell density module 514 may access the machine learning model(s) from model database 146 and may provide each epithelium tile to the machine learning model(s) as an input. The machine learning model(s) may output an epithelium-immune cell density for that epithelium tile and/or a value indicating a number of immune cells detected within that epithelium tile. In the latter example, where the value is output by the machine learning model(s), epithelium-immune cell density module 514 may be configured to calculate the epithelium-immune cell density based on the number of immune cells detected, an area of the tile, and/or an area of the tile including depictions of epithelial cells.

Stroma-immune cell density module 516 may be configured calculate a stroma-immune cell density for each of the stroma tiles. In some embodiments, the stroma-immune cell density for a stroma tile may be calculated based on a number of immune cells detected within the stroma tiles. Stroma tiles having greater quantities of immune cells within tumor stroma may have a greater stroma-immune cell density than stroma tiles having fewer immune cells. In some embodiments, stroma-immune cell density module 516 may be implement one or more machine learning models to determine the number of immune cells within each stroma tile. In some embodiments, the machine learning models may include a computer vision model trained to recognize biological objects, such as immune cells, within an image tile. For example, the machine learning model may be a CNN trained to detect immune cells within an image. The machine learning models may be stored in model database 146. In some embodiments, the machine learning models implemented by stroma-immune cell density module 516 to calculate the stroma-immune cell density may be the same or similar to the machine learning models implemented by epithelium-immune cell density module 514 to calculate the epithelium-immune cell density. In some embodiments, stroma-immune cell density module 516 may access the machine learning model(s) from model database 146 and provide each stroma tile to the machine learning model(s) as an input. The machine learning model(s) may output a stroma-immune cell density for that stroma tile and/or a value indicating a number of immune cells detected within that stroma tile. In the latter example, where the value is output by the machine learning model(s), stroma-immune cell density module 516 may be configured to calculate the stroma-immune cell density based on the number of immune cells detected, an area of the tile, and/or an area of the tile including depictions of stromal cells.

In some embodiments, the machine learning models may be trained to detect different types of biological objects within an image tile. The one or more machine learning models are trained using training data including a plurality of training images and labels indicating a type of biological object or types of biological objects depicted within each of the plurality of training images, a quantity of each depicted biological object, a location of each depicted biological object, or other information related to the biological objects depicted within the image. In some embodiments, the training images may be of a same or similar size as that of image tiles 606 of FIG. 6. In some embodiments, the training images may be whole slide images divided into tiles. The type of biological objects that the machine learning models are be trained to detect may include immune cells, epithelial cells, stromal cells, or other types of biological objects, or combinations thereof.

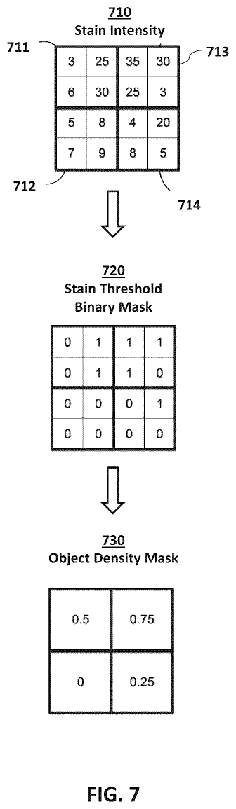

FIG. 7 illustrates an example of an immune cell density calculation, in accordance with various embodiments. For example, the process described by FIG. 7 may be used to calculate an epithelium-immune cell density and/or a stroma-immune cell density of a tile. As seen in FIG. 7, three masks 710, 720, and 730 are illustrated. Each of masks 710, 720, and 730 may have been generated from a digital pathology image, such as digital pathology image 604 of FIG. 6. In some embodiments, masks 710, 720, and 730 may be generated using epithelium-immune cell density module 514 and/or stroma-immune cell density module 516, or other suitable components of second pipeline subsystem 114.

In some embodiments, mask 710 may be a stain intensity mask for a digital pathology image. In the illustrated example, the digital pathology image and mask 710 may be divided into four tiles 711-714. Each of tiles 711-714 may include four pixels. Each pixel may be associated with a stain intensity value that corresponds to the intensity of a particular stain (e.g., the intensity of color channels known to be reflective of stain performance). For example, the northwest tile, tile 711, may include stain intensity values: 3, 25, 6, and 30; the southwest tile, tile 712, may include stain intensity values: 5, 8, 7, and 9; the northeast tile, tile 713 may include stain intensity values: 35, 30, 25, and 3; and the southeast tile, tile 714, may include stain intensity values: 4, 20, 8, and 5. Each of the stain intensity values may be reflective of the performance of the stain (e.g., the rate of absorption or expression of the stain by the biological objects depicted in the corresponding pixels of the digital pathology image). The stain intensity values can be used to determine which biological objects are shown in the tiles and the frequency of their appearance.

In some embodiments, mask 720 may be a stain thresholded binary mask for stain intensity mask 710. Each individual pixel value of stain intensity mask 710 may be compared to a predetermined and customizable threshold for the stain of interest. The threshold value can be selected accordingly to protocol reflective of the expected level of expression of stain intensity corresponding to a confirmed depiction of the correct biological object. The stain intensity values and threshold values can be absolute values (e.g., a stain intensity value above 20) or relative values (e.g., setting the threshold at the top 30% of stain intensity values). Additionally, the stain intensity values can be normalized according to historical values (e.g., based on overall performance of the stain on a number of previous analyses) or based on the digital pathology image at hand (e.g., to account for brightness differences and other imaging changes that may cause the image to inaccurately display the correct stain intensity). In stain thresholded binary mask 720, the threshold may be set to a stain intensity value of 20 and applied across all pixels within stain intensity mask 710. The result may be a pixel-level binary mask with ‘1’ indicating that the pixel had a stain intensity at or exceeding the threshold value and ‘0’ indicating that the pixel did not satisfy the requisite stain intensity.

In some embodiments, mask 730 may be an object density mask on the tile-level. Based on the assumption that stain intensity levels above the threshold correlate to depiction of a particular biological object within the digital pathology image, operations may be performed on the stain thresholded binary mask 720 to reflect the density of biological object within each tile. In the example object density mask 730, the operations include summing the values of the stain thresholded binary mask 720 within each tile and dividing by the number of pixels within the tile. As an example, the northwest tile, tile 711, may include two pixels above the threshold stain intensity value out of a total of four pixels, therefore the value in object density mask 730 for the northwest tile is 0.5. Similar operations may be applied across all of tiles 711-714. Additional operations can be performed to, for example, preserve locality with each tile, such as sub-tile segmentation and preservation of coordinates of each sub-tile within the lattice. As described herein, object density mask 730 can be used as the basis for calculation of spatial-distribution metrics (described in greater detail below with respect to third pipeline subsystem 116). It will be appreciated that the example depicted in FIG. 7 is simplified for discussion purposes only. The number of pixels within each tile and the number of tiles within each digital pathology image can be greatly expanded and adjusted as needed based on computational efficiency and accuracy requirements.

Returning to FIG. 5, density binning module 518 may be configured to bin the epithelium tiles into an epithelium set of bins based on each epithelium tile's epithelium-immune cell density and bin the stroma tiles into a stroma set of bins based on each stroma tile's stroma-immune cell density. The epithelium set of bins and the stroma set of bins may each include a predetermined number of bins corresponding to a particular range of densities. A tile may be “binned” into one of the bins based on whether the tile is an epithelium tile, a stroma tile, or a stroma tile and an epithelium tile, and the corresponding epithelium-immune cell density and/or stroma-immune cell density calculated for that tile. In some embodiments, the epithelium set of bins and the stroma set of bins may include a same quantity of bins each encompassing a same predefined range of densities.

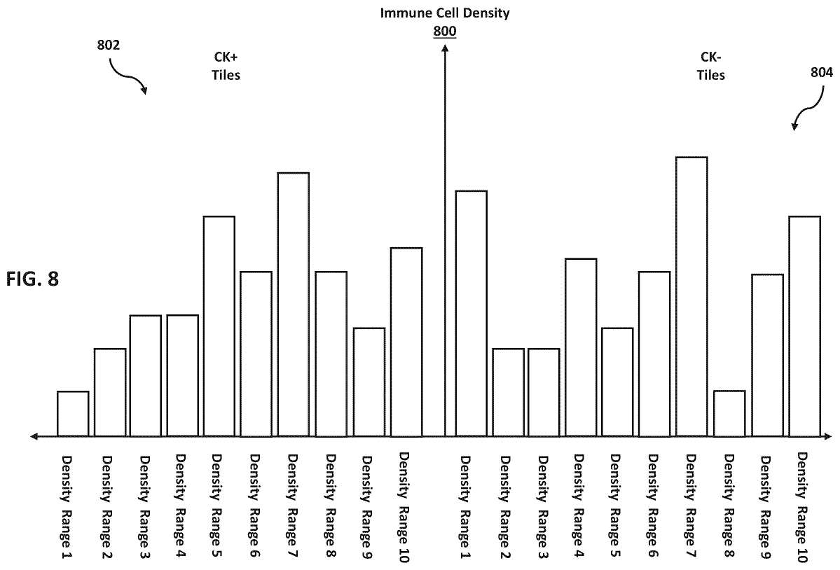

As an example, with reference to FIG. 8, an immune cell density distribution 800 may include epithelium set of bins 802 and stroma set of bins 804. An epithelium tile binned into one of epithelium set of bins 802 may have an epithelium-immune cell density falling within a density range associated with that bin. Similarly, a stroma tile binned into one of stroma set of bins 804 may have a stroma-immune cell density falling within a density range associated with that bin. In other words, a bin may be defined by a lower density threshold, T1, and an upper density threshold, T2, and a tile determined to have an immune cell density that is less threshold T2 but greater than or equal to threshold T1 may be allocated to that bin. Allocating a tile to a bin may refer to incrementing a count of tiles that have an immune cell density falling within that bin's density range. In some embodiments, allocating immune cell density distribution 800 may include two data structures: one representing epithelium set of bins 802 and one representing stroma set of bins 804. The data structures may have a number of elements equal to the number of bins in each set of bins 802, 804. A value of each element in each data structure may correspond to a number of tiles determined to have an epithelium/stroma-immune cell density within a density range associated with that bin, an average epithelium/stroma immune cell density of all of the tiles within that density range, or a minimum/maximum epithelium/stroma immune cell density of the tiles within that density range, and the like. In some embodiments, the values for each element of the data structures may be normalized (e.g., based on a total number of tiles, an average epithelium immune cell density across all bins, etc.).

In some embodiments, epithelium set of bins 802 and stroma set of bins 804 may each include a same number of bins. For example, epithelium set of bins 802 may include ten bins and stroma set of bins 804 may also include ten bins. However, persons of ordinary skill in the art will recognize that other quantities of bins may be used. In this example, the corresponding data structures representing epithelium set of bins 802 and stroma set of bins 804 may include a same number of elements. Each of the ten bins may be defined by a corresponding density range. In some embodiments, the density ranges of epithelium set of bins 802 and stroma set of bins 804 may be the same. For example, a first bin from epithelium set of bins 802 may encompass a first density range [T1-T2], and a first bin from stroma set of bins 804 may also encompass the first density range [T1-T2]. Similarly, a second bin from epithelium set of bins 802 may encompass a second density range [T2-T3], and a second bin from stroma set of bins 804 may also encompass the second density range [T2-T3].

Immune cell density distribution 800 may be formed by determining a number of epithelium tiles that have an epithelium-immune cell density within each of the density ranges of epithelium set of bins 802 and a number of stroma tiles that have a stroma-immune cell density within each of the density ranges of stroma set of bins 804. As an example, epithelium set of bins 802 may be defined by ten density ranges: a first density range comprising epithelium-immune cell densities between 0.0-0.005, a second density range comprising epithelium-immune cell densities between 0.005-0.01, a third density range comprising epithelium-immune cell densities between 0.01-0.02, a fourth density range comprising epithelium-immune cell densities between 0.02-0.04, a fifth density range comprising epithelium-immune cell densities between 0.04-0.06, a sixth density range comprising epithelium-immune cell densities between 0.06-0.08, a seventh density range comprising epithelium-immune cell densities between 0.08-0.12, an eighth density range comprising epithelium-immune cell densities between 0.12-0.16, a ninth density range comprising epithelium-immune cell densities between 0.16-0.2, and a tenth density range comprising epithelium-immune cell densities between 0.2-2.0. Stroma set of bins 804 may be defined by ten density ranges: a first density range comprising stroma-immune cell densities between 0.0-0.005, a second density range comprising stroma-immune cell densities between 0.005-0.01, a third density range comprising stroma-immune cell densities between 0.01-0.02, a fourth density range comprising stroma-immune cell densities between 0.02-0.04, a fifth density range comprising stroma-immune cell densities between 0.04-0.06, a sixth density range comprising stroma-immune cell densities between 0.06-0.08, a seventh density range comprising stroma-immune cell densities between 0.08-0.12, an eighth density range comprising stroma-immune cell densities between 0.12-0.16, a ninth density range comprising stroma-immune cell densities between 0.16-0.2, and a tenth density range comprising stroma-immune cell densities between 0.2-2.0.

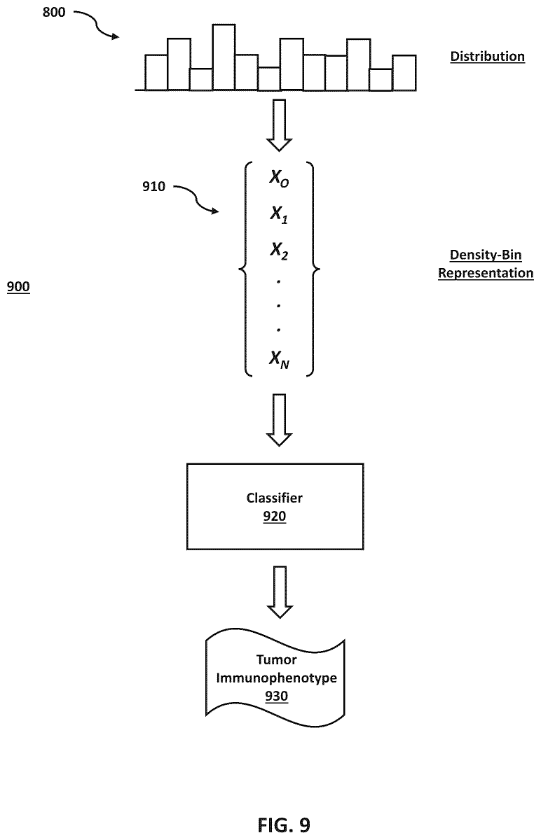

Returning to FIG. 5, density-bin representation module 520 may be configured to generate a density-bin representation based on immune cell density distribution 800. In some embodiments, the density-bin representation may represent epithelium set of bins 802 and stroma set of bins 804, which form immune cell density distribution 800. The density-bin representation may include a plurality of elements corresponding to each bin of epithelium set of bins 802 and each bin of stroma set of bins 804. As an example, with reference to FIG. 9, immune cell distribution 800 of FIG. 8 may be transformed into density-bin representation 910 via process 900. Density-bin representation 910 may include elements X₀-X_{N}, where N represents a total number of bins in immune cell density distribution 800. For example, if epithelium set of bins 802 and stroma set of bins 804 include ten bins, N=20. Considering the example where immune cell density distribution 800 includes two data structures respectively representing epithelium set of bins 802 and stroma set of bins 804, each data structure may include N/2 elements (in the scenario where the number of bins is equal across bins 802, 804). A value of each element (e.g., elements X₀-X_{N}) in each data structure may correspond to a number of tiles determined to have an epithelium/stroma-immune cell density within a density range associated with that bin, an average epithelium/stroma immune cell density of all of the tiles within that density range, or a minimum/maximum epithelium/stroma immune cell density of the tiles within that density range, and the like. In some embodiments, the values for each element (e.g., elements X₀-X_{N}) in the data structures may be normalized (e.g., based on a total number of tiles, an average epithelium immune cell density across all bins, etc.).

In some embodiments, density-bin representation module 520 may transform immune cell density distribution 800 into density-bin representation 910. In some embodiments, density-bin representation 910 may be a feature vector that can be input to a classifier 920 to determine a tumor immunophenotype of an image (e.g., image 604 of FIG. 6). For example, the feature vector may be formed of the data structures comprising elements X₀-X_{N}.

Returning to FIG. 5, classification module 522 may be configured to implement a classifier, such as classifier 920 of FIG. 9. During process 900, classification module 522 may receive the density-bin representation (e.g., density-bin representation 910) and input the density-bin representation into the classifier. The classifier may be trained to output a tumor immunophenotype (e.g., tumor immunophenotype 930) of the biological sample depicted by digital pathology image 604 of FIG. 6. In some embodiments, the classifier is a multi-class classifier configured to classify an image into one of a set of tumor immunophenotypes. The number of classes may depend on the number of tumor immunophenotype classifications. For example, classifier 920 may be trained to classify an image into one of a set of tumor immunophenotypes based on density-bin representation 910. The set of tumor immunophenotypes may include the tumor immunophenotypes: desert, excluded, and inflamed. In some embodiments, classifier 920 implemented by classification module 522 may be a support vector machine (SVM), a random forest (RF) classifier, a decision tree classifier, a logistic regression classifier, a k-nearest neighbor (kNN) classifier, or another type of classifier.

In some embodiments, an embedding may be generated based on density-bin representation 910. The embedding may be mapped into an embedding space using a trained encoder. In this example, tumor immunophenotype 930 may be determined based on a distance between the mapped location of the embedding in the embedding space and one or more clusters of embeddings, each cluster being associated with a tumor immunophenotype. A tumor immunophenotype associated with the cluster determined to be closest (e.g., having a minimum L2 distance) to the embedding may be assigned to that image, indicating that the tumor depicted by the image is classified as being the assigned tumor immunophenotype.

Returning to FIG. 5, classifier training module 524 may be configured to train a classifier to predict a tumor immunophenotype of an image of a tumor based on a density-bin representation generated for that image. In some embodiments, classifier training module 524 may train the classifier using training data generated from a first plurality of patients participating in a first clinical trial. Classifier training module 524 may further be configured to validate the classifier using validation data generated from a second plurality of patients participating in a second clinical trial.

In some embodiments, classifier training module 524 may access a first plurality of images from image database 142. The first plurality of images may include images of tumors from patients participating in a first clinical trial. For example, the first clinical trial may include patients having advanced NSCLC who have progressed on a platinum-based chemotherapy regiment and who were 1:1 randomized to receive either a first immunotherapy (e.g., atezolizumab) or a second immunotherapy (e.g., docetaxel). The number of patients participating in the first clinical trial may be 100 or more patients, 200 or more patients, 300 or more patients, etc. In some embodiments, biological samples may be obtained for at least some of the patients of the first clinical trial. One biological sample may be obtained for each of the patients. The biological samples may include tumor lesions including tumor stroma and tumor epithelium. The biological samples may be stained using a dual-stain, such as a panCK-CD8 stain, to highlight epithelial cells (e.g., CK+ cells) forming tumor epithelium, stromal cells (e.g., CK− cells) forming tumor stroma, and immune cells (e.g., CD+ T cells).

Each sample may be imaged using image scanner 240 to obtain a first plurality of images. These images may be stored in image database 142. The images may be digital pathology images, such as whole-slide images. In some embodiments, classifier training module 524 and/or other components of second pipeline subsystem 114 may be configured to divide each of the first plurality of images into tiles and identify which of those tiles are epithelium tiles and/or stroma tiles. For each image, an epithelium-immune cell density may be calculated for the epithelium tiles and a stroma-immune cell density may be calculated for the stroma tiles and an immune cell density distribution may be generated for the image by binning the epithelium tiles into an epithelium set of bins based on each epithelium tile's corresponding epithelium-immune cell density and by binning the stroma tiles into a stroma set of bins based on each stroma tile's corresponding stroma-immune cell density. A density-bin representation may be generated for each image based on that image's corresponding epithelium set of bins and stroma set of bins. Therefore, for each of the first plurality of images, a corresponding density-bin representation may be obtained.

In some embodiments, classifier training module 524 may be configured to generate training data including the first plurality of images and, for each image, a label indicating a predetermined tumor immunophenotype of the tumor depicted by that image. In some embodiments, the predetermined tumor immunophenotype may be assigned by a trained pathologist. The training data may be stored in training data database 144. Classifier training module 524 may select a classifier to be trained from model database 146 and may be configured to train the classifier using the training data stored in training data database 144. The classifier may be a multi-class classifier, such as a three-class SVM. Classifier training module 524 may optimize hyperparameters of the classifier using an optimizer, such as the Adam optimizer.

After the classifier has been trained using the training data, it may be evaluated using validation data. Classifier training module 524 may be configured to generate the validation data using a second plurality of images. The second plurality of images may also be stored in image database 142. The second plurality of images may include images of tumors from patients participating in a second clinical trial. As an example, the second clinical trial may include patients having advanced NSCLC. As another example, the second clinical trial may include patients having metastatic TNBC who were randomized to receive a first therapy (e.g., atezolizumab plus nab-paclitaxel) or a second therapy (e.g., a placebo plus nab-paclitaxel). The number of patients participating in the first clinical trial may be 500 or more patients, 750 or more patients, 1,000 or more patients, etc. In some embodiments, biological samples may be obtained for at least some of the patients of the second clinical trial. One biological sample may be obtained for each of the patients. The biological samples may include tumor lesions including tumor stroma and tumor epithelium. The biological samples may be stained using a dual-stain, such as a panCK-CD8 stain, to highlight epithelial cells (e.g., CK+ cells) forming tumor epithelium, stromal cells (e.g., CK− cells) forming tumor stroma, and immune cells (e.g., CD+ T cells).

Similar to the process described above for the first clinical trial, validation data may be generated based on a second plurality of images of the biological samples of at least some of the patients of the second clinical trial. The second plurality of images may be used to generate density-bin representations. In some embodiments, labels indicating a predetermined tumor immunophenotype of the biological sample may be assigned by a trained pathologist. Classifier training module 524 may use the validation data to evaluate the accuracy of the trained classifier. If the classifier does not predict the tumor immunophenotype with at least a threshold level of accuracy, then classifier training module 524 may retrain the classifier. However, if the classifier is determined to have a threshold level of accuracy, it may be deployed for determining a tumor immunophenotype of an input image. As seen in FIG. 9, classifier 920 may correspond to the trained classifier. Classifier 920 may output tumor immunophenotype 930 based on density-bin representation 910, which may be derived from a digital pathology image of a tumor (e.g., digital pathology image 604 of biological sample 602).

In some embodiments, tumor immunophenotype 930 may be one of a set of tumor immunophenotypes. For example, the tumor immunophenotypes may include desert, excluded, and inflamed. In some embodiments, a tumor depicted by a digital pathology image may be classified as the tumor immunophenotype desert based on an epithelium-immune cell density calculated for that image satisfying a desert epithelium-immune cell density threshold criterion and a stroma-immune cell density for that image also satisfying a desert stroma-immune cell density threshold criterion. As an example, the desert epithelium-immune cell density threshold criterion being satisfied may include the epithelium-immune cell density being within a first threshold range of epithelium-immune cell densities and the desert stroma-immune cell density threshold criterion being satisfied may include the stroma-immune cell density being within a first threshold range of stroma-immune cell densities. A tumor depicted by a digital pathology image may be classified as the tumor immunophenotype excluded based on an epithelium-immune cell density for that image satisfying an excluded epithelium-immune cell density threshold criterion and a stroma-immune cell density for that image satisfying an excluded stroma-immune cell density threshold criterion. For example, the excluded epithelium-immune cell density threshold criterion being satisfied may include the epithelium-immune cell density being within a second threshold range of epithelium-immune cell densities and the excluded stroma-immune cell density threshold criterion being satisfied may include the stroma-immune cell density being within a second threshold range of stroma-immune cell densities. A tumor depicted by a digital pathology image may be classified as the tumor immunophenotype inflamed based on an epithelium-immune cell density of that image satisfying an inflamed epithelium-immune cell density threshold criterion and a stroma-immune cell density of that image satisfying an inflamed stroma-immune cell density threshold criterion. For example, the inflamed epithelium-immune cell density threshold criterion being satisfied may include the epithelium-immune cell density being within a third threshold range of epithelium-immune cell densities and the inflamed stroma-immune cell density threshold criterion being satisfied may include the stroma-immune cell density being within a third threshold range of stroma-immune cell densities.

In some embodiments, the machine learning techniques that can be used in the systems/subsystems/modules of second pipeline subsystem 114 may include, but are not limited to (which is not to suggest that any other list is limiting), any of the following: Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, or separable convolutions (e.g., depth-separable convolutions, spatial separable convolutions).

The second pipeline implemented by second pipeline subsystem 114 may use a supervised classification approach to predict tumor immunophenotypes. As described above, a density-bin representation may be generated based on an immune cell density distribution of epithelium tiles and stroma tiles. For example, the density-bin representation may be a 20-dimensional feature vector. In some embodiments, the 20-dimensional feature vector (representing the binned immune cell density ranges) may be projected into a two-dimensional Uniform Manifold Approximation and Projection (UMAP) space. In some embodiments, images depicting tumors of patients participating in the example first clinical trial (as described above) may be used to train a multi-class classifier to predict tumor immunophenotype. The trained classifier may be validated on the images of tumors from patients participating in the example second clinical trial and/or the example third clinical trial, as seen, for example, with reference to the second row of density plots of plots 3500 from FIG. 35A. The weighted Cohen's kappa for the second density pipeline, as computed for the example second clinical trial, was determined to be 0.562 and, the weighted Cohen's kappa for the second density pipeline, as computed for the example third clinical trial, was determined to be 0.628.

In some embodiments, the second pipeline implemented by second pipeline subsystem 114 can use a weakly supervised multiple instance learning-based classification approach to predict tumor immunophenotypes. For example, the second pipeline can involve dividing, using the tile generation module 510, a histology image into a plurality of tiles. Each tile can depict a distinct structure within the imaged tissue and can be referred to as an “instance.” The histology image, treated as a collective entity, can serve as a “bag” encompassing multiple instances, and can be labeled as “positive” or “negative” for association with a tumor immunophenotype. In some embodiments, the tumor immunophenotype of the histology image can be automatically determined using a machine learning model trained via multiple instance learning. For example, the second pipeline can involve determining, using the classification module 522 and/or the classifier training module 524, a tumor immunophenotype (e.g., tumor immunophenotype 930) of the histology image using a classifier (e.g., classifier 920) trained via multiple instance learning. In some embodiments, an attention score mechanism can be used to identify which instances within a bag contribute significantly to making a positive prediction and/or label. This attention-score-based process can aid in emphasizing the most relevant and discriminative regions within the histology image, contributing to the model and/or algorithm's ability to discern intricate patterns associated with the tumor immunophenotype. For example, attention scores can be derived from the features of each instance (e.g., by assessing their similarity in relation to features of other instances within the same bag). Instances that are more similar to other instances may be less strongly associated with a positive label, thereby having a lower attention score. For example, in a histology image containing only healthy cells, the healthy cells may all resemble one another, and there may be no reason to pay greater attention to one image tile over another. Conversely, instances that are more distinct may be more strongly associated with a positive label, thereby having a higher attention score. For example, in a histology image containing both healthy cells and tumor cells, the tumor cells may stand out from the healthy cells, and the multiple instance machine-learning model may pay greater attention to the image tiles containing the standout tumor cells when labeling the bag as “positive.” An instance-based model and/or instance-based algorithm can be trained to predict the tumor immunophenotype based on the attention scores. For example, a histology image containing both healthy cells and tumor cells may be labeled as “positive” based on the high attention scores associated with image tiles depicting tumor cells within the histology image, and the overall histology image can be labeled as containing a specific tumor type as a result.

### Third Pipeline

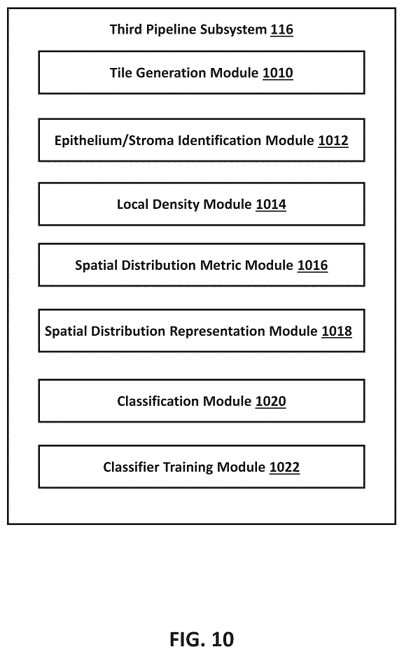

FIG. 10 illustrates an example third pipeline subsystem 116, in accordance with various embodiments. Third pipeline subsystem 116 may implement a third pipeline for determining a tumor immunophenotype of a tumor depicted by a digital pathology image. Third pipeline subsystem 116 may include a tile generation module 1010, an epithelium/stroma identification module 1012, a local density module 1014, a spatial distribution metric module 1016, a spatial distribution representation module 1018, a classification module 1020, a classifier training module 1022, or other components.

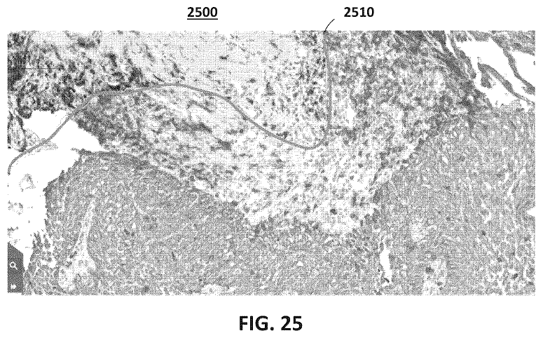

In some embodiments, tile generation module 1010 may be configured to receive an image depicting a tumor. The image may be a digital pathology image captured using a digital pathology imaging system, such as scanner 240. In some embodiments, tile generation module 1010 may be configured to annotate a digital pathology image. As an example, with reference to FIG. 25, digital pathology image 2500 shows a line 2510 separating the bottom portion of digital pathology image 2500 from the top portion. The separation may indicate that the bottom portion of digital pathology image 2500 is associated with a tumor bed (e.g., including tumor epithelium and stroma) while a portion of digital pathology image 2500 above line 2510 may not be associated with the tumor bed. As described herein, line 2510 can be generated by tile generation module 1010 and/or by digital pathology image generation subsystem 110 or may be received by tile generation module 1010 from a manual evaluation. Moreover, annotated digital pathology image 2500 can be provided as a form of output from tile generation module 1010. As described herein, multiple forms of output can be provided as a mechanism allowing reviewers to better under the approach adopted by tile generation module 1010 and how it arrived at its ultimate conclusions. Outputs indicating the segmentation into tumor epithelium and tumor stroma may be a first step to ensuring that tile generation module 1010 correctly interpreted the digital pathology image and sample.

In some embodiments, tile generation module 1010 may be configured to receive an image depicting a tumor and divide the image into a plurality of tiles. The image may be a digital pathology image captured using a digital pathology imaging system, such as scanner 240. As an example, with reference to FIG. 6, tile generation module 1010 may receive a digital pathology image 604 of a biological sample 602 that has been imaged by image scanner 240 (previously described with reference to FIG. 2). Tile generation module 1010 may segment digital pathology image 604 into tiles 606. In some embodiments, tiles 606 can be non-overlapping (e.g., each tile includes pixels of digital pathology image 604 not included in any other tile) or overlapping (e.g., each tile includes some portion of pixels of digital pathology image 604 that are included in at least one other tile). Features such as whether tiles 606 overlap, in addition to a size of each tile and the stride window (e.g., the image distance or number of pixels between a tile and a subsequent tile) can increase or decrease the data set for analysis, with more tiles (e.g., through overlapping or smaller tiles) increasing the potential resolution of eventual outputs and visualizations. In some embodiments, tile generation module 1010 may define a set of tiles for an image where each tile is of a predefined size and/or an offset between tiles is predefined. Furthermore, tile generation module 1010 may generate multiple sets of tiles of varying size, overlap, step size, etc., for each digital pathology image. In some embodiments, digital pathology image 604 itself can contain tile overlap, which may result from the imaging technique. In some embodiments, tile segmentation without overlapping tiles can balance tile processing requirements and can avoid influencing embedding generation and/or weighting value generation. A tile size or tile offset can be determined, for example, by calculating one or more performance metrics (e.g., precision, recall, accuracy, and/or error) for each size/offset and by selecting a tile size and/or offset associated with one or more performance metrics above a predetermined threshold and/or associated with one or more optimal (e.g., high precision, highest recall, highest accuracy, and/or lowest error) performance metric(s).

Tile generation module 1010 may further be configured to define a tile size. The tile size may be determined based on a type of abnormality being detected. For example, tile generation module 1010 may be configured to set the tile size for segmentation of digital pathology image 604 based on the types of tissue abnormalities present in biological sample 602. Tile generation module 1010 may also customize the tile size based on the tissue abnormalities to be detected/searched for to optimize detection. In some embodiments, tile generation module 1010 may determine that, when the tissue abnormalities include inflammation or necrosis in lung tissue, the tile size should be reduced to increase the scanning rate. In some embodiments, tile generation module 1010 may determine that, when the tissue abnormalities include abnormalities with Kupffer cells in liver tissues, the tile size should be increased to increase the opportunities for second pipeline subsystem 114 to analyze the Kupffer cells holistically. In some embodiments, tile generation module 1010 may define a set of tiles where a number of tiles in the set, a size of the tiles of the set, a resolution of the tiles for the set, or other related properties, for each image may be defined and held constant for each of one or more images. As an example, each of tiles 606 may have a size of approximately 16,000 μm².

In some embodiments, tile generation module 1010 may be configured to receive digital pathology image 604 of biological sample 602 (e.g., a tumor). Digital pathology image 604 of biological sample 602 may include (at least a portion of) a whole slide image (WSI). For example, as mentioned above, digital pathology image generation subsystem 110 may be configured to produce a WSI of a biological sample (e.g., a tumor). In some embodiments, digital pathology image generation subsystem 110 may generate multiple digital pathology images of biological sample 602 at different settings. For example, image scanner 240 may capture images of biological sample 602 at multiple magnification levels (e.g., 5×, 10×, 20×, 40×, etc.). These images may be provided to third pipeline subsystem 116 as a stack, and an operator may determine which image or images from the stack to be used for the subsequent analysis. The biological sample may be prepared, sliced, stained, and subsequently imaged to produce the WSI. The biological sample may include a biopsy of a tumor. For example, the biological sample may include tumors from NSCLC clinical trials and/or TNBC clinical trials.

In some embodiments, a region of interest of digital pathology image 604 may be identified prior to tiling. For example, a pathologist may manually define the region of interest (ROI) in the tumor. The ROI may be defined using a digital pathology image viewing system at a particular magnification (e.g., 4×). As another example, a machine learning model may be used to define the ROI in the tumor lesion. In this example, a human (e.g., pathologist) may be able to review the machine-defined ROI (e.g., to confirm that the defined ROI is accurate). The defined ROI may exclude areas of necrosis. This is important because some staining agents can label normal epithelial cells, not just tumor epithelium.

In some embodiments, one or more stains may be applied to biological sample 602 prior to digital pathology image 604 being captured by image scanner 240. The stains cause different objects of biological sample 602 to turn different colors. For example, one stain (e.g., CD8) may cause immune cells to turn one color, while another stain (e.g., panCK) may cause tumor epithelium and tumor stroma to turn another color. Therefore, the first stain may highlight immune cells, whereas the second stain may highlight, as well as distinguish, tumor epithelium and tumor stroma.

In some embodiments, a color deconvolution may be performed to digital pathology image 604. The color deconvolution may separate out each color channel images from digital pathology image 604, obtaining a plurality of color channel images. In this example, tiles 606 can be produced for each color channel image. In some embodiments, a hue-saturation-value (HSV) thresholding model may be used to isolate the color channel images. Each color channel image may highlight different biological objects. For example, a first color channel image may highlight and distinguish epithelial cells forming tumor epithelium and stromal cells forming tumor stroma and a second color channel image may highlight immune cells. Referring again to FIGS. 24A-24E, tumor epithelium and tumor stroma may be represented by the regions of purple, while immune cells may be represented by brown spots. Brown spots located within regions of tumor epithelium may correspond to immune cells that have enter the tumor epithelium, whereas brown spots within regions of tumor stroma may correspond to immune cells that have not entered the tumor epithelium.

Returning to FIG. 10, epithelium/stroma identification module 1012 may be configured to identify epithelium tiles and stroma tiles from tiles 606. In some embodiments, epithelium/stroma identification module 1012 may scan the digital pathology image using a sliding window to determine whether a portion of the digital pathology image included within the sliding window depicts tumor epithelium, tumor stroma, or both tumor epithelium and tumor stroma. In some embodiments, epithelium/stroma identification module 1012 may analyze each tile 606 to determine whether that tile includes depictions of epithelial cells (e.g., CK+ cells), stroma cells (e.g., CK− cells), immune cells (e.g., CD8+ T cells), or other cells, or combinations thereof.

Epithelium/stroma identification module 1012 may be configured to identify one or more of tiles 606 as being an epithelium tile based on a portion of that tile satisfying an epithelium-tile criterion. The epithelium-tile criterion may be satisfied if the portion of the tile depicting tumor epithelium (e.g., an area of the tile encompassing pixels highlighting epithelial cells) is greater than or equal to a first threshold area (e.g., 10% or more of a tile's area, 25% or more of a tile's area, 40% or more of a tile's area, 50% or more of a tile's area, etc.). For example, if 25% or more of a tile's area is determined to include depictions of epithelial cells, that tile may be classified as an epithelium tile. In some embodiments, the epithelium-tile criterion may be satisfied if a ratio of epithelial cells over total tumor cells in the tile is greater than or equal to a threshold ratio. For example, for a given tile of a digital pathology image, if the area pixels representing epithelial cells (e.g., CK+ pixels) divided by the area of the sum of the pixels representing epithelial cells and the pixels representing stromal cells (e.g., CK− pixels) is greater than or equal to the threshold ratio (e.g., threshold ratio=0.25), that tile may be classified as being an epithelium tile. Each of tiles 606 determined to be an epithelium tile may be tagged with an epithelium tile label (e.g., metadata indicating that the corresponding tile is an epithelium tile). The metadata may also indicate spatial information about the epithelium tile, such as the tile's position relative to digital pathology image 604 and the other tiles 606 and/or a location of depictions of particular types of biological objects, such as epithelial cells (e.g., pixel coordinates of a pixel having a pixel hue, saturation, and/or value associated with that of an epithelial cell).

Epithelium/stroma identification module 1012 may be configured to identify one or more of tiles 606 as being a stroma tile based on a portion of that tile satisfying a stroma-tile criterion. The stroma-tile criterion may be satisfied if the portion of the tile depicting tumor stroma (e.g., an area of the tile encompassing pixels highlighting stromal cells) is greater than or equal to a first threshold area (e.g., 10% or more of a tile's area, 25% or more of a tile's area, 40% or more of a tile's area, 50% or more of a tile's area, etc.). For example, if 25% or more of a tile's area is determined to include depictions of stromal cells, that tile may be classified as a stroma tile. In some embodiments, the stroma-tile criterion may be satisfied if a ratio of stroma cells over total tumor cells in the tile is greater than or equal to a threshold ratio. For example, for a given tile of a digital pathology image, if the area pixels representing stromal cells (e.g., CK− pixels) divided by the area of the sum of the pixels representing epithelial cells and the pixels representing stromal cells (e.g., CK− pixels) is greater than or equal to the threshold ratio (e.g., threshold ratio=0.25), that tile may be classified as being a stroma tile. Alternatively, a tile that is not classified as an epithelium tile may be classified as being a stroma tile (e.g., if the tile does not satisfy the epithelium-tile criterion). Each of tiles 606 determined to be a stroma tile may be tagged with a stroma tile label (e.g., metadata indicating that the corresponding tile is a stroma tile). The metadata may also indicate spatial information about the stroma tile, such as the tile's position relative to digital pathology image 604 and the other tiles 606 and/or a location of depictions of particular types of biological objects, such as stromal cells (e.g., pixel coordinates of a pixel having a pixel hue, saturation, and/or value associated with that of a stromal cell).

In some embodiments, a tile may be classified as being both an epithelium tile and a stroma tile. A tile may satisfy both the epithelium-tile criterion and the stroma-tile criterion. For example, a portion of a tile depicting tumor epithelium (e.g., an area of the tile encompassing pixels highlighting epithelial cells) being greater than or equal to a first threshold area (e.g., 10% or more of a tile's area, 25% or more of a tile's area, 40% or more of a tile's area, 50% or more of a tile's area, etc.) and the same or different portion of the tile depicting tumor stroma (e.g., an area of the tile encompassing pixels highlighting stromal cells) being greater than or equal to a second threshold area (e.g., 10% or more of a tile's area, 25% or more of a tile's area, 40% or more of a tile's area, 50% or more of a tile's area, etc.) may indicate that this tile should be classified as being an epithelium tile and a stroma tile. In some embodiments, the first threshold area and the second threshold area may be the same or they may differ. Metadata may be stored in association with a tile. The metadata may indicate that the tile has been classified as being both an epithelium tile and a stroma tile. Furthermore, some embodiments may include at least some of the epithelium tiles depicting regions of tumor stroma and at least some of the stroma tiles depicting regions of tumor epithelium.

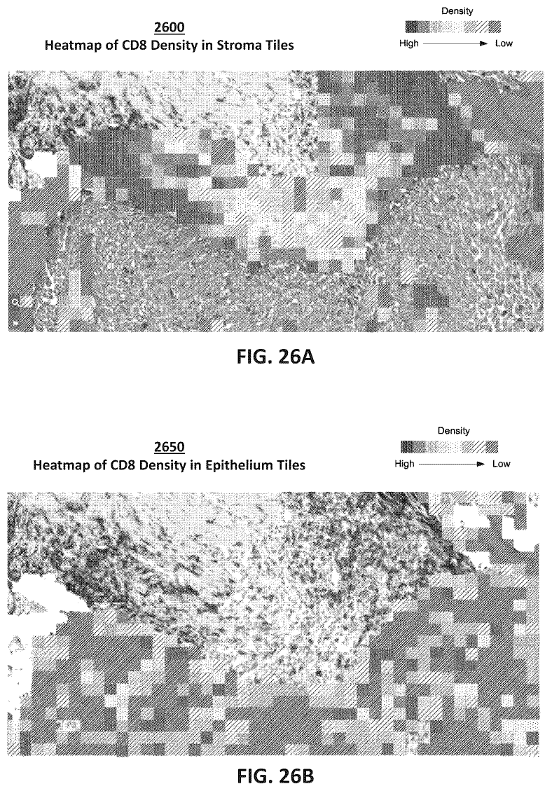

In some embodiments, as seen with reference to FIGS. 26A-26B, epithelium/stroma identification module 1012 may obtain tiles generated by tile generation module 1010 based on an input digital pathology image (e.g., annotated digital pathology image 2500). In some embodiments, epithelium/stroma identification module 1012 may be configured to perform a pixel-based segmentation and generated initial density metrics (e.g., generated an object density mask for the digital pathology image). In the example depicted in FIGS. 26A-26B, epithelium/stroma identification module 1012 may be configured to determine epithelium tiles and stroma tiles from the tiles of digital pathology image 2500. As described below with respect to local density module 1014, an immune cell (e.g., CD8+ T cell) density may be calculated in stroma tiles (e.g., CK− tumor cells) and epithelium tiles (e.g., CK+ tumor cells).

Returning to FIG. 10, local density module 1014 may be configured to determine one or more local density measurements associated with a local density of one or more types of biological object. For example, local density module 1014 may calculate a density of epithelial cells in a given tile, a density of stromal cells in a tile, a density of immune cells (e.g., an epithelium-immune cell density) co-localized with epithelial cells in a tile (e.g., an epithelium-immune cell density), a density of immune cells co-localized with stromal cells in a tile (e.g., a stroma-immune cell density), or other local densities, or combinations thereof.

In some embodiments, local density module 1014 may determine an epithelial cell density of a tile based on a number of epithelial cells detected within the tile. The more epithelial cells present within a tile, the greater the epithelial cell density of that tile. In some embodiments, a tile may be classified as an epithelium tile if an area of the tile satisfies a threshold area criterion. For example, the threshold area criterion being satisfied may comprise a number of pixels whose intensity is greater than or equal to a threshold intensity value being greater than or equal to a threshold number of pixels. The threshold intensity value may be associated with pixels depicting epithelial cells, and the number of pixels being greater than the threshold intensity value may indicate that a threshold area is encompassed by pixels depicting epithelial cells

In some embodiments, local density module 1014 may determine a stromal cell density of a tile based on a number of stromal cells detected within the tile. The more stromal cells present within a tile, the greater the stromal cell density of that tile. In some embodiments, a tile may be classified as a stroma tile if an area of the tile satisfies a threshold area criterion. For example, the threshold area criterion being satisfied may comprise a number of pixels whose intensity is greater than or equal to a threshold intensity value being greater than or equal to a threshold number of pixels. The threshold intensity value may be associated with pixels depicting stromal cells, and the number of pixels being greater than the threshold intensity value may indicate that a threshold area is encompassed by pixels depicting stromal cells.

In some embodiments, local density module 1014 may an epithelium-immune cell density for each of the epithelium tiles. In some embodiments, the epithelium-immune cell density for an epithelium tile may be calculated based on a number of immune cells detected within the epithelium tiles. Epithelium tiles having greater quantities of immune cells within tumor epithelium may have a greater epithelium-immune cell density than epithelium tiles having fewer immune cells. In some embodiments, local density module 1014 may be implement one or more machine learning models to determine the number of immune cells within each epithelium tile. In some embodiments, the machine learning models may include a computer vision model trained to recognize biological objects, such as immune cells, within an image tile. For example, the machine learning model may be a CNN trained to detect immune cells within an image. The machine learning models may be stored in model database 146. In some embodiments local density module 1014 may access the machine learning model(s) from model database 146 and may provide each epithelium tile to the machine learning model(s) as an input. The machine learning model(s) may output an epithelium-immune cell density for that epithelium tile and/or a value indicating a number of immune cells detected within that epithelium tile. In the latter example, where the value is output by the machine learning model(s), local density module 1014 may be configured to calculate the epithelium-immune cell density based on the number of immune cells detected, an area of the tile, and/or an area of the tile including depictions of epithelial cells.

Local density module 1014 may also be configured calculate a stroma-immune cell density for each of the stroma tiles. In some embodiments, the stroma-immune cell density for a stroma tile may be calculated based on a number of immune cells detected within the stroma tiles. Stroma tiles having greater quantities of immune cells within tumor stroma may have a greater stroma-immune cell density than stroma tiles having fewer immune cells. In some embodiments, local density module 1014 may be implement one or more machine learning models to determine the number of immune cells within each stroma tile. In some embodiments, the machine learning models may include a computer vision model trained to recognize biological objects, such as immune cells, within an image tile. For example, the machine learning model may be a CNN trained to detect immune cells within an image. The machine learning models may be stored in model database 146. In some embodiments, the machine learning models implemented by local density module 1014 to calculate the stroma-immune cell density may be the same or similar to the machine learning models implemented by local density module 1014 to calculate the epithelium-immune cell density. In some embodiments, local density module 1014 may access the machine learning model(s) from model database 146 and provide each stroma tile to the machine learning model(s) as an input. The machine learning model(s) may output a stroma-immune cell density for that stroma tile and/or a value indicating a number of immune cells detected within that stroma tile. In the latter example, where the value is output by the machine learning model(s), local density module 1014 may be configured to calculate the stroma-immune cell density based on the number of immune cells detected, an area of the tile, and/or an area of the tile including depictions of stromal cells.

In some embodiments, machine learning models may be trained to detect different types of biological objects within an image tile. The one or more machine learning models are trained using training data including a plurality of training images and labels indicating a type of biological object or types of biological objects depicted within each of the plurality of training images, a quantity of each depicted biological object, a location of each depicted biological object, or other information related to the biological objects depicted within the image. In some embodiments, the training images may be of a same or similar size as that of image tiles 606 of FIG. 6. In some embodiments, the training images may be whole slide images divided into tiles. The type of biological objects that the machine learning models are be trained to detect may include immune cells, epithelial cells, stromal cells, or other types of biological objects, or combinations thereof.

FIG. 7 illustrates an example of an immune cell density calculation, in accordance with various embodiments. For example, the process described by FIG. 7 may be used to calculate an epithelium-immune cell density and/or a stroma-immune cell density of a tile. As seen in FIG. 7, three masks 710, 720, and 730 are illustrated. Each of masks 710, 720, and 730 may have been generated from a digital pathology image, such as digital pathology image 604 of FIG. 6. In some embodiments, masks 710, 720, and 730 may be generated using local density module 1014 or other suitable components of second pipeline subsystem 114.

In some embodiments, mask 710 may be a stain intensity mask for a digital pathology image. In the illustrated example, the digital pathology image and mask 710 may be divided into four tiles 711-714. Each of tiles 711-714 may include four pixels. Each pixel may be associated with a stain intensity value that corresponds to the intensity of a particular stain (e.g., the intensity of color channels known to be reflective of stain performance). For example, the northwest tile, tile 711, may include stain intensity values: 3, 25, 6, and 30; the southwest tile, tile 712, may include stain intensity values: 5, 8, 7, and 9; the northeast tile, tile 713 may include stain intensity values: 35, 30, 25, and 3; and the southeast tile, tile 714, may include stain intensity values: 4, 20, 8, and 5. Each of the stain intensity values may be reflective of the performance of the stain (e.g., the rate of absorption or expression of the stain by the biological objects depicted in the corresponding pixels of the digital pathology image). The stain intensity values can be used to determine which biological objects are shown in the tiles and the frequency of their appearance.

In some embodiments, mask 720 may be a stain thresholded binary mask for stain intensity mask 710. Each individual pixel value of stain intensity mask 710 may be compared to a predetermined and customizable threshold for the stain of interest. The threshold value can be selected accordingly to protocol reflective of the expected level of expression of stain intensity corresponding to a confirmed depiction of the correct biological object. The stain intensity values and threshold values can be absolute values (e.g., a stain intensity value above 20) or relative values (e.g., setting the threshold at the top 30% of stain intensity values). Additionally, the stain intensity values can be normalized according to historical values (e.g., based on overall performance of the stain on a number of previous analyses) or based on the digital pathology image at hand (e.g., to account for brightness differences and other imaging changes that may cause the image to inaccurately display the correct stain intensity). In stain thresholded binary mask 720, the threshold may be set to a stain intensity value of 20 and applied across all pixels within stain intensity mask 710. The result may be a pixel-level binary mask with ‘1’ indicating that the pixel had a stain intensity at or exceeding the threshold value and ‘0’ indicating that the pixel did not satisfy the requisite stain intensity.

In some embodiments, mask 730 may be an object density mask on the tile-level. Based on the assumption that stain intensity levels above the threshold correlate to depiction of a particular biological object within the digital pathology image, operations may be performed on the stain thresholded binary mask 720 to reflect the density of biological object within each tile. In the example object density mask 730, the operations include summing the values of the stain thresholded binary mask 720 within each tile and dividing by the number of pixels within the tile. As an example, the northwest tile, tile 711, may include two pixels above the threshold stain intensity value out of a total of four pixels, therefore the value in object density mask 730 for the northwest tile is 0.5. Similar operations may be applied across all of tiles 711-714. Additional operations can be performed to, for example, preserve locality with each tile, such as sub-tile segmentation and preservation of coordinates of each sub-tile within the lattice. As described herein, object density mask 730 can be used as the basis for calculation of spatial-distribution metrics (described in greater detail below with respect to third pipeline subsystem 116). It will be appreciated that the example depicted in FIG. 7 is simplified for discussion purposes only. The number of pixels within each tile and the number of tiles within each digital pathology image can be expanded and adjusted as needed based on computational efficiency and accuracy requirements.

Returning to FIG. 10, spatial distribution metric module 1016 may be configured to generate one or more spatial distribution metrics. In some embodiments, the spatial distribution metrics may describe an input image of a tumor, such as digital pathology image 604 of FIG. 6. The spatial distribution metrics may be generated based on the local density measurements calculated by local density module 1014. For example, the spatial distribution metrics may be calculated based on an epithelial cell density, a stromal cell density, an epithelium-immune cell density, and a stroma-immune cell density.

In some embodiments, spatial distribution metric module 1016 may be configured to generate heatmaps of biological object densities for one or more types of biological objects. For example, one type of biological object may be immune cells, and the generated heatmaps may indicate immune cell densities in the tumor epithelium (e.g., epithelium-immune cell density) and/or the tumor stroma (e.g., stroma-immune cell density). As seen, for example, with reference to FIGS. 26A-26B, spatial distribution metric module 1016 may create heatmap visualizations 2600 and heatmap visualization 2650 based on the respective object density metrics. For example, heatmap visualization 2600 may illustrate a stroma-immune cell density (e.g., the density of CD8+ T cells in CK− tumor cells). Heatmap visualization 2650 may illustrate an epithelium-immune cell density (e.g., the density of CD8+ T cells in CK+ tumor cells). Heatmap visualization 2600 and heatmap visualization 2650 can assist pathologists in systematically categorizing the sample shown in the digital pathology image and also assist in understanding the tumor immunophenotype that will be assigned by third pipeline subsystem 116.

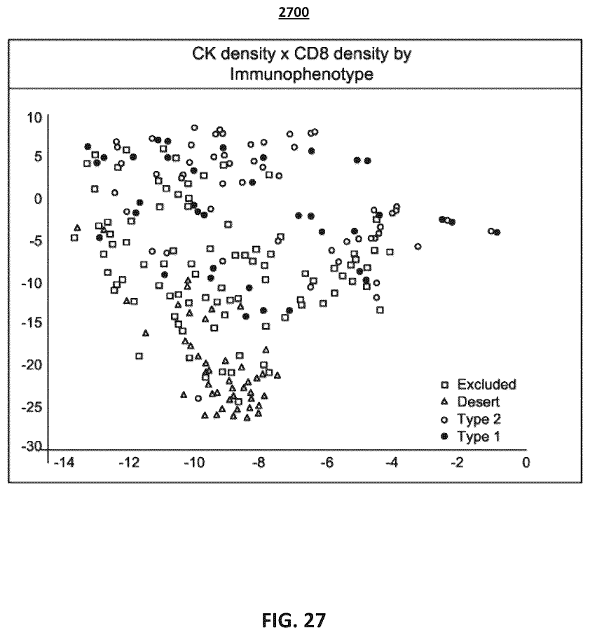

FIG. 27 illustrates a plot 2700 of biological object density bins by tumor immunophenotype, in accordance with various embodiments. For example, plot 2700 illustrates epithelial cell densities (e.g., CK+ tile densities) and stromal cell densities (e.g., CK− densities) plotted against immune cell densities (e.g., CD8+ T cell densities). Plot 2700 illustrates a first and naïve interpretation of the density scores that can be generated by local density module 1014. Although certain trends may be determinable from plot 2700, such as the clustering of desert immunophenotypes lower in the y-axis and the prevalence of inflamed immunophenotypes higher in both the x-axis and y-axis, it is difficult to draw further conclusions as additional clusters cannot be determined. Thus, plot 2700 demonstrates the limitations of previous forms of analysis and the motivation of development of additional techniques to automatically classify digital pathology images and the tiles derived therefrom. These additional techniques include, for example, the integration of advanced spatial distribution metrics derived from the density values.

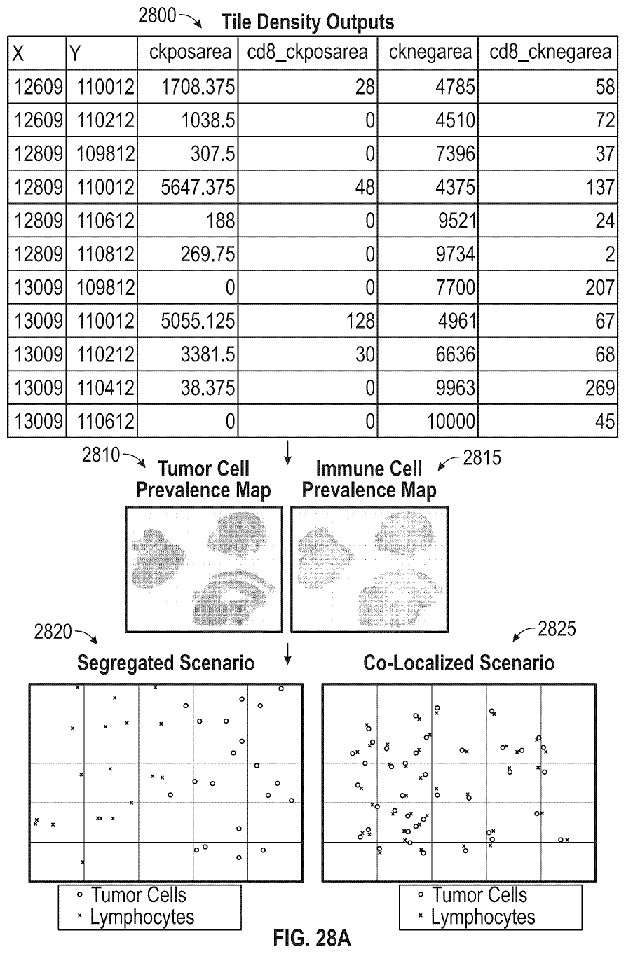

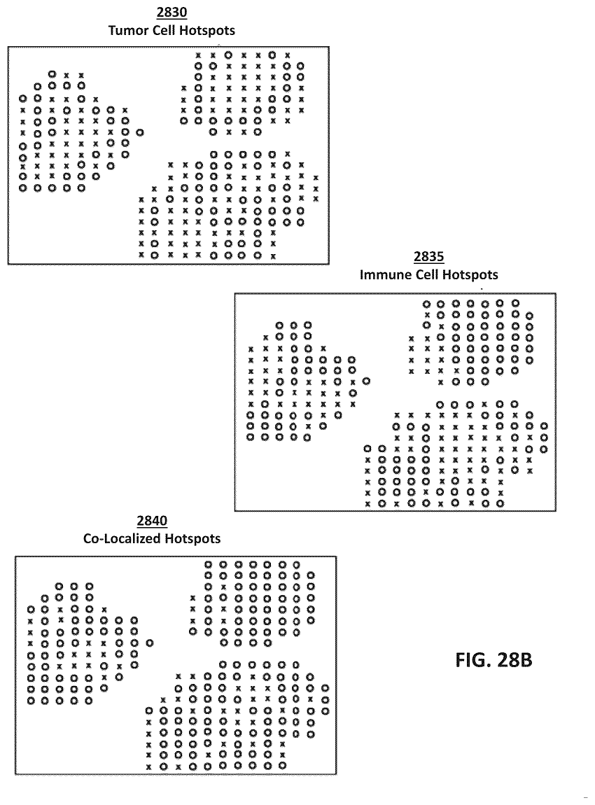

FIG. 28A depicts an application of an areal analysis framework implemented by spatial distribution metric module 1016, in accordance with various embodiments. In particular, the areal analysis framework may be used to process a digital pathology image of a stained sample section. Densities of particular types of biological objects (e.g., tumor cells and T cells) may be detected, as described above, to produce biological object data, an example of which is shown in table 2800. In some embodiments, the output biological object data may include coordinates of individual tiles (e.g., epithelium tiles and stroma tiles) within the lattice formed by tile generation module 1010, and areas of the tiles associated with each of the biological objects of interest. As an example, when the biological objects of interest include immune cells, epithelial cells, and stromal cells (e.g., CD8+ T cells, CK+ tumor cells, CK− tumor cells), the output biological data may include areas of the epithelium tiles (e.g., tiles associated with CK+ tumor cells), areas of the stroma tiles (e.g., tiles associated with CK− tumor cells), areas of the epithelium tiles and the immune cells (e.g., tiles associated with CK+ tumor cells and CD8+ T cells), and areas of the stroma tiles and the immune cells (e.g., tiles associated with CK− tumor cells and CD8+ T cells).

In some embodiments, spatial distribution metric module 1016 may generate a spatial lattice having a defined number of columns and a defined number of rows that can be used to divide the digital pathology image into tiles. For each tile, a number or density of biological object depictions within the region can be identified, such as by using the density accounting techniques described herein. For each biological object type, the collection of region-specific biological object densities—the mapping of which tiles, at which locations, contain specific density values—can be defined as the biological object type's lattice data. FIG. 28A illustrates a particular embodiment of lattice data 2810 for depictions of a first type of biological object, tumor epithelial cells (e.g., CK+ tumor cells), and lattice data 2815 for depictions of a second type of biological object, immune cells (e.g., CD8+ T cells). Each of the lattice data is shown, for purposes of illustration, as being overlaid on a representation of a digital pathology image of a stained section. In some embodiments, lattice data can be defined to include, for each region in the lattice, a prevalence value defined to equal counts for the region divided by total counts across all regions. Thus, regions within which there are no biological objects of a given type may be assigned a prevalence value of 0, while regions within which there is at least one biological object of a given type may be assigned a positive non-zero prevalence value.

In some embodiments, identical amounts of biological objects (e.g., lymphocytes) in two different contexts (e.g., tumors) do not necessarily imply the characterization or degree of characterization (e.g., the same immune infiltration). Instead, how the biological object depictions of a first type are distributed in relation to biological object depictions of a second type can indicate a functional state. Therefore, characterizing proximity of biological object depictions of the same and different types can reflect more information.

The Morisita-Horn Index is an ecological measure of similarity (e.g., overlap) in biological or ecological systems. The Morisita-Horn index (MH) may be used to characterize the bi-variate relationship or co-localization between two populations of biological object depictions (e.g., of two types), and can be defined by Equation 1:

$$M H = \frac{2 {Σ}_{i}^{n} {z}_{i}^{l} {z}_{i}^{t}}{{\sum }_{i}^{n} {( {z}_{i}^{l} )}^{2} + {\sum }_{i}^{n} {( {z}_{i}^{t} )}^{2}} . \qquad Equation 1$$

In Equation 1, zᵢ^{l}, zᵢ^{t} denote the prevalence of biological object depictions of a first type and biological object depictions of a second type at the square grids i, respectively. In FIG. 28A, lattice data 2810 shows exemplary prevalence values zᵢ^{t} of depictions of a first type of biological object across grid points, and lattice data 2815 shows exemplary prevalence values zᵢ^{l} of depictions of a second type of biological object of the across grid points.

The Morisita-Horn Index is defined to be 0 when individual lattice regions do not include biological object depictions of both types (indicating that the distributions of different biological object types are spatially separated). For example, the Morisita-Horn Index would be 0 when considering the illustrative spatially separate distributions or segregated distributions shown in illustrative first scenario 2820. The Morisita-Horn Index is defined to be 1 when a distribution of a first biological object type across lattice regions matches (or is a scaled version of) a distribution of a second biological object type across lattice regions. For example, the Morisita-Horn Index would be close to 1 when considering the illustrative highly co-localized distributions shown in illustrative second scenario 2825.

In the example of FIG. 28A, the Morisita-Horn Index calculated using lattice data 2810 and lattice data 2815 is 0.47. A value of 0.47 may be considered to be a high Morisita-Horn Index value, indicating that the depictions of biological objects of the first type and second type were highly colocalized.

Another spatial distribution metric that may be calculated by spatial distribution metric module 1016 may be the Jaccard index (J) and the Sorensen index (L), which are similar and closely related to each other, can be defined by Equations 2 and 3, respectively:

$$J = \frac{{\sum }_{i}^{n} min ( {z}_{i}^{l} , {z}_{i}^{t} )}{{\sum }_{i}^{n} ( {z}_{i}^{l} + {z}_{i}^{t} ) - {Σ}_{i}^{n} min ( {z}_{i}^{l} , {z}_{i}^{t} )} , \qquad Equation 2$$

$$L = \frac{2 {\sum }_{i}^{n} min ( {z}_{i}^{l} , {z}_{i}^{t} )}{{\sum }_{i}^{n} ( {z}_{i}^{l} + {z}_{i}^{t} )} . \qquad Equation 3$$

In Equations 2 and 3, where zᵢ^{l}, zᵢ^{t} denotes the prevalence of biological object depictions of a first type and biological object depictions of a second type at the square grids i, respectively, and min(a, b) returns the minimum value between a and b.

Another spatial distribution metric that can characterize a spatial distribution of biological object depictions is Moran's Index, which is a measure of spatial autocorrelation. Moran's Index is the correlation coefficient for the relationship between a first variable and a second variable at neighboring spatial units. The first variable can be defined as prevalence of depictions of biological objects of a first type and the second variable can be defined as prevalence of depictions of biological objects of a second type, so as to quantify the extent that the two types of biological object depictions are interspersed in digital pathology images.

Moran's Index, I, can be defined using Equation 4:

$$I = \frac{n}{{\sum }_{i}^{n} {Σ}_{j}^{n} {w}_{i j}} ( {\sum }_{i}^{n} {\sum }_{j}^{n} {w}_{ij} ( {x}_{i} ) ( {y}_{j} ) ) . \qquad Equation 4$$

In Equation 4, xᵢ, yᵢ denote the standardized prevalence of biological object depictions of the first type (e.g., tumor cells) at areal unit i, and the standardized prevalence of biological object depictions of the second type (e.g., lymphocytes) at areal unit j. The wᵢⱼ is the binary weight for areal units i and j−wᵢⱼ is 1 if two units neighbor, and 0 otherwise. A first-order scheme can be used to define neighborhood structure. Moran's I can be derived separately for biological object depictions of different types of biological objects.

Moran's Index is defined to be equal to −1 when biological object depictions are perfectly dispersed across a lattice (and thus having a negative spatial autocorrelation); and to be 1 when biological object depictions are tightly clustered (and thus having a positive autocorrelation). Moran's Index is defined to be 0 when an object distribution matches a random distribution. The areal representation of particular biological object depiction types thus facilitates generating a grid that supports calculation of a Moran's Index for each biological object type. In some embodiments, in which two or more types of biological object depictions are being identified and tracked, a difference between the Moran's Index calculated for each of the two or more types of biological object depictions can provide an indication of colocation (e.g., with differences near zero indicating colocation) between those types of biological object depictions.

Yet another example spatial distribution metric is Geary's C, also known as Geary's contiguity ratio, which is measure of spatial autocorrelation or an attempt to determine if adjacent observations of the same phenomenon are correlated. Geary's C is inversely related to Moran's Index, but it is not identical. While Moran's Index is a measure of global spatial autocorrelation, Geary's C is more sensitive to local spatial autocorrelation. Geary's C can be defined using Equation 5:

$$C = \frac{n - 1}{2 {Σ}_{i}^{n} {Σ}_{j}^{n} {w}_{ij}} \frac{{Σ}_{i}^{n} {Σ}_{j}^{n} {{w}_{ij} ( {z}_{i} - {z}_{j} )}^{2}}{{{Σ}_{i}^{n} ( {z}_{i} - {z}_{j} )}^{2}} . \qquad Equation 5$$

In Equation 5, zᵢ, zⱼ denote the prevalence of either biological object depictions of a first type or a second type at the square grids i, j, and wᵢⱼ is the same as defined aforementioned.

Still yet another spatial distribution metric that can characterize a spatial distribution of biological object depictions is the Bhattacharyya coefficient (“B coefficient”), which is an approximate measure of an overlap between two statistical samples. In general, the B coefficient can be used to determine the relative closeness of the two statistical samples. It is used to measure the separability of classes in the classification.

Given probability distributions p and q over the same domain X (e.g., distributions of depictions of two types of biological objects within the same digital pathology image), the B coefficient is defined using Equation 6:

$$BC ( p , q ) = {\sum }_{x ∈ x} \sqrt{p ( x ) q ( x )} . \qquad Equation 6$$

In Equation 6, 0≤BC≤1. The Bhattacharyya coefficient is used to determine the Bhattacharyya, D_{B} (p, q)=−ln(BC(p, q)), where 0≤D_{B}≤∞. Note that DB does not obey the triangle inequality, but the Hellinger distance, \sqrt{1−BC(p, q)} does obey the triangle inequality. The B coefficient increases with the number of partitions in the domain that have members from both samples (e.g., with the number of tiles in the digital pathology image that have depictions or suitable density of two or more types of biological object depictions). The B coefficient is therefore larger still with each partition in the domain that has a significant overlap of the samples, e.g., with each partition that contains a large number of the members of the two samples. The choice of the number of partitions is variable and can be customized to the number of members in each sample. To maintain accuracy, care is taken to avoid selecting too few partitions and overestimating the overlap region as well as taking too many partitions and creating partitions with no members despite being in a densely populated sample space. The B coefficient will be 0 if there is no overlap at all between the two samples of biological object depictions.

Returning to FIG. 28A, lattice data 2810 and lattice data 2815 can be further processed to generate hotspot data 2830 corresponding to detected depictions of a first type of biological object and hotspot data 2835 corresponding to detected depictions of a second type of biological object, respectively.

In FIG. 28B, hotspot data 2830 and hotspot data 2835 indicate the regions that were determined to be hotspots for the respective types of detected depictions of biological objects. The regions that were detected as hotspots are shown as circles and the regions that were determined not to be hotspots as an ‘x.’ Hotspot data 2830, 2835 is defined for each region associated with a non-zero object count. Hotspot data 2830, 2835 can also include binary values that indicate whether a given region was identified as being a hotspot or not. In addition to hotspot data and analysis, cold spot data and analysis can be conducted.

With respect to depictions of biological objects, hotspot data 2830, 2835 can be generated for each biological object type by determining a Getis-Ord local statistic for each region associated with a non-zero object count for the biological object type. Getis-Ord hotspot/cold spot analysis can be used to identify statistically significant hotspots/cold spots of tumor cells or lymphocytes, where hotspots are the areal units with a statistically significantly high value of prevalence of depictions of biological objects compared to the neighboring areal units and cold spots are the areal units with a statistically significantly low value of prevalence of depictions of biological objects compared to neighboring areal units. The value and determination what makes a hotspot/cold spot region compared the neighboring regions can be selected according to user preference, and, in particular can be selected according to a rules-based approach or learned model. For example, the number and/or type of biological object depictions detected, the absolute number of depictions, and other factors can be considered. The Getis-Ord local statistic is a z-score and can be defined, for a square grid i using Equation 7:

$${G}_{i}^{*} = \frac{{\sum }_{j = 1}^{n} {ω}_{i j} {z}_{j} - \overline{z} {\sum }_{j = 1}^{n} {ω}_{i j}}{s \sqrt{\frac{n {\sum }_{j = 1}^{n} {ω}_{i j}^{2} - {( {\sum }_{j = 1}^{n} {ω}_{i j} )}^{2}}{n - 1}}} . \qquad Equation 7$$

In Equation 7, i represents an individual region (specific row-column combination) in the lattice, n is the number of row and column combinations (i.e., number of regions) in the lattice, ωᵢⱼ is the spatial weight between i and j, zⱼ is the prevalence of biological object depictions of a given type in a region, z̅ is the average object prevalence of the given type across regions, and S is defined by Equation 8:

$$S = \sqrt{\frac{{\sum }_{j = 1}^{n} {z}_{j}^{2}}{n} - {( \overline{z} )}^{2}} . \qquad Equation 8$$

The Getis-Ord local statistics can be transformed to binary values by determining whether each statistic exceeds a threshold. For example, a threshold can be set to 0.16. The threshold can be selected according to user preference, and in particular can be set according to rule-based or machine-learned approaches.

A logical AND function can be used to identify the regions that are identified as being a hotspot for more than one type of depictions of biological objects. For example, colocalized hotspot data 2840 indicates the regions that were identified as being a hotspot for two types of biological object depictions (shown as circles symbols in FIG. 28B). A high ratio of a number of regions identified as being a co-localized hotspot relative to a number of hotspot regions identified for a given object type (e.g., for tumor-cell objects) can indicate that biological object depictions of the given type share spatial characteristics with the other object type. Meanwhile, a low ratio at or near zero can be consistent with spatial segregation of biological objects of the different types.

Returning to FIG. 10, spatial distribution representation module 1018 may be configured to generate a spatial density representation based on the one spatial distribution metrics generated by spatial distribution metric module 1016. In some embodiments, the spatial distribution metrics used to generate the spatial distribution representation may include:
Spatial co-localization features of CK and CD8 in epithelium tiles (e.g., CK+ tiles) and stroma tiles (e.g., CK− tiles)
   B coefficient;
   Morisita-Horn Index;
   Jaccard Index;
   Sorensen index;
   Moran's Index;
   Co-localized Getis-Ord hotspot;
   CD8/CK area ratio;
Spatial distribution features:
   CK and CD8 density in epithelium (e.g., CK+ tiles) and stroma tiles (e.g., CK− tiles), respectively;
   Local and global Moran's Index;
   Local and global Geary's C Index;
   Local and global Getis-Ord hotspot;
Intra-tumor lymphocyte ratio;
The ratio of co-localized spots (e.g., hotspots, cold spots, non-significant spots) for the type of biological object depictions over the number of spots (e.g., hotspots, cold spots, non-significant spots) for a first type of the biological object depictions, with spots (e.g., hotspots, cold spots, non-significant spots) defined using Getis-Ord local statistics; and
Features obtained by variogram fitting of two types of biological object depictions (e.g., tumor cells and lymphocytes).

In some embodiments, the spatial distribution representation may be a feature vector having M elements. Each element may correspond to one of the spatial distribution metrics and/or a parameter of a spatial distribution metric. For example, the spatial density representation may be a 50-dimensional feature vector. In some embodiments, an embedding may be generated based on the spatial density representation. The embedding may be mapped into an embedding space using a trained encoder. In this example, the tumor immunophenotype may be determined based on a distance between the mapped location of the embedding in the embedding space and one or more clusters of embeddings, each cluster being associated with a tumor immunophenotype.

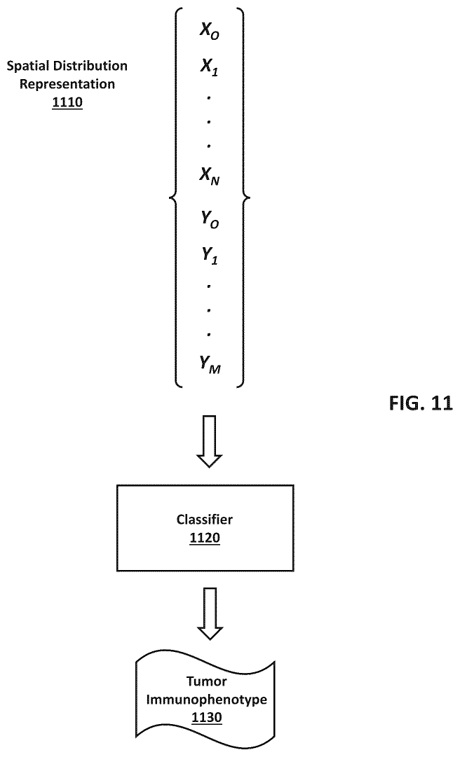

Classification module 1020 may be configured to implement a classifier trained to determine a tumor immunophenotype for a digital pathology image depicting a tumor based on a spatial distribution representation generated for the digital pathology image. Classification module 1020 may be configured to implement a classifier, such as classifier 1120 of FIG. 11. Classifier 1120 may receive a spatial distribution representation 1110 and input spatial distribution representation 1110 into classifier 1120. In some embodiments, classifier 1120 may be trained to output a tumor immunophenotype (e.g., tumor immunophenotype 1130) of the biological sample depicted by a digital pathology image (e.g., digital pathology image 604 of FIG. 6). In some embodiments, the classifier is a multi-class classifier configured to classify an image into one of a set of tumor immunophenotypes. The number of classes may depend on the number of tumor immunophenotype classifications. For example, classifier 1120 may be trained to classify an image into one of a set of tumor immunophenotypes based on spatial distribution presentation 1110. The set of tumor immunophenotypes may include the tumor immunophenotypes of desert, excluded, and inflamed. In some embodiments, classifier 1120 implemented by classification module 1020 may be a support vector machine (SVM), a random forest (RF) classifier, a decision tree classifier, a logistic regression classifier, a k-nearest neighbor (kNN) classifier, or another type of classifier.

Classifier training module 1022 may be configured to train the classifier used by classification module 1020 to predict a tumor immunophenotype of an image of a tumor based on a spatial distribution representation generated for that image. In some embodiments, classifier training module 1022 may train the classifier using training data generated from a first plurality of patients participating in a first clinical trial. Classifier training module 1022 may further be configured to validate the classifier using validation data generated from a second plurality of patients participating in a second clinical trial.

In some embodiments, classifier training module 1022 may access a first plurality of images from image database 142. The first plurality of images may include images of tumors from patients participating in a first clinical trial. For example, the first clinical trial may include patients having advanced NSCLC who have progressed on a platinum-based chemotherapy regiment and who were 1:1 randomized to receive either a first immunotherapy (e.g., atezolizumab) or a second immunotherapy (e.g., docetaxel). The number of patients participating in the first clinical trial may be 100 or more patients, 200 or more patients, 300 or more patients, etc. In some embodiments, biological samples may be obtained for at least some of the patients of the first clinical trial. One biological sample may be obtained for each of the patients. The biological samples may include tumor lesions including tumor stroma and tumor epithelium. The biological samples may be stained using a dual-stain, such as a panCK-CD8 stain, to highlight tumor epithelium, tumor stroma, and immune cells.

Each sample may be imaged using image scanner 240 to obtain a first plurality of images. These images may be stored in image database 142. The images may be digital pathology images, such as whole-slide images. In some embodiments, classifier training module 1022 and/or other components of third pipeline subsystem 116 may be configured to divide each of the first plurality of images into tiles and identify which of those tiles are epithelium tiles and/or stroma tiles. For each image, an epithelium-immune cell density may be calculated for the epithelium tiles and a stroma-immune cell density may be calculated for the stroma tiles. One or more spatial distribution metrics may be calculated based on the epithelium-immune cell densities and the stroma-immune cell densities. A spatial distribution representation may be generated for each image based on that image's corresponding spatial distribution metrics. Therefore, for each of the first plurality of images, a corresponding spatial distribution representation may be obtained.

In some embodiments, classifier training module 1022 may be configured to generate training data including the first plurality of images and, for each image, a label indicating a predetermined tumor immunophenotype of the tumor depicted by that image. In some embodiments, the predetermined tumor immunophenotype may be determined by a trained pathologist. The training data may be stored in training data database 144. Classifier training module 1022 may select a classifier to be trained from model database 146 and may be configured to train the classifier using the training data stored in training data database 144. The classifier may be a multi-class classifier, such as a three-class random forest decision tree classifier. Classifier training module 1022 may optimize hyperparameters of the classifier using an optimizer, such as the Adam optimizer.

After the classifier has been trained using the training data, it may be evaluated using validation data. Classifier training module 1022 may be configured to generate the validation data using a second plurality of images. The second plurality of images may also be stored in image database 142. The second plurality of images may include images of tumors from patients participating in a second clinical trial. As an example, the second clinical trial may include patients having advanced NSCLC. As another example, the second clinical trial may include patients having metastatic TNBC who were randomized to receive a first therapy (e.g., atezolizumab plus nab-paclitaxel) or a second therapy (e.g., a placebo plus nab-paclitaxel). The number of patients participating in the first clinical trial may be 500 or more patients, 750 or more patients, 1,000 or more patients, etc. In some embodiments, biological samples may be obtained for at least some of the patients of the second clinical trial. One biological sample may be obtained for each of the patients. The biological samples may include tumor lesions including tumor stroma and tumor epithelium. The biological samples may be stained using a dual-stain, such as a panCK-CD8 stain, to highlight tumor epithelium, tumor stroma, and immune cells. Similar to the process described above for the first clinical trial, validation data may be generated based on a second plurality of images of the biological samples of at least some of the patients of the second clinical trial. The second plurality of images may be used to generate spatial distribution representations. In some embodiments, labels indicating a predetermined tumor immunophenotype of the biological sample may be assigned by a trained pathologist. Classifier training module 1022 may use the validation data to evaluate the accuracy of the trained classifier. If the classifier does not predict the tumor immunophenotype with at least a threshold level of accuracy, then classifier training module 1022 may retrain the classifier. However, if the classifier is determined to have a threshold level of accuracy, it may be deployed for determining a tumor immunophenotype of an input image. The classifier may output a tumor immunophenotype based on the spatial distribution representation, which may be derived from a digital pathology image of a tumor (e.g., tumor immunophenotype 1130 output by classifier 1120 based on spatial distribution representation 1110, which may be generated based on digital pathology image 604 of biological sample 602).

In some embodiments, the tumor immunophenotype may be one of a set of tumor immunophenotypes. For example, the tumor immunophenotypes may include desert, excluded, and inflamed. In some embodiments, a tumor depicted by a digital pathology image may be classified as the tumor immunophenotype desert based on an epithelium-immune cell density calculated for that image satisfying a desert epithelium-immune cell density threshold criterion and a stroma-immune cell density for that image also satisfying a desert stroma-immune cell density threshold criterion. As an example, the desert epithelium-immune cell density threshold criterion being satisfied may include the epithelium-immune cell density being within a first threshold range of epithelium-immune cell densities and the desert stroma-immune cell density threshold criterion being satisfied may include the stroma-immune cell density being within a first threshold range of stroma-immune cell densities. A tumor depicted by a digital pathology image may be classifies as the tumor immunophenotype excluded based on an epithelium-immune cell density for that image satisfying an excluded epithelium-immune cell density threshold criterion and a stroma-immune cell density for that image satisfying an excluded stroma-immune cell density threshold criterion. For example, the excluded epithelium-immune cell density threshold criterion being satisfied may include the epithelium-immune cell density being within a second threshold range of epithelium-immune cell densities and the excluded stroma-immune cell density threshold criterion being satisfied may include the stroma-immune cell density being within a second threshold range of stroma-immune cell densities. A tumor depicted by a digital pathology image may be classifies as the tumor immunophenotype inflamed based on an epithelium-immune cell density of that image satisfying an inflamed epithelium-immune cell density threshold criterion and a stroma-immune cell density of that image satisfying an inflamed stroma-immune cell density threshold criterion. For example, the inflamed epithelium-immune cell density threshold criterion being satisfied may include the epithelium-immune cell density being within a third threshold range of epithelium-immune cell densities and the inflamed stroma-immune cell density threshold criterion being satisfied may include the stroma-immune cell density being within a third threshold range of stroma-immune cell densities.

The metrics chosen can correspond to multiple frameworks (e.g., areal-process analysis framework). For each subject, a label can be defined to indicate secondary determinations, such as object density metrics and/or assigned immunophenotype. The machine-learning models, including but not limited to a logistic-regression model, can be trained and tested with the paired input data and labels, using repeated nested cross-validation. As an example, for each of 5 data folds, the model can be trained on the remaining 4 folds and tested on the remaining fold to calculate an area under an ROC.

In embodiments with limited sample size, adaptable techniques to evaluate model performance can be used. As a non-limiting example, a nested Monte Carlo Cross Validation (nMCCV) can be used to evaluate the model performance. The same enrichment procedure can be repeated B times by randomly splitting with the same proportion between training, validation, and test sets, to produce an ensemble of score function and threshold {(S̅_{b},\hat{q}ⱼ)}_{b=1}^{B}. For the i-th subject, the ensembled responder status can be evaluated by averaging, among the repetitions where i is randomized to test set, the membership of responder group for i, and thresholding by 0.5. Hazard ratio or Odds ratio together with a 95% confidence interval and p-value can be calculated on the aggregated test subjects.

After spatial distribution metric module 1016 generates the spatial distribution metrics, the spatial-distribution metrics, density values, and other generated data can be used to assign an immunophenotype to the sample. As described herein, the designation of the immunophenotype can be provided by a machine-learned model trained in a supervising training process where labeled digital pathology images are provided along with their spatial-distribution metrics. Through the training process, the classifier implemented by classification module 1020 can learn to categorize digital pathology images using classifier training module 1022 and their corresponding samples, into selected immunophenotyping groups.

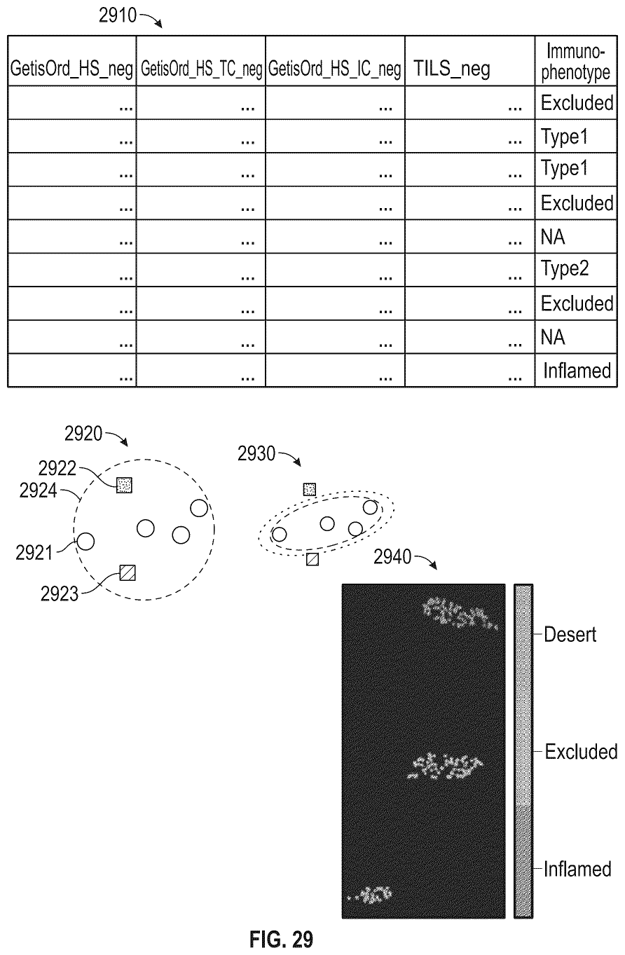

FIG. 29 illustrates one visualization of the training and use of the machine-learned models implemented by classification module 1020. Classifier training module 1022 may generate training data for training the classifier. The training set may include values for the various spatial distribution metrics discussed herein. For training purposes, training data set 2910 may include an immunophenotype that has been assigned to the samples, such as manually by a pathologist. Each digital pathology image can be projected into a multi-variable space, with an axis for each of the spatial-distribution metrics and/or variations or derivation therefrom. With the supplied labels, classifier training module 1022 can train a machine-learned model (e.g., a classifier) to identify clusters of the digital pathology images (and corresponding samples) within the multi-variable space. In this formulation, the task of labeling a previously unseen data point can be approximated by determining to which cluster the new data point belong.

FIG. 29 further illustrates challenges of identifying the proper mechanism for identifying the clustering criteria. Plot 2920 shows data points for several digital pathology images, plotted on a two-dimensional Cartesian grid. Circular points 2921 designate a first type of label, squares points designate two different labels that are each distinct from the first type of label. The label can equate to an immunophenotype. A first attempt to group the points can involve, for example, a Euclidean nearest neighbor approach. In such an approach, all points within a certain radius 2924 can be labeled with the target label type. While in this example this neighborhood indeed captures all of the points 2921 associated with the first type of label, it also captures the two imposter data points 2922 and 2923 (illustrated as squares). To accurately capture only points associated with the first label type within the neighborhood, additional measures of similarity may be used. In one example, this can include the consideration of additional metrics (e.g., adding additional axes of similarity). As such in plot 2930, a hyperplane through the multi-variable space represented by the data (e.g., in training data set 2910), can effectively differentiate target points 2921 from the imposter data points 2922 and 2923. Moreover, additional distance metrics besides the Euclidean distance metric can be used to define the nearest neighbors and the resulting clusters.

Plot 2940 shows the idealized result, especially in comparison to plot 2700 shown in FIG. 27. In plot 2940, neat groupings of the data have been identified, differentiating between, in this example, desert, excluded, and inflamed immunophenotypes based on the input data. The relationship between the spatial-distance metrics used to create these groupings and the groupings themselves can more clearly be seen than using mere density measurements alone (as shown in plot 2700 of FIG. 27).

In some embodiments, classifier training module 1022 can train a machine-learning model, such as a classifier, to process a digital pathology image of a biopsy section from a subject, to predict an assessment of a condition of the subject from the digital pathology image. As an example, using the techniques described herein, third pipeline subsystem 116 can generate a variety of spatial-distribution metrics and predict an immunophenotype for the digital pathology image. From this input, a regression machine-learning model can be trained predict, for example, suspected patient outcomes, assessments of related patient condition factors, availability or eligibility for selected treatments, and other related recommendations.

A biopsy can be collected from each of multiple subjects having the condition. The sample can be fixed, embedded, sliced, stained, and imaged according to the subject matter disclosed herein. Depictions and densities of specified types of biological objects (e.g., tumor cells, lymphocytes), can be detected. Classifier training module 1022 of third pipeline subsystem 116 can use a trained set of machine-learned models to process images to quantify the density of biological objects of interest. For each subject of the multiple subjects, a label can be generated so as to indicate whether the condition exhibited specified features and/or indicate certain secondary labels (e.g., immunophenotype) applied by third pipeline subsystem 116. In the context of predicting an overall assessment of the condition of the subject, labels such as immunophenotype may be considered secondary as they can inform the overall assessment.

In some embodiments, the machine learning techniques that can be used in the systems/subsystems/modules of third pipeline subsystem 116 may include, but are not limited to (which is not to suggest that any other list is limiting), any of the following: Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, or separable convolutions (e.g., depth-separable convolutions, spatial separable convolutions).

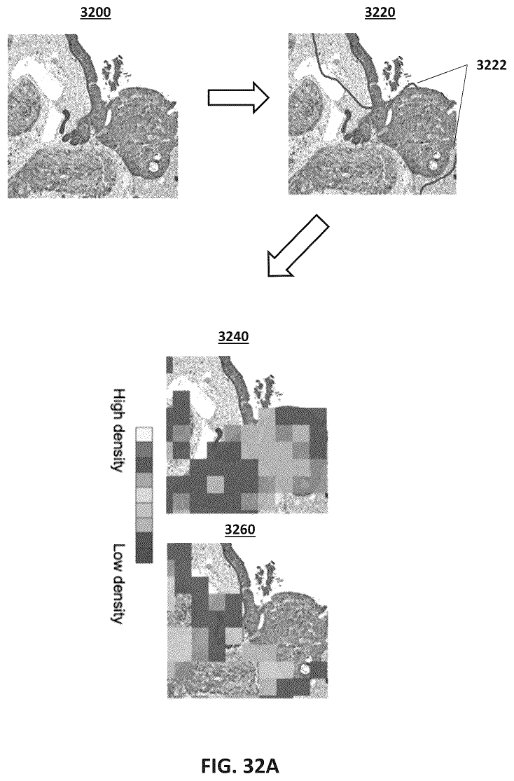

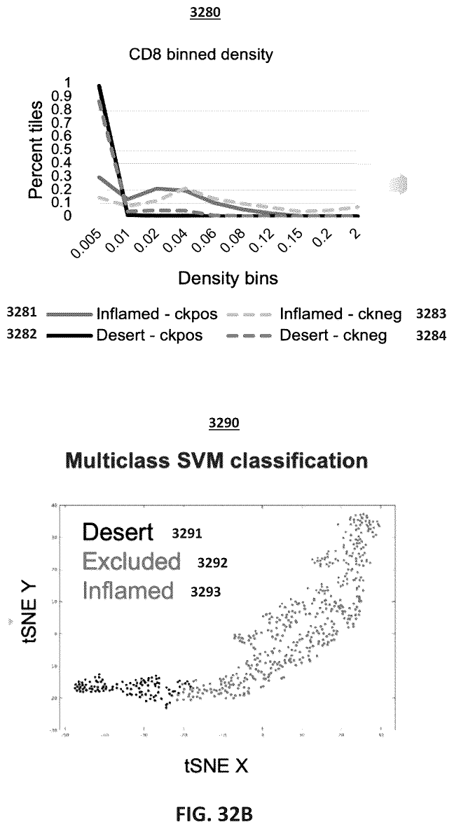

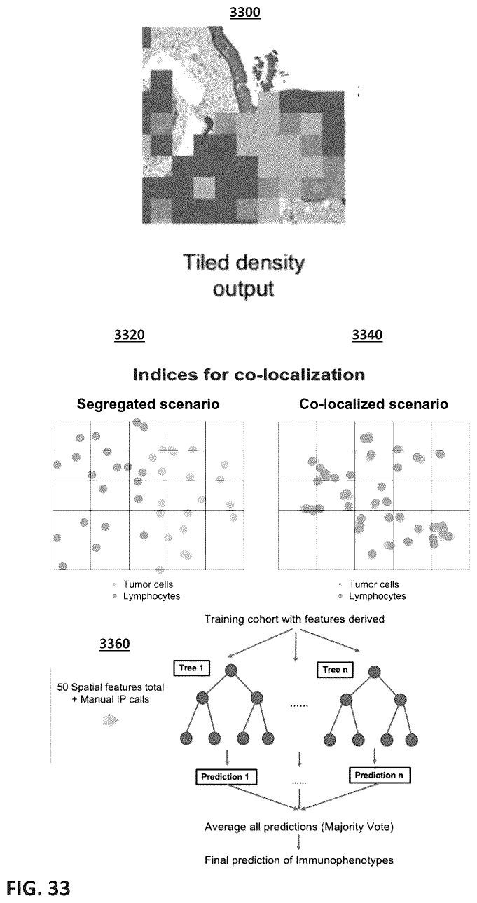

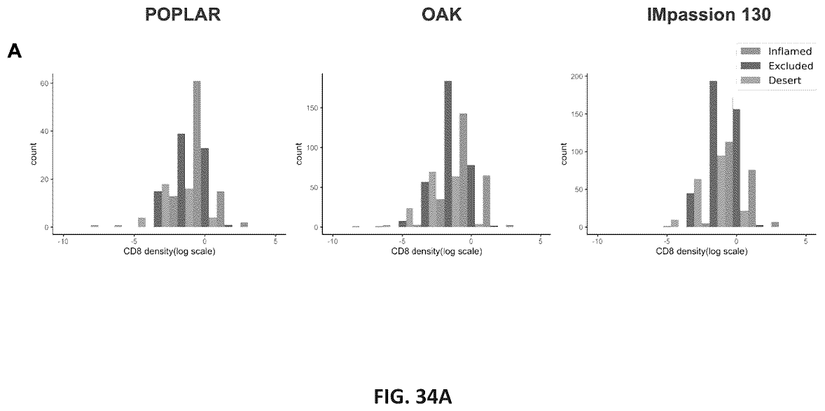

FIGS. 32A-32B illustrates an example of a tile-based analysis of immune cell density within regions of tumor epithelium and tumor stroma, as well as a tumor immunophenotype output by a classifier based on the tile-based analysis, in accordance with various embodiments. For example, image 3200 may represent a whole slide image (or a portion thereof). A trained pathologist and/or machine learning model may generate annotated image 3220 including annotations (e.g., the blue line 3222) delineating regions of a biological sample including tumor and regions not including tumor. Image 3240 represents an epithelium-immune cell density for epithelium tiles within annotated image 3220. For example, the color legend indicates the density value of the epithelium-immune cell density of a given epithelium tile (e.g., an average density of CD8+ T cells in CK+ tiles). Image 3260 represents a stroma-immune cell density for stroma tiles within annotated image 3220. For example, the color legend indicates the density value of the stroma-immune cell density of a given stroma tile (e.g., an average density of CD8+ T cells in CK− tiles). The third pipeline implemented by third pipeline subsystem 116 may evaluate both the more granular spatial pattern and density of immune infiltrates. For example, as described above and further exemplified in FIGS. 32A-32B, epithelial cell densities, stromal cell densities, epithelium-immune cell densities, and stroma-immune cell densities may be calculated in each tile of digital pathology image 3200 (or annotated image 3220). For example, image 3240 illustrates epithelial cell densities for tiles of a tumor depicted by annotated image 3220, and image 3260 illustrates stromal cell densities for stromal tiles of annotated image 3220. Plot 3280 illustrates an immune cell binned density plot as a function of the density ranges of the bins distributed within the epithelium set of bins and the stroma set of bins. As seen in plot 3280, the tumor immunophenotypes of inflamed and desert are depicted for epithelium tiles (e.g., CK+ tiles represented by solid orange line 3281 and solid black line 3282, respectively) and stroma tiles (e.g., CK− tiles represented by dashed orange line 3283 and dashed black line 3284 respectively). Graph 3290 of FIG. 32B illustrates an example UMAP distribution of the outputs of classifier implemented by the second pipeline, with the black data points 3291 corresponding to the tumor immunophenotype desert, the pink data points 3292 corresponding to the tumor immunophenotype excluded, and the orange data points 3293 corresponding to the tumor immunophenotype inflamed.

A two-dimensional density distribution 3300, as seen in FIG. 33, may be generated indicating a density of tumor cells and immune cells. To quantitate spatial patterns of epithelial cells and/or stromal cells with immune cells, such as cell co-localization indices 3320 and 3340 of FIG. 33, a presence of cell “hotspots” generated. A collection of lattice-based spatial distribution metrics may be computed, as described above, using the third pipeline. From the spatial distribution metrics, a spatial distribution representation may be generated. The spatial distribution metric may be a 50-dimensional feature vector, as seen in graph 3360, which can be used to predict a tumor immunophenotype. The spatial distribution metric may be calculated for each image of a plurality of images of tumors from patients participating in the example first clinical trial (as described above). The spatial distribution metrics may be calculated and, along with manually assigned tumor immunophenotype labels for those images, used to train a classifier to predict tumor immunophenotype. The trained classifier may then be validated using spatial distribution representations generated from images of tumors from patients participating in the example second clinical trial and/or the example third clinical trial.

As seen from the row C of plots (as shown in FIG. 34C) from FIGS. 34A-34D, these spatial distribution representation features (e.g., the elements of the spatial distribution representation) alone do not clearly differentiate samples into the set of tumor immunophenotypes (e.g., desert, excluded, inflamed). In some embodiments, the classifier from the third pipeline, trained on the example first clinical trial, may relates spatial features to both manual tumor immunophenotype calls and patient immunotherapy responses. For example, as seen from the third row of density plots 3500 from FIG. 35A, the tumor immunophenotyping for the example second clinical trial produced a weighted Cohen's kappa of 0.573, and the example third clinical trial produced a weighted Cohen's kappa of 0.456. Furthermore, the distribution of manually determined tumor immunophenotypes with those determined via the third pipeline is illustrated from the bar charts of FIGS. 35A-35C for the example second clinical trial and the example third clinical trial.

In some embodiments, the third pipeline implemented by third pipeline subsystem 116 can use a weakly supervised multiple instance learning-based classification approach to predict tumor immunophenotypes. For example, the third pipeline can involve dividing, using the tile generation module 1010, a histology image into a plurality of tiles. Each tile can depict a distinct structure within the imaged tissue and can be referred to as an “instance.” The histology image, treated as a collective entity, can serve as a “bag” encompassing multiple instances, and can be labeled as “positive” or “negative” for association with a tumor immunophenotype. In some embodiments, the third pipeline can involve determining, using the classification module 1020 and/or the classifier training module 1022, a tumor immunophenotype (e.g., tumor immunophenotype 1130) of the histology image using a classifier (e.g., classifier 1120) trained via multiple instance learning. In some embodiments, an attention score mechanism can be used to identify which instances within a bag contribute significantly to making a positive prediction and/or label. This attention-score-based process can aid in emphasizing the most relevant and discriminative regions within the histology image, contributing to the model and/or algorithm's ability to discern intricate patterns associated with the tumor immunophenotype. For example, attention scores can be derived from the features of each instance (e.g., by assessing their similarity in relation to features of other instances within the same bag). Instances that are more similar to other instances may be less strongly associated with a positive label, thereby having a lower attention score. For example, in a histology image containing only healthy cells, the healthy cells may all resemble one another, and there may be no reason to pay greater attention to one image tile over another. Conversely, instances that are more distinct may be more strongly associated with a positive label, thereby having a higher attention score. For example, in a histology image containing both healthy cells and tumor cells, the tumor cells may stand out from the healthy cells, and the multiple instance machine-learning model may pay greater attention to the image tiles containing the standout tumor cells when labeling the bag as “positive.” An instance-based model and/or instance-based algorithm can be trained to predict the tumor immunophenotype based on the attention scores. For example, a histology image containing both healthy cells and tumor cells may be labeled as “positive” based on the high attention scores associated with image tiles depicting tumor cells within the histology image, and the overall histology image can be labeled as containing a specific tumor type as a result.

### Fourth Pipeline

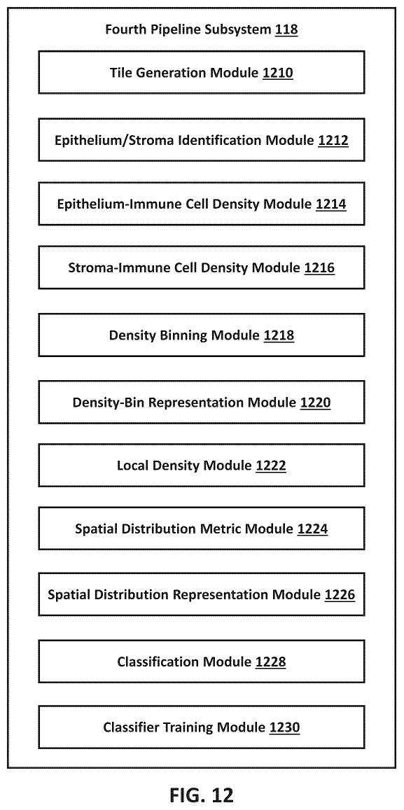

FIG. 12 illustrates an example fourth pipeline subsystem 118, in accordance with various embodiments. Fourth pipeline subsystem 118 may implement a fourth pipeline for determining a tumor immunophenotype of a tumor depicted by a digital pathology image. Fourth pipeline subsystem 118 may include a tile generation module 1210, an epithelium/stroma identification module 1212, an epithelium-immune cell density module 1214, a stroma-immune cell density module 1216, a density binning module 1218, a density-bin representation module 1220, a local density module 1222, a spatial distribution metric module 1224, a spatial distribution representation module 1226, a classification module 1228, a classifier training module 1230, or other components.

In some embodiments, tile generation module 1210 may be configured to receive an image depicting a tumor and divide the image into a plurality of tiles. The image may be a digital pathology image captured using a digital pathology imaging system, such as scanner 240. As an example, with reference to FIG. 6, tile generation module 1210 may receive a digital pathology image 604 of a biological sample 602 that has been imaged by image scanner 240 (previously described with reference to FIG. 2). Tile generation module 1210 may segment digital pathology image 604 into tiles 606. In some embodiments, tiles 606 can be non-overlapping (e.g., each tile includes pixels of digital pathology image 604 not included in any other tile) or overlapping (e.g., each tile includes some portion of pixels of digital pathology image 604 that are included in at least one other tile). Features such as whether tiles 606 overlap, in addition to a size of each tile and the stride window (e.g., the image distance or number of pixels between a tile and a subsequent tile) can increase or decrease the data set for analysis, with more tiles (e.g., through overlapping or smaller tiles) increasing the potential resolution of eventual outputs and visualizations. In some embodiments, tile generation module 1210 may define a set of tiles for an image where each tile is of a predefined size and/or an offset between tiles is predefined. Furthermore, tile generation module 1210 may generate multiple sets of tiles of varying size, overlap, step size, etc., for each digital pathology image. In some embodiments, digital pathology image 604 itself can contain tile overlap, which may result from the imaging technique. In some embodiments, tile segmentation without overlapping tiles can balance tile processing requirements and can avoid influencing embedding generation and/or weighting value generation. A tile size or tile offset can be determined, for example, by calculating one or more performance metrics (e.g., precision, recall, accuracy, and/or error) for each size/offset and by selecting a tile size and/or offset associated with one or more performance metrics above a predetermined threshold and/or associated with one or more optimal (e.g., high precision, highest recall, highest accuracy, and/or lowest error) performance metric(s).

Tile generation module 1210 may further be configured to define a tile size. The tile size may be determined based on a type of abnormality being detected. For example, tile generation module 1210 may be configured to set the tile size for segmentation of digital pathology image 604 based on the types of tissue abnormalities present in biological sample 602. Tile generation module 1210 may also customize the tile size based on the tissue abnormalities to be detected/searched for to optimize detection. In some embodiments, tile generation module 1210 may determine that, when the tissue abnormalities include inflammation or necrosis in lung tissue, the tile size should be reduced to increase the scanning rate. In some embodiments, tile generation module 1210 may determine that, when the tissue abnormalities include abnormalities with Kupffer cells in liver tissues, the tile size should be increased to increase the opportunities for fourth pipeline subsystem 118 to analyze the Kupffer cells holistically. In some embodiments, tile generation module 1210 may define a set of tiles where a number of tiles in the set, a size of the tiles of the set, a resolution of the tiles for the set, or other related properties, for each image may be defined and held constant for each of one or more images. As an example, each of tiles 606 may have a size of approximately 16,000 μm².

In some embodiments, tile generation module 1210 may be configured to receive digital pathology image 604 of biological sample 602 (e.g., a tumor). Digital pathology image 604 of biological sample 602 may include (at least a portion of) a whole slide image (WSI). For example, as mentioned above, digital pathology image generation subsystem 110 may be configured to produce a WSI of a biological sample (e.g., a tumor). In some embodiments, digital pathology image generation subsystem 110 may generate multiple digital pathology images of biological sample 602 at different settings. For example, image scanner 240 may capture images of biological sample 602 at multiple magnification levels (e.g., 5×, 10×, 20×, 40×, etc.). These images may be provided to fourth pipeline subsystem 118 as a stack, and an operator may determine which image or images from the stack to be used for the subsequent analysis. The biological sample may be prepared, sliced, stained, and subsequently imaged to produce the WSI. The biological sample may include a biopsy of a tumor. For example, the biological sample may include tumors from NSCLC clinical trials and/or TNBC clinical trials.

In some embodiments, a region of interest of digital pathology image 604 may be identified prior to tiling. For example, a pathologist may manually define the region of interest (ROI) in the tumor. The ROI may be defined using a digital pathology image viewing system at a particular magnification (e.g., 4×). As another example, a machine learning model may be used to define the ROI in the tumor lesion. In this example, a human (e.g., pathologist) may be able to review the machine-defined ROI (e.g., to confirm that the defined ROI is accurate). The defined ROI may exclude areas of necrosis. This is important because some staining agents can label normal epithelial cells, not just tumor epithelium.

In some embodiments, one or more stains may be applied to biological sample 602 prior to digital pathology image 604 being captured by image scanner 240. The stains cause different objects of biological sample 602 to turn different colors. For example, one stain (e.g., CD8) may cause immune cells to turn one color, while another stain (e.g., panCK) may cause tumor epithelium and tumor stroma to turn another color. Therefore, the first stain may highlight immune cells, whereas the second stain may highlight, as well as distinguish, tumor epithelium and tumor stroma.

In some embodiments, a color deconvolution may be performed to digital pathology image 604. The color deconvolution may separate out each color channel images from digital pathology image 604, obtaining a plurality of color channel images. In this example, tiles 606 can be produced for each color channel image. In some embodiments, a hue-saturation-value (HSV) thresholding model may be used to isolate the color channel images. Each color channel image may highlight different biological objects. For example, a first color channel image may highlight and distinguish epithelial cells forming tumor epithelium and stromal cells forming tumor stroma and a second color channel image may highlight immune cells. Referring again to FIGS. 24A-24E, tumor epithelium and tumor stroma may be represented by the regions of purple, while immune cells may be represented by brown spots. Brown spots located within regions of tumor epithelium may correspond to immune cells that have enter the tumor epithelium, whereas brown spots within regions of tumor stroma may correspond to immune cells that have not entered the tumor epithelium.

Returning to FIG. 5, epithelium/stroma identification module 1212 may be configured to identify epithelium tiles and stroma tiles from tiles 606. In some embodiments, epithelium/stroma identification module 1212 may scan the digital pathology image using a sliding window to determine whether a portion of the digital pathology image included within the sliding window depicts tumor epithelium, tumor stroma, or both tumor epithelium and tumor stroma. In some embodiments, epithelium/stroma identification module 1212 may analyze each tile 606 to determine whether that tile includes depictions of epithelial cells (e.g., CK+ cells), stroma cells (e.g., CK− cells), immune cells (e.g., CD8+ T cells), or other cells, or combinations thereof.

Epithelium/stroma identification module 1212 may be configured to identify one or more of tiles 606 as being an epithelium tile based on a portion of that tile satisfying an epithelium-tile criterion. The epithelium-tile criterion may be satisfied if the portion of the tile depicting tumor epithelium (e.g., an area of the tile encompassing pixels highlighting epithelial cells) is greater than or equal to a first threshold area (e.g., 10% or more of a tile's area, 25% or more of a tile's area, 40% or more of a tile's area, 50% or more of a tile's area, etc.). For example, if 25% or more of a tile's area is determined to include depictions of epithelial cells, that tile may be classified as an epithelium tile. Each of tiles 606 determined to be an epithelium tile may be tagged with an epithelium tile label (e.g., metadata indicating that the corresponding tile is an epithelium tile). The metadata may also indicate spatial information about the epithelium tile, such as the tile's position relative to digital pathology image 604 and the other tiles 606 and/or a location of depictions of particular types of biological objects, such as epithelial cells (e.g., pixel coordinates of a pixel having a pixel hue, saturation, and/or value associated with that of an epithelial cell).

Epithelium/stroma identification module 1212 may be configured to identify one or more of tiles 606 as being a stroma tile based on a portion of that tile satisfying a stroma-tile criterion. The stroma-tile criterion may be satisfied if the portion of the tile depicting tumor stroma (e.g., an area of the tile encompassing pixels highlighting stromal cells) is greater than or equal to a first threshold area (e.g., 10% or more of a tile's area, 25% or more of a tile's area, 40% or more of a tile's area, 50% or more of a tile's area, etc.). For example, if 25% or more of a tile's area is determined to include depictions of stromal cells, that tile may be classified as a stroma tile. Each of tiles 606 determined to be a stroma tile may be tagged with a stroma tile label (e.g., metadata indicating that the corresponding tile is a stroma tile). The metadata may also indicate spatial information about the stroma tile, such as the tile's position relative to digital pathology image 604 and the other tiles 606 and/or a location of depictions of particular types of biological objects, such as stromal cells (e.g., pixel coordinates of a pixel having a pixel hue, saturation, and/or value associated with that of a stromal cell).

In some embodiments, a tile may be classified as being both an epithelium tile and a stroma tile. A tile may satisfy both the epithelium-tile criterion and the stroma-tile criterion. For example, a portion of a tile depicting tumor epithelium (e.g., an area of the tile encompassing pixels highlighting epithelial cells) being greater than or equal to a first threshold area (e.g., 10% or more of a tile's area, 25% or more of a tile's area, 40% or more of a tile's area, 50% or more of a tile's area, etc.) and the same or different portion of the tile depicting tumor stroma (e.g., an area of the tile encompassing pixels highlighting stromal cells) being greater than or equal to a second threshold area (e.g., 10% or more of a tile's area, 25% or more of a tile's area, 40% or more of a tile's area, 50% or more of a tile's area, etc.) may indicate that this tile should be classified as being an epithelium tile and a stroma tile. In some embodiments, the first threshold area and the second threshold area may be the same or they may differ. Metadata may be stored in association with a tile. The metadata may indicate that the tile has been classified as being both an epithelium tile and a stroma tile. Furthermore, some embodiments may include at least some of the epithelium tiles depicting regions of tumor stroma and at least some of the stroma tiles depicting regions of tumor epithelium.

Returning to FIG. 5, epithelium-immune cell density module 1214 may be configured calculate an epithelium-immune cell density for each of the epithelium tiles. In some embodiments, the epithelium-immune cell density for an epithelium tile may be calculated based on a number of immune cells detected within the epithelium tiles. Epithelium tiles having greater quantities of immune cells within tumor epithelium may have a greater epithelium-immune cell density than epithelium tiles having fewer immune cells. In some embodiments, epithelium-immune cell density module 1214 may be implement one or more machine learning models to determine the number of immune cells within each epithelium tile. In some embodiments, the machine learning models may include a computer vision model trained to recognize biological objects, such as immune cells, within an image tile. For example, the machine learning model may be a CNN trained to detect immune cells within an image. The machine learning models may be stored in model database 146. In some embodiments, epithelium-immune cell density module 1214 may access the machine learning model(s) from model database 146 and may provide each epithelium tile to the machine learning model(s) as an input. The machine learning model(s) may output an epithelium-immune cell density for that epithelium tile and/or a value indicating a number of immune cells detected within that epithelium tile. In the latter example, where the value is output by the machine learning model(s), epithelium-immune cell density module 1214 may be configured to calculate the epithelium-immune cell density based on the number of immune cells detected, an area of the tile, and/or an area of the tile including depictions of epithelial cells.

Stroma-immune cell density module 1216 may be configured calculate a stroma-immune cell density for each of the stroma tiles. In some embodiments, the stroma-immune cell density for a stroma tile may be calculated based on a number of immune cells detected within the stroma tiles. Stroma tiles having greater quantities of immune cells within tumor stroma may have a greater stroma-immune cell density than stroma tiles having fewer immune cells. In some embodiments, stroma-immune cell density module 1216 may be implement one or more machine learning models to determine the number of immune cells within each stroma tile. In some embodiments, the machine learning models may include a computer vision model trained to recognize biological objects, such as immune cells, within an image tile. For example, the machine learning model may be a CNN trained to detect immune cells within an image. The machine learning models may be stored in model database 146. In some embodiments, the machine learning models implemented by stroma-immune cell density module 1216 to calculate the stroma-immune cell density may be the same or similar to the machine learning models implemented by epithelium-immune cell density module 1214 to calculate the epithelium-immune cell density. In some embodiments, stroma-immune cell density module 1216 may access the machine learning model(s) from model database 146 and provide each stroma tile to the machine learning model(s) as an input. The machine learning model(s) may output a stroma-immune cell density for that stroma tile and/or a value indicating a number of immune cells detected within that stroma tile. In the latter example, where the value is output by the machine learning model(s), stroma-immune cell density module 1216 may be configured to calculate the stroma-immune cell density based on the number of immune cells detected, an area of the tile, and/or an area of the tile including depictions of stromal cells.

In some embodiments, the machine learning models may be trained to detect different types of biological objects within an image tile. The one or more machine learning models are trained using training data including a plurality of training images and labels indicating a type of biological object or types of biological objects depicted within each of the plurality of training images, a quantity of each depicted biological object, a location of each depicted biological object, or other information related to the biological objects depicted within the image. In some embodiments, the training images may be of a same or similar size as that of image tiles 606 of FIG. 6. In some embodiments, the training images may be whole slide images divided into tiles. The type of biological objects that the machine learning models are be trained to detect may include immune cells, epithelial cells, stromal cells, or other types of biological objects, or combinations thereof.

FIG. 7 illustrates an example of an immune cell density calculation, in accordance with various embodiments. For example, the process described by FIG. 7 may be used to calculate an epithelium-immune cell density and/or a stroma-immune cell density of a tile. As seen in FIG. 7, three masks 710, 720, and 730 are illustrated. Each of masks 710, 720, and 730 may have been generated from a digital pathology image, such as digital pathology image 604 of FIG. 6. In some embodiments, masks 710, 720, and 730 may be generated using epithelium-immune cell density module 1214 and/or stroma-immune cell density module 1216, or other suitable components of fourth pipeline subsystem 118.

In some embodiments, mask 710 may be a stain intensity mask for a digital pathology image. In the illustrated example, the digital pathology image and mask 710 may be divided into four tiles 711-714. Each of tiles 711-714 may include four pixels. Each pixel may be associated with a stain intensity value that corresponds to the intensity of a particular stain (e.g., the intensity of color channels known to be reflective of stain performance). For example, the northwest tile, tile 711, may include stain intensity values: 3, 25, 6, and 30; the southwest tile, tile 712, may include stain intensity values: 5, 8, 7, and 9; the northeast tile, tile 713 may include stain intensity values: 35, 30, 25, and 3; and the southeast tile, tile 714, may include stain intensity values: 4, 20, 8, and 5. Each of the stain intensity values may be reflective of the performance of the stain (e.g., the rate of absorption or expression of the stain by the biological objects depicted in the corresponding pixels of the digital pathology image). The stain intensity values can be used to determine which biological objects are shown in the tiles and the frequency of their appearance.

In some embodiments, mask 720 may be a stain thresholded binary mask for stain intensity mask 710. Each individual pixel value of stain intensity mask 710 may be compared to a predetermined and customizable threshold for the stain of interest. The threshold value can be selected accordingly to protocol reflective of the expected level of expression of stain intensity corresponding to a confirmed depiction of the correct biological object. The stain intensity values and threshold values can be absolute values (e.g., a stain intensity value above 20) or relative values (e.g., setting the threshold at the top 30% of stain intensity values). Additionally, the stain intensity values can be normalized according to historical values (e.g., based on overall performance of the stain on a number of previous analyses) or based on the digital pathology image at hand (e.g., to account for brightness differences and other imaging changes that may cause the image to inaccurately display the correct stain intensity). In stain thresholded binary mask 720, the threshold may be set to a stain intensity value of 20 and applied across all pixels within stain intensity mask 710. The result may be a pixel-level binary mask with ‘1’ indicating that the pixel had a stain intensity at or exceeding the threshold value and ‘0’ indicating that the pixel did not satisfy the requisite stain intensity.

In some embodiments, mask 730 may be an object density mask on the tile-level. Based on the assumption that stain intensity levels above the threshold correlate to depiction of a particular biological object within the digital pathology image, operations may be performed on the stain thresholded binary mask 720 to reflect the density of biological object within each tile. In the example object density mask 730, the operations include summing the values of the stain thresholded binary mask 720 within each tile and dividing by the number of pixels within the tile. As an example, the northwest tile, tile 711, may include two pixels above the threshold stain intensity value out of a total of four pixels, therefore the value in object density mask 730 for the northwest tile is 0.5. Similar operations may be applied across all of tiles 711-714. Additional operations can be performed to, for example, preserve locality with each tile, such as sub-tile segmentation and preservation of coordinates of each sub-tile within the lattice. As described herein, object density mask 730 can be used as the basis for calculation of spatial-distribution metrics (described in greater detail below with respect to fourth pipeline subsystem 118). It will be appreciated that the example depicted in FIG. 7 is simplified for discussion purposes only. The number of pixels within each tile and the number of tiles within each digital pathology image can be expanded and adjusted as needed based on computational efficiency and accuracy requirements.

Returning to FIG. 5, density binning module 1218 may be configured to bin the epithelium tiles into an epithelium set of bins based on each epithelium tile's epithelium-immune cell density and bin the stroma tiles into a stroma set of bins based on each stroma tile's stroma-immune cell density. The epithelium set of bins and the stroma set of bins may each include a predetermined number of bins corresponding to a particular range of densities. A tile may be “binned” into one of the bins based on whether the tile is an epithelium tile, a stroma tile, or a stroma tile and an epithelium tile, and the corresponding epithelium-immune cell density and/or stroma-immune cell density calculated for that tile. In some embodiments, the epithelium set of bins and the stroma set of bins may include a same quantity of bins each encompassing a same predefined range of densities.

As an example, with reference to FIG. 8, an immune cell density distribution 800 may include epithelium set of bins 802 and stroma set of bins 804. An epithelium tile binned into one of epithelium set of bins 802 may have an epithelium-immune cell density falling within a density range associated with that bin. Similarly, a stroma tile binned into one of stroma set of bins 804 may have a stroma-immune cell density falling within a density range associated with that bin. In other words, a bin may be defined by a lower density threshold, T1, and an upper density threshold, T2, and a tile determined to have an immune cell density that is less threshold T2 but greater than or equal to threshold T1 may be allocated to that bin. Allocating a tile to a bin may refer to incrementing a count of tiles that have an immune cell density falling within that bin's density range. In some embodiments, allocating immune cell density distribution 800 may include two data structures: one representing epithelium set of bins 802 and one representing stroma set of bins 804. The data structures may have a number of elements equal to the number of bins in each set of bins 802, 804. A value of each element in each data structure may correspond to a number of tiles determined to have an epithelium/stroma-immune cell density within a density range associated with that bin, an average epithelium/stroma immune cell density of all of the tiles within that density range, or a minimum/maximum epithelium/stroma immune cell density of the tiles within that density range, and the like. In some embodiments, the values for each element of the data structures may be normalized (e.g., based on a total number of tiles, an average epithelium immune cell density across all bins, etc.).

In some embodiments, epithelium set of bins 802 and stroma set of bins 804 may each include a same number of bins. For example, epithelium set of bins 802 may include ten bins and stroma set of bins 804 may also include ten bins. However, persons of ordinary skill in the art will recognize that other quantities of bins may be used. In this example, the corresponding data structures representing epithelium set of bins 802 and stroma set of bins 804 may include a same number of elements. Each of the ten bins may be defined by a corresponding density range. In some embodiments, the density ranges of epithelium set of bins 802 and stroma set of bins 804 may be the same. For example, a first bin from epithelium set of bins 802 may encompass a first density range [T1-T2], and a first bin from stroma set of bins 804 may also encompass the first density range [T1-T2]. Similarly, a second bin from epithelium set of bins 802 may encompass a second density range [T2-T3], and a second bin from stroma set of bins 804 may also encompass the second density range [T2-T3].

Immune cell density distribution 800 may be formed by determining a number of epithelium tiles that have an epithelium-immune cell density within each of the density ranges of epithelium set of bins 802 and a number of stroma tiles that have a stroma-immune cell density within each of the density ranges of stroma set of bins 804. As an example, epithelium set of bins 802 may be defined by ten density ranges: a first density range comprising epithelium-immune cell densities between 0.0-0.005, a second density range comprising epithelium-immune cell densities between 0.005-0.01, a third density range comprising epithelium-immune cell densities between 0.01-0.02, a fourth density range comprising epithelium-immune cell densities between 0.02-0.04, a fifth density range comprising epithelium-immune cell densities between 0.04-0.06, a sixth density range comprising epithelium-immune cell densities between 0.06-0.08, a seventh density range comprising epithelium-immune cell densities between 0.08-0.12, an eighth density range comprising epithelium-immune cell densities between 0.12-0.16, a ninth density range comprising epithelium-immune cell densities between 0.16-0.2, and a tenth density range comprising epithelium-immune cell densities between 0.2-2.0. Stroma set of bins 804 may be defined by ten density ranges: a first density range comprising stroma-immune cell densities between 0.0-0.005, a second density range comprising stroma-immune cell densities between 0.005-0.01, a third density range comprising stroma-immune cell densities between 0.01-0.02, a fourth density range comprising stroma-immune cell densities between 0.02-0.04, a fifth density range comprising stroma-immune cell densities between 0.04-0.06, a sixth density range comprising stroma-immune cell densities between 0.06-0.08, a seventh density range comprising stroma-immune cell densities between 0.08-0.12, an eighth density range comprising stroma-immune cell densities between 0.12-0.16, a ninth density range comprising stroma-immune cell densities between 0.16-0.2, and a tenth density range comprising stroma-immune cell densities between 0.2-2.0.

Returning to FIG. 5, density-bin representation module 1220 may be configured to generate a density-bin representation based on immune cell density distribution 800. In some embodiments, the density-bin representation may represent epithelium set of bins 802 and stroma set of bins 804, which form immune cell density distribution 800. The density-bin representation may include a plurality of elements corresponding to each bin of epithelium set of bins 802 and each bin of stroma set of bins 804. As an example, with reference to FIG. 9, immune cell distribution 800 of FIG. 8 may be transformed into density-bin representation 910. Density-bin representation 910 may include elements X₀-X_{N}, where N represents a total number of bins in immune cell density distribution 800. For example, if epithelium set of bins 802 and stroma set of bins 804 include ten bins, N=20. Considering the example where immune cell density distribution 800 includes two data structures respectively representing epithelium set of bins 802 and stroma set of bins 804, each data structure may include N/2 elements (in the scenario where the number of bins is equal across bins 802, 804). A value of each element (e.g., elements X₀-X_{N}) in each data structure may correspond to a number of tiles determined to have an epithelium/stroma-immune cell density within a density range associated with that bin, an average epithelium/stroma immune cell density of all of the tiles within that density range, or a minimum/maximum epithelium/stroma immune cell density of the tiles within that density range, and the like. In some embodiments, the values for each element (e.g., elements X₀-X_{N}) in the data structures may be normalized (e.g., based on a total number of tiles, an average epithelium immune cell density across all bins, etc.).

In some embodiments, density-bin representation module 1220 may transform immune cell density distribution 800 into density-bin representation 910. In some embodiments, density-bin representation 910 may be a feature vector that can be input to a classifier 920 to determine a tumor immunophenotype of an image (e.g., image 604 of FIG. 6). For example, the feature vector may be formed of the data structures comprising elements X₀-X_{N}.

In some embodiments, local density module 1222 may be configured to determine one or more local density measurements associated with a local density of one or more types of biological object. For example, local density module 1222 may calculate a density of epithelial cells in a given tile, a density of stromal cells in a tile, a density of immune cells (e.g., an epithelium-immune cell density) co-localized with epithelial cells in a tile (e.g., an epithelium-immune cell density), a density of immune cells co-localized with stromal cells in a tile (e.g., a stroma-immune cell density), or other local densities, or combinations thereof. In some embodiments, local density module 1222 may operate in conjunction with epithelium-immune cell density module 1214 and/or stroma-immune cell density module 1216 to calculate the epithelium-immune cell density and the stroma-immune cell density of a given tile (e.g., an epithelium tile and/or a stroma tile). In some embodiments, local density module 1222 may calculate an epithelial cell density and a stroma cell density for a tile and may obtain the epithelium-immune cell density and/or the stroma-immune cell density from epithelium-immune cell density module 1214 and/or stroma-immune cell density module 1216, respectively.

In some embodiments, local density module 1222 may determine an epithelial cell density of a tile based on a number of epithelial cells detected within the tile. The more epithelial cells present within a tile, the greater the epithelial cell density of that tile. In some embodiments, a tile may be classified as an epithelium tile if an area of the tile satisfies a threshold area criterion. For example, the threshold area criterion being satisfied may comprise a number of pixels whose intensity is greater than or equal to a threshold intensity value being greater than or equal to a threshold number of pixels. The threshold intensity value may be associated with pixels depicting epithelial cells, and the number of pixels being greater than the threshold intensity value may indicate that a threshold area is encompassed by pixels depicting epithelial cells

In some embodiments, local density module 1222 may determine a stromal cell density of a tile based on a number of stromal cells detected within the tile. The more stromal cells present within a tile, the greater the stromal cell density of that tile. In some embodiments, a tile may be classified as a stroma tile if an area of the tile satisfies a threshold area criterion. For example, the threshold area criterion being satisfied may comprise a number of pixels whose intensity is greater than or equal to a threshold intensity value being greater than or equal to a threshold number of pixels. The threshold intensity value may be associated with pixels depicting stromal cells, and the number of pixels being greater than the threshold intensity value may indicate that a threshold area is encompassed by pixels depicting stromal cells.

In some embodiments, spatial distribution metric module 1224 may be configured to generate one or more spatial distribution metrics. In some embodiments, the spatial distribution metrics may describe an input image of a tumor, such as digital pathology image 604 of FIG. 6. The spatial distribution metrics may be generated based on the local density measurements calculated by local density module 1222. For example, the spatial distribution metrics may be calculated based on an epithelial cell density, a stromal cell density, an epithelium-immune cell density, and a stroma-immune cell density.

In some embodiments, spatial distribution metric module 1224 may be configured to generate heatmaps of biological object densities for one or more types of biological objects. For example, one type of biological object may be immune cells, and the generated heatmaps may indicate immune cell densities in the tumor epithelium (e.g., epithelium-immune cell density) and/or the tumor stroma (e.g., stroma-immune cell density). As seen, for example, with reference to FIGS. 26A-26B, spatial distribution metric module 1016 may create heatmap visualizations 2600 and heatmap visualization 2650 based on the respective object density metrics. For example, heatmap visualization 2600 may illustrate a stroma-immune cell density (e.g., the density of CD8+ T cells in CK− tumor cells). Heatmap visualization 2650 may illustrate an epithelium-immune cell density (e.g., the density of CD8+ T cells in CK+ tumor cells). Heatmap visualization 2600 and heatmap visualization 2650 can assist pathologists in systematically categorizing the sample shown in the digital pathology image and also assist in understanding the tumor immunophenotype that will be assigned by fourth pipeline subsystem 118.

FIG. 27 illustrates a plot 2700 of biological object density bins by tumor immunophenotype, in accordance with various embodiments. For example, plot 2700 illustrates epithelial cell densities (e.g., CK+ tile densities) and stromal cell densities (e.g., CK− densities) plotted against immune cell densities (e.g., CD8+ T cell densities). Plot 2700 illustrates a first and naïve interpretation of the density scores that can be generated by local density module 1222. Although certain trends may be determinable from plot 2700, such as the clustering of desert immunophenotypes lower in the y-axis and the prevalence of inflamed immunophenotypes higher in both the x-axis and y-axis, it is difficult to draw further conclusions as additional clusters cannot be determined. Thus, plot 2700 demonstrates the limitations of previous forms of analysis and the motivation of development of additional techniques to automatically classify digital pathology images and the tiles derived therefrom. These additional techniques include, for example, the integration of advanced spatial distribution metrics derived from the density values.

FIG. 28B depicts an application of an areal analysis framework implemented by spatial distribution metric module 1224, in accordance with various embodiments. In particular, the areal analysis framework may be used to process a digital pathology image of a stained sample section. Densities of particular types of biological objects (e.g., tumor cells and T cells) may be detected, as described above, to produce biological object data, an example of which is shown in table 2800. In some embodiments, the output biological object data may include coordinates of individual tiles (e.g., epithelium tiles and stroma tiles) within the lattice formed by tile generation module 1210, and areas of the tiles associated with each of the biological objects of interest. As an example, when the biological objects of interest include immune cells, epithelial cells, and stromal cells (e.g., CD8+ T cells, CK+ tumor cells, CK− tumor cells), the output biological data may include areas of the epithelium tiles (e.g., tiles associated with CK+ tumor cells), areas of the stroma tiles (e.g., tiles associated with CK-tumor cells), areas of the epithelium tiles and the immune cells (e.g., tiles associated with CK+ tumor cells and CD8+ T cells), and areas of the stroma tiles and the immune cells (e.g., tiles associated with CK− tumor cells and CD8+ T cells).

In some embodiments, spatial distribution metric module 1224 may generate a spatial lattice having a defined number of columns and a defined number of rows that can be used to divide the digital pathology image into tiles. For each tile, a number or density of biological object depictions within the region can be identified, such as by using the density accounting techniques described herein. For each biological object type, the collection of region-specific biological object densities the mapping of which tiles, at which locations, contain specific density values can be defined as the biological object type's lattice data. FIG. 28A illustrates a particular embodiment of lattice data 2810 for depictions of a first type of biological object, tumor epithelial cells (e.g., CK+ tumor cells), and lattice data 2815 for depictions of a second type of biological object, immune cells (e.g., CD8+ T cells). Each of the lattice data is shown, for purposes of illustration, as being overlaid on a representation of a digital pathology image of a stained section. In some embodiments, lattice data can be defined to include, for each region in the lattice, a prevalence value defined to equal counts for the region divided by total counts across all regions. Thus, regions within which there are no biological objects of a given type may be assigned a prevalence value of 0, while regions within which there is at least one biological object of a given type may be assigned a positive non-zero prevalence value.

In some embodiments, identical amounts of biological objects (e.g., lymphocytes) in two different contexts (e.g., tumors) do not necessarily imply the characterization or degree of characterization (e.g., the same immune infiltration). Instead, how the biological object depictions of a first type are distributed in relation to biological object depictions of a second type can indicate a functional state. Therefore, characterizing proximity of biological object depictions of the same and different types can reflect more information.

The Morisita-Horn Index is an ecological measure of similarity (e.g., overlap) in biological or ecological systems. The Morisita-Horn index (MH) may be used to characterize the bi-variate relationship or co-localization between two populations of biological object depictions (e.g., of two types), and can be defined by Equation 1:

$$M H = \frac{2 {Σ}_{i}^{n} {z}_{i}^{l} {z}_{i}^{t}}{{\sum }_{i}^{n} {( {z}_{i}^{l} )}^{2} + {\sum }_{i}^{n} {( {z}_{i}^{t} )}^{2}} . \qquad Equation 1$$

In Equation 1, zᵢ^{l}, zᵢ^{t} denote the prevalence of biological object depictions of a first type and biological object depictions of a second type at the square grids i, respectively. In FIG. 28A, lattice data 2810 shows exemplary prevalence values zᵢ^{t} of depictions of a first type of biological object across grid points, and lattice data 2815 shows exemplary prevalence values zᵢ^{l} of depictions of a second type of biological object of the across grid points.

The Morisita-Horn Index is defined to be 0 when individual lattice regions do not include biological object depictions of both types (indicating that the distributions of different biological object types are spatially separated). For example, the Morisita-Horn Index would be 0 when considering the illustrative spatially separate distributions or segregated distributions shown in illustrative first scenario 2820. The Morisita-Horn Index is defined to be 1 when a distribution of a first biological object type across lattice regions matches (or is a scaled version of) a distribution of a second biological object type across lattice regions. For example, the Morisita-Horn Index would be close to 1 when considering the illustrative highly co-localized distributions shown in illustrative second scenario 2825.

In the example of FIG. 28A, the Morisita-Horn Index calculated using lattice data 2810 and lattice data 2815 is 0.47. A value of 0.47 may be considered to be a high Morisita-Horn Index value, indicating that the depictions of biological objects of the first type and second type were highly colocalized.

Another spatial distribution metric that may be calculated by spatial distribution metric module 1224 may be the Jaccard index (J) and the Sorensen index (L), which are similar and closely related to each other, can be defined by Equations 2 and 3, respectively:

$$J = \frac{{\sum }_{i}^{n} min ( {z}_{i}^{l} , {z}_{i}^{t} )}{{\sum }_{i}^{n} ( {z}_{i}^{l} + {z}_{i}^{t} ) - {Σ}_{i}^{n} min ( {z}_{i}^{l} , {z}_{i}^{t} )} , \qquad Equation 2$$

$$L = \frac{2 {\sum }_{i}^{n} min ( {z}_{i}^{l} , {z}_{i}^{t} )}{{\sum }_{i}^{n} ( {z}_{i}^{l} + {z}_{i}^{t} )} . \qquad Equation 3$$

In Equations 2 and 3, where zᵢ^{l}, zᵢ^{t} denotes the prevalence of biological object depictions of a first type and biological object depictions of a second type at the square grids i, respectively, and min(a, b) returns the minimum value between a and b.

Another spatial distribution metric that can characterize a spatial distribution of biological object depictions is Moran's Index, which is a measure of spatial autocorrelation. Moran's Index is the correlation coefficient for the relationship between a first variable and a second variable at neighboring spatial units. The first variable can be defined as prevalence of depictions of biological objects of a first type and the second variable can be defined as prevalence of depictions of biological objects of a second type, so as to quantify the extent that the two types of biological object depictions are interspersed in digital pathology images.

Moran's Index, I, can be defined using Equation 4:

$$I = \frac{n}{{\sum }_{i}^{n} {Σ}_{j}^{n} {w}_{i j}} ( {\sum }_{i}^{n} {\sum }_{j}^{n} {w}_{ij} ( {x}_{i} ) ( {y}_{j} ) ) . \qquad Equation 4$$

In Equation 4, xᵢ, yᵢ denote the standardized prevalence of biological object depictions of the first type (e.g., tumor cells) at areal unit i, and the standardized prevalence of biological object depictions of the second type (e.g., lymphocytes) at areal unit j. The wᵢⱼ is the binary weight for areal units i and j−wᵢⱼ is 1 if two units neighbor, and 0 otherwise. A first-order scheme can be used to define neighborhood structure. Moran's I can be derived separately for biological object depictions of different types of biological objects.

Moran's Index is defined to be equal to −1 when biological object depictions are perfectly dispersed across a lattice (and thus having a negative spatial autocorrelation); and to be 1 when biological object depictions are tightly clustered (and thus having a positive autocorrelation). Moran's Index is defined to be 0 when an object distribution matches a random distribution. The areal representation of particular biological object depiction types thus facilitates generating a grid that supports calculation of a Moran's Index for each biological object type. In some embodiments, in which two or more types of biological object depictions are being identified and tracked, a difference between the Moran's Index calculated for each of the two or more types of biological object depictions can provide an indication of colocation (e.g., with differences near zero indicating colocation) between those types of biological object depictions.

Yet another example spatial distribution metric is Geary's C, also known as Geary's contiguity ratio, which is measure of spatial autocorrelation or an attempt to determine if adjacent observations of the same phenomenon are correlated. Geary's C is inversely related to Moran's Index, but it is not identical. While Moran's Index is a measure of global spatial autocorrelation, Geary's C is more sensitive to local spatial autocorrelation. Geary's C can be defined using Equation 5:

$$C = \frac{n - 1}{2 {Σ}_{i}^{n} {Σ}_{j}^{n} {w}_{ij}} \frac{{Σ}_{i}^{n} {Σ}_{j}^{n} {{w}_{ij} ( {z}_{i} - {z}_{j} )}^{2}}{{{Σ}_{i}^{n} ( {z}_{i} - {z}_{j} )}^{2}} . \qquad Equation 5$$

In Equation 5, zᵢ, zⱼ denote the prevalence of either biological object depictions of a first type or a second type at the square grids i, j, and wᵢⱼ is the same as defined aforementioned.

Still yet another spatial distribution metric that can characterize a spatial distribution of biological object depictions is the Bhattacharyya coefficient (“B coefficient”), which is an approximate measure of an overlap between two statistical samples. In general, the B coefficient can be used to determine the relative closeness of the two statistical samples. It is used to measure the separability of classes in the classification.

Given probability distributions p and q over the same domain X (e.g., distributions of depictions of two types of biological objects within the same digital pathology image), the B coefficient is defined using Equation 6:

$$BC ( p , q ) = {\sum }_{x ∈ x} \sqrt{p ( x ) q ( x )} . \qquad Equation 6$$

In Equation 6, 0≤BC≤1. The Bhattacharyya coefficient is used to determine the Bhattacharyya, D_{B} (p, q)=−ln(BC(p, q)), where 0≤D_{B}≤∞. Note that DB does not obey the triangle inequality, but the Hellinger distance, \sqrt{1−BC(p, q)} does obey the triangle inequality. The B coefficient increases with the number of partitions in the domain that have members from both samples (e.g., with the number of tiles in the digital pathology image that have depictions or suitable density of two or more types of biological object depictions). The B coefficient is therefore larger still with each partition in the domain that has a significant overlap of the samples, e.g., with each partition that contains a large number of the members of the two samples. The choice of the number of partitions is variable and can be customized to the number of members in each sample. To maintain accuracy, care is taken to avoid selecting too few partitions and overestimating the overlap region as well as taking too many partitions and creating partitions with no members despite being in a densely populated sample space. The B coefficient will be 0 if there is no overlap at all between the two samples of biological object depictions.

Returning to FIG. 28A, lattice data 2810 and lattice data 2815 can be further processed to generate hotspot data 2830 corresponding to detected depictions of a first type of biological object and hotspot data 2835 corresponding to detected depictions of a second type of biological object, respectively.

In FIG. 28B, hotspot data 2830 and hotspot data 2835 indicate the regions that were determined to be hotspots for the respective types of detected depictions of biological objects. The regions that were detected as hotspots are shown as circles and the regions that were determined not to be hotspots as an ‘x.’ Hotspot data 2830, 2835 is defined for each region associated with a non-zero object count. Hotspot data 2830, 2835 can also include binary values that indicate whether a given region was identified as being a hotspot or not. In addition to hotspot data and analysis, cold spot data and analysis can be conducted.

With respect to depictions of biological objects, hotspot data 2830, 2835 can be generated for each biological object type by determining a Getis-Ord local statistic for each region associated with a non-zero object count for the biological object type. Getis-Ord hotspot/cold spot analysis can be used to identify statistically significant hotspots/cold spots of tumor cells or lymphocytes, where hotspots are the areal units with a statistically significantly high value of prevalence of depictions of biological objects compared to the neighboring areal units and cold spots are the areal units with a statistically significantly low value of prevalence of depictions of biological objects compared to neighboring areal units. The value and determination what makes a hotspot/cold spot region compared the neighboring regions can be selected according to user preference, and, in particular can be selected according to a rules-based approach or learned model. For example, the number and/or type of biological object depictions detected, the absolute number of depictions, and other factors can be considered. The Getis-Ord local statistic is a z-score and can be defined, for a square grid i using Equation 7:

$${G}_{i}^{*} = \frac{{\sum }_{j = 1}^{n} {ω}_{i j} {z}_{j} - \overline{z} {\sum }_{j = 1}^{n} {ω}_{i j}}{s \sqrt{\frac{n {\sum }_{j = 1}^{n} {ω}_{i j}^{2} - {( {\sum }_{j = 1}^{n} {ω}_{i j} )}^{2}}{n - 1}}} . \qquad Equation 7$$

In Equation 7, i represents an individual region (specific row-column combination) in the lattice, n is the number of row and column combinations (i.e., number of regions) in the lattice, ωᵢⱼ is the spatial weight between i and j, z₁ is the prevalence of biological object depictions of a given type in a region, z is the average object prevalence of the given type across regions, and S is defined by Equation 8:

$$S = \sqrt{\frac{{\sum }_{j = 1}^{n} {z}_{j}^{2}}{n} - {( \overline{z} )}^{2}} . \qquad Equation 8$$

The Getis-Ord local statistics can be transformed to binary values by determining whether each statistic exceeds a threshold. For example, a threshold can be set to 0.16. The threshold can be selected according to user preference, and in particular can be set according to rule-based or machine-learned approaches.

A logical AND function can be used to identify the regions that are identified as being a hotspot for more than one type of depictions of biological objects. For example, colocalized hotspot data 2840 indicates the regions that were identified as being a hotspot for two types of biological object depictions (shown as circles symbols in FIG. 28B). A high ratio of a number of regions identified as being a co-localized hotspot relative to a number of hotspot regions identified for a given object type (e.g., for tumor-cell objects) can indicate that biological object depictions of the given type share spatial characteristics with the other object type. Meanwhile, a low ratio at or near zero can be consistent with spatial segregation of biological objects of the different types.

Returning to FIG. 12, spatial distribution representation module 1226 may be configured to generate a spatial density representation based on the one spatial distribution metrics generated by spatial distribution metric module 1224. In some embodiments, the spatial distribution metrics used to generate the spatial distribution representation may include:
Intra-tumor lymphocyte ratio;
Morisita-Horn Index;
Jaccard Index;
Sorensen index;
B coefficient;
Moran's Index;
Geary's C;
The ratio of co-localized spots (e.g., hotspots, cold spots, non-significant spots) for the type of biological object depictions over the number of spots (e.g., hotspots, cold spots, non-significant spots) for a first type of the biological object depictions, with spots (e.g., hotspots, cold spots, non-significant spots) defined using Getis-Ord local statistics; and
Features obtained by variogram fitting of two types of biological object depictions (e.g., tumor cells and lymphocytes).

In some embodiments, the spatial distribution representation may be a feature vector having M elements. Each element may correspond to one of the spatial distribution metrics and/or a parameter of a spatial distribution metric. For example, the spatial density representation may be a 50-dimensional feature vector. In some embodiments, an embedding may be generated based on the spatial density representation. The embedding may be mapped into an embedding space using a trained encoder. In this example, the tumor immunophenotype may be determined based on a distance between the mapped location of the embedding in the embedding space and one or more clusters of embeddings, each cluster being associated with a tumor immunophenotype.

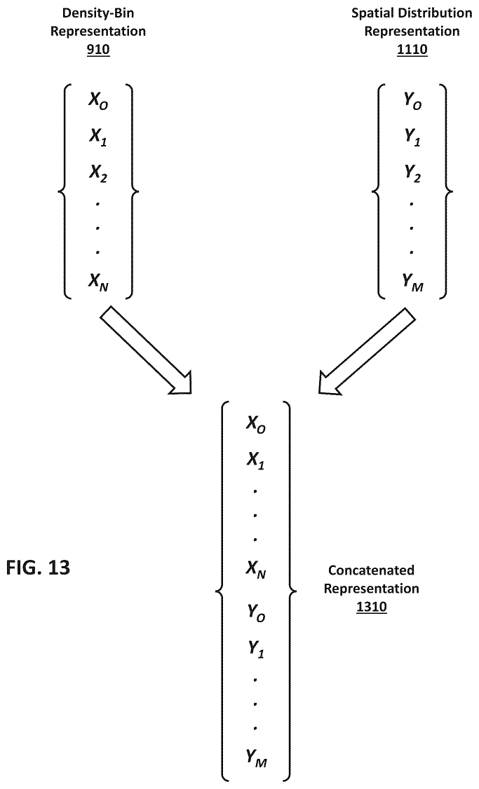

Classification module 1228 may be configured to combine the density-bin representation and the spatial distribution representation. In some embodiments, classification module 1228 may be configured to concatenate the density-bin representation and the spatial distribution representation to obtain a concatenated representation. As an example, with reference to FIG. 13, density-bin representation 910 and spatial distribution representation 1110 may be concatenated to obtain a concatenated representation 1310. Concatenated representation 1310 may include the elements from density-bin representation 910 and the elements from spatial distribution representation 1110. For example, density-bin representation 910 may include elements {X₀-X_{N}} and spatial distribution representation 1110 may include elements {Y₀-Y_{M}}. Thus, classification module 1228 may be configured to generate concatenated representation 1310 including elements {X₀, . . . , X_{N}, Y₀, . . . , Y_{M}}. The concatenation operation combines two or more strings of elements, appending one string to an end of another string. The result is a string having dimensions equal to the aggregate of the dimensions of each individual string. In the example of FIG. 13, if density-bin representation 910 is a 20-element feature vector and spatial distribution representation 1110 is a 50-element feature vector, then concatenated representation 1310 may be a 70-element feature vector. However, persons of ordinary skill in the art will recognize that the dimensions described above may differ for different embodiments, and the aforementioned is exemplary.

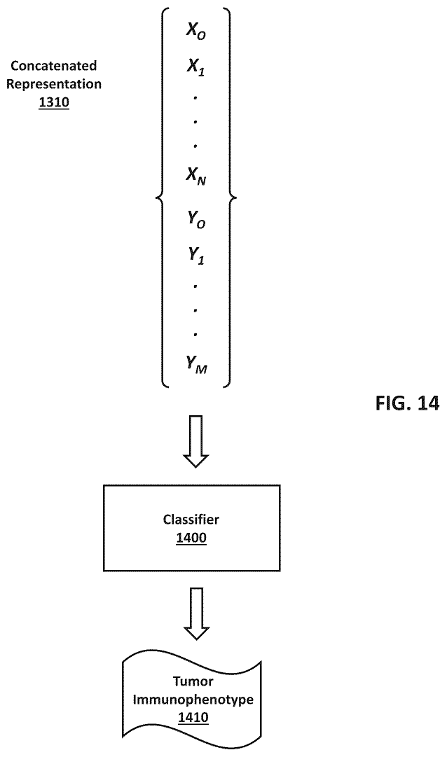

Classification module 1228 may also be configured to implement a classifier trained to determine a tumor immunophenotype for a digital pathology image depicting a tumor based on a concatenated representation generated for a digital pathology image. Classification module 1228 may be configured to implement a classifier, such as classifier 1400 of FIG. 14. Classifier 1400 may be the same or similar to classifier 920 of FIG. 9 and/or classifier 1120 of FIG. 11, and the previous descriptions may apply.

In some embodiments, classification module 1228 may receive the concatenated representation and input the concatenated distribution representation into the classifier. For example, classification module 1228 may provide concatenated representation 1310 to classifier 1400, which may be trained to output a predicted tumor immunophenotype 1410. The classifier may be trained to output a tumor immunophenotype (e.g., tumor immunophenotype 1410) of the biological sample depicted by the input digital pathology image (e.g., digital pathology image 604 of FIG. 6). In some embodiments, the classifier is a multi-class classifier configured to classify an image into one of a set of tumor immunophenotypes. The number of classes may depend on the number of tumor immunophenotype classifications. For example, classifier 1400 may be trained to classify an image into one of a set of tumor immunophenotypes based on concatenated representation 1310. The set of tumor immunophenotypes may include the tumor immunophenotypes of desert, excluded, and inflamed. In some embodiments, classifier 1400 implemented by classification module 1228 may be a support vector machine (SVM), a random forest (RF) classifier, a decision tree classifier, a logistic regression classifier, a k-nearest neighbor (kNN) classifier, or another type of classifier.

Classifier training module 1230 may be configured to train the classifier used by classification module 1228 to predict a tumor immunophenotype of an image of a tumor based on a concatenated representation generated for that image. In some embodiments, classifier training module 1230 may train the classifier (e.g., classifier 1400 of FIG. 14) using training data generated from a first plurality of patients participating in a first clinical trial. Classifier training module 1230 may further be configured to validate the classifier using validation data generated from a second plurality of patients participating in a second clinical trial.

In some embodiments, classifier training module 1230 may access a first plurality of images from image database 142. The first plurality of images may include images of tumors from patients participating in a first clinical trial. For example, the first clinical trial may include patients having advanced NSCLC who have progressed on a platinum-based chemotherapy regiment and who were 1:1 randomized to receive either a first immunotherapy (e.g., atezolizumab) or a second immunotherapy (e.g., docetaxel). The number of patients participating in the first clinical trial may be 100 or more patients, 200 or more patients, 300 or more patients, etc. In some embodiments, biological samples may be obtained for at least some of the patients of the first clinical trial. One biological sample may be obtained for each of the patients. The biological samples may include tumor lesions including tumor stroma and tumor epithelium. The biological samples may be stained using a dual-stain, such as a panCK-CD8 stain, to highlight tumor epithelium, tumor stroma, and immune cells.

Each sample may be imaged using image scanner 240 to obtain a first plurality of images. These images may be stored in image database 142. The images may be digital pathology images, such as whole-slide images. In some embodiments, classifier training module 1230 and/or other components of fourth pipeline subsystem 118 may be configured to divide each of the first plurality of images into tiles and identify which of those tiles are epithelium tiles and/or stroma tiles. For each image, an epithelium-immune cell density may be calculated for the epithelium tiles and a stroma-immune cell density may be calculated for the stroma tiles. An immune cell density distribution may be created including an epithelium set of bins and a stroma set of bins each spanning a respective density range. Epithelium tiles having epithelium-immune cell densities within a density range of a particular bin of the epithelium set of bins may be allocated to that bin, and stroma tiles having stroma-immune cell densities within a density range of a particular bin of the stroma set of bins may be allocated to that bin. A density-bin representation of the immune cell density distribution, based on the epithelium set of bins and the stroma set of bins may be generated. Additionally, one or more spatial distribution metrics may be calculated based on the epithelium-immune cell densities and the stroma-immune cell densities. A spatial distribution representation may be generated for each image based on that image's corresponding spatial distribution metrics. Therefore, for each of the first plurality of images, a corresponding spatial distribution representation may be obtained. For each image, the corresponding density-bin representation and the spatial distribution representation may be concatenated to obtain a concatenated representation

In some embodiments, classifier training module 1230 may be configured to generate training data including the first plurality of images and, for each image, a concatenated representation and a label indicating a predetermined tumor immunophenotype of the tumor depicted by that image. In some embodiments, the predetermined tumor immunophenotype may be determined by a trained pathologist. The training data may be stored in training data database 144. Classifier training module 1230 may select a classifier to be trained from model database 146 and may be configured to train the classifier using the training data stored in training data database 144. The classifier may be a multi-class classifier, such as a three-class random forest decision tree classifier. Classifier training module 1230 may optimize hyperparameters of the classifier using an optimizer, such as the Adam optimizer.

After the classifier has been trained using the training data, it may be tested using validation data. Classifier training module 1230 may be configured to generate the validation data using a second plurality of images. The second plurality of images may also be stored in image database 142. The second plurality of images may include images of tumors from patients participating in a second clinical trial. As an example, the second clinical trial may include patients having advanced NSCLC. As another example, the second clinical trial may include patients having metastatic TNBC who were randomized to receive a first therapy (e.g., atezolizumab plus nab-paclitaxel) or a second therapy (e.g., a placebo plus nab-paclitaxel). The number of patients participating in the first clinical trial may be 500 or more patients, 750 or more patients, 1,000 or more patients, etc. In some embodiments, biological samples may be obtained for at least some of the patients of the second clinical trial. One biological sample may be obtained for each of the patients. The biological samples may include tumor lesions including tumor stroma and tumor epithelium. The biological samples may be stained using a dual-stain, such as a panCK-CD8 stain, to highlight tumor epithelium, tumor stroma, and immune cells. Similar to the process described above for the first clinical trial, validation data may be generated based on a second plurality of images of the biological samples of at least some of the patients of the second clinical trial. The second plurality of images may be used to generate concatenated representations. In some embodiments, labels indicating a predetermined tumor immunophenotype of the biological sample may be assigned by a trained pathologist. Classifier training module 1230 may use the validation data to test the accuracy of the trained classifier. Thus, the validation data may include the second plurality of images, the concatenated representations for each of the images, and a label assigned to that image indicating a predetermined tumor immunophenotype. If the classifier does not predict the tumor immunophenotype with at least a threshold level of accuracy, classifier training module 1230 may retrain the classifier. However, if the classifier is determined to have a threshold level of accuracy, it may be deployed for determining a tumor immunophenotype of an input image. The classifier may output a tumor immunophenotype based on the concatenated distribution representation, which may be derived from a digital pathology image of a tumor (e.g., digital pathology image 604 of biological sample 602).

In some embodiments, the tumor immunophenotype may be one of a set of tumor immunophenotypes. For example, the tumor immunophenotypes may include desert, excluded, and inflamed. In some embodiments, a tumor depicted by a digital pathology image may be classified as the tumor immunophenotype desert based on an epithelium-immune cell density calculated for that image satisfying a desert epithelium-immune cell density threshold criterion and a stroma-immune cell density for that image also satisfying a desert stroma-immune cell density threshold criterion. As an example, the desert epithelium-immune cell density threshold criterion being satisfied may include the epithelium-immune cell density being within a first threshold range of epithelium-immune cell densities and the desert stroma-immune cell density threshold criterion being satisfied may include the stroma-immune cell density being within a first threshold range of stroma-immune cell densities. A tumor depicted by a digital pathology image may be classified as the tumor immunophenotype excluded based on an epithelium-immune cell density for that image satisfying an excluded epithelium-immune cell density threshold criterion and a stroma-immune cell density for that image satisfying an excluded stroma-immune cell density threshold criterion. For example, the excluded epithelium-immune cell density threshold criterion being satisfied may include the epithelium-immune cell density being within a second threshold range of epithelium-immune cell densities and the excluded stroma-immune cell density threshold criterion being satisfied may include the stroma-immune cell density being within a second threshold range of stroma-immune cell densities. A tumor depicted by a digital pathology image may be classified as the tumor immunophenotype inflamed based on an epithelium-immune cell density of that image satisfying an inflamed epithelium-immune cell density threshold criterion and a stroma-immune cell density of that image satisfying an inflamed stroma-immune cell density threshold criterion. For example, the inflamed epithelium-immune cell density threshold criterion being satisfied may include the epithelium-immune cell density being within a third threshold range of epithelium-immune cell densities and the inflamed stroma-immune cell density threshold criterion being satisfied may include the stroma-immune cell density being within a third threshold range of stroma-immune cell densities.

The metrics chosen can correspond to multiple frameworks (e.g., areal-process analysis framework). For each subject, a label can be defined to indicate secondary determinations, such as object density metrics and/or assigned immunophenotype. The machine-learning models, including but not limited to a logistic-regression model, can be trained and tested with the paired input data and labels, using repeated nested cross-validation. As an example, for each of 5 data folds, the model can be trained on the remaining 4 folds and tested on the remaining fold to calculate an area under an ROC.

In embodiments with limited sample size, adaptable techniques to evaluate model performance can be used. As a non-limiting example, a nested Monte Carlo Cross Validation (nMCCV) can be used to evaluate the model performance. The same enrichment procedure can be repeated B times by randomly splitting with the same proportion between training, validation, and test sets, to produce an ensemble of score function and threshold {(S̅_{b},\hat{q}ⱼ)}_{b=1}^{B}. For the i-th subject, the ensembled responder status can be evaluated by averaging, among the repetitions where i is randomized to test set, the membership of responder group for i, and thresholding by 0.5. Hazard ratio or Odds ratio together with a 95% confidence interval and p-value can be calculated on the aggregated test subjects.

As described herein, the designation of the immunophenotype can be provided by a machine-learned model trained in a supervising training process where labeled digital pathology images are provided along with their concatenated representations. Through the training process, the classifier implemented by classification module 1228 can learn to categorize digital pathology images using classifier training module 1230 and their corresponding samples, into selected immunophenotyping groups.

In some embodiments, classifier training module 1230 can train a machine-learning model, such as a classifier, to process a digital pathology image of a biopsy section from a subject, to predict an assessment of a condition of the subject from the digital pathology image. As an example, using the techniques described herein, fourth pipeline subsystem 118 can generate a variety of concatenated representations based on density-bin distributions and spatial distribution metrics and predict an immunophenotype for the digital pathology image. From this input, a regression machine-learning model can be trained predict, for example, suspected patient outcomes, assessments of related patient condition factors, availability or eligibility for selected treatments, and other related recommendations.

In some embodiments, the machine learning techniques that can be used in the systems/subsystems/modules of fourth pipeline subsystem 118 may include, but are not limited to (which is not to suggest that any other list is limiting), any of the following: Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, or separable convolutions (e.g., depth-separable convolutions, spatial separable convolutions).

A biopsy can be collected from each of multiple subjects having the condition. The sample can be fixed, embedded, sliced, stained, and imaged according to the subject matter disclosed herein. Depictions and densities of specified types of biological objects (e.g., tumor cells, lymphocytes), can be detected. Classifier training module 1230 of fourth pipeline subsystem 118 can use a trained set of machine-learned models to process images to quantify the density of biological objects of interest. For each subject of the multiple subjects, a label can be generated so as to indicate whether the condition exhibited specified features and/or indicate certain secondary labels (e.g., immunophenotype) applied by fourth pipeline subsystem 118. In the context of predicting an overall assessment of the condition of the subject, labels such as immunophenotype may be considered secondary as they can inform the overall assessment.

In some embodiments, one or more machine learning models implemented by first pipeline subsystem 112, second pipeline subsystem 114, third pipeline subsystem 116, and/or fourth pipeline subsystem 118. The machine learning models can be trained and customized for use in particular settings. For example, a machine learning model implemented by a tile generation module (e.g., tile generation modules 510, tile generation module 1010, tile generation module 1210) specifically trained for use in providing insights relating to specific types of tissue (e.g., lung, heart, blood, liver, etc.). As another example, the machine learning model implemented by a tile generation module (e.g., tile generation modules 510, tile generation module 1010, tile generation module 1210) can be trained to assist with safety assessment, for example in determining levels or degrees of toxicity associated with drugs or other potential therapeutic treatments. Once trained for use in a specific subject matter or use case, the machine learning model is not necessarily limited to that use case. Training may be performed in a particular context, e.g., toxicity assessment, due to a larger set of at least partially labeled or annotated images.

The machine learning techniques that can be used by first pipeline subsystem 112, second pipeline subsystem 114, third pipeline subsystem 116, and/or fourth pipeline subsystem 118 may include, but are not limited to (which is not to suggest that any other list is limiting), any of the following: Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, or separable convolutions (e.g., depth-separable convolutions, spatial separable convolutions).

The fourth pipeline implemented by fourth pipeline subsystem 118 may combine the detailed spatial features of the third pipeline with granular immune cell density of the second pipeline to achieve an improve tumor immunophenotype classification pipeline. Two criteria may be used to assess the performance of the fourth pipeline: (i) automated tumor immunophenotype classification agreement with manual tumor immunophenotype classification and the OS, PFS, and/or other survival metric log-rank test results.

Manual tumor immunophenotype classification techniques based on spatial distribution and/or immune cell density (e.g., CD8+ T effector cells) performed on the example second clinical trial and the example third clinical trial indicated longer patient overall survival (OS) for patient's classified into the tumor immunophenotype of inflamed. Patients classified into the tumor immunophenotype of desert had the lowest OS, while patients classified into the tumor immunophenotype of excluded exhibited intermediate outcomes, as seen, for example, by plots 3100 and 3150 of FIGS. 31A-31B. In this example, the median OS for the tumor immunophenotypes of inflamed, excluded, and desert were 16.4, 13.5, and 9.46 months, respectively, for the example second clinical trial, while the media OS for the same tumor immunophenotypes were not reached, 22.83, and 15.61 months, respectively, for the example third clinical trial.

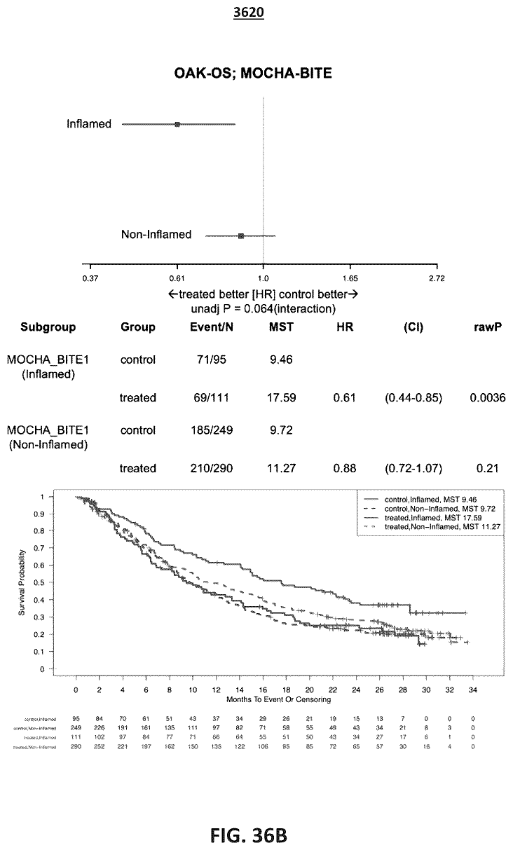

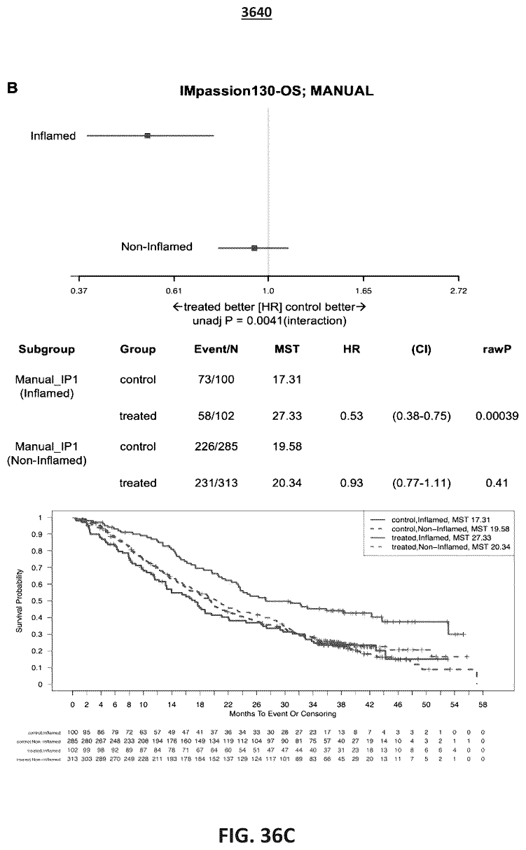

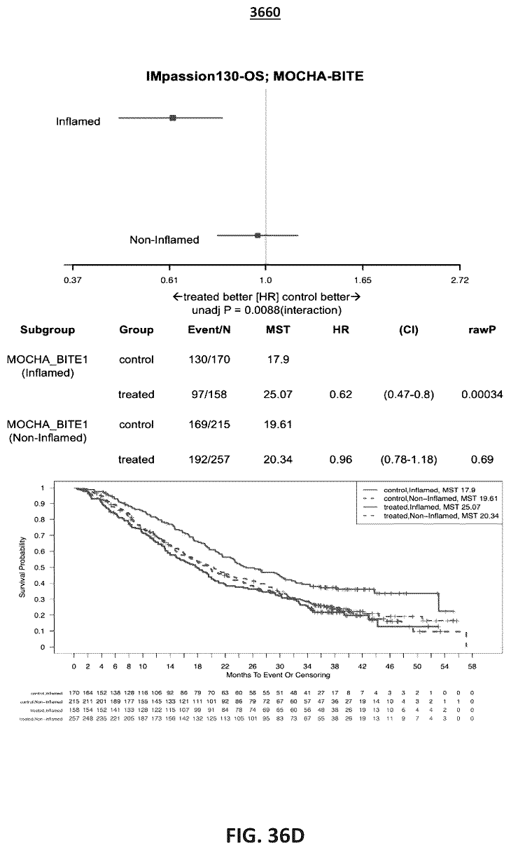

FIGS. 36A-36D illustrate example plots 3600-3660 describing the association of tumor immunophenotype classification with immunotherapy outcome, in accordance with various embodiments. For example, plots 3600 and 3620 illustrate the association of manual tumor immunophenotyping and automated tumor immunophenotyping to immunotherapy decisions for the example second clinical trial. Plots 3640 and 3660 illustrate the association of manual tumor immunophenotyping and automated tumor immunophenotyping to immunotherapy decisions for the example third clinical trial. In particular, plots 3600-3660 are forest plot analyses and Kaplan-Meier curves for patients classified into the tumor immunophenotypes of inflamed or non-inflamed (e.g., desert, excluded) based on data from the example second and third clinical trials. In plots 3600-3660, the vertical line of no effect in the forest plots indicates a hazard ratio (HR) of 1, shown are the number of patients in the control and treatment groups for each tumor immunophenotype category, median survival times (MST) with confidence intervals and p-values.

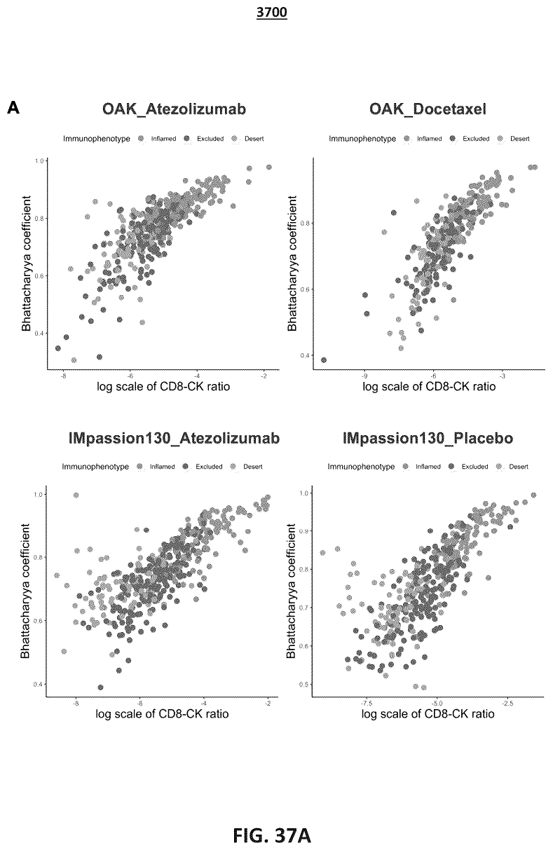

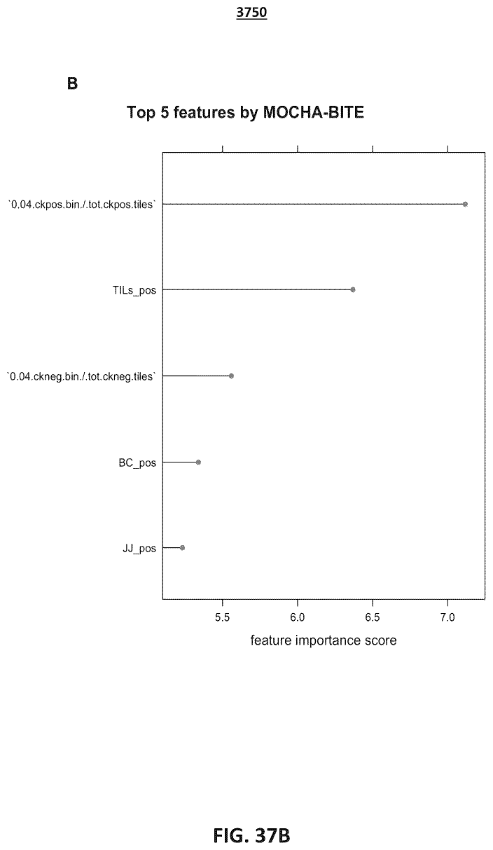

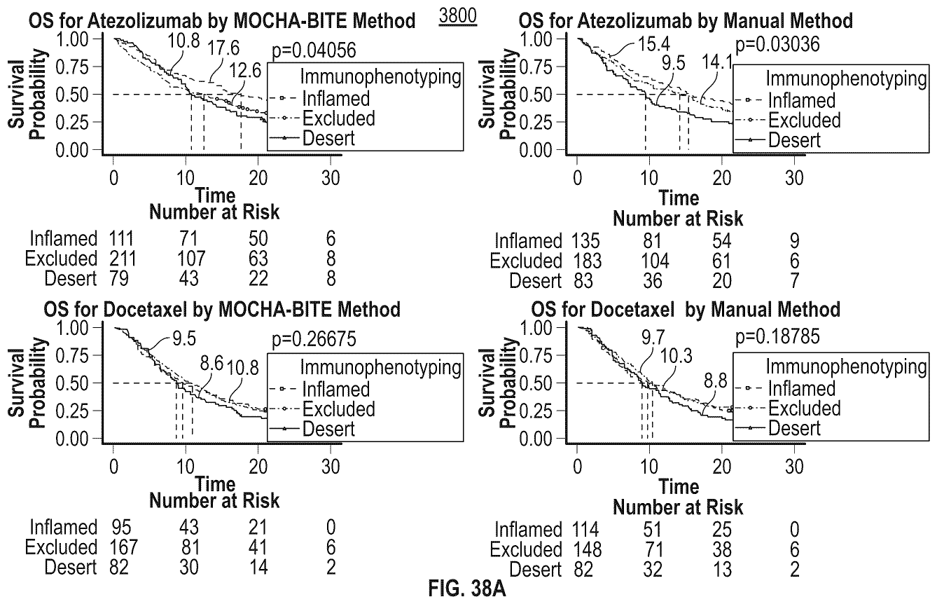

FIG. 37A illustrates plots 3700 of the Bhattacharyya coefficient for tumor immunophenotype classes for the example second and third clinical trials calculated using the fourth pipeline, in accordance with various embodiments. Each of plots 3700 represents one of two example immunotherapies for one of the example second and third clinical trials. The Bhattacharyya coefficient, in plots 3700, is computed as a function of the CD8/CK ratio.

FIG. 37B illustrates a chart 3750 of a top N features identified by the fourth pipeline, in accordance with various embodiments. For example, chart 3750 ranks these features by feature importance score.

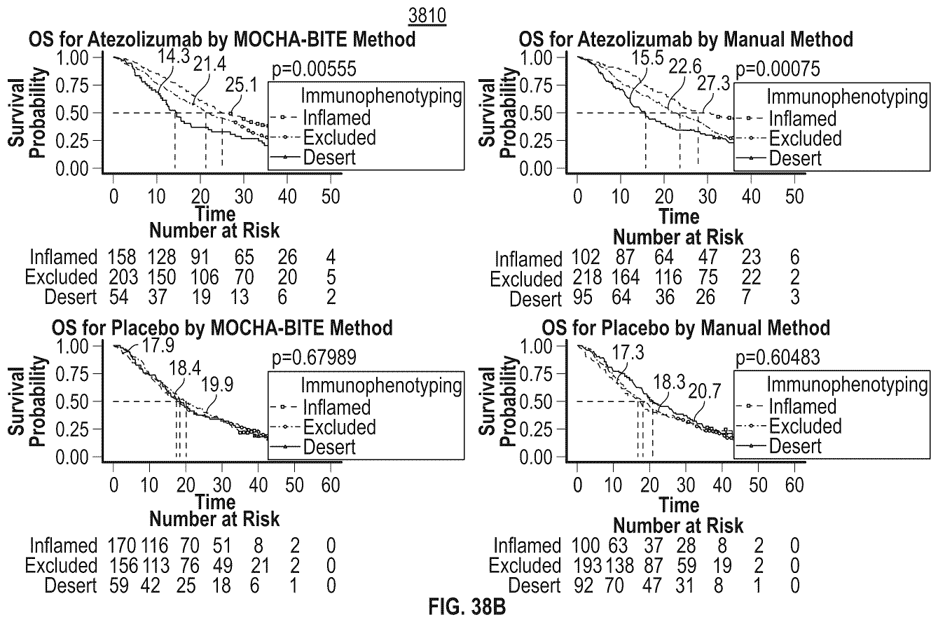

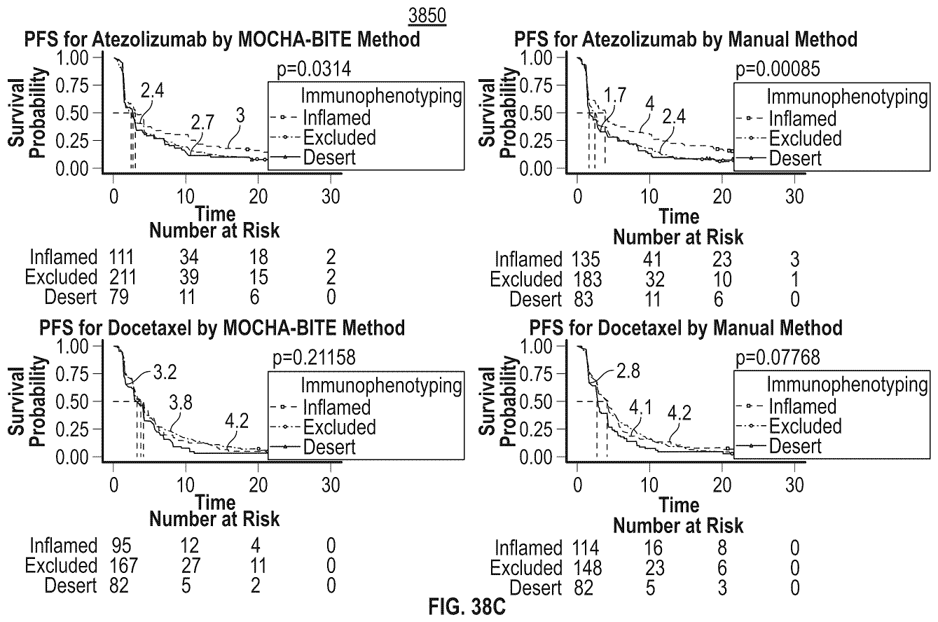

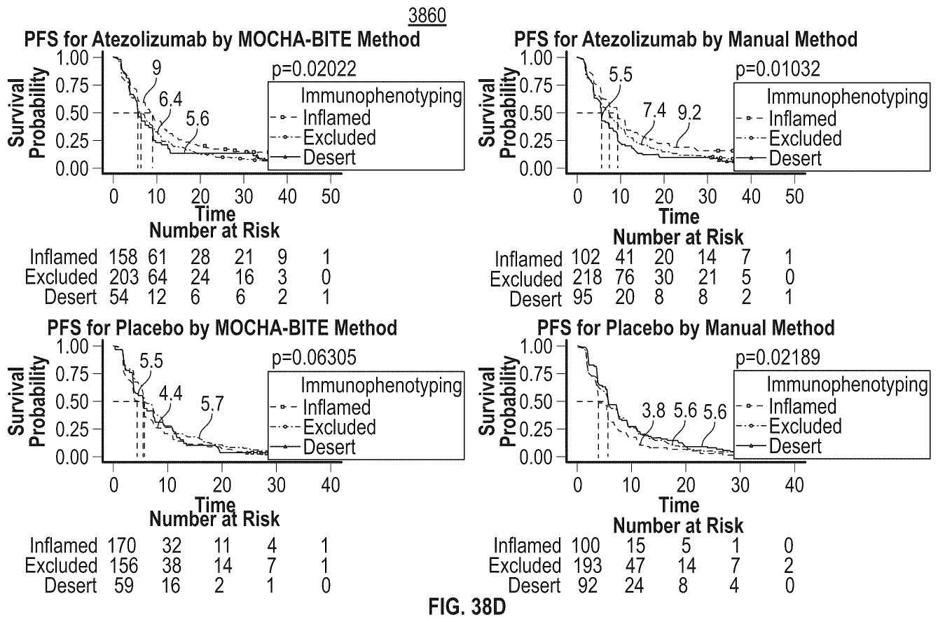

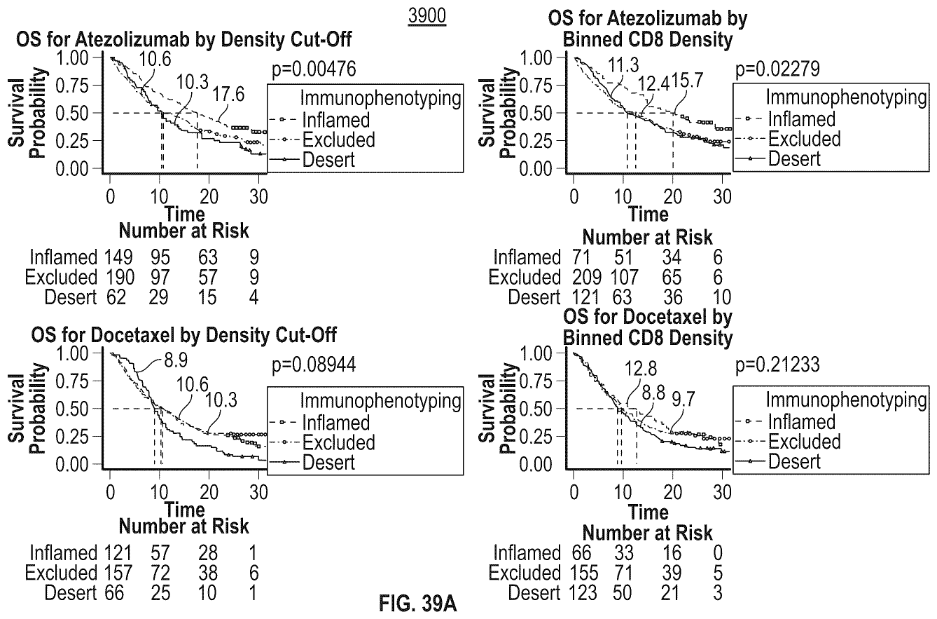

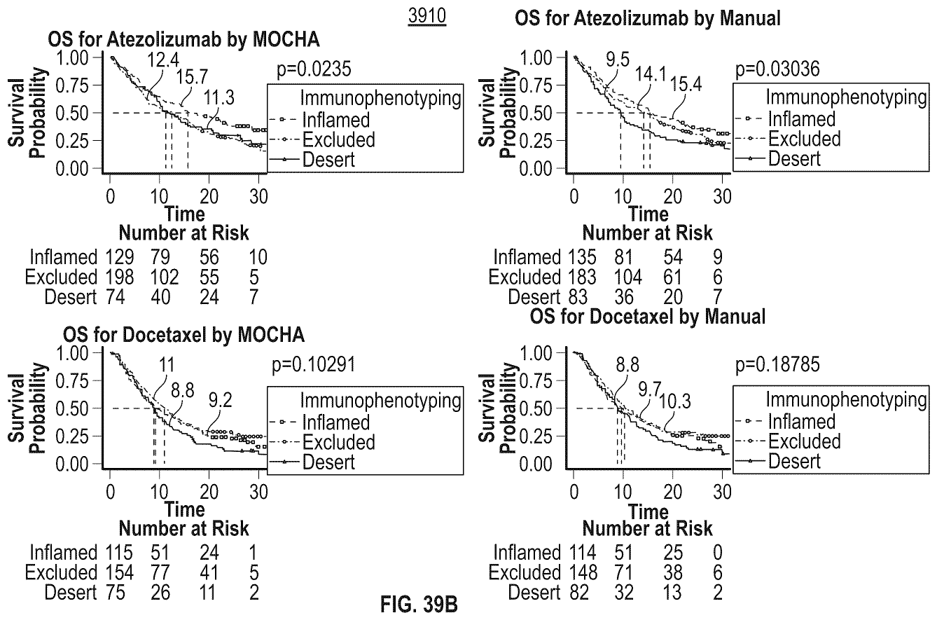

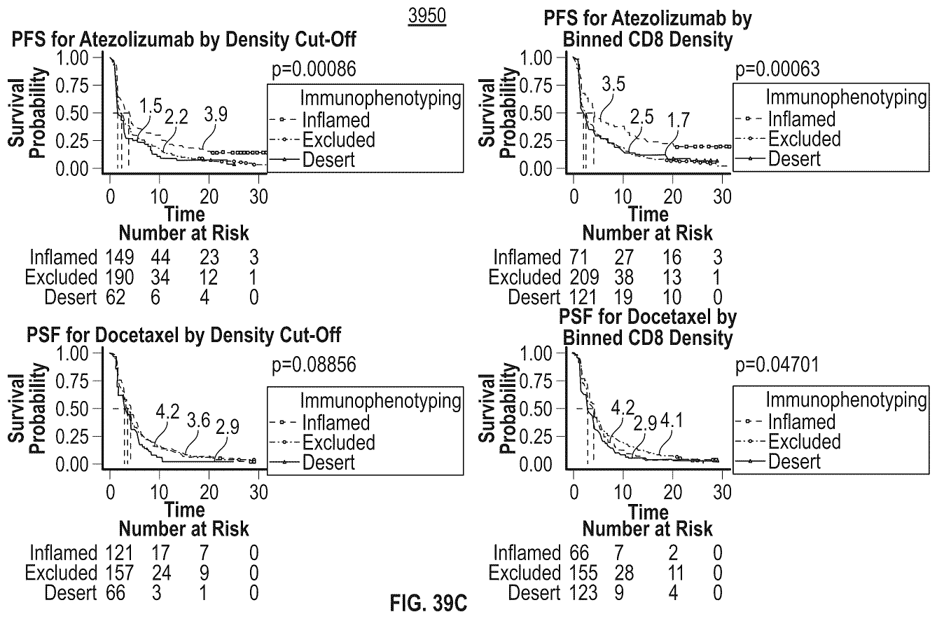

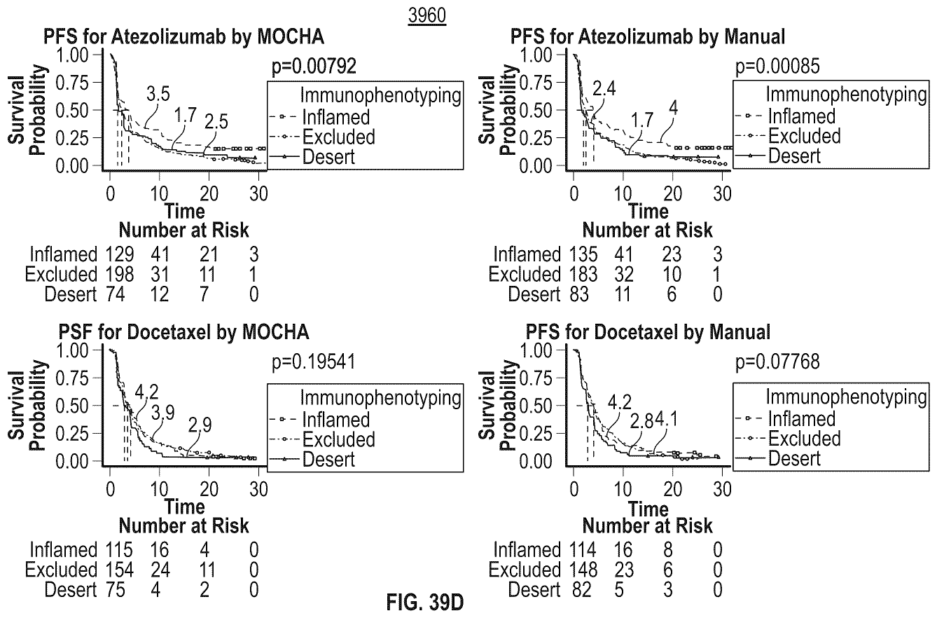

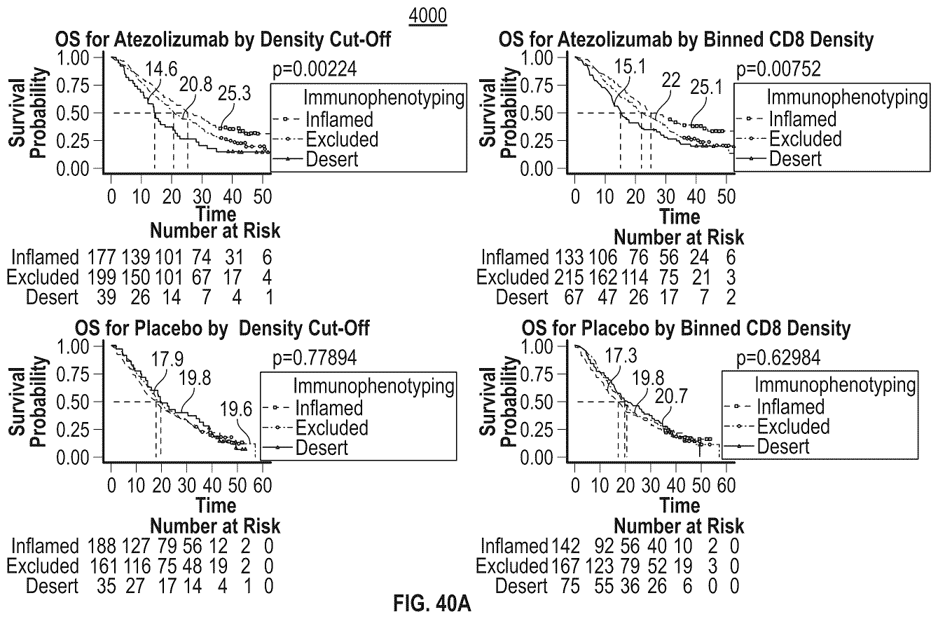

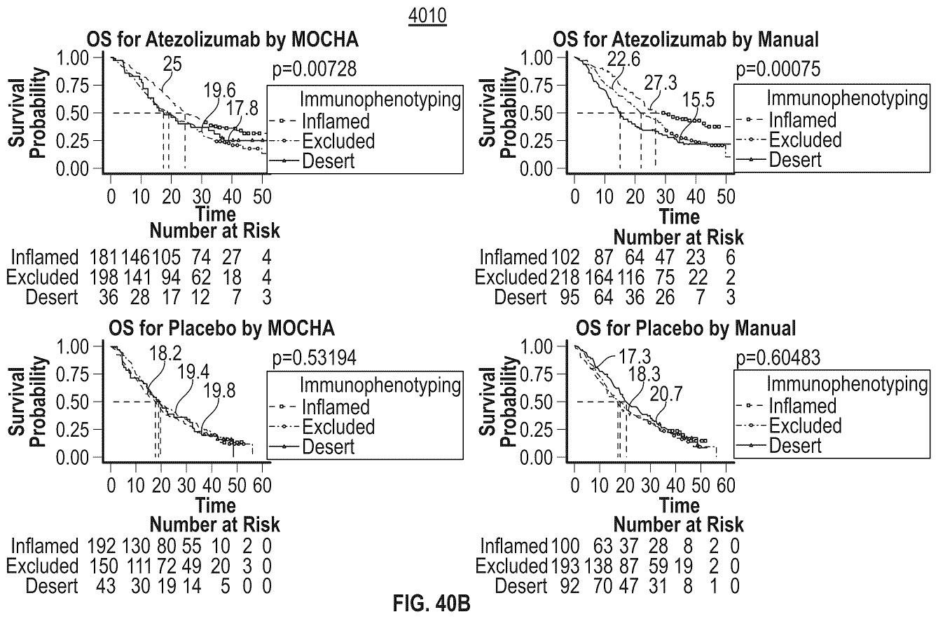

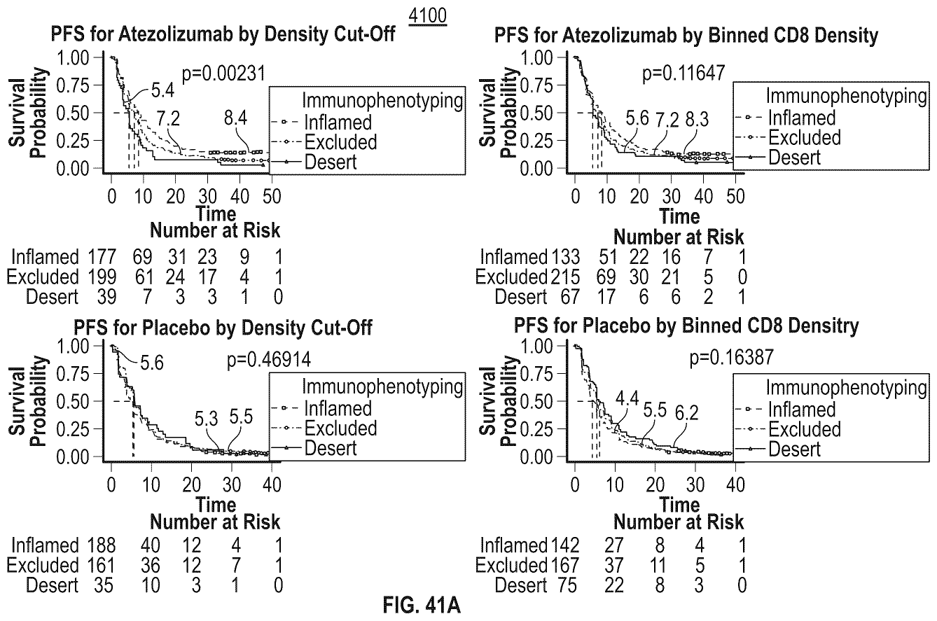

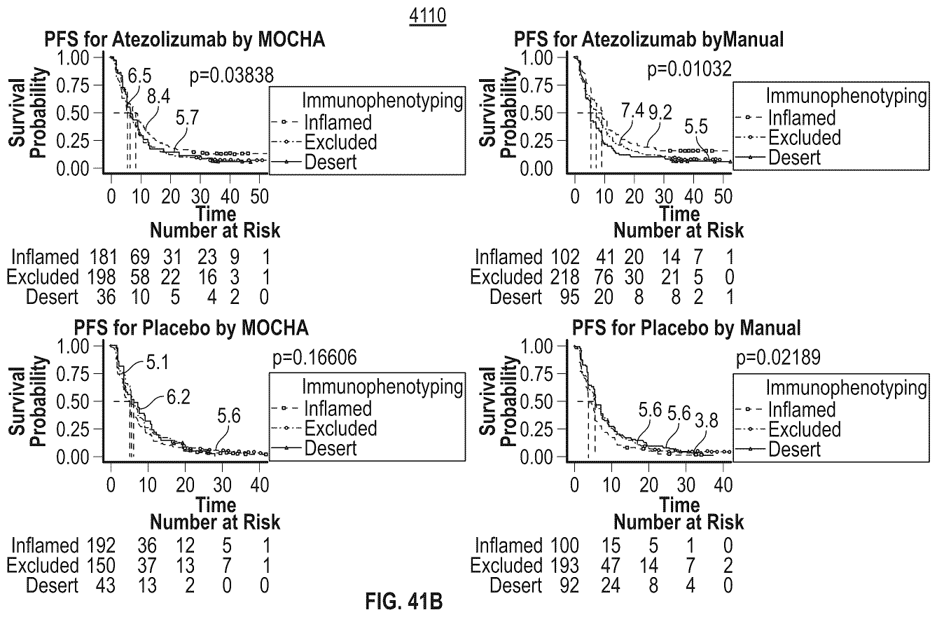

As seen from the plots 3500 of FIG. 35A, OS plots 3800 of FIG. 38A, OS plots 3810 of FIG. 38B, PFS plots 3850 of FIG. 38C, PFS plots 3860 of FIG. 38D, OS plots 3900 of FIG. 39A, OS plots 3910 of FIG. 39B, PFS plots 3950 of FIG. 39C, PFS plots 3960 of FIG. 39D, OS plots 4000 of FIG. 40A, OS plots 4010 of FIG. 40B, PFS plots 4100 of FIG. 41A, and PFS plots 4110 of FIG. 41B, for the example second clinical trial and the example third clinical trial, the fourth pipeline outperforms the first, second, and third pipelines. For example, the Cohen's kappa of the fourth pipeline for predicting a tumor immunophenotype was 0.582 for the example second clinical trial and 0.575 for the example third clinical trial. The fourth pipeline, trained on the example first clinical trial, yielded significant log-rank test results when comparing the set of tumor immunophenotypes (e.g., desert, excluded, inflamed) when analyzing for OS and PFS in the example second clinical trial and the example third clinical trial, as shown by plots 3500 of FIG. 35A, OS plots 3800 of FIG. 38A, OS plots 3810 of FIG. 38B, PFS plots 3850 of FIG. 38C, PFS plots 3860 of FIG. 38D, OS plots 3900 of FIG. 39A, OS plots 3910 of FIG. 39B, PFS plots 3950 of FIG. 39C, PFS plots 3960 of FIG. 39D, OS plots 4000 of FIG. 40A, OS plots 4010 of FIG. 40B, PFS plots 4100 of FIG. 41A, and PFS plots 4110 of FIG. 41B. For example, for patients of the example second clinical trial treated with a first immunotherapy (e.g., atezolizumab), the median OS was 10.8, 12.6 and 17.6 months for the tumor immunophenotypes desert, excluded, and inflamed tumors, respectively. The log-rank tests suggested the separation of OS curves among the set of tumor immunophenotypes was significant (p=0.04056). Furthermore, the median OS for manual tumor immunophenotype categorization was 9.5, 14.1, and 15.4 months, respectively the set of tumor immunophenotypes, as seen from the first group of four plots 3600 of FIG. 36. Neither tumor immunophenotype categorization showed statistically significant differences for OS for patients receiving a second immunotherapy (e.g., docetaxel), with a significant of p=0.26675 and 0.18785 when tumor immunophenotyping is implemented by the fourth pipeline and manually, respectively.

Similarly, when analyzing the example third clinical trial using the fourth pipeline, the median OS was 14.3, 21.4 and 25.1 months for the tumor immunophenotypes of desert, excluded, and inflamed IP, respectively. Manual tumor immunophenotype categorization of the same data yielded a median OS of 15.5, 22.6 and 27.3 months, respectively. Separation of the curves was statistically significant for both categorization methods (e.g., p=0.00555 and p=0.00075, respectively). No significant differences between tumor immunophenotyping were detected for patients receiving chemotherapy only (e.g., on the placebo arm), as seen in the second group of four plots 3600 from FIG. 36.

Still further, the effects of tumor immunophenotyping using the fourth pipeline or using manual tumor immunophenotyping based on PFS were observed for patients treated with a first immunotherapy (e.g., atezolizumab) in the example first, second, or third clinical trial, but was less pronounced compared to OS, as seen from plots 3700 of FIG. 37.

In some embodiments, the fourth pipeline implemented by fourth pipeline subsystem 118 can use a weakly supervised multiple instance learning-based classification approach to predict tumor immunophenotypes. For example, the fourth pipeline can involve dividing, using the tile generation module 1210, a histology image into a plurality of tiles. Each tile can depict a distinct structure within the imaged tissue and can be referred to as an “instance.” The histology image, treated as a collective entity, can serve as a “bag” encompassing multiple instances, and can be labeled as “positive” or “negative” for association with a tumor immunophenotype. In some embodiments, the fourth pipeline can involve determining, using the classification module 1228 and/or the classifier training module 1230, a tumor immunophenotype (e.g., tumor immunophenotype 1410) of the histology image using a classifier (e.g., classifier 1400) trained via multiple instance learning. In some embodiments, an attention score mechanism can be used to identify which instances within a bag contribute significantly to making a positive prediction and/or label. This attention-score-based process can aid in emphasizing the most relevant and discriminative regions within the histology image, contributing to the model and/or algorithm's ability to discern intricate patterns associated with the tumor immunophenotype. For example, attention scores can be derived from the features of each instance (e.g., by assessing their similarity in relation to features of other instances within the same bag). Instances that are more similar to other instances may be less strongly associated with a positive label, thereby having a lower attention score. For example, in a histology image containing only healthy cells, the healthy cells may all resemble one another, and there may be no reason to pay greater attention to one image tile over another. Conversely, instances that are more distinct may be more strongly associated with a positive label, thereby having a higher attention score. For example, in a histology image containing both healthy cells and tumor cells, the tumor cells may stand out from the healthy cells, and the multiple instance machine-learning model may pay greater attention to the image tiles containing the standout tumor cells when labeling the bag as “positive.” An instance-based model and/or instance-based algorithm can be trained to predict the tumor immunophenotype based on the attention scores. For example, a histology image containing both healthy cells and tumor cells may be labeled as “positive” based on the high attention scores associated with image tiles depicting tumor cells within the histology image, and the overall histology image can be labeled as containing a specific tumor type as a result.

Analyzing spatial statistics associated with digital pathology images has known technical benefits in different clinical use cases for solid tumors and/or liquid tumors. The techniques described herein illustrate the technical benefits of the disclosed pipelines to determine and assign tumor immunophenotypes to tumors based on digital pathology image analysis. The techniques described herein also provide validation for the using the various disclosed pipelines to predict tumor immunophenotype using digital pathology image analysis. The techniques described herein improve upon existing techniques, such as the use of an “immunoscore” or traditional TIL scoring. Additional technical advantages of the disclosed pipelines include an agnostic tumor immunophenotype determination process that removes subjective bias from immunophenotyping that can be introduced when performed using human evaluation and an increased speed and accuracy in determining tumor immunophenotype using digital pathology image analysis.

The present techniques enable unambiguous identification of epithelial cells forming tumor epithelium and stromal cells forming tumor stroma, as well as the detection and labeling of the relevant immune cell types (e.g., CD8+ T cells). These techniques further enable analysis for two different epithelial tumor types. Furthermore, the disclosed pipelines do not solely rely on resection specimens with representation of tumor center and invasive edges. As diverse specimen types are typically encountered in the clinical trial setting (e.g., from resections, excisional and needle core biopsies, etc.), the disclosed pipelines advantageously have a minimum requirement of viable, invasive tumor and associated stroma. This provides the technical benefit of expanding the number of available specimens for analysis as well as minimizing the introduction of unwanted bias toward clinical outcome.

Furthermore, the use of the disclosed pipelines has predictive value in determining tumor immunophenotypes. This includes focusing on intra-epithelial CD8+ effector cells in the analysis of each pipeline. The predictive value provided by the disclosed pipelines in classifying tumors into a set of tumor immunophenotypes, including inflamed, excluded, and desert can be observed with pre-defined, but subjectively implemented, density thresholds. The disclosed pipelines automate the tumor immunophenotyping process using immunohistochemically stained tumor sections, which provides the advantage of allowing for analysis of large sample cohorts, scalability, and the ability to address inter- and intra-observer variability. The disclosed pipelines showed that a combinatorial approach outperforms individual pipelines in two distinct tumor types. The training of the various pipelines was primarily evaluated using NSCLC clinical trial data; however, the disclosed pipelines were also successfully implemented during the analysis of TNBC clinical trial data. These evaluations further did not require additional machine-learning efforts, however further machine learning models may be used to automate additional aspects of the disclosed pipelines (e.g., biological object type detection). Extension to other tumor types (and other immune cell phenotypes) may be straightforward as long as the relevant cell types can be unambiguously identified.

Some existing techniques use deep learning-based Al models to segment tumor regions on H&E-stained images or on IHC stained images that lack a tumor marker. However, differing from these existing techniques, the present application describes a simple morphological approach that works on a tumor marker believed to be more robust and generalizable than ML approaches not extensively trained on multiple indications. Manual tumor immunophenotyping is typically performed using cutoffs on sections stained only for immune cells, and not on H&E.

The disclosed pipelines can be expanded to other epithelial tumor types without the need to train a new algorithm for recognition of tumor regions or tumor cells in a separate indication. Importantly, two distinct approaches on different patient cohorts show that the spatial distribution of immune cells has predictive value for CI-based therapies suggesting biologically relevant mechanisms that deserve further exploration.

The disclosed pipelines further allowable for the evaluation of immune cell density distributions for immune cells other than CD8+ T effector cells as long as they can be identified by immunohistochemical means. This can be beneficial as certain CI therapies targeting molecules other than PD-1 and PD-L1 can then be used. Furthermore, the disclosed pipelines can allow for expansion into a multiplexed methodology with the identification of more than one immune cell phenotype.

One or more of first pipeline subsystem 112, second pipeline subsystem 114, third pipeline subsystem 116, and/or fourth pipeline subsystem 118 may be configured to train one or more machine learning models. For example, tile generation modules 510, tile generation module 1010, tile generation module 1210, as well as, in some examples, components of first pipeline subsystem 112, may implement one or more machine learning models. Thus, one or more subsystems of computing system 102 may be configured to perform a training process to training machine learning models, which may then be deployed by other components.

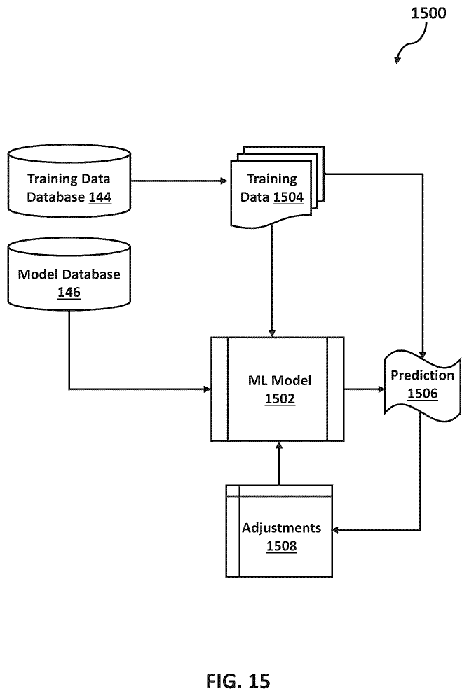

As an example, with reference to FIG. 15, one or more subsystems (e.g., first pipeline subsystem 112, second pipeline subsystem 114, third pipeline subsystem 116, fourth pipeline subsystem 118) of computing system 102 may be configured to perform process 1500 to train a machine learning model 1502. Persons of ordinary skill in the art will recognize that other training processes may be used to train a machine learning model used by components of system 100. For example, some models may use contrastive learning. Thus, process 1500 should not be construed as limiting the disclosed embodiments to particular training processes. Furthermore, process 1500 is not restricted to one type of machine learning model. In process 1500, training data 1504 may be retrieved from training data database 144. Different training data may be used to train different types of machine learning models. Furthermore, validation data may also be stored in training data database 144. The training data and the validation data may be identified and retrieved prior to the training process beginning.

In some embodiments, training data 1504 may include images depicting biological samples. For example, the images may depict tumor regions of patients diagnosed with NSCLC. Training data 1504 may include whole slide images. The whole slide images may be split into image tiles (using a process the same or similar to the image tiling techniques described in FIG. 6). Training data 1504 may include these image tiles. One or more subsystems of computing system 102 may select a to-be-trained machine learning model (e.g., machine learning model 1502), which may be retrieved from model database 146. Machine learning model 1502 may be selected based on a type of biological sample being analyzed, an immunophenotype to be identified, an immunotherapy to be determined, and/or other criteria. One or more subsystems of computing system 102 may select training data 1504, which may be retrieved from training data database 144. One or more subsystems of computing system 102 may select training data 1504 from training data stored in training data database 144 based on a type of machine learning model that was selected.

One or more subsystems of computing system 102 may provide training data 1504 to machine learning model 1502. Training data 1504 may include images depicting biological samples. For example, the images may depict tumor regions of patients diagnosed with NSCLC. Training data 1504 may be input to machine learning model 1502, which may generate a prediction 1506. Prediction 1506 may indicate, amongst other information, characteristics of the biological samples depicted by the images in training data 1504.

Prediction 1506 may be compared to a ground truth identified from training data 1504. As mentioned above, the images included in training data 1504 may include labels. These labels may indicate characteristics of the biological sample (e.g., cellular structures identified). Therefore, prediction 1506 may indicate whether machine learning model 1502 correctly identified the characteristics. One or more subsystems of computing system 102 may be configured to compare a given image's label with prediction 1506 for that image. One or more subsystems of computing system 102 may further determine one or more adjustments 1508 to be made to one or more hyper-parameters of machine learning model 1502. The adjustments to the hyper-parameters may be to improve predictive capabilities of machine learning model 1502. For example, based on the comparison, one or more subsystems of computing system 102 may adjust weights and/or biases of one or more nodes of machine learning model 1502. Process 1500 may repeat until an accuracy of machine learning model 1502 reaches a predefined accuracy level (e.g., 95% accuracy or greater, 99% accuracy or greater, etc.), at which point machine learning model 1502 may be stored in model database 146 as a trained machine learning model. The accuracy of machine learning model 1502 may be determined based on a number of correct predictions (e.g., prediction 1506).

### Example Flowcharts

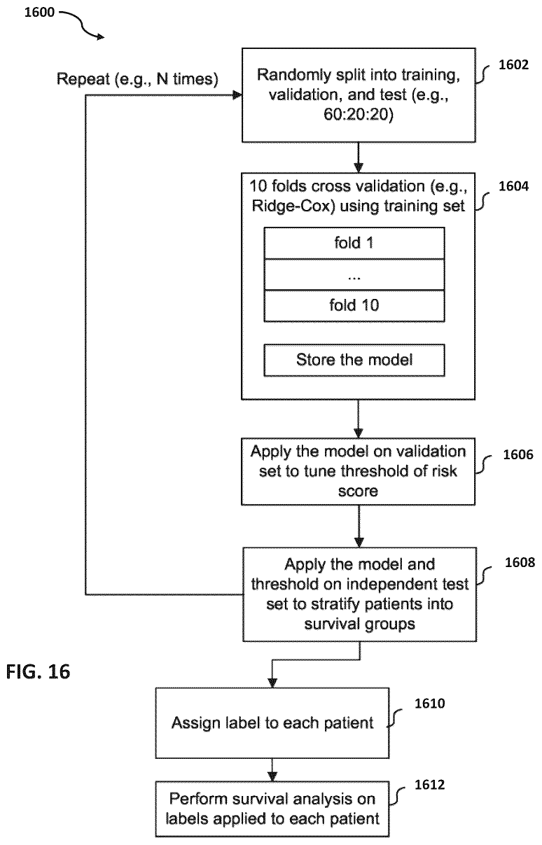

FIG. 16 is an illustrative flowchart of process 1600 describing an overall workflow for predictive analysis, in accordance with various embodiments. In some embodiments, process 1600 may be implemented by one or more subsystems of computing system 102, such as first pipeline subsystem 112, second pipeline subsystem 114, third pipeline subsystem 116, fourth pipeline subsystem 118, or modules included therein, or other components of system 100, or combinations thereof. More specifically, in order to assign each subject in the study cohort a label, a nested Monte Carlo Cross-Validation (nMCCV) modeling strategy can be used to overcome overfitting.

Process 1600 may begin at operation 1602. At operation 1602, for each subject (e.g., patient), a data set can be split into training, validation, and test data portions in 60:20:20 proportions. At operation 1604, 10-fold cross-validation Ridge-Cox (L2 regularized Cox model) can be performed using the training set to produce 10 models (having a same model architecture). A particular model across the 10 produced models can be selected based on the 10-fold training data and stored. At operation 1606, the particular model can then be applied on the validation set to tune a specified variable. For example, the variable can identify a threshold for a risk score. At operation 1608, the threshold and particular model can then be applied to the independent test set to generate a vote for the subject predicting whether the subject is stratified into a longer or shorter survival group. The data splitting, training, cut-off identification and vote generation (operations 1602-1608) can be repeated N (e.g., N=1000) times. At operation 1610, the subject may be assigned to one of a longer survival group or a shorter survival group based on the votes. For example, operation 1610 can include assigning a subject to a longer survival group or shorter survival group by determining which group was associated with the majority of votes. At operation 1612, a survival analysis can then be performed of the longer/shorter survival group subjects. It will be appreciated that similar procedures to apply a wide variety of labels to data, based on the outcomes of interest, can be applied any suitable clinical evaluation or eligibility study.

The comprehensive model based on immune cell density distributions, spatial statistics, and spatial-distribution metrics, and/or concatenated representations used in the analysis of this example empowers an analytical pipeline that generates system-level knowledge of, in this case, immunophenotype decided based on intra-tumoral density by modeling histopathology images as spatial data assisted through pixel-based segmentation. This effect is not limited to particular treatment evaluations but can be applied in many scenarios where the necessary ground truth data in available. Using spatial statistics to characterize histopathology images, and other digital pathology images, can be useful in the clinical setting to predict treatment outcomes and to thus inform treatment selection.

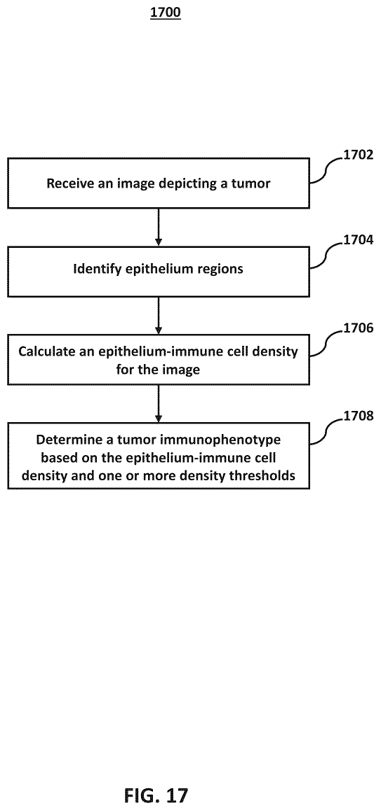

FIG. 17 is an illustrative flowchart of an exemplary process 1700 for determining a tumor immunophenotype using a first pipeline, in accordance with various embodiments. In some embodiments, process 1700 may be implemented by one or more modules and components of first pipeline subsystem 112. However, in some embodiments, additional or alternative components of system 100 may be employed to execute one or more aspects of process 1700.

In some embodiments, process 1700 may begin at operation 1702. In operation 1702, an image depicting a tumor may be received. In some embodiments, the image is a digital pathology image captured using a digital pathology imaging system (e.g., image scanner 240). The image may be a whole slide image or a portion of a whole slide image. The whole slide image may be annotated to identify tumor lesions, and the image may be extracted from the whole slide image. In some embodiments, a dual-stain may be applied to a sample of the tumor prior to the image being captured. The dual-stain may include a first stain and a second stain. For example, the first stain may distinguish and highlight tumor epithelium from tumor stroma and the second stain may highlights immune cell. In some embodiments, the first stain may be a pan-cytokeratin (panCK) stain used for highlighting the tumor epithelium (e.g., CK+ tumor cells) and tumor stroma (e.g., CK− tumor cells), and the second stain may be a cluster of differentiation 8 (CD8) stain used for highlighting immune cells (e.g., CD8+ T cells).

In operation 1704, one or more regions of the image depicting tumor epithelium may be identified. In some embodiments, the regions may be identified by scanning the image using a sliding window. For each portion of the image included within the sliding window, that portion may be classified as a region depicting tumor epithelium based on the portion satisfying a tumor epithelium criterion and/or a region depicting tumor stroma based on the portion satisfying a tumor stroma criterion. The tumor epithelium criterion being satisfied may include at least a threshold amount of the portion depicting tumor epithelium. For example, the threshold amount may be 25% of the portion of the image. The tumor stroma criterion being satisfied may include at least a threshold amount of the portion depicting tumor stroma. For example, the threshold amount may be 25% of the portion of the image. In some embodiments, classifying the portion as a region of tumor epithelium, a region of tumor stroma, and/or identifying immune cells within either of these regions, a color deconvolution may be performed to the image to obtain a plurality of color channel images. For example, the plurality of color images may include a first color channel image highlighting tumor epithelium and tumor stroma and a second color channel image highlighting immune cells. In some embodiments, a region depicting tumor epithelium may also depict tumor stroma. Thus, a region can be a tumor epithelium region and/or a tumor stroma region.

In operation 1706, an epithelium-immune cell density may be calculated for the image. The epithelium-immune cell density for the image may be calculated based on a number of immune cells detected within the one or more regions of the image depicting the tumor epithelium. In some embodiments, immune cells within the tumor epithelium regions may be determined by a trained pathologist. In some embodiments, a color deconvolution process may be performed to the image to obtain a plurality of color channels, where one color channel highlights tumor epithelium and another color channel highlights immune cells. The different color channel images may then be analyzed to determine the number of immune cells present within each region depicting tumor epithelium. In some embodiments, the number of immune cells detected within each of the one or more regions of the image may be determined using one or more machine learning models. The one or more machine learning models may include a computer vision model trained to recognize immune cells. An example computer vision model that may be used to recognize immune cells within tumor epithelium regions of an image may be a convolutional neural network (CNN). In some embodiments, the epithelium-immune cell density that is calculate may be an average epithelium-immune cell density. For example, for each region depicting tumor epithelium, an epithelium-immune cell density may be calculated. These epithelium-immune cell densities may be averaged together across the digital pathology image to obtain the average epithelium-immune cell density for the image.

In operation 1708, a tumor immunophenotype for the image may be determined based on the epithelium-immune cell density and one or more density thresholds. The density thresholds may indicate which tumor immunophenotype of a set of tumor immunophenotypes the image should be classified into. In some embodiments, the density thresholds may include a first density threshold and the tumor immunophenotypes may include inflamed and non-inflamed. If the epithelium-immune cell density of the image (e.g., the average epithelium-immune cell density) is greater than or equal to the first density threshold, then the image depicting the tumor may be classified into the tumor immunophenotype inflamed. However, if the epithelium-immune cell density of the image (e.g., the average epithelium-immune cell density) is less than the first density threshold, then the image may be classified into the tumor immunophenotype non-inflamed. In some embodiments, the density thresholds may include a first density threshold and a second density threshold, and the tumor immunophenotypes may include desert, excluded, and inflamed. If the epithelium-immune cell density of the image (e.g., the average epithelium-immune cell density) is greater than or equal to the first density threshold, then the image depicting the tumor may be classified into the tumor immunophenotype, “inflamed.” If the epithelium-immune cell density of the image (e.g., the average epithelium-immune cell density) is less than the first density threshold and greater than or equal to the second density threshold, then the image depicting the tumor may be classified into the tumor immunophenotype, “excluded.” If the epithelium-immune cell density of the image (e.g., the average epithelium-immune cell density) is less than the first density threshold and the second density threshold, then the image depicting the tumor may be classified into the tumor immunophenotype, “desert.”

In some embodiments, patients classified into the tumor immunophenotype inflamed may receive a first immunotherapy. Thus, the ability to identify which patients will receive a particular immunotherapy may depend on the immunophenotyping process.

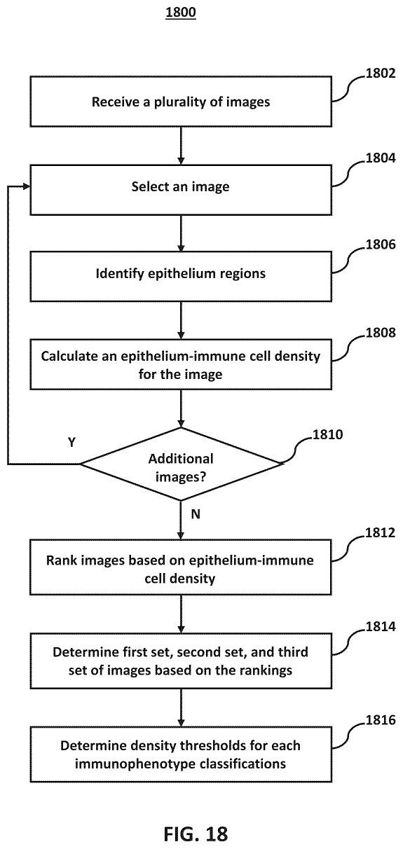

FIG. 18 is an illustrative flowchart of a process 1800 for determining density thresholds used for determining a tumor immunophenotype, in accordance with various embodiments. In some embodiments, process 1800 may be implemented by one or more modules and components of first pipeline subsystem 112. For example, process 1800 may be performed by density threshold determination module 316 of first pipeline subsystem 112, as described above. However, in some embodiments, additional or alternative components of system 100 may be employed to execute one or more aspects of process 1800.

In some embodiments, process 1800 may begin at operation 1802. In operation 1802, a plurality of images may be received. Each of the images may correspond to a patient from a plurality of patients participating in a clinical trial. In some embodiments, the images may be stored in image database 142.

In operation 1804, an image from the received images may be selected. In operation 1806, one or more regions of the selected image depicting tumor epithelium may be identified. In operation 1808, an epithelium-immune cell density for the selected image may be calculated based on a number of immune cells detected within the regions depicting tumor epithelium. Operations 1806 and 1808 may be the same or similar to operations 1704 and 1706, respectively, of FIG. 17, and the previous description may apply. In some embodiments, the epithelium-immune cell density calculated at operation 1808 may be an average epithelium-immune cell density for the selected image.

In operation 1810, a determination may be made as to whether any additional images from the received plurality of images at operation 1802 are to be analyzed. If so, process 1800 may return to operation 1804 where a different image from the received plurality of images may be selected and operations 1806-1810 may repeat. However, if at operation 1810 it is determined that the images received at operation 1802 have been analyzed, then process 1800 may proceed to operation 1812.

In operation 1812, the plurality of images may be ranked based on each image's epithelium-immune cell density. For example, with reference to FIG. 4, ranking 400 may include a ranking of images 402 based each image's average epithelium-immune cell density. In some embodiments, the ranking (e.g., ranking 400) may be based on each image's average epithelium-immune cell density.

In operation 1814, a first set, a second set, and a third set of images may be determined based on the ranking. The first set may include images from the ranking having epithelium-immune cell densities in a bottom 20% of the ranking, the second set may include images from the ranking having epithelium-immune cell densities in a next 40% of the ranking, and the third set may include images from the ranking having epithelium-immune cell densities in a top 40% of the ranking. In the example of FIG. 4, three tumor immunophenotypes may be used. If, however, only two tumor immunophenotypes are used (e.g., inflamed or non-inflamed), then the first set of images may include the bottom 60% of ranking 400 and the second set of images may include an upper 40% of ranking 400.

In operation 1816, density thresholds for each tumor immunophenotype classification may be determined. For example, first threshold 404 may indicate whether an image is to be classified as being inflamed or non-inflamed and second threshold 406 may indicate whether an image classified as being non-inflamed should be assigned to the tumor immunophenotypes desert or excluded. For example, epithelium-immune cell densities greater than or equal to first density threshold 404 may be classified into the tumor immunophenotype inflamed, epithelium-immune cell densities less than first density threshold 404 and greater than or equal to second density threshold 406 may be classified into the tumor immunophenotype excluded, and epithelium-immune cell densities less than first density threshold 404 and second density threshold 406 may be classified into the tumor immunophenotype desert.

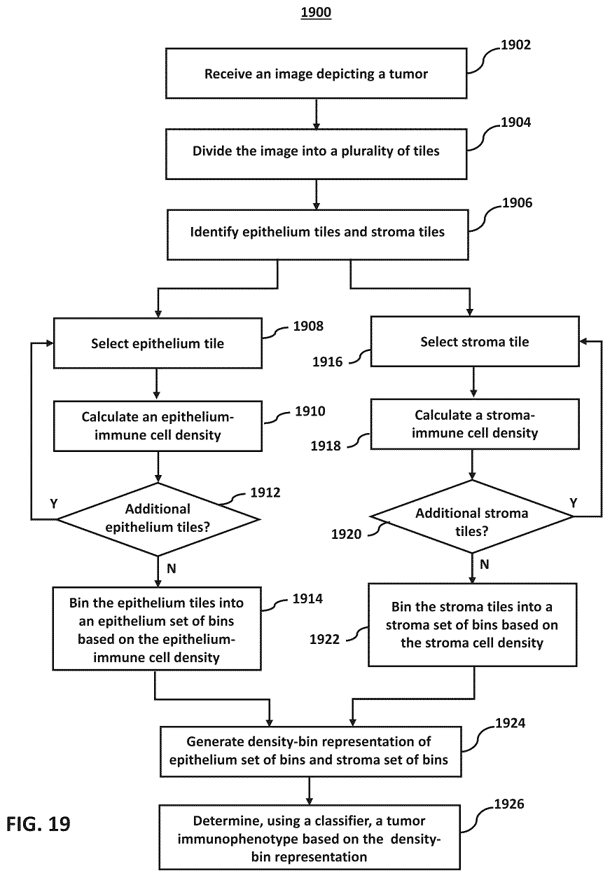

FIG. 19 is an illustrative flowchart of an exemplary process 1900 for determining a tumor immunophenotype using a second pipeline, in accordance with various embodiments. In some embodiments, process 1900 may be implemented by one or more modules and components of second pipeline subsystem 114. However, in some embodiments, additional or alternative components of system 100 may be employed to execute one or more aspects of process 1900.

In some embodiments, process 1900 may begin at operation 1902. At operation 1902, an image depicting a tumor may be received. In some embodiments, the image is a digital pathology image captured using a digital pathology imaging system (e.g., image scanner 240). The image may be a whole slide image or a portion of a whole slide image. The whole slide image may be annotated to identify tumor lesions, and the image may be extracted from the whole slide image. In some embodiments, a dual-stain may be applied to a sample of the tumor prior to the image being captured. The dual-stain may include a first stain and a second stain. For example, the first stain may distinguish and highlight tumor epithelium from tumor stroma and the second stain may highlights immune cell. In some embodiments, the first stain may be a pan-cytokeratin (panCK) stain used for highlighting the tumor epithelium (e.g., CK+ tumor cells) and tumor stroma (e.g., CK− tumor cells), and the second stain may be a cluster of differentiation 8 (CD8) stain used for highlighting immune cells (e.g., CD8+ T cells).

In operation 1904, the image may be divided into a plurality of tiles. The tiles may be overlapping or non-overlapping. For example, with reference to FIG. 6, digital pathology image 604 may be divided into tiles 606.

In operation 1906, epithelium tiles and stroma tiles may be identified from the plurality of tiles. In some embodiments, a tile may be classified as an epithelium tile based on a portion of the tile depicting tumor epithelium satisfying an epithelium-tile criterion. The epithelium-tile criterion being satisfied may include the portion of the tile depicting tumor epithelium being greater than or equal to a first threshold area. For example, the first threshold area may include 25% of the tile. In some embodiments, a tile may be classified as a stroma tile based on a portion of the tile depicting tumor stroma satisfying a stroma-tile criterion. The stroma-tile criterion being satisfied may include the portion of the tile depicting the tumor stroma being greater than or equal to a second threshold area. For example, the second threshold area may include 25% of the tile. Thus, in some embodiments, a tile may be classified as being both an epithelium tile and a stroma tile.

In operation 1908, an epithelium tile may be selected from the identified epithelium tiles. In operation 1910, an epithelium-immune cell density may be calculated for the selected epithelium tile. In some embodiments, the epithelium-immune cell density may be calculated by determining a number of immune cells detected within the selected epithelium tile. The epithelium-immune cell density for the selected tile may be calculated based on a number of immune cells detected within the selected epithelium tile. In some embodiments, immune cells within epithelium tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected epithelium tile. In some embodiments, the number of immune cells detected within the selected epithelium tile may be determined using one or more machine learning models. The one or more machine learning models may include a computer vision model trained to recognize immune cells. An example computer vision model that may be used to recognize immune cells within tumor epithelium regions of an image may be a convolutional neural network (CNN).

In operation 1912, a determination may be made as to whether any additional epithelium tiles are to be analyzed. If so, process 1900 may return to operation 1908 where another epithelium tile may be selected and operations 1910-1912 may repeat. However, if at operation 1912 it is determined that no additional epithelium tiles are to be analyzed, then process 1900 may proceed to operation 1914.

In operation 1914, the epithelium tiles may be binned into an epithelium set of bins based on the epithelium-immune cell density of each epithelium tile. For example, the epithelium tiles may be binned into one of epithelium set of bins 802 of FIG. 8. In some embodiments, binning an epithelium tile into one of the epithelium set of bins may include determining a density range for each bin of the epithelium set of bins. Each epithelium tile of the plurality of tiles may be allocated to one of the epithelium set of bins based on the epithelium-immune cell density of the epithelium tile. In some embodiments, the epithelium set of bins may include ten bins. For example, the epithelium set of bins may be defined by ten different density ranges: a first density range comprising epithelium-immune cell densities of 0.0-0.005, a second density range comprising epithelium-immune cell densities of 0.005-0.01, a third density range comprising epithelium-immune cell densities of 0.01-0.02, a fourth density range comprising epithelium-immune cell densities of 0.02-0.04, a fifth density range comprising epithelium-immune cell densities of 0.04-0.06, a sixth density range comprising epithelium-immune cell densities of 0.06-0.08, a seventh density range comprising epithelium-immune cell densities of 0.08-0.12, an eighth density range comprising epithelium-immune cell densities of 0.12-0.16, a ninth density range comprising epithelium-immune cell densities of 0.16-0.2, and a tenth density range comprising epithelium-immune cell densities of 0.2-2.0.

In operation 1916, a stroma tile may be selected from the identified stroma tiles. In operation 1918, a stroma-immune cell density may be calculated for the selected stroma tile. In some embodiments, the stroma-immune cell density may be calculated by determining a number of immune cells detected within the selected stroma tile. The stroma-immune cell density for the selected tile may be calculated based on a number of immune cells detected within the selected stroma tile. In some embodiments, immune cells within stroma tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected stroma tile. In some embodiments, the number of immune cells detected within the selected stroma tile may be determined using one or more machine learning models. The one or more machine learning models may include a computer vision model trained to recognize immune cells. An example computer vision model that may be used to recognize immune cells within tumor stroma regions of an image may be a convolutional neural network (CNN).

In operation 1920, a determination may be made as to whether any additional stroma tiles are to be analyzed. If so, process 1900 may return to operation 1916 where another stroma tile may be selected and operations 1918-1920 may repeat. However, if at operation 1920 it is determined that no additional stroma tiles are to be analyzed, then process 1900 may proceed to operation 1922.

In operation 1922, the stroma tiles may be binned a stroma set of bins based on the epithelium-immune cell density of each epithelium tile. For example, the stroma tiles may be binned into one of stroma set of bins 804 of FIG. 8. In some embodiments, binning stroma tile into one of the stroma set of bins may include determining a density range for each bin of the stroma set of bins. Each stroma tile of the plurality of tiles may be allocated to one of the stroma set of bins based on the stroma-immune cell density of the stroma tile. In some embodiments, the stroma set of bins may include ten bins. For example, the stroma set of bins may be defined by ten different density ranges: a first density range comprising stroma-immune cell densities of 0.0-0.005, a second density range comprising stroma-immune cell densities of 0.005-0.01, a third density range comprising stroma-immune cell densities of 0.01-0.02, a fourth density range comprising stroma-immune cell densities of 0.02-0.04, a fifth density range comprising stroma-immune cell densities of 0.04-0.06, a sixth density range comprising stroma-immune cell densities of 0.06-0.08, a seventh density range comprising stroma-immune cell densities of 0.08-0.12, an eighth density range comprising stroma-immune cell densities of 0.12-0.16, a ninth density range comprising stroma-immune cell densities of 0.16-0.2, and a tenth density range comprising stroma-immune cell densities of 0.2-2.0.

In some embodiments, operations 1908-1914 and operations 1916-1922 may be performed in parallel or sequentially.

In operation 1924, a density-bin representation may be generated based on the epithelium set of bins and the stroma set of bins. The density-bin representation may include a plurality of elements corresponding to each bin of the epithelium set of bins and each bin of the stroma set of bins. As an example, with reference to FIG. 9, density-bin representation 910 may be generated based on immune cell density distribution 800 formed of epithelium set of bins 802 and stroma set of bins 804. In some embodiments, the density-bin representation may include 20-elements forming a 20-dimensional feature vector.

In operation 1926, a tumor immunophenotype may be determined based on the density-bin representation. In some embodiments, a trained classifier may be used to determine the tumor immunophenotype. For example, classifier 920 may be used to determine tumor immunophenotype 930 based on density-bin representation 910. In some embodiments, the classifier (e.g., classifier 920) may be a multi-class classifier configured to classify an image into one of a set of tumor immunophenotypes. Some example classifiers that may be used include, but are not limited to, a trained support vector machine (SVM), a random forest (RF) classifier, a decision tree classifier, a logistic regression classifier, or a k-nearest neighbor (kNN) classifier.

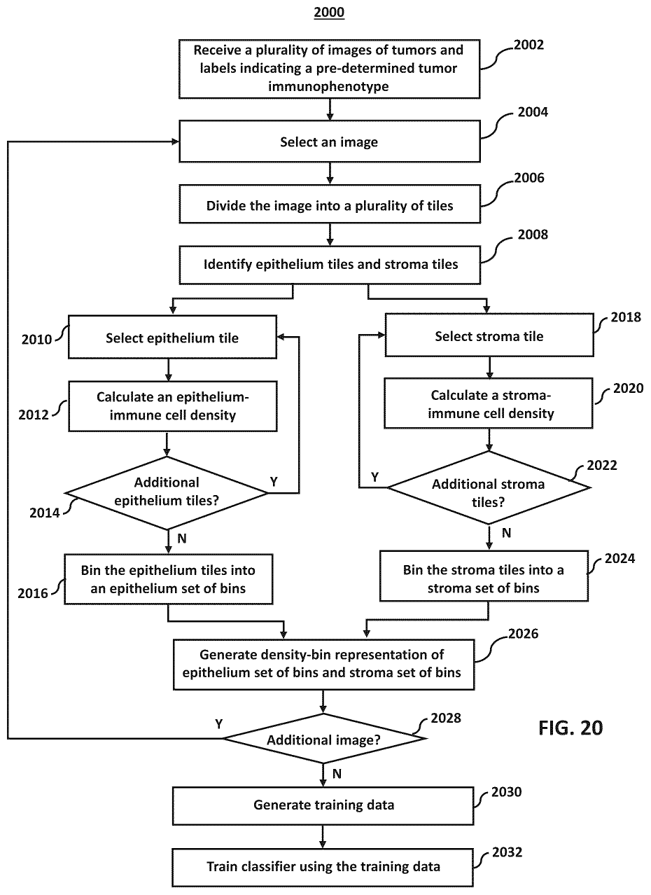

FIG. 20 is an illustrative flowchart of a process 2000 for training a classifier to determine a tumor immunophenotype, in accordance with various embodiments. In some embodiments, process 2000 may be implemented by one or more modules and components of second pipeline subsystem 114. For example, process 2000 may be performed by classifier training module 524 of second pipeline subsystem 114, as described above. However, in some embodiments, additional or alternative components of system 100 may be employed to execute one or more aspects of process 2000.

In some embodiments, process 2000 may begin at operation 2002. In operation 2002, a plurality of images may be received. Each of the images may correspond to a patient from a plurality of patients participating in a clinical trial. In some embodiments, the plurality of images may include labels indicating a pre-determined tumor immunophenotype. For example, each image may include metadata that labels the tumor immunophenotype of the tumor depicted by the image. The tumor immunophenotype may be pre-determined by a trained pathologist, using one or more machine learning models, or a combination thereof. The tumor immunophenotype may be selected, for example, by the trained pathologist from a set of tumor immunophenotypes including desert, excluded, and inflamed. In some embodiments, the images may be stored in image database 142.

In operation 2004, an image from the received images may be selected.

In operation 2006, the image may be divided into a plurality of tiles. The tiles may be overlapping or non-overlapping. For example, with reference to FIG. 6, digital pathology image 604 may be divided into tiles 606.

In operation 2008, epithelium tiles and stroma tiles may be identified from the plurality of tiles. In some embodiments, a tile may be classified as an epithelium tile based on a portion of the tile depicting tumor epithelium satisfying an epithelium-tile criterion. The epithelium-tile criterion being satisfied may include the portion of the tile depicting tumor epithelium being greater than or equal to a first threshold area. For example, the first threshold area may include 25% of the tile. In some embodiments, a tile may be classified as a stroma tile based on a portion of the tile depicting tumor stroma satisfying a stroma-tile criterion. The stroma-tile criterion being satisfied may include the portion of the tile depicting the tumor stroma being greater than or equal to a second threshold area. For example, the second threshold area may include 25% of the tile. Thus, in some embodiments, a tile may be classified as being both an epithelium tile and a stroma tile.

In operation 2010, an epithelium tile may be selected from the identified epithelium tiles. In operation 2012, an epithelium-immune cell density may be calculated for the selected epithelium tile. In some embodiments, the epithelium-immune cell density may be calculated by determining a number of immune cells detected within the selected epithelium tile. The epithelium-immune cell density for the selected tile may be calculated based on a number of immune cells detected within the selected epithelium tile. In some embodiments, immune cells within epithelium tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected epithelium tile. In some embodiments, the number of immune cells detected within the selected epithelium tile may be determined using one or more machine learning models. The one or more machine learning models may include a computer vision model trained to recognize immune cells. An example computer vision model that may be used to recognize immune cells within tumor epithelium regions of an image may be a convolutional neural network (CNN).

In operation 2014, a determination may be made as to whether any additional epithelium tiles are to be analyzed. If so, process 2000 may return to operation 2010 where another epithelium tile may be selected and operations 2012-2014 may repeat. However, if at operation 2014 it is determined that no additional epithelium tiles are to be analyzed, then process 2000 may proceed to operation 2016.

In operation 2016, the epithelium tiles may be binned into an epithelium set of bins based on the epithelium-immune cell density of each epithelium tile. For example, the epithelium tiles may be binned into one of epithelium set of bins 802 of FIG. 8. In some embodiments, binning an epithelium tile into one of the epithelium set of bins may include determining a density range for each bin of the epithelium set of bins. Each epithelium tile of the plurality of tiles may be allocated to one of the epithelium set of bins based on the epithelium-immune cell density of the epithelium tile. In some embodiments, the epithelium set of bins may include ten bins. For example, the epithelium set of bins may be defined by ten different density ranges: a first density range comprising epithelium-immune cell densities of 0.0-0.005, a second density range comprising epithelium-immune cell densities of 0.005-0.01, a third density range comprising epithelium-immune cell densities of 0.01-0.02, a fourth density range comprising epithelium-immune cell densities of 0.02-0.04, a fifth density range comprising epithelium-immune cell densities of 0.04-0.06, a sixth density range comprising epithelium-immune cell densities of 0.06-0.08, a seventh density range comprising epithelium-immune cell densities of 0.08-0.12, an eighth density range comprising epithelium-immune cell densities of 0.12-0.16, a ninth density range comprising epithelium-immune cell densities of 0.16-0.2, and a tenth density range comprising epithelium-immune cell densities of 0.2-2.0.

In operation 2018, a stroma tile may be selected from the identified stroma tiles. In operation 2020, a stroma-immune cell density may be calculated for the selected stroma tile. In some embodiments, the stroma-immune cell density may be calculated by determining a number of immune cells detected within the selected stroma tile. The stroma-immune cell density for the selected tile may be calculated based on a number of immune cells detected within the selected stroma tile. In some embodiments, immune cells within stroma tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected stroma tile. In some embodiments, the number of immune cells detected within the selected stroma tile may be determined using one or more machine learning models. The one or more machine learning models may include a computer vision model trained to recognize immune cells. An example computer vision model that may be used to recognize immune cells within tumor stroma regions of an image may be a convolutional neural network (CNN).

In operation 2022, a determination may be made as to whether any additional stroma tiles are to be analyzed. If so, process 2000 may return to operation 2018 where another stroma tile may be selected and operations 2020-2022 may repeat. However, if at operation 2022 it is determined that no additional stroma tiles are to be analyzed, then process 2000 may proceed to operation 2024.

In operation 2024, the stroma tiles may be binned a stroma set of bins based on the epithelium-immune cell density of each epithelium tile. For example, the stroma tiles may be binned into one of stroma set of bins 804 of FIG. 8. In some embodiments, binning stroma tile into one of the stroma set of bins may include determining a density range for each bin of the stroma set of bins. Each stroma tile of the plurality of tiles may be allocated to one of the stroma set of bins based on the stroma-immune cell density of the stroma tile. In some embodiments, the stroma set of bins may include ten bins. For example, the stroma set of bins may be defined by ten different density ranges: a first density range comprising stroma-immune cell densities of 0.0-0.005, a second density range comprising stroma-immune cell densities of 0.005-0.01, a third density range comprising stroma-immune cell densities of 0.01-0.02, a fourth density range comprising stroma-immune cell densities of 0.02-0.04, a fifth density range comprising stroma-immune cell densities of 0.04-0.06, a sixth density range comprising stroma-immune cell densities of 0.06-0.08, a seventh density range comprising stroma-immune cell densities of 0.08-0.12, an eighth density range comprising stroma-immune cell densities of 0.12-0.16, a ninth density range comprising stroma-immune cell densities of 0.16-0.2, and a tenth density range comprising stroma-immune cell densities of 0.2-2.0.

In some embodiments, operations 2010-2016 and operations 2018-2024 may be performed in parallel or sequentially.

In operation 2026, a density-bin representation may be generated based on the epithelium set of bins and the stroma set of bins. The density-bin representation may include a plurality of elements corresponding to each bin of the epithelium set of bins and each bin of the stroma set of bins. As an example, with reference to FIG. 9, density-bin representation 910 may be generated based on immune cell density distribution 800 formed of epithelium set of bins 802 and stroma set of bins 804. In some embodiments, the density-bin representation may include 20-elements forming a 20-dimensional feature vector.

In operation 2028, a determination may be made as to whether any additional images from the plurality of images received in operation 2002 are to be analyzed. If so, then process 2000 may return to operation 2004 where another image from the received plurality of images may be selected and operations 2006-2028 may be repeated. However, if at operation 2028 it is determined that no additional images are to be analyzed, process 2000 may proceed to operation 2030. Persons of ordinary skill in the art will recognize that multiple images may be selected at operation 2004, and operations 2006-2026 may be performed for these images in parallel using one or more processors of a computing system. The parallelization of the image analysis process may decrease processing time at the expense of increased computational cost; however, this is not to imply that each image from the received plurality of images needs to be analyzed sequentially.

In operation 2030, training data may be generated. The training data may include a plurality of density-bin representations each representing an epithelium-immune cell density distribution and a stroma-immune cell density distribution (e.g., distribution 800 of FIG. 8) of an image from the plurality of images. In some embodiments, the training data may also include the labels indicating the pre-determined tumor immunophenotype of each image. For example, the training data may comprise tuples of an image from the plurality of images depicting tumors, the density-bin representation generated for that image, and the label of the pre-determined tumor immunophenotype of the tumor depicted by that image.

In operation 2032, a classifier may be trained to predict a tumor immunophenotype of an image using the training data. In some embodiments, the classifier may be a multi-class classifier configured to classify an image into one of a set of tumor immunophenotypes. Some example classifiers that may be used include, but are not limited to, a trained support vector machine (SVM), a random forest (RF) classifier, a decision tree classifier, a logistic regression classifier, a k-nearest neighbor (kNN) classifier, or other classifiers.

In some embodiments, the set of tumor immunophenotypes may include the tumor immunophenotypes of desert, excluded, and inflamed. In some embodiments, an image depicting a tumor may be classified as being the tumor immunophenotype desert based on an epithelium-immune cell density satisfying a desert epithelium-immune cell density threshold criterion and a stroma-immune cell density satisfying a desert stroma-immune cell density threshold criterion. The desert epithelium-immune cell density threshold criterion being satisfied may include the epithelium-immune cell density being within a first threshold range of epithelium-immune cell densities. The desert stroma-immune cell density threshold criterion being satisfied may include the stroma-immune cell density being within a first threshold range of stroma-immune cell densities.

In some embodiments, an image depicting a tumor may be classified as being the tumor immunophenotype excluded based on an epithelium-immune cell density satisfying an excluded epithelium-immune cell density threshold criterion and a stroma-immune cell density satisfying an excluded stroma-immune cell density threshold criterion. The excluded epithelium-immune cell density threshold criterion being satisfied may include the epithelium-immune cell density being within a second threshold range of epithelium-immune cell densities. The excluded stroma-immune cell density threshold criterion being satisfied may include the stroma-immune cell density being within a second threshold range of stroma-immune cell densities.

In some embodiments, an image depicting a tumor may be classified as being the tumor immunophenotype inflamed based on an epithelium-immune cell density satisfying an inflamed epithelium-immune cell density threshold criterion and a stroma-immune cell density satisfying an inflamed stroma-immune cell density threshold criterion. The inflamed epithelium-immune cell density threshold criterion being satisfied may include the epithelium-immune cell density being within a third threshold range of epithelium-immune cell densities. The inflamed stroma-immune cell density threshold criterion being satisfied may include the stroma-immune cell density being within a third threshold range of stroma-immune cell densities.

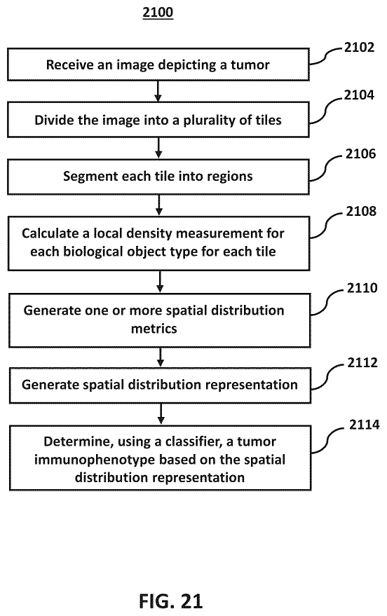

FIG. 21 is an illustrative flowchart of an exemplary process 2100 for determining a tumor immunophenotype using a third pipeline, in accordance with various embodiments. In some embodiments, process 2100 may be implemented by one or more modules and components of third pipeline subsystem 116. However, in some embodiments, additional or alternative components of system 100 may be employed to execute one or more aspects of process 2100.

In some embodiments, process 2100 may begin at operation 2102. At operation 2102, an image depicting a tumor may be received. In some embodiments, the image is a digital pathology image captured using a digital pathology imaging system (e.g., image scanner 240). The image may be a whole slide image or a portion of a whole slide image depicting a tissue sample. The whole slide image may be annotated to identify tumor lesions, and the image may be extracted from the whole slide image. In some embodiments, a dual-stain may be applied to a sample of the tumor prior to the image being captured. The dual-stain may include a first stain and a second stain. For example, the first stain may distinguish and highlight tumor epithelium from tumor stroma and the second stain may highlights immune cell. In some embodiments, the first stain may be a pan-cytokeratin (panCK) stain used for highlighting the tumor epithelium (e.g., CK+ tumor cells) and tumor stroma (e.g., CK− tumor cells), and the second stain may be a cluster of differentiation 8 (CD8) stain used for highlighting immune cells (e.g., CD8+ T cells). In some embodiments, the image may depict a stained section of a biological sample from a patient exhibiting a medical condition (e.g., NSCLC).

In operation 2104, the image may be divided into a plurality of tiles. The tiles may be overlapping or non-overlapping. For example, with reference to FIG. 6, digital pathology image 604 may be divided into tiles 606.

In operation 2106, each of the tiles may be segmented into regions based on the stains applied to the biological sample. For example, tiles may be segmented into regions based on how different biological objects within the sample react to the applied stains. The biological objects may include tumor epithelium, tumor stroma, immune cells, or other objects. In some embodiments, a pixel-based segmentation approach may be used to segment and classify regions of a tile based on how those regions react to the particular stains applied to the sample. For example, each pixel within a given tile may be classified as belonging to or containing one or more depictions of the biological objects based on a color of the region. As an illustrative example, pixels with a threshold intensity in one or more first color channels may be associated with a first biological object type (e.g., tumor epithelium, tumor stroma) and pixels associated with a threshold intensity in one or more second color channels may be associated with a second biological object type (e.g., immune cells). The threshold intensity and the color channels may be selected based on the stains used, as different stains may cause different biological objects to be highlighted (e.g., panCK stains may highlight CK+ and CK− regions while CD8 stains may highlight CD8+ T cells). In some embodiments, regions of a tile may be associated with a particular biological object type based on an image segmentation algorithm's confidence score.

In operation 2108, a local density measurement may be calculated for each of the different biological object types for each of the tiles. For example, for a given tile, a local density measurement may be calculated for tumor epithelium (e.g., CK+ density), tumor stroma (e.g., CK− density), immune cells within the tumor epithelium (e.g., CD8+ T cell density within CK+ regions), immune cells within tumor stroma (e.g., CD8+ T cell density within CK− regions), or other densities. In some embodiments, a data structure may be generated that includes object information characterizing the biological object depictions. For example, the data structure may identify a location of the biological object depictions and/or location of the tile within a lattice of the image (e.g., the digital pathology image depicting the stained tumor sample). The data structure may also identify a type of biological object (e.g., lymphocyte, tumor cell, etc.) that corresponds to the depicted biological object. In some embodiments, the local density measurement may be calculated based on a number of regions (e.g., pixels) of a tile classified with each of the stains (e.g., the panCK stain, the CD8 stain). In some embodiments, the local density measurement of each of the biological object types for each tile may include a representation of an absolute or relative quantity of biological object depictions of a first type (e.g., tumor epithelium cells, tumor stroma cells) of the biological object types identified as being located within the tile and an absolute or relative quantity of biological object depictions of a second type of the biological object types (e.g., immune cells) identified as being located within the tile. For example, in an example where there are two biological object types of interest, the local density measurement can reflect the absolute number or percentage of the regions of each tile that are associated with each of the two biological object types. This value can be divided by the overall number of regions within the tile to give a percentage of the tile associated with each of the biological object types. In some embodiments, the local density measurement can be expressed as an area value based on a known conversion between the size of pixels of the digital pathology image and the corresponding size of the biological sample.

In operation 2110, one or more spatial distribution metrics may be generated for the biological object types in the image based on the local density measurements for each tile. Each spatial distribution metric may characterize a degree to which at least part of the first set of biological objects is depicted as being interspersed with at least part of the second set of biological objects. For example, the degree with which immune cells are distributed within regions of tumor epithelium may be related to the degree of tumor epithelium cells within that same tile. Some example spatial distribution metrics that may be generate include, but are not limited to, the Jaccard index, the Sorensen index, the Bhattacharyya coefficient, Moran's index, the Geary's contiguity ratio, the Morisita-Horn index, a metric defined based on a hotspot/cold spot analysis, or other metrics, or combinations thereof.

In operation 2112, a spatial distribution representation may be generated based on the one or more spatial distribution metrics. The spatial distribution representation may be a feature vector and/or embedding representing the local density measurements and the spatial distribution metrics. For example, each element of the spatial distribution representation may correspond to a value of a corresponding spatial distribution metric. In some embodiments, the spatial distribution metrics may be input to an encoder to generate the spatial distribution representation. The spatial distribution representation, in this example, may be an embedding that is projected into an embedding space or feature space defined by the spatial distribution metrics (e.g., having axes based on the spatial distribution metrics). The projection and feature space can be based on a machine-learning model trained to generate embeddings in an appropriate feature space. As an example, the embedding/feature vector may be a 50-dimensional embedding/feature vector, where each element corresponds to a spatial distribution metric.

In operation 2114, a tumor immunophenotype may be determined using a classifier based on the spatial distribution representation. In some embodiments, the classifier may be trained to predict a tumor immunophenotype based on a spatial distribution representation input to the classifier. For example, the tumor immunophenotype may be determined based on a position of the spatial distribution representation of the digital pathology image within the embedding space. The classifier may assign a tumor immunophenotype to the biological sample (e.g., tumor) depicted by the digital pathology image based on a proximity of the position of the spatial distribution representation in the embedding space to a position of one or more clusters of spatial distribution representations associated with particular tumor immunophenotypes. These neighboring spatial distribution representations can have pre-assigned or predetermined immunophenotype classifications. The digital pathology image can be assigned an immunophenotype based on the immunophenotypes of the nearest neighbors in the feature space. For example, a cluster having a smallest L2 distance to the position of the spatial distribution representation in the embedding space may indicate that the image is the most similar to that cluster's corresponding tumor immunophenotype.

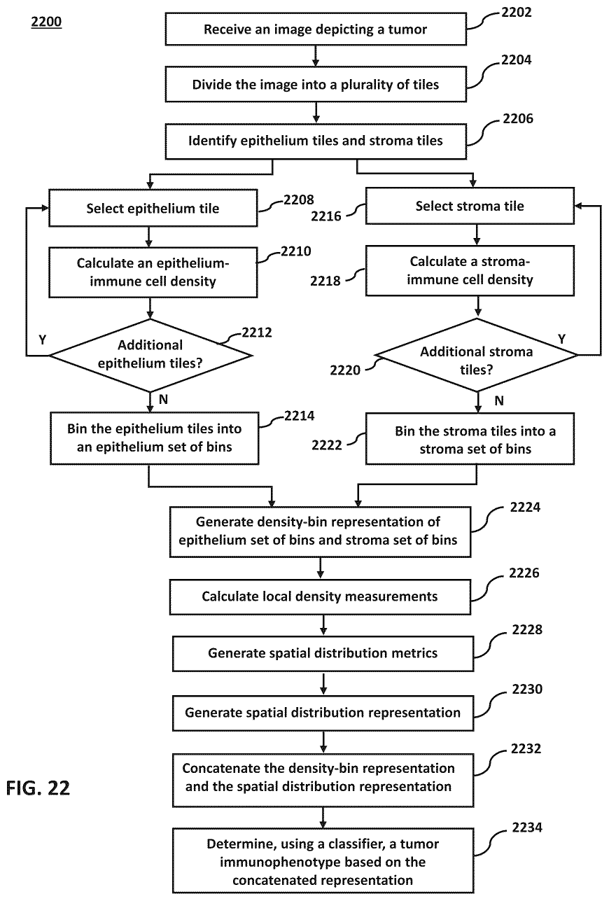

FIG. 22 is an illustrative flowchart of an exemplary process 2200 for determining a tumor immunophenotype using a fourth pipeline, in accordance with various embodiments. In some embodiments, process 2200 may be implemented by one or more modules and components of fourth pipeline subsystem 118. However, in some embodiments, additional or alternative components of system 100 may be employed to execute one or more aspects of process 2200.

In some embodiments, process 2200 may begin at operation 2202. At operation 2202, an image depicting a tumor may be received. In some embodiments, the image is a digital pathology image captured using a digital pathology imaging system (e.g., image scanner 240). The image may be a whole slide image or a portion of a whole slide image. The whole slide image may be annotated to identify tumor lesions, and the image may be extracted from the whole slide image. In some embodiments, a dual-stain may be applied to a sample of the tumor prior to the image being captured. The dual-stain may include a first stain and a second stain. For example, the first stain may distinguish and highlight tumor epithelium from tumor stroma and the second stain may highlights immune cell. In some embodiments, the first stain may be a pan-cytokeratin (panCK) stain used for highlighting the tumor epithelium (e.g., CK+ tumor cells) and tumor stroma (e.g., CK− tumor cells), and the second stain may be a cluster of differentiation 8 (CD8) stain used for highlighting immune cells (e.g., CD8+ T cells).

In operation 2204, the image may be divided into a plurality of tiles. The tiles may be overlapping or non-overlapping. For example, with reference to FIG. 6, digital pathology image 604 may be divided into tiles 606.

In operation 2206, epithelium tiles and stroma tiles may be identified from the plurality of tiles. In some embodiments, a tile may be classified as an epithelium tile based on a portion of the tile depicting tumor epithelium satisfying an epithelium-tile criterion. The epithelium-tile criterion being satisfied may include the portion of the tile depicting tumor epithelium being greater than or equal to a first threshold area. For example, the first threshold area may include 25% of the tile. In some embodiments, a tile may be classified as a stroma tile based on a portion of the tile depicting tumor stroma satisfying a stroma-tile criterion. The stroma-tile criterion being satisfied may include the portion of the tile depicting the tumor stroma being greater than or equal to a second threshold area. For example, the second threshold area may include 25% of the tile. Thus, in some embodiments, a tile may be classified as being both an epithelium tile and a stroma tile.

In operation 2208, an epithelium tile may be selected from the identified epithelium tiles. In operation 2210, an epithelium-immune cell density may be calculated for the selected epithelium tile. In some embodiments, the epithelium-immune cell density may be calculated by determining a number of immune cells detected within the selected epithelium tile. The epithelium-immune cell density for the selected tile may be calculated based on a number of immune cells detected within the selected epithelium tile. In some embodiments, immune cells within epithelium tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected epithelium tile. In some embodiments, the number of immune cells detected within the selected epithelium tile may be determined using one or more machine learning models. The one or more machine learning models may include a computer vision model trained to recognize immune cells. An example computer vision model that may be used to recognize immune cells within tumor epithelium regions of an image may be a convolutional neural network (CNN).

In operation 2212, a determination may be made as to whether any additional epithelium tiles are to be analyzed. If so, process 2200 may return to operation 2208 where another epithelium tile may be selected and operations 2210-2212 may repeat. However, if at operation 2212 it is determined that no additional epithelium tiles are to be analyzed, then process 2200 may proceed to operation 2214.

In operation 2214, the epithelium tiles may be binned into an epithelium set of bins based on the epithelium-immune cell density of each epithelium tile. For example, the epithelium tiles may be binned into one of epithelium set of bins 802 of FIG. 8. In some embodiments, binning an epithelium tile into one of the epithelium set of bins may include determining a density range for each bin of the epithelium set of bins. Each epithelium tile of the plurality of tiles may be allocated to one of the epithelium set of bins based on the epithelium-immune cell density of the epithelium tile. In some embodiments, the epithelium set of bins may include ten bins. For example, the epithelium set of bins may be defined by ten different density ranges: a first density range comprising epithelium-immune cell densities of 0.0-0.005, a second density range comprising epithelium-immune cell densities of 0.005-0.01, a third density range comprising epithelium-immune cell densities of 0.01-0.02, a fourth density range comprising epithelium-immune cell densities of 0.02-0.04, a fifth density range comprising epithelium-immune cell densities of 0.04-0.06, a sixth density range comprising epithelium-immune cell densities of 0.06-0.08, a seventh density range comprising epithelium-immune cell densities of 0.08-0.12, an eighth density range comprising epithelium-immune cell densities of 0.12-0.16, a ninth density range comprising epithelium-immune cell densities of 0.16-0.2, and a tenth density range comprising epithelium-immune cell densities of 0.2-2.0.

In operation 2216, a stroma tile may be selected from the identified stroma tiles. In operation 2218, a stroma-immune cell density may be calculated for the selected stroma tile. In some embodiments, the stroma-immune cell density may be calculated by determining a number of immune cells detected within the selected stroma tile. The stroma-immune cell density for the selected tile may be calculated based on a number of immune cells detected within the selected stroma tile. In some embodiments, immune cells within stroma tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected stroma tile. In some embodiments, the number of immune cells detected within the selected stroma tile may be determined using one or more machine learning models. The one or more machine learning models may include a computer vision model trained to recognize immune cells. An example computer vision model that may be used to recognize immune cells within tumor stroma regions of an image may be a convolutional neural network (CNN).

In operation 2220, a determination may be made as to whether any additional stroma tiles are to be analyzed. If so, process 2200 may return to operation 2216 where another stroma tile may be selected and operations 2218-2220 may repeat. However, if at operation 2220 it is determined that no additional stroma tiles are to be analyzed, then process 2200 may proceed to operation 2222.

In operation 2222, the stroma tiles may be binned a stroma set of bins based on the epithelium-immune cell density of each epithelium tile. For example, the stroma tiles may be binned into one of stroma set of bins 804 of FIG. 8. In some embodiments, binning stroma tile into one of the stroma set of bins may include determining a density range for each bin of the stroma set of bins. Each stroma tile of the plurality of tiles may be allocated to one of the stroma set of bins based on the stroma-immune cell density of the stroma tile. In some embodiments, the stroma set of bins may include ten bins. For example, the stroma set of bins may be defined by ten different density ranges: a first density range comprising stroma-immune cell densities of 0.0-0.005, a second density range comprising stroma-immune cell densities of 0.005-0.01, a third density range comprising stroma-immune cell densities of 0.01-0.02, a fourth density range comprising stroma-immune cell densities of 0.02-0.04, a fifth density range comprising stroma-immune cell densities of 0.04-0.06, a sixth density range comprising stroma-immune cell densities of 0.06-0.08, a seventh density range comprising stroma-immune cell densities of 0.08-0.12, an eighth density range comprising stroma-immune cell densities of 0.12-0.16, a ninth density range comprising stroma-immune cell densities of 0.16-0.2, and a tenth density range comprising stroma-immune cell densities of 0.2-2.0.

In some embodiments, operations 2208-2214 and operations 2216-2222 may be performed in parallel or sequentially.

In operation 2224, a density-bin representation may be generated based on the epithelium set of bins and the stroma set of bins. The density-bin representation may include a plurality of elements corresponding to each bin of the epithelium set of bins and each bin of the stroma set of bins. As an example, with reference to FIG. 9, density-bin representation 910 may be generated based on immune cell density distribution 800 formed of epithelium set of bins 802 and stroma set of bins 804. In some embodiments, the density-bin representation may include 20-elements forming a 20-dimensional feature vector.

In operation 2226, a local density measurement may be calculated for each of the different biological object types for each of the tiles. For example, for a given tile (e.g., epithelium tile, stroma tile), a local density measurement may be calculated (e.g., CK+ density, CK− density, CD8+ T cell density within CK+ regions, CD8+ T cell density within CK− regions, etc.). The local densities can indicate whether one or more types of biological objects (e.g., CD8+ T cells, CK+ tumor cells, CK− tumor cells) re more predominant in that particular tile. This classification can indicate, for example, the presence or absence of the different types of biological objects (e.g., tiles can be classified as positive or negative for CD8+ T cells and positive or negative for CK+ tumor cells). In some embodiments, a data structure may be generated that includes object information characterizing the biological object depictions. For example, the data structure may identify a location of the biological object depictions and/or location of the tile within a lattice of the image (e.g., the digital pathology image depicting the stained tumor sample). The data structure may also identify a type of biological object (e.g., lymphocyte, tumor cell, etc.) that corresponds to the depicted biological object. In some embodiments, the local density measurement may be calculated based on a number of regions (e.g., pixels) of a tile classified with each of the stains (e.g., the panCK stain, the CD8 stain). In some embodiments, the local density measurement of each of the biological object types for each tile may include a representation of an absolute or relative quantity of biological object depictions of a first type (e.g., tumor epithelium cells, tumor stroma cells) of the biological object types identified as being located within the tile and an absolute or relative quantity of biological object depictions of a second type of the biological object types (e.g., immune cells) identified as being located within the tile. For example, in an example where there are two biological object types of interest, the local density measurement can reflect the absolute number or percentage of the regions of each tile that are associated with each of the two biological object types. This value can be divided by the overall number of regions within the tile to give a percentage of the tile associated with each of the biological object types. In some embodiments, the local density measurement can be expressed as an area value based on a known conversion between the size of pixels of the digital pathology image and the corresponding size of the biological sample.

In operation 2228, one or more spatial distribution metrics may be generated describing the image based on the epithelium-immune cell density of one or more epithelium tiles and the stroma-immune cell density of one or more of the stroma tiles. Each spatial distribution metric may characterize a degree to which at least part of the first set of biological objects is depicted as being interspersed with at least part of the second set of biological objects. For example, the degree with which immune cells are distributed within regions of tumor epithelium may be related to the degree of tumor epithelium cells within that same tile. Some example spatial distribution metrics that may be generate include, but are not limited to, the Jaccard index, the Sorensen index, the Bhattacharyya coefficient, Moran's index, the Geary's contiguity ratio, the Morisita-Horn index, a metric defined based on a hotspot/cold spot analysis, or other metrics, or combinations thereof.

In operation 2230, a spatial distribution representation may be generated based on the one or more spatial distribution metrics. The spatial distribution representation may be a feature vector and/or embedding representing the local density measurements and the spatial distribution metrics. For example, each element of the spatial distribution representation may correspond to a value of a corresponding spatial distribution metric. In some embodiments, the spatial distribution metrics may be input to an encoder to generate the spatial distribution representation. The spatial distribution representation, in this example, may be an embedding that is projected into an embedding space or feature space defined by the spatial distribution metrics (e.g., having axes based on the spatial distribution metrics). The projection and feature space can be based on a machine-learning model trained to generate embeddings in an appropriate feature space. As an example, the embedding/feature vector may be a 50-dimensional embedding/feature vector, where each element corresponds to a spatial distribution metric.

In operation 2232, the density-bin representation and the spatial distribution representation may be concatenated to obtain a concatenated representation. As an example, with reference to FIG. 13, density-bin representation 910 may be concatenated with spatial distribution representation 1110 to obtain concatenated representation 1310. The concatenated representation may have a dimensionality equal to the aggregate of the dimensionality of the density-bin representation and the spatial distribution representation. For example, with reference again to FIG. 13, if density-bin representation 910 has N elements and spatial distribution representation 1110 has M elements, then concatenated representation 1310 may have N+M elements.

In operation 2234, a tumor immunophenotype may be determined based on the concatenated representation. In some embodiments, a trained classifier may be used to determine the tumor immunophenotype. For example, classifier 1400 may be used to determine tumor immunophenotype 1410 based on concatenated representation 1310. In some embodiments, the classifier (e.g., classifier 1400) may be a multi-class classifier configured to classify an image into one of a set of tumor immunophenotypes. Some example classifiers that may be used include, but are not limited to, a trained support vector machine (SVM), a random forest (RF) classifier, a decision tree classifier, a logistic regression classifier, or a k-nearest neighbor (kNN) classifier.

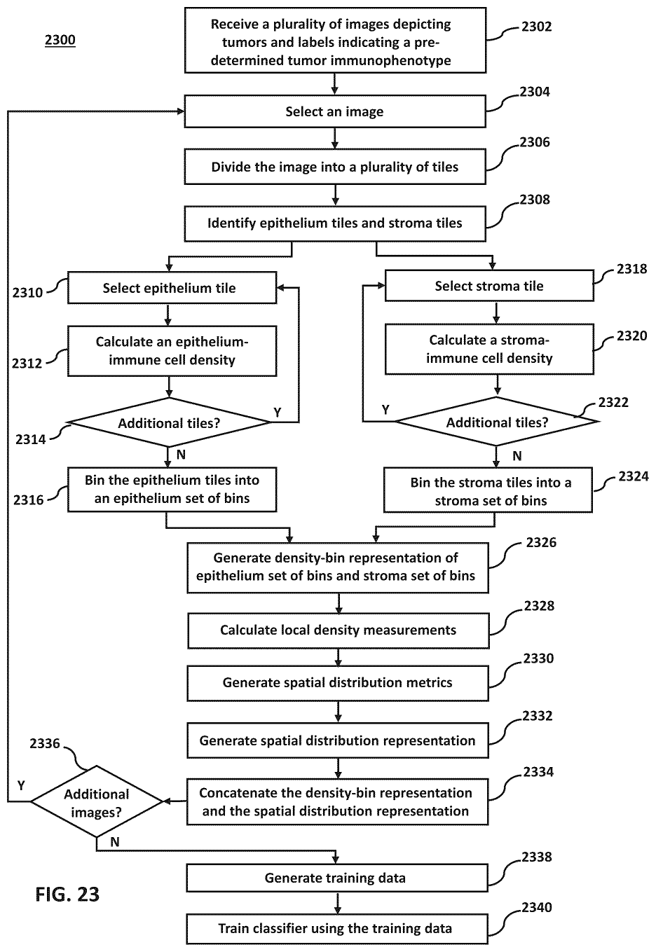

FIG. 23 is an illustrative flowchart of a process 2300 for training a classifier to determine a tumor immunophenotype, in accordance with various embodiments. In some embodiments, process 2300 may be implemented by one or more modules and components of fourth pipeline subsystem 118. For example, process 2300 may be performed by classifier training module 1230 of fourth pipeline subsystem 118, as described above. However, in some embodiments, additional or alternative components of system 100 may be employed to execute one or more aspects of process 2300.

In some embodiments, process 2300 may begin at operation 2302. In operation 2302, a plurality of images may be received. Each of the images may correspond to a patient from a plurality of patients participating in a clinical trial. In some embodiments, the plurality of images may include labels indicating a pre-determined tumor immunophenotype. For example, each image may include metadata that labels the tumor immunophenotype of the tumor depicted by the image. The tumor immunophenotype may be pre-determined by a trained pathologist, using one or more machine learning models, or a combination thereof. The tumor immunophenotype may be selected, for example, by the trained pathologist from a set of tumor immunophenotypes including desert, excluded, and inflamed. In some embodiments, the images may be stored in image database 142.

In operation 2304, an image from the received images may be selected. In operation 2306, the image may be divided into a plurality of tiles. The tiles may be overlapping or non-overlapping. For example, with reference to FIG. 6, digital pathology image 604 may be divided into tiles 606.

In operation 2308, epithelium tiles and stroma tiles may be identified from the plurality of tiles. In some embodiments, a tile may be classified as an epithelium tile based on a portion of the tile depicting tumor epithelium satisfying an epithelium-tile criterion. The epithelium-tile criterion being satisfied may include the portion of the tile depicting tumor epithelium being greater than or equal to a first threshold area. For example, the first threshold area may include 25% of the tile. In some embodiments, a tile may be classified as a stroma tile based on a portion of the tile depicting tumor stroma satisfying a stroma-tile criterion. The stroma-tile criterion being satisfied may include the portion of the tile depicting the tumor stroma being greater than or equal to a second threshold area. For example, the second threshold area may include 25% of the tile. Thus, in some embodiments, a tile may be classified as being both an epithelium tile and a stroma tile.

In operation 2310, an epithelium tile may be selected from the identified epithelium tiles. In operation 2312, an epithelium-immune cell density may be calculated for the selected epithelium tile. In some embodiments, the epithelium-immune cell density may be calculated by determining a number of immune cells detected within the selected epithelium tile. The epithelium-immune cell density for the selected tile may be calculated based on a number of immune cells detected within the selected epithelium tile. In some embodiments, immune cells within epithelium tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected epithelium tile. In some embodiments, the number of immune cells detected within the selected epithelium tile may be determined using one or more machine learning models. The one or more machine learning models may include a computer vision model trained to recognize immune cells. An example computer vision model that may be used to recognize immune cells within tumor epithelium regions of an image may be a convolutional neural network (CNN).

In operation 2314, a determination may be made as to whether any additional epithelium tiles are to be analyzed. If so, process 2300 may return to operation 2310 where another epithelium tile may be selected and operations 2312-2314 may repeat. However, if at operation 2314 it is determined that no additional epithelium tiles are to be analyzed, then process 2300 may proceed to operation 2316.

In operation 2316, the epithelium tiles may be binned into an epithelium set of bins based on the epithelium-immune cell density of each epithelium tile. For example, the epithelium tiles may be binned into one of epithelium set of bins 802 of FIG. 8. In some embodiments, binning an epithelium tile into one of the epithelium set of bins may include determining a density range for each bin of the epithelium set of bins. Each epithelium tile of the plurality of tiles may be allocated to one of the epithelium set of bins based on the epithelium-immune cell density of the epithelium tile. In some embodiments, the epithelium set of bins may include ten bins. For example, the epithelium set of bins may be defined by ten different density ranges: a first density range comprising epithelium-immune cell densities of 0.0-0.005, a second density range comprising epithelium-immune cell densities of 0.005-0.01, a third density range comprising epithelium-immune cell densities of 0.01-0.02, a fourth density range comprising epithelium-immune cell densities of 0.02-0.04, a fifth density range comprising epithelium-immune cell densities of 0.04-0.06, a sixth density range comprising epithelium-immune cell densities of 0.06-0.08, a seventh density range comprising epithelium-immune cell densities of 0.08-0.12, an eighth density range comprising epithelium-immune cell densities of 0.12-0.16, a ninth density range comprising epithelium-immune cell densities of 0.16-0.2, and a tenth density range comprising epithelium-immune cell densities of 0.2-2.0.

In operation 2318, a stroma tile may be selected from the identified stroma tiles. In operation 2320, a stroma-immune cell density may be calculated for the selected stroma tile. In some embodiments, the stroma-immune cell density may be calculated by determining a number of immune cells detected within the selected stroma tile. The stroma-immune cell density for the selected tile may be calculated based on a number of immune cells detected within the selected stroma tile. In some embodiments, immune cells within stroma tiles may be determined by a trained pathologist. In some embodiments, a color deconvolution process may be performed to the image to obtain a plurality of color channels, where one color channel highlights tumor epithelium and another color channel highlights immune cells. The different color channel images, and subsequently obtain tiles, may then be analyzed to determine the number of immune cells present within the selected stroma tile. In some embodiments, the number of immune cells detected within the selected stroma tile may be determined using one or more machine learning models. The one or more machine learning models may include a computer vision model trained to recognize immune cells. An example computer vision model that may be used to recognize immune cells within tumor stroma regions of an image may be a convolutional neural network (CNN).

In operation 2322, a determination may be made as to whether any additional stroma tiles are to be analyzed. If so, process 2300 may return to operation 2318 where another stroma tile may be selected and operations 2320-2322 may repeat. However, if at operation 2322 it is determined that no additional stroma tiles are to be analyzed, then process 2300 may proceed to operation 2324.

In operation 2324, the stroma tiles may be binned a stroma set of bins based on the epithelium-immune cell density of each epithelium tile. For example, the stroma tiles may be binned into one of stroma set of bins 804 of FIG. 8. In some embodiments, binning stroma tile into one of the stroma set of bins may include determining a density range for each bin of the stroma set of bins. Each stroma tile of the plurality of tiles may be allocated to one of the stroma set of bins based on the stroma-immune cell density of the stroma tile. In some embodiments, the stroma set of bins may include ten bins. For example, the stroma set of bins may be defined by ten different density ranges: a first density range comprising stroma-immune cell densities of 0.0-0.005, a second density range comprising stroma-immune cell densities of 0.005-0.01, a third density range comprising stroma-immune cell densities of 0.01-0.02, a fourth density range comprising stroma-immune cell densities of 0.02-0.04, a fifth density range comprising stroma-immune cell densities of 0.04-0.06, a sixth density range comprising stroma-immune cell densities of 0.06-0.08, a seventh density range comprising stroma-immune cell densities of 0.08-0.12, an eighth density range comprising stroma-immune cell densities of 0.12-0.16, a ninth density range comprising stroma-immune cell densities of 0.16-0.2, and a tenth density range comprising stroma-immune cell densities of 0.2-2.0.

In some embodiments, operations 2310-2316 and operations 2318-2324 may be performed in parallel or sequentially.

In operation 2326, a density-bin representation may be generated based on the epithelium set of bins and the stroma set of bins. The density-bin representation may include a plurality of elements corresponding to each bin of the epithelium set of bins and each bin of the stroma set of bins. As an example, with reference to FIG. 9, density-bin representation 910 may be generated based on immune cell density distribution 800 formed of epithelium set of bins 802 and stroma set of bins 804. In some embodiments, the density-bin representation may include 20-elements forming a 20-dimensional feature vector.

In operation 2328, a local density measurement may be calculated for each of the different biological object types for each of the tiles. For example, for a given tile (e.g., epithelium tile, stroma tile), a local density measurement may be calculated (e.g., CK+ density, CK− density, CD8+ T cell density within CK+ regions, CD8+ T cell density within CK− regions, etc.). The local densities can indicate whether one or more types of biological objects (e.g., CD8+ T cells, CK+ tumor cells, CK− tumor cells) re more predominant in that particular tile. This classification can indicate, for example, the presence or absence of the different types of biological objects (e.g., tiles can be classified as positive or negative for CD8+ T cells and positive or negative for CK+ tumor cells). In some embodiments, a data structure may be generated that includes object information characterizing the biological object depictions. For example, the data structure may identify a location of the biological object depictions and/or location of the tile within a lattice of the image (e.g., the digital pathology image depicting the stained tumor sample). The data structure may also identify a type of biological object (e.g., lymphocyte, tumor cell, etc.) that corresponds to the depicted biological object. In some embodiments, the local density measurement may be calculated based on a number of regions (e.g., pixels) of a tile classified with each of the stains (e.g., the panCK stain, the CD8 stain). In some embodiments, the local density measurement of each of the biological object types for each tile may include a representation of an absolute or relative quantity of biological object depictions of a first type (e.g., tumor epithelium cells, tumor stroma cells) of the biological object types identified as being located within the tile and an absolute or relative quantity of biological object depictions of a second type of the biological object types (e.g., immune cells) identified as being located within the tile. For example, in an example where there are two biological object types of interest, the local density measurement can reflect the absolute number or percentage of the regions of each tile that are associated with each of the two biological object types. This value can be divided by the overall number of regions within the tile to give a percentage of the tile associated with each of the biological object types. In some embodiments, the local density measurement can be expressed as an area value based on a known conversion between the size of pixels of the digital pathology image and the corresponding size of the biological sample.

In operation 2330, one or more spatial distribution metrics may be generated describing the image based on the epithelium-immune cell density of one or more epithelium tiles and the stroma-immune cell density of one or more of the stroma tiles. Each spatial distribution metric may characterize a degree to which at least part of the first set of biological objects is depicted as being interspersed with at least part of the second set of biological objects. For example, the degree with which immune cells are distributed within regions of tumor epithelium may be related to the degree of tumor epithelium cells within that same tile. Some example spatial distribution metrics that may be generate include, but are not limited to, the Jaccard index, the Sorensen index, the Bhattacharyya coefficient, Moran's index, the Geary's contiguity ratio, the Morisita-Horn index, a metric defined based on a hotspot/cold spot analysis, or other metrics, or combinations thereof.

In operation 2332, a spatial distribution representation may be generated based on the one or more spatial distribution metrics. The spatial distribution representation may be a feature vector and/or embedding representing the local density measurements and the spatial distribution metrics. For example, each element of the spatial distribution representation may correspond to a value of a corresponding spatial distribution metric. In some embodiments, the spatial distribution metrics may be input to an encoder to generate the spatial distribution representation. The spatial distribution representation, in this example, may be an embedding that is projected into an embedding space or feature space defined by the spatial distribution metrics (e.g., having axes based on the spatial distribution metrics). The projection and feature space can be based on a machine-learning model trained to generate embeddings in an appropriate feature space. As an example, the embedding/feature vector may be a 50-dimensional embedding/feature vector, where each element corresponds to a spatial distribution metric.

In operation 2334, the density-bin representation and the spatial distribution representation may be concatenated to obtain a concatenated representation. As an example, with reference to FIG. 13, density-bin representation 910 may be concatenated with spatial distribution representation 1110 to obtain concatenated representation 1310. The concatenated representation may have a dimensionality equal to the aggregate of the dimensionality of the density-bin representation and the spatial distribution representation. For example, with reference again to FIG. 13, if density-bin representation 910 has N elements and spatial distribution representation 1110 has M elements, then concatenated representation 1310 may have N+M elements.

In operation 2336, a determination may be made as to whether any additional images from the plurality of images received in operation 2302 are to be analyzed. If so, then process 2300 may return to operation 2304 where another image from the received plurality of images may be selected and operations 2306-2334 may be repeated. However, if at operation 2336 it is determined that no additional images are to be analyzed, process 2300 may proceed to operation 2338. Persons of ordinary skill in the art will recognize that multiple images may be selected at operation 2304, and operations 2306-2326 may be performed for these images in parallel using one or more processors of a computing system. The parallelization of the image analysis process may decrease processing time at the expense of increased computational cost, however this is not to imply that each image from the received plurality of images needs to be analyzed sequentially.

In operation 2338, training data may be generated. The training data may include a plurality of concatenated representations (e.g., concatenated representation 1310) of an image from the plurality of images. In some embodiments, the training data may also include the labels indicating the pre-determined tumor immunophenotype of each image. For example, the training data may comprise tuples of an image from the plurality of images depicting tumors, the concatenated representation generated for that image, and the label of the pre-determined tumor immunophenotype of the tumor depicted by that image.

In operation 2340, a classifier may be trained to predict a tumor immunophenotype of an image using the training data. In some embodiments, the classifier may be a multi-class classifier configured to classify an image into one of a set of tumor immunophenotypes. Some example classifiers that may be used include, but are not limited to, a trained support vector machine (SVM), a random forest (RF) classifier, a decision tree classifier, a logistic regression classifier, a k-nearest neighbor (kNN) classifier, or other classifiers.

In some embodiments, the set of tumor immunophenotypes may include the tumor immunophenotypes of desert, excluded, and inflamed. In some embodiments, an image depicting a tumor may be classified as being the tumor immunophenotype desert based on an epithelium-immune cell density satisfying a desert epithelium-immune cell density threshold criterion and a stroma-immune cell density satisfying a desert stroma-immune cell density threshold criterion. The desert epithelium-immune cell density threshold criterion being satisfied may include the epithelium-immune cell density being within a first threshold range of epithelium-immune cell densities. The desert stroma-immune cell density threshold criterion being satisfied may include the stroma-immune cell density being within a first threshold range of stroma-immune cell densities.

In some embodiments, an image depicting a tumor may be classified as being the tumor immunophenotype excluded based on an epithelium-immune cell density satisfying an excluded epithelium-immune cell density threshold criterion and a stroma-immune cell density satisfying an excluded stroma-immune cell density threshold criterion. The excluded epithelium-immune cell density threshold criterion being satisfied may include the epithelium-immune cell density being within a second threshold range of epithelium-immune cell densities. The excluded stroma-immune cell density threshold criterion being satisfied may include the stroma-immune cell density being within a second threshold range of stroma-immune cell densities.

In some embodiments, an image depicting a tumor may be classified as being the tumor immunophenotype inflamed based on an epithelium-immune cell density satisfying an inflamed epithelium-immune cell density threshold criterion and a stroma-immune cell density satisfying an inflamed stroma-immune cell density threshold criterion. The inflamed epithelium-immune cell density threshold criterion being satisfied may include the epithelium-immune cell density being within a third threshold range of epithelium-immune cell densities. The inflamed stroma-immune cell density threshold criterion being satisfied may include the stroma-immune cell density being within a third threshold range of stroma-immune cell densities.

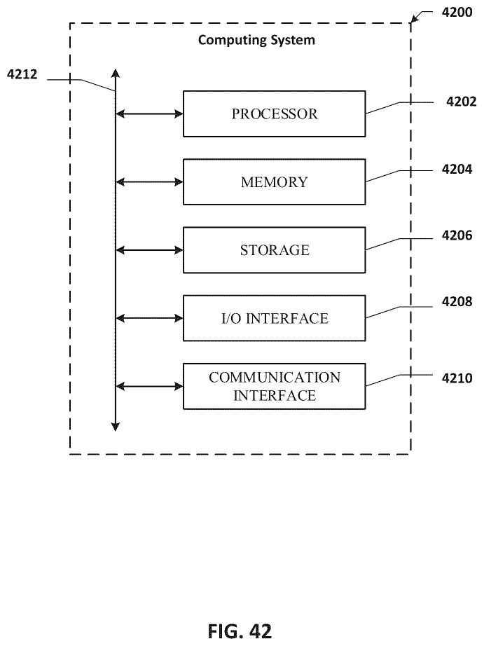

FIG. 42 illustrates an example computer system 4200. In some embodiments, one or more computer systems 4200 perform one or more steps of one or more methods described or illustrated herein. In some embodiments, one or more computer systems 4200 provide functionality described or illustrated herein. In some embodiments, software running on one or more computer systems 4200 performs one or more steps of one or more methods described or illustrated herein or provides functionality described or illustrated herein. Particular embodiments include one or more portions of one or more computer systems 4200. Herein, reference to a computer system may encompass a computing device, and vice versa, where appropriate. Moreover, reference to a computer system may encompass one or more computer systems, where appropriate.

This disclosure contemplates any suitable number of computer systems 4200. This disclosure contemplates computer system 4200 taking any suitable physical form. As example and not by way of limitation, computer system 4200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 4200 may include one or more computer systems 4200; be unitary or distributed; span multiple locations; span multiple machines; span multiple data centers; or reside in a cloud, which may include one or more cloud components in one or more networks. Where appropriate, one or more computer systems 4200 may perform without substantial spatial or temporal limitation one or more steps of one or more methods described or illustrated herein. As an example, and not by way of limitation, one or more computer systems 4200 may perform in real time or in batch mode one or more steps of one or more methods described or illustrated herein. One or more computer systems 4200 may perform at various times or at different locations one or more steps of one or more methods described or illustrated herein, where appropriate.

In some embodiments, computer system 4200 includes a processor 4202, memory 4204, storage 4206, an input/output (I/O) interface 4208, a communication interface 4210, and a bus 4212. Although this disclosure describes and illustrates a particular computer system having a particular number of components in a particular arrangement, this disclosure contemplates any suitable computer system having any suitable number of any suitable components in any suitable arrangement.

In some embodiments, processor 4202 includes hardware for executing instructions, such as those making up a computer program. As an example, and not by way of limitation, to execute instructions, processor 4202 may retrieve (or fetch) the instructions from an internal register, an internal cache, memory 4204, or storage 4206; decode and execute them; and then write one or more results to an internal register, an internal cache, memory 4204, or storage 4206. In some embodiments, processor 4202 may include one or more internal caches for data, instructions, or addresses. This disclosure contemplates processor 4202 including any suitable number of any suitable internal caches, where appropriate. As an example, and not by way of limitation, processor 4202 may include one or more instruction caches, one or more data caches, and one or more translation lookaside buffers (TLBs). Instructions in the instruction caches may be copies of instructions in memory 4204 or storage 4206, and the instruction caches may speed up retrieval of those instructions by processor 4202. Data in the data caches may be copies of data in memory 4204 or storage 4206 for instructions executing at processor 4202 to operate on; the results of previous instructions executed at processor 4202 for access by subsequent instructions executing at processor 4202 or for writing to memory 4204 or storage 4206; or other suitable data. The data caches may speed up read or write operations by processor 4202. The TLBs may speed up virtual-address translation for processor 4202. In some embodiments, processor 4202 may include one or more internal registers for data, instructions, or addresses. This disclosure contemplates processor 4202 including any suitable number of any suitable internal registers, where appropriate. Where appropriate, processor 4202 may include one or more arithmetic logic units (ALUs); be a multi-core processor; or include one or more processors 4202. Although this disclosure describes and illustrates a particular processor, this disclosure contemplates any suitable processor.

In some embodiments, memory 4204 includes main memory for storing instructions for processor 4202 to execute or data for processor 4202 to operate on. As an example, and not by way of limitation, computer system 4200 may load instructions from storage 4206 or another source (such as, for example, another computer system 4200) to memory 4204. Processor 4202 may then load the instructions from memory 4204 to an internal register or internal cache. To execute the instructions, processor 4202 may retrieve the instructions from the internal register or internal cache and decode them. During or after execution of the instructions, processor 4202 may write one or more results (which may be intermediate or final) to the internal register or internal cache. Processor 4202 may then write one or more of those results to memory 4204. In some embodiments, processor 4202 executes only instructions in one or more internal registers or internal caches or in memory 4204 (as opposed to storage 4206 or elsewhere) and operates only on data in one or more internal registers or internal caches or in memory 4204 (as opposed to storage 4206 or elsewhere). One or more memory buses (which may each include an address bus and a data bus) may couple processor 4202 to memory 4204. Bus 4212 may include one or more memory buses, as described below. In some embodiments, one or more memory management units (MMUs) reside between processor 4202 and memory 4204 and facilitate accesses to memory 4204 requested by processor 4202. In some embodiments, memory 4204 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 4204 may include one or more memories 3404, where appropriate. Although this disclosure describes and illustrates particular memory, this disclosure contemplates any suitable memory.

In some embodiments, storage 4206 includes mass storage for data or instructions. As an example, and not by way of limitation, storage 4206 may include a hard disk drive (HDD), a floppy disk drive, flash memory, an optical disc, a magneto-optical disc, magnetic tape, or a Universal Serial Bus (USB) drive or a combination of two or more of these. Storage 4206 may include removable or non-removable (or fixed) media, where appropriate. Storage 4206 may be internal or external to computer system 4200, where appropriate. In some embodiments, storage 4206 is non-volatile, solid-state memory. In some embodiments, storage 4206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 4206 taking any suitable physical form. Storage 4206 may include one or more storage control units facilitating communication between processor 4202 and storage 4206, where appropriate. Where appropriate, storage 4206 may include one or more storages 3406. Although this disclosure describes and illustrates particular storage, this disclosure contemplates any suitable storage.

In some embodiments, I/O interface 4208 includes hardware, software, or both, providing one or more interfaces for communication between computer system 4200 and one or more I/O devices. Computer system 4200 may include one or more of these I/O devices, where appropriate. One or more of these I/O devices may enable communication between a person and computer system 4200. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 4208 for them. Where appropriate, I/O interface 4208 may include one or more device or software drivers enabling processor 4202 to drive one or more of these I/O devices. I/O interface 4208 may include one or more I/O interfaces 4208, where appropriate. Although this disclosure describes and illustrates a particular I/O interface, this disclosure contemplates any suitable I/O interface.

In some embodiments, communication interface 4210 includes hardware, software, or both providing one or more interfaces for communication (such as, for example, packet-based communication) between computer system 4200 and one or more other computer systems 4200 or one or more networks. As an example, and not by way of limitation, communication interface 4210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 4210 for it. As an example, and not by way of limitation, computer system 4200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 4200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 4200 may include any suitable communication interface 4210 for any of these networks, where appropriate. Communication interface 4210 may include one or more communication interfaces 4210, where appropriate. Although this disclosure describes and illustrates a particular communication interface, this disclosure contemplates any suitable communication interface.

In some embodiments, bus 4212 includes hardware, software, or both coupling components of computer system 4200 to each other. As an example and not by way of limitation, bus 4212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 4212 may include one or more buses 4112, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### EXAMPLE EMBODIMENTS

Embodiments disclosed herein may include:

1. A method for determining a tumor immunophenotype, comprising: receiving an image of a tumor; identifying one or more regions of the image depicting tumor epithelium; calculating an epithelium-immune cell density for the image based on a number of immune cells detected within the one or more regions of the image depicting the tumor epithelium; and determining a tumor immunophenotype of the image based on the epithelium-immune cell density and at least a first density threshold for classifying images into one of a set of tumor immunophenotypes.
2. The method of embodiment 1, wherein identifying the one or more regions comprises: scanning the image using a sliding window; and for each portion of the image included within the sliding window: classifying the portion as at least one of a region depicting tumor epithelium or a region depicting tumor stroma.
3. The method of embodiment 2, wherein the portion is classified as a region depicting tumor epithelium based on a tumor epithelium criterion being satisfied.
4. The method of embodiment 3, wherein the tumor epithelium criterion being satisfied comprises at least a threshold amount of the portion depicting tumor epithelium.
5. The method of embodiment 4, wherein the threshold amount comprises 25% of the portion of the image.
6. The method of any one of embodiments 2-5, wherein the portion is classified as a region depicting tumor stroma based on a tumor stroma criterion being satisfied.
7. The method of embodiment 6, wherein the tumor stroma criterion being satisfied comprises at least a threshold amount of the portion depicting tumor stroma.
8. The method of embodiment 7, wherein the threshold amount comprises 25% of the portion of the image.
9. The method of any one of embodiments 7-8, wherein classifying the portion comprises: performing a color deconvolution to the image to obtain a plurality of color channel images, wherein the plurality of color channel images comprises: a first color channel image highlighting tumor epithelium and tumor stroma; and a second color channel image highlighting immune cells.
10. The method of embodiment 9, wherein a dual-stain is applied to a sample of the tumor, the dual-stain comprising a first stain and a second stain, wherein the first stain distinguishes tumor epithelium from tumor stroma and the second stain highlights immune cells.
11. The method of embodiment 10, wherein the first stain comprises a pan-cytokeratin (panCK) stain used for highlighting the tumor epithelium and tumor stroma, and wherein the second stain comprises a cluster of differentiation 8 (CD8) stain used for highlighting immune cells.
12. The method of any one of embodiments 9-11, wherein performing the color deconvolution comprises applying a hue-saturation-value (HSV) model to the image.
13. The method of any one of embodiments 1-12, wherein calculating the epithelium-immune cell density comprises: determining the number of immune cells detected within each of the one or more regions of the image using one or more machine learning models.
14. The method of embodiment 13, wherein the one or more machine learning models comprise a computer vision model trained to recognize immune cells.
15. The method of embodiment 14, wherein the computer vision model comprises a convolutional neural network (CNN).
16. The method of any one of embodiments 1-15, wherein: the first density threshold is determined based on a ranking of a plurality of images of tumors; each image of the plurality of images is associated with a patient of a plurality of patients participating in a first clinical trial; and each image of the plurality of images includes a label indicating a pre-determined tumor immunophenotype of the image.
17. The method of embodiment 16, further comprising: for each of the plurality of images: identifying one or more regions of the image depicting tumor epithelium; and calculating an epithelium-immune cell density for the image based on a number of immune cells detected within the one or more regions of the image depicting the tumor epithelium; and generating the ranking based on the epithelium-immune cell density of each of the plurality of images.
18. The method of embodiment 17, wherein the epithelium-immune cell density of each of the plurality of images comprises an average epithelium-immune cell density.
19. The method of embodiment 18, further comprising: for each image of the plurality of images: determining the number of immune cells within each of the one or more regions of the image depicting tumor epithelium; and calculating a local epithelium-immune cell density for each of the one or more regions of the image based on the number of immune cells detected within the region; and generating the average epithelium-immune cell density based on the local epithelium-immune cell density of each of the one or more regions.
20. The method of any one of embodiments 16-19, further comprising: determining a first set of images from the plurality of images based on the ranking; determining a second set of images from the plurality of images based on the ranking; and determining a third set of images from the plurality of images based on the ranking.
21. The method of embodiment 20, wherein: the first set of images includes one or more images of the plurality of images that are included in a first percentage of the ranking; the second set of images includes one or more images of the plurality of images that are included in a second percentage of the ranking; and the third set of images includes one or more images of the plurality of images that are included in a third percentage of the ranking.
22. The method of embodiment 21, wherein the first percentage of the ranking comprises a first twenty percent of the plurality of images in the ranking, the second percentage of the ranking comprises a subsequent forty percent of the plurality of images in the ranking, and the third percentage of the ranking comprises a remaining forty percent of the plurality of images in the ranking.
23. The method of any one of embodiments 21-22, further comprising: determining the first density threshold based on an epithelium-immune cell density of the one or more images in the third set of images.
24. The method of embodiment 23, further comprising: determining a second density threshold based on an epithelium-immune cell density of the one or more images in the first set of images and the one or more images in the second set of images.
25. The method of embodiment 24, wherein the tumor immunophenotype is: desert based on the epithelium-immune cell density of the image being less than the second density threshold; excluded based on the epithelium-immune cell density of the image being greater than or equal to the second density threshold and less than the first density threshold; or inflamed based on the epithelium-immune cell density being greater than or equal to the first density threshold.
26. The method of embodiment 25, further comprising: training a classifier to determine a tumor immunophenotype of an image input to the classifier based on a calculated epithelium-immune cell density of the image input to the classifier, the first density threshold, and the second density threshold.
27. The method of embodiment 26, further comprising: generating training data comprising the plurality of images, the label associated with each of the plurality of images, and the calculated epithelium-immune cell density of the image, wherein the classifier is trained with the training data.
28. The method of any one of embodiments 1-27, further comprising: training one or more machine learning models to detect biological objects, wherein the one or more machine learning models are trained using training data comprising a plurality of images and labels indicating a type of biological object depicted within each of the plurality of images, wherein the epithelium-immune cell density is calculated based on the one or more machine learning models.
29. The method of embodiment 28, wherein the biological objects include at least one of immune cells, tumor epithelium cells, or tumor stroma cells.
30. The method of any one of embodiments 1-29, wherein the image comprises a digital pathology image captured using a digital pathology imaging system.
31. A non-transitory computer-readable medium storing computer program instructions that, when executed by one or more processors, effectuates the method of any one of embodiments 1-30.
32. A system, comprising: one or more processors programmed to execute the method of any one of embodiments 1-30.
33. A method for determining a tumor immunophenotype, comprising: receiving an image depicting a tumor; dividing the image into a plurality of tiles; identifying epithelium tiles and stroma tiles from the plurality of tiles; calculating an epithelium-immune cell density for each of the epithelium tiles based on a number of immune cells detected within each of the epithelium tiles; calculating a stroma-immune cell density for each of the stroma tiles based on a number of immune cells detected within each of the stroma tiles; binning the epithelium tiles into an epithelium set of bins based on the epithelium-immune cell density of each epithelium tile; binning the stroma tiles into a stroma set of bins based on the stroma-immune cell density of each stroma tile; generating a density-bin representation of the epithelium set of bins and the stroma set of bins, wherein the density-bin representation includes a plurality of elements corresponding to each bin of the epithelium set of bins and each bin of the stroma set of bins; and determining a tumor immunophenotype of the image based on the density-bin representation.
34. The method of embodiment 33, wherein at least some of the epithelium tiles depict regions of tumor stroma and at least some of the stroma tiles depict regions of tumor epithelium.
35. The method of any one of embodiments 33-34, wherein a first stain and a second stain are applied to a sample of the tumor prior to the image being captured.
36. The method of embodiment 35, wherein the first stain highlights immune cells and the second stain highlights tumor epithelium and tumor stroma.
37. The method of embodiment 36, wherein the first stain comprises a cluster of differentiation 8 (CD8) stain used for highlighting immune cells and the second stain comprises a pan-cytokeratin (panCK) stain used for highlighting the tumor epithelium.
38. The method of any one of embodiments 35-37, wherein identifying the epithelium tiles comprises: classifying each tile of the plurality of tiles as being an epithelium tile based on a portion of the tile depicting tumor epithelium satisfying an epithelium-tile criterion.
39. The method of embodiment 38, wherein identifying the stroma tiles comprises: classifying each tile of the plurality of tiles as being a stroma tile based on a portion of the tile depicting tumor stroma satisfying a stroma-tile criterion.
40. The method of embodiment 39, wherein: the epithelium-tile criterion being satisfied comprises the portion of the tile depicting tumor epithelium being greater than or equal to a first threshold area; and the stroma-tile criterion being satisfied comprises the portion of the tile depicting the tumor stroma being greater than or equal to a second threshold area.
41. The method of embodiment 40, wherein: the first threshold area and the second threshold area comprise 25% of the tile.
42. The method of any one of embodiments 33-41, further comprising: determining a number of immune cells detected within each of the epithelium tiles using one or more machine learning models; and determining a number of immune cells detected within each of the stroma tiles using the one or more machine learning models.
43. The method of embodiment 42, wherein the one or more machine learning models are trained to detect depictions of one or more types of biological objects within an image.
44. The method of embodiment 43, wherein the one or more machine learning models comprise a computer vision model.
45. The method of embodiment 44, wherein the computer vision model comprises a convolutional neural network (CNN).
46. The method of any one of embodiments 42-45, further comprising: training the one or more machine learning models to detect biological objects, wherein the one or more machine learning models are trained using training data comprising a plurality of training images and labels indicating a type of biological object depicted within each of the plurality of training images.
47. The method of embodiment 46, wherein the one or more types of biological objects comprise at least one of immune cells, stroma cells forming tumor stroma, or epithelial cells forming tumor epithelium.
48. The method of any one of embodiments 33-47, wherein binning comprising: determining a density range for each bin of the epithelium set of bins and the stroma set of bins, wherein each tile of the plurality of tiles is allocated to one of the epithelium set of bins or one of the stroma set of bins based on the epithelium-immune cell density of the tile or the stroma-immune cell density of the tile.
49. The method of embodiment 48, wherein the epithelium set of bins and the stroma set of bins each include ten bins.
50. The method of embodiment 49, wherein epithelium set of bins are defined by: a first density range comprising epithelium-immune cell densities of 0.0-0.005, a second density range comprising epithelium-immune cell densities of 0.005-0.01, a third density range comprising epithelium-immune cell densities of 0.01-0.02, a fourth density range comprising epithelium-immune cell densities of 0.02-0.04, a fifth density range comprising epithelium-immune cell densities of 0.04-0.06, a sixth density range comprising epithelium-immune cell densities of 0.06-0.08, a seventh density range comprising epithelium-immune cell densities of 0.08-0.12, an eighth density range comprising epithelium-immune cell densities of 0.12-0.16, a ninth density range comprising epithelium-immune cell densities of 0.16-0.2, and a tenth density range comprising epithelium-immune cell densities of 0.2-2.0.
51. The method of embodiment 50, wherein stroma set of bins are defined by: a first density range comprising stroma-immune cell densities of 0.0-0.005, a second density range comprising stroma-immune cell densities of 0.005-0.01, a third density range comprising stroma-immune cell densities of 0.01-0.02, a fourth density range comprising stroma-immune cell densities of 0.02-0.04, a fifth density range comprising stroma-immune cell densities of 0.04-0.06, a sixth density range comprising stroma-immune cell densities of 0.06-0.08, a seventh density range comprising stroma-immune cell densities of 0.08-0.12, an eighth density range comprising stroma-immune cell densities of 0.12-0.16, a ninth density range comprising stroma-immune cell densities of 0.16-0.2, and a tenth density range comprising stroma-immune cell densities of 0.2-2.0.
52. The method of embodiment 49, wherein the density range for a bin of the epithelium set of bins or the stroma set of bins is defined by a lower density threshold and an upper density threshold, and an epithelium tile or a stroma tile is allocated to the bin based on an epithelium-immune cell density or a stroma-immune cell density, respectively, being less than the upper density threshold and greater than or equal to the lower density threshold.
53. The method of any one of embodiments 33-52, further comprising: generating training data comprising a plurality of density-bin representations each representing an epithelium-immune cell density distribution and a stroma-immune cell density distribution of one of a plurality of images depicting tumors, wherein each density-bin representation of the plurality of density-bin representations includes a label indicating a pre-determined tumor immunophenotype of the corresponding image of the plurality of images.
54. The method of embodiment 53, further comprising: training a classifier to predict a tumor immunophenotype of an input image based on the training data, wherein the predicted tumor immunophenotype is one of a set of tumor immunophenotypes, wherein the tumor immunophenotype is determined based on the trained classifier.
55. The method of embodiment 54, wherein the set of tumor immunophenotypes comprises: desert based on an epithelium-immune cell density satisfying a desert epithelium-immune cell density threshold criterion and a stroma-immune cell density satisfying a desert stroma-immune cell density threshold criterion; excluded based on an epithelium-immune cell density satisfying an excluded epithelium-immune cell density threshold criterion and a stroma-immune cell density satisfying an excluded stroma-immune cell density threshold criterion; and inflamed based on an epithelium-immune cell density satisfying an inflamed epithelium-immune cell density threshold criterion and a stroma-immune cell density satisfying an inflamed stroma-immune cell density threshold criterion.
56. The method of embodiment 55, wherein: the desert epithelium-immune cell density threshold criterion being satisfied comprises the epithelium-immune cell density being within a first threshold range of epithelium-immune cell densities; and the desert stroma-immune cell density threshold criterion being satisfied comprises the stroma-immune cell density being within a first threshold range of stroma-immune cell densities.
57. The method of any one of embodiments 55-56, wherein: the excluded epithelium-immune cell density threshold criterion being satisfied comprises the epithelium-immune cell density being within a second threshold range of epithelium-immune cell densities; and the excluded stroma-immune cell density threshold criterion being satisfied comprises the stroma-immune cell density being within a second threshold range of stroma-immune cell densities.
58. The method of any one of embodiments 55-57, wherein: the inflamed epithelium-immune cell density threshold criterion being satisfied comprises the epithelium-immune cell density being within a third threshold range of epithelium-immune cell densities; and the inflamed stroma-immune cell density threshold criterion being satisfied comprises the stroma-immune cell density being within a third threshold range of stroma-immune cell densities.
59. The method of any one of embodiments 54-58, wherein the classifier is one of a support vector machine (SVM), a random forest (RF) classifier, a decision tree classifier, a logistic regression classifier, or a k-nearest neighbor (kNN) classifier.
60. The method of any one of embodiments 54-59, wherein the classifier comprises a multi-class classifier configured to classify an image into one of a set of tumor immunophenotypes.
61. The method of any one of embodiments 33-60, wherein each tile of the plurality of tiles has a size of approximately 16,000 μm².
62. The method of any one of embodiments 33-61, wherein the plurality of tiles are overlapping or non-overlapping.
63. The method of any one of embodiments 33-62, wherein the density-bin representation comprises a 20-dimensional feature vector.
64. The method of embodiment 63, wherein the 20-dimensional feature vector comprises 20 elements, each of the 20 elements corresponding to a bin from the epithelium set of bins and the stroma set of bins.
65. The method of any one of embodiments 33-64, wherein the image comprises a whole slide image.
66. The method of any one of embodiments 33-65, wherein the image comprises a digital pathology image captured using a digital pathology imaging system.
67. A non-transitory computer-readable medium storing computer program instructions that, when executed by one or more processors, effectuate the method of any one of embodiments 33-66.
68. A system, comprising: one or more processors programmed to execute the method of any one of embodiments 33-66.
69. A method for determining a tumor immunophenotype, comprising: receiving an image depicting a tumor; dividing the image into a plurality of tiles; identifying epithelium tiles and stroma tiles from the plurality of tiles; calculating an epithelium-immune cell density for each of the epithelium tiles based on a number of immune cells detected within each of the epithelium tiles; calculating a stroma-immune cell density for each of the stroma tiles based on a number of immune cells detected within each tile of the stroma tiles; generating a density-bin representation based on the epithelium-immune cell density of one or more epithelium tiles and the stroma-immune cell density of one or more stroma tiles; generating one or more spatial distribution metrics describing the image based on the epithelium-immune cell density of each of the epithelium tiles and the stroma-immune cell density of each of the stroma tiles; generating a spatial distribution representation based on the one or more spatial distribution metrics for each of the plurality of tiles; concatenating the density-bin representation and the spatial distribution representation to obtain a concatenated representation; and determining a tumor immunophenotype of the image based on the concatenated representation.
70. The method of embodiment 69, further comprising: projecting the concatenated representation into a feature space, wherein the tumor immunophenotype is based on a position of the concatenated representation in the feature space.
71. The method of embodiment 70, wherein determining the tumor immunophenotype comprises: determining a distance in the feature space between the projected concatenated representation and clusters of concatenated representations, each cluster being associated with one of a set of tumor immunophenotypes; and assigning the tumor immunophenotype to the image based on the distance between the projected concatenated representation and a cluster of concatenated representations associated with the tumor immunophenotype.
72. The method of any one of embodiments 69-71, wherein the one or more spatial distribution metrics comprise: a Jaccard index, a Sorensen index, a Bhattacharyya coefficient, a Moran's index, a Geary's contiguity ratio, a Morisita-Horn index, or a metric defined based on a hotspot/cold spot analysis.
73. The method of any one of embodiments 69-72, wherein the density-bin representation comprises a 20-element vector and the spatial distribution representation comprises a 50-element vector.
74. The method of any one of embodiments 69-73, further comprising: generating training data comprising a plurality of concatenated representations respectively corresponding to one of a plurality of images depicting tumors, wherein each concatenated representation includes a label indicating a pre-determined tumor immunophenotype of the corresponding image of the plurality of images.
75. The method of embodiment 74, further comprising: training a classifier to predict a tumor immunophenotype of an input image based on the training data, wherein the predicted tumor immunophenotype is one of a set of tumor immunophenotypes, wherein determining the tumor immunophenotype comprises: determining, using the trained classifier, the tumor immunophenotype based on the concatenated representation.
76. The method of embodiment 75, wherein the classifier comprises a multi-class classifier, each class corresponding to one of the set of tumor immunophenotypes.
77. The method of embodiment 76, wherein the classifier comprises one of a support vector machine (SVM), a random forest (RF) classifier, a decision tree classifier, a logistic regression classifier, or a k-nearest neighbor (kNN) classifier.
78. The method of any one of embodiments 69-77, wherein at least some of the epithelium tiles depict regions of tumor stroma and at least some of the stroma tiles depict regions of tumor epithelium.
79. The method of any one of embodiments 69-78, wherein a first stain and a second stain are applied to a sample of the tumor prior to the image being captured.
80. The method of embodiment 79, wherein the first stain highlights immune cells and the second stain highlights tumor epithelium and tumor stroma.
81. The method of embodiment 80, wherein the first stain comprises a cluster of differentiation 8 (CD8) stain used for highlighting immune cells and the second stain comprises a pan-cytokeratin (panCK) stain used for highlighting tumor epithelium.
82. The method of any one of embodiments 69-81, wherein identifying the epithelium tiles comprises: classifying one or more of the plurality of tiles as being an epithelium tile based on a portion of each of the one or more tiles depicting tumor epithelium satisfying an epithelium-tile criterion.
83. The method of embodiment 82, wherein identifying the stroma tiles comprises: classifying one or more of the plurality of tiles as being a stroma tile based on a portion of each of the one or more tiles depicting tumor stroma satisfying a stroma-tile criterion.
84. The method of embodiment 83, wherein: the epithelium-tile criterion being satisfied comprises the portion of a tile depicting tumor epithelium being greater than or equal to a first threshold area; and the stroma-tile criterion being satisfied comprises the portion of a tile depicting the tumor stroma being greater than or equal to a second threshold area.
85. The method of embodiment 84, wherein: the first threshold area and the second threshold area comprise 25% of the tile.
86. The method of any one of embodiments 69-85, further comprising: determining a number of immune cells detected within each of the epithelium tiles using one or more machine learning models; and determining a number of immune cells detected within each of the stroma tiles using the one or more machine learning models.
87. The method of embodiment 86, wherein the one or more machine learning models are trained to detect depictions of one or more types of biological objects within an image.
88. The method of embodiment 87, wherein the one or more machine learning models comprise a computer vision model.
89. The method of embodiment 88, wherein the computer vision model comprises a convolutional neural network (CNN).
90. The method of any one of embodiments 86-89, further comprising: training the one or more machine learning models to detect one or more types of biological objects, wherein the one or more machine learning models are trained using training data comprising a plurality of training images and labels indicating a type of biological object depicted within each of the plurality of training images.
91. The method of embodiment 90, wherein the one or more types of biological objects comprise at least one of immune cells, stroma cells forming tumor stroma, or epithelial cells forming tumor epithelium.
92. The method of any one of embodiments 69-91, further comprising: binning the epithelium tiles into an epithelium set of bins based on the epithelium-immune cell density of each epithelium tile; and binning the stroma tiles into a stroma set of bins based on the stroma-immune cell density of each stroma tile, wherein the density-bin representation is generated based on the epithelium set of bins and the stroma set of bins.
93. The method of embodiment 92, wherein the density-bin representation includes a plurality of elements corresponding to each bin of the epithelium set of bins and each bin of the stroma set of bins.
94. The method of any one of embodiments 92-93, wherein binning comprising: determining a range of densities for each bin of the epithelium set of bins and the stroma set of bins, wherein each tile of the plurality of tiles is allocated to one of the epithelium set of bins or one of the stroma set of bins based on the epithelium-immune cell density of the tile or the stroma-immune cell density of the tile.
95. The method of any one of embodiments 92-94, wherein the epithelium set of bins and the stroma set of bins each include ten bins.
96. The method of embodiment 95, wherein the ten bins are defined by: a first range epithelium-immune cell densities or stroma-immune cell densities of 0.0-0.005, a second range epithelium-immune cell densities or stroma-immune cell densities of 0.005-0.01, a third range epithelium-immune cell densities or stroma-immune cell densities of 0.01-0.02, a fourth range epithelium-immune cell densities or stroma-immune cell densities of 0.02-0.04, a fifth range epithelium-immune cell densities or stroma-immune cell densities of 0.04-0.06, a sixth range epithelium-immune cell densities or stroma-immune cell densities of 0.06-0.08, a seventh range epithelium-immune cell densities or stroma-immune cell densities of 0.08-0.12, an eighth range epithelium-immune cell densities or stroma-immune cell densities of 0.12-0.16, a ninth range epithelium-immune cell densities or stroma-immune cell densities of 0.16-0.2, and a tenth range epithelium-immune cell densities or stroma-immune cell densities of 0.2-2.0.
97. The method of embodiment 94, wherein the range of densities for a bin of the epithelium set of bins or the stroma set of bins is defined by a lower density threshold and an upper density threshold, and an epithelium tile or a stroma tile is allocated to the bin based on an epithelium-immune cell density or a stroma-immune cell density, respectively, being less than the upper density threshold and greater than or equal to the lower density threshold.
98. The method of any one of embodiments 69-97, wherein each tile of the plurality of tiles has a size of approximately 16,000 μm².
99. The method of any one of embodiments 69-98, wherein the image comprises a whole slide image.
100. The method of any one of embodiments 69-99, wherein the plurality of tiles are overlapping or non-overlapping.
101. The method of any one of embodiments 69-100, wherein the image comprises a digital pathology image captured using a digital pathology imaging system.
102. The method of any one of embodiments 69-101, wherein the tumor immunophenotype is one of a set of tumor immunophenotypes.
103. The method of embodiment 102, wherein the set of tumor immunophenotypes comprises: desert based on an epithelium-immune cell density satisfying a desert epithelium-immune cell density threshold criterion and a stroma-immune cell density satisfying a desert stroma-immune cell density threshold criterion; excluded based on an epithelium-immune cell density satisfying an excluded epithelium-immune cell density threshold criterion and a stroma-immune cell density satisfying an excluded stroma-immune cell density threshold criterion; and inflamed based on an epithelium-immune cell density satisfying an inflamed epithelium-immune cell density threshold criterion and a stroma-immune cell density satisfying an inflamed stroma-immune cell density threshold criterion.
104. The method of embodiment 103, wherein: the desert epithelium-immune cell density threshold criterion being satisfied comprises the epithelium-immune cell density being within a first threshold range of epithelium-immune cell densities; and the desert stroma-immune cell density threshold criterion being satisfied comprises the stroma-immune cell density being within a first threshold range of stroma-immune cell densities.
105. The method of any one of embodiments 103-104, wherein: the excluded epithelium-immune cell density threshold criterion being satisfied comprises the epithelium-immune cell density being within a second threshold range of epithelium-immune cell densities; and the excluded stroma-immune cell density threshold criterion being satisfied comprises the stroma-immune cell density being within a second threshold range of stroma-immune cell densities.
106. The method of any one of embodiments 103-105, wherein: the inflamed epithelium-immune cell density threshold criterion being satisfied comprises the epithelium-immune cell density being within a third threshold range of epithelium-immune cell densities; and the inflamed stroma-immune cell density threshold criterion being satisfied comprises the stroma-immune cell density being within a third threshold range of stroma-immune cell densities.
107. A non-transitory computer-readable medium storing computer program instructions that, when executed by one or more processors, effectuate the method of any one of embodiments 69-108. A system, comprising: one or more processors programmed to execute the method of any one of embodiments 69-106.

1. A method for determining a tumor immunophenotype, comprising:
receiving an image of a tumor;
identifying one or more regions of the image depicting tumor epithelium;
calculating an epithelium-immune cell density for the image based on a number of immune cells detected within the one or more regions of the image depicting the tumor epithelium; and
determining a tumor immunophenotype of the image based on the epithelium-immune cell density and at least a first density threshold for classifying images into one of a set of tumor immunophenotypes.

2. The method of claim 1, wherein identifying the one or more regions comprises:
scanning the image using a sliding window; and
for each portion of the image included within the sliding window:
classifying the portion as at least one of a region depicting tumor epithelium or a region depicting tumor stroma.

3.-12. (canceled)

13. The method of claim 1, wherein calculating the epithelium-immune cell density comprises:
determining the number of immune cells detected within each of the one or more regions of the image using one or more machine learning models.

14. The method of claim 13, wherein the one or more machine learning models comprise a computer vision model trained to recognize immune cells.

15. (canceled)

16. The method of claim 1, wherein:
the first density threshold is determined based on a ranking of a plurality of images of tumors;
each image of the plurality of images is associated with a patient of a plurality of patients participating in a first clinical trial; and
each image of the plurality of images includes a label indicating a pre-determined tumor immunophenotype of the image.

17. The method of claim 16, further comprising:
for each of the plurality of images:
identifying one or more regions of the image depicting tumor epithelium; and
calculating an epithelium-immune cell density for the image based on a number of immune cells detected within the one or more regions of the image depicting the tumor epithelium; and
generating the ranking based on the epithelium-immune cell density of each of the plurality of images.

18.-25. (canceled)

26. The method of claim 1, further comprising:
training a classifier to determine a tumor immunophenotype of an image input to the classifier based on a calculated epithelium-immune cell density of the image input to the classifier, the first density threshold, and a second density threshold.

27. (canceled)

28. The method of claim 1, further comprising:
training one or more machine learning models to detect biological objects, wherein the one or more machine learning models are trained using training data comprising a plurality of images and labels indicating a type of biological object depicted within each of the plurality of images, wherein the epithelium-immune cell density is calculated based on the one or more machine learning models.

29. (canceled)

30. The method of claim 1, wherein the image comprises a digital pathology image captured using a digital pathology imaging system.

31.-32. (canceled)

33. A method for determining a tumor immunophenotype, comprising:
receiving an image depicting a tumor;
dividing the image into a plurality of tiles;
identifying epithelium tiles and stroma tiles from the plurality of tiles;
calculating an epithelium-immune cell density for each of the epithelium tiles based on a number of immune cells detected within each of the epithelium tiles;
calculating a stroma-immune cell density for each of the stroma tiles based on a number of immune cells detected within each of the stroma tiles;
binning the epithelium tiles into an epithelium set of bins based on the epithelium-immune cell density of each epithelium tile;
binning the stroma tiles into a stroma set of bins based on the stroma-immune cell density of each stroma tile;
generating a density-bin representation of the epithelium set of bins and the stroma set of bins, wherein the density-bin representation includes a plurality of elements corresponding to each bin of the epithelium set of bins and each bin of the stroma set of bins; and
determining a tumor immunophenotype of the image based on the density-bin representation.

34.-41. (canceled)

42. The method of claim 33, further comprising:
determining a number of immune cells detected within each of the epithelium tiles using one or more machine learning models; and
determining a number of immune cells detected within each of the stroma tiles using the one or more machine learning models.

43. (canceled)

44. The method of claim 42, wherein the one or more machine learning models comprise a computer vision model.

45. (canceled)

46. The method of claim 42, further comprising:
training the one or more machine learning models to detect biological objects, wherein the one or more machine learning models are trained using training data comprising a plurality of training images and labels indicating a type of biological object depicted within each of the plurality of training images.

47. (canceled)

48. The method of claim 33, wherein binning comprising:
determining a density range for each bin of the epithelium set of bins and the stroma set of bins, wherein each tile of the plurality of tiles is allocated to one of the epithelium set of bins or one of the stroma set of bins based on the epithelium-immune cell density of the tile or the stroma-immune cell density of the tile.

49.-52. (canceled)

53. The method of claim 33, further comprising:
generating training data comprising a plurality of density-bin representations each representing an epithelium-immune cell density distribution and a stroma-immune cell density distribution of one of a plurality of images depicting tumors, wherein each density-bin representation of the plurality of density-bin representations includes a label indicating a pre-determined tumor immunophenotype of the corresponding image of the plurality of images.

54. The method of claim 53, further comprising:
training a classifier to predict a tumor immunophenotype of an input image based on the training data, wherein the predicted tumor immunophenotype is one of a set of tumor immunophenotypes, wherein the tumor immunophenotype is determined based on the trained classifier.

55.-62. (canceled)

63. The method of claim 33, wherein the density-bin representation comprises a 20-dimensional feature vector.

64. (canceled)

65. The method of claim 33, wherein the image comprises a whole slide image.

66. The method of claim 33, wherein the image comprises a digital pathology image captured using a digital pathology imaging system.

67.-68. (canceled)

69. A method for determining a tumor immunophenotype, comprising:
receiving an image depicting a tumor;
dividing the image into a plurality of tiles;
identifying epithelium tiles and stroma tiles from the plurality of tiles;
calculating an epithelium-immune cell density for each of the epithelium tiles based on a number of immune cells detected within each of the epithelium tiles;
calculating a stroma-immune cell density for each of the stroma tiles based on a number of immune cells detected within each tile of the stroma tiles;
generating a density-bin representation based on the epithelium-immune cell density of one or more epithelium tiles and the stroma-immune cell density of one or more stroma tiles;
generating one or more spatial distribution metrics describing the image based on the epithelium-immune cell density of each of the epithelium tiles and the stroma-immune cell density of each of the stroma tiles;
generating a spatial distribution representation based on the one or more spatial distribution metrics for each of the plurality of tiles;
concatenating the density-bin representation and the spatial distribution representation to obtain a concatenated representation; and
determining a tumor immunophenotype of the image based on the concatenated representation.

70. The method of claim 69, further comprising:
projecting the concatenated representation into a feature space, wherein the tumor immunophenotype is based on a position of the concatenated representation in the feature space.

71. The method of claim 70, wherein determining the tumor immunophenotype comprises:
determining a distance in the feature space between the projected concatenated representation and clusters of concatenated representations, each cluster being associated with one of a set of tumor immunophenotypes; and
assigning the tumor immunophenotype to the image based on the distance between the projected concatenated representation and a cluster of concatenated representations associated with the tumor immunophenotype.

72. (canceled)

73. The method of claim 69, wherein the density-bin representation comprises a 20-element vector and the spatial distribution representation comprises a 50-element vector.

74. The method of claim 69, further comprising:
generating training data comprising a plurality of concatenated representations respectively corresponding to one of a plurality of images depicting tumors, wherein each concatenated representation includes a label indicating a pre-determined tumor immunophenotype of the corresponding image of the plurality of images.

75. The method of claim 74, further comprising:
training a classifier to predict a tumor immunophenotype of an input image based on the training data, wherein the predicted tumor immunophenotype is one of a set of tumor immunophenotypes, wherein determining the tumor immunophenotype comprises:
determining, using the trained classifier, the tumor immunophenotype based on the concatenated representation.

76.-85. (canceled)

86. The method of claim 69, further comprising:
determining a number of immune cells detected within each of the epithelium tiles using one or more machine learning models; and
determining a number of immune cells detected within each of the stroma tiles using the one or more machine learning models.

87. (canceled)

88. The method of claim 86, wherein the one or more machine learning models comprise a computer vision model.

89. (canceled)

90. The method of claim 88, further comprising:
training the one or more machine learning models to detect one or more types of biological objects, wherein the one or more machine learning models are trained using training data comprising a plurality of training images and labels indicating a type of biological object depicted within each of the plurality of training images.

91. (canceled)

92. The method of claim 69, further comprising:
binning the epithelium tiles into an epithelium set of bins based on the epithelium-immune cell density of each epithelium tile; and
binning the stroma tiles into a stroma set of bins based on the stroma-immune cell density of each stroma tile, wherein the density-bin representation is generated based on the epithelium set of bins and the stroma set of bins.

93.-100. (canceled)

101. The method of claim 69, wherein the image comprises a digital pathology image captured using a digital pathology imaging system.

102.-108. (canceled)
